(19) United States
(12) Patent Application Publication
(10) Pub. No.: US 2002/0150891 A1
(43) Pub. Date: Oct. 17, 2002

(54) DIAGNOSTIC AND THERAPEUTIC COMPOSITIONS AND METHODS WHICH UTILIZE THE T CELL RECEPTOR BETA GENE REGION

(76) Inventors: LEROY E. HOOD, SEATTLE, WA (US); LEE ROWEN, SEATTLE, WA (US)

Correspondence Address: Jane E. R. Potter, Esq., Seed Intellectual Property Law Group PLLC, 701 fifth Avenue,, Suite 6300, Seattle, WA, 98104-7092, US

(21) Appl. No.: 09/263,959

(22) Filed: Mar. 5, 1999

Related U.S. Application Data
(63) Continuation of application No. 08/531,241, filed on Sep. 19, 1995, now abandoned.
Application No. 08/531,241 is a continuation-in-part of application No. 08/309,335, filed on Sep. 19, 1994, now abandoned.

(51) Int. Cl. C12Q 1/68; G01N 33/53; A01N 43/04

(52) U.S. Cl. 435/6; 435/7.1; 514/44

(57) ABSTRACT

The present invention provides isolated nucleic acid molecules encoding a variety of Vβ genes (e.g., Vβ25, Vβ26, Vβ27, Vβ28, Vβ29, Vβ30 or Vβ31) as well as both 5′ and 3′ sequences which flank a T cell receptor β gene. Also provided are kits of primers and kits of antibodies. Further, the present invention also provides methods for diagnosing organ transplant rejection, as well as methods for determining a correlation between a disease or disease susceptibility and a selected polymorphism.

### CROSS-REFERENCE TO RELATED APPLICATIONS

This application is a continuation-in-part of U.S. patent application Ser. No. 08/309,335, filed Sep. 19, 1994, which application is hereby incorporated by reference.

### TECHNICAL FIELD

The present invention relates generally to cell-surface receptors, and more specifically, to compositions and methods which utilize T cell receptor Vβ genes.

### BACKGROUND OF THE INVENTION

T cell receptors (“TCR”) are the primary antigen binding receptors on T lymphoid cells. Briefly, these receptors, unlike the antigen receptor on B cells, bind antigen in the context of class I or class II molecules of the Major Histocompatability Complex (“MHC”). Depending on the type of T cell, binding of antigen to the receptor in the context of MHC I or MHC II molecules triggers either a cytotoxic response which directly lyses target cells, or a helper response which provides necessary factors for B cell stimulation.

T cell receptors are heterodimers which are composed of an α or γ chain and a β or δ chain. Of the combination of chains that may form receptors, the αβ complex is by far the most common, being present on at least 95% of peripheral blood T cells.

The structure of T cell receptor chains is very similar to the structure of other members of the immunoglobulin gene superfamily (Davis and Bjorkman, Nature 334:395-402, 1988; Chothia et al., EMBO J. 7:3745-3755, 1988; Yanagi et al., Nature 308:145, 1984; Hedrick et al., Nature 308:149, 1984; Chien et al., Nature 312:31, 1984; Saito et al., Nature 312:36, 1984; Sim et al., Nature 312:771, 1984). In particular, similar to immunoglobins, T cell receptor chains can be divided into a variable region that binds antigen and a constant (C) region that serves to attach the complex to the membrane. T cell receptor variable regions are encoded by multiple gene segments, including variable (V), diversity (D), and joining (J) for the β and δ chains, and V and J for the α and γ chains (see reviews by Hunkapiller and Hood, Adv. Immunol. 44:1, 1989; Davis, Ann. Rev. Biochem. 59:475, 1990). During differentiation of T lymphoid cells, the gene segments of the variable region undergo physical rearrangement to yield either a V(D)J or a VJ configuration. The C region remains separately encoded. A complete T cell receptor chain is then synthesized following splicing of a mRNA transcript (see FIG. 1).

The T cell receptor β gene family maps to chromosome 7 (Barker et al., Science 226:348, 1984; Caccia et al., Cell 37:1091, 1984). Briefly, there are two Cβ genes (“Cβ1” and “Cβ2”) approximately 10 kb apart (Toyonaga et al., Proc. Natl. Acad. Sci. USA 82:8624, 1985). Upstream of the Cβ1 gene segment is a cluster of six functional Jβ gene segments, and upstream of the Cβ2 gene segment is a cluster of seven functional Jβ gene segments. A single Dβ gene segment lies upstream of each Jβ gene cluster. (Id.) The number of Vβ genes have been estimated to be between 60 (Concannon et al., Proc. Natl. Acad. Sci. USA 83:6598, 1986) and 100 (Kimura et al., J. Exp. Med. 164:739, 1986). About 60 distinct Vβ genes have been identified by DNA sequence analysis of cDNA and RT-PCR (reverse polymerase chain reaction) generated clones (Toyonaga and Mak, Ann. Rev. Immunol. 5:585, 1987; Wilson et al., Immunol. Rev. 101:149, 1988; Robinson, J. Immunol. 146:4392, 1991; Ferradini et al., Eur. J. Immunol 21:935, 1991; Li et al., J. Exp. Med. 171:221, 1990). The Vβ gene segments have been categorized into approximately 20 subfamilies on the basis of at least 75% sequence identity. Each subfamily contains from one to a few members.

While at first blush it may appear that cDNA and PCR cloning techniques have yielded substantial information on the number and organization of the Vβ gene family, complete, detailed information of these genes necessary for diagnostic and therapeutic applications is in fact lacking. In particular, most of the Vβ gene sequences have been determined from rearranged genes, and thus, DNA sequences of the intervening sequences, flanking sequences (including promoter and recombination signal sequences) and precise 3′ end of the Vβ gene segments is lacking for many of the identified genes. Moreover, identification of new Vβ genes utilizing techniques such as random cDNA cloning and PCR amplification has been impeded due to the low frequency with which particular Vβ gene segments are rearranged and expressed, as well as the inability to design primers which are specific for identical β gene segments with an unknown DNA sequence. The importance of having the complete sequence of the human β T cell receptor gene family lies in the fact that only with the complete sequence can each Vβ gene segment be individually analyzed by PCR analysis. Because many of the Vβ gene segments fall into related Vβ subfamilies (75% or more similar at the DNA level), only when all of the Vβ sequences and flanking regions are known can unique PCR pairs be designed for each Vβ gene segments. To date, only about 4% of the genomic sequence which encodes the β gene family has been identified.

Inability to obtain complete detailed information of the Vβ gene family has hampered assessment of the T cell receptor's role in the immune responsiveness of an individual. Briefly, immune responsiveness is influenced by the repertoire of antigen receptors which are expressed by T cells in the periphery. For example, if a particular V gene is absent in the periphery, there may be a lack of responsiveness to some antigen. Indeed, mice which have deletions of part of the Vβ gene region are in fact unresponsive to certain antigens. (Behlke et al., Proc. Natl. Acad. Sci. USA 83:767, 1986). This repertoire of antigen receptors is shaped by both positive selection and negative selection of T cells in the thymus. In particular, positive thymic selection preserves T cells that have an affinity for self-MHC molecules, while negative selection removes T cells that are self-reactive.

This observed linkage between the Vβ repertoire and immune responsiveness has led to efforts to detect genetic influences on immune responsiveness in humans. In particular, several common techniques have been utilized in order to assess the peripheral repertoire in normal individuals, normal tissues, and diseased tissues and individuals. For example, monoclonal antibodies to the variable region of a β chain have been utilized in attempts to assess the linkage between a particular Vβ, and immune responsiveness. While antibodies are easy to use and large populations can be examined, only a few antibodies are available. Moreover, specificity is difficult to characterize because the full complement of all Vβs is not yet known. Similarly, molecular techniques to detect Vβ gene expression, such as RNase probe protection and quantitative PCR, are limited by low levels of a particular mRNA, as well as by lack of knowledge as to the entire genomic sequence of the β gene family.

Superimposed upon these difficulties of analysis are complications which arise from the presence of polymorphisms or allelic variants. Briefly, polymorphisms may be found in both coding and non-coding regions. Non-coding polymorphisms in the Vβ gene region include deletions (Seboun et al., J. Exp. Med. 170:1263, 1989), restriction fragment length polymorphisms (RFLP) (Concannon et al., Am. J. Genet. 47:45, 1990; Posnett, Immunol. Today 11:368, 1990) and simple sequence repeats and other repetitive elements (e.g., Alu repeats, LINEs, see review by Posnett, Immunol. Today 11:368, 1990). Short and long interspersed nuclear elements (SINEs and LINEs), as well as Alu sequences, have also been identified in the CδCα region (Hood et al., Genome Analysis 5:63, 1993). Assessment of the linkage of immune responsiveness with a particular Vβ has been limited because antibodies have been raised against only a few known coding regions, and nucleic acid sequences have been identified primarily only for coding regions. In particular, although there are many apparent “hot spots” of recombination or gene conversion (Nickerson et al., Genomics 12: 377, 1992), determination of whether particular V gene segments are associated with particular immune diseases (e.g., multiple sclerosis) has been impeded due to the lack of a battery or series of genetic markers that span the entire Vβ region, and which are in at least partial linkage disequilibrium.

Although rarer than non-coding polymorphisms, structural variants of the beta chain may also have a direct effect on immune responsiveness and thus disease association. In particular, since T cell receptors recognize antigen in the context of MHC, any amino acid alterations in a region that binds antigen or MHC could alter T cell responsiveness. Of the few known Vβ polymorphisms, one, in Vβ6.7, is in a region of the molecule thought to be involved in superantigen binding.

Given the T cell receptor's critical role in initiating specific immune responses, it has been suggested that such receptors play a major role in autoimmune disease, cancer, and other T-cell mediated diseases. For example, expression of certain Vβ elements has been suggested to confer susceptibility of mice to experimental autoimmune encephalomyelitis (EAE), a disease model for multiple sclerosis. In particular, the direct role of two distinct Vβ gene segments in initiating EAE has been demonstrated by the prevention and treatment of EAE utilizing particular Vβ-specific antibodies (Zaller et al., J. Exp. Med. 171:1943, 1990). In humans, certain Vβ gene segments have also been suggested to be associated with autoimmune diseases such as rheumatoid arthritis (Paliard et al., Science 253:325, 1991; Howell et al., Proc. Natl. Acad. Sci. USA 88:10921, 1991; Sottini et al., Eur. J. Immunol. 21:461, 1991; Uematsu et al., Proc. Natl. Acad. Sci. USA 88:8534, 1991; Marguerie et al., Immunol. Today 338:336, 1992), Sjögren's syndrome (Sumida et al., J. Clin. Invest. 89:681, 1992), and multiple sclerosis (Ben-Nun et al., Proc. Natl. Acad. Sci. USA 88:2466, 1991; Kotzin et al., Proc. Natl. Acad. Sci. USA 88:9161, 1991; Wucherpfennig et al., Science, 248:1016, 1990; Oksenberg et al., Nature 362:68-70, 1993). Such studies, however, have not been deemed to be conclusive, since these studies have been performed mainly either by the tedious procedure of expanding of antigen-reactive T cell clones and subsequent mRNA analysis, or by PCR of cDNA from diseased tissues. PCR analvsis in these studies was limited to only a subset of the Vβ gene segments due to the limited availability of sequences for designing unique primers.

Genetic susceptibility may also be manifested through genomic DNA which encodes structural and regulatory elements. This type of susceptibility may not always be detected as a protein variant, but may be associated with a polymorphism, especially those in non-coding regions. Many types of sequence polymorphisms are present in the genome, including for example, RFLPs, deletions, insertions, and variations in the number of repeat units of either simple repeats (e.g., dinucleotides such as CAₙ) or more complex repeats (e.g., VNTRs, LINES, SINES). RFLPs result either from single base changes in a restriction enzyme recognition sequence or variations of numbers of repeat sequences found on that particular restriction fragment. Estimates on the frequency of single base polymorphism in the human genome ranges from 1 in 200 bp to 1 in 1000 bp (Cooper et al., Hum. Genet. 69:201, 1985; Miyamoto et al., Proc. Natl. Acad. Sci. USA 85:7627, 1988). Not every single base change occurs in a restriction site; in one study, only 5 of 16 identified substitutions in the Cα, Cβ, and proII/thr t-RNA gene regions would have been detected as a RFLP (Nickerson et al., Genomics 12:377, 1992). The most accurate method for identifying DNA polymorphisms of either base substitution or repeats is direct determination of the DNA sequence.

Identification of polymorphisms is critical to determining disease susceptibility. To date, disease association studies have been limited, in part, by the restricted number of RFLP markers. These studies have generally been uninformative because of both the limited number of defined polymorphisms, and the lack of linkage disequilibrium across the TCR gene region (Robinson and Kindt, Proc. Natl. Acad. Sci. USA 82:3804, 1985). As examples, studies on myasthenia gravis (Smith et al., Ann. N.T Acad. Sci. 505:388, 1987), Graves' disease (Weetman et al., Hum. Immunol. 20:167, 1987), rheumatoid arthritis (Keystone et al., Arthritis Rheum. 31:1555, 1988; Mittenburg et al., Scand. J. Immunol 31:121, 1990), and Type I diabetes (Hibberd et al., Diabetic Med. 9:929, 1992) have suggested a role for TCR polymorphisms. Other studies have failed to find an association (Concannon et al., Am. J. Hum. Genet. 47:45, 1990; Hillert et al., J. Neuroimmunol. 31:141, 1991).

In summary, improved diagnosis of disease has been limited because less than 4% of the genomic sequence of Vβ genes is known. The present invention provides for the first time a complete genomic sequence of the β gene family, thereby allowing the production of diagnostics suitable for interrogating Vβ genes, as well as for generating therapeutic compositions. Further, other related advantages which are described in more detail below are also provided.

### SUMMARY OF THE INVENTION

Briefly stated, the present invention provides novel compositions and methods which are based upon knowledge of the entire genomic sequence of the Vβ gene region. Within one aspect of the present invention, isolated nucleic acid molecules are provided which encode Vβ25, Vβ26, Vβ27, Vβ28, Vβ29, Vβ30 or Vβ31.

Within other aspects, isolated nucleic acid molecules are provided comprising: a portion of a 5′ flanking sequence greater than 500 bp upstream of a Vβ gene coding region, with the proviso that the isolated nucleic acid molecule is not the 5′ flanking sequence of the Vβ gene for BV6S10, BV21S1 or BV21S4; a portion of a 5′ flanking sequence greater than 300 bp upstream of a Vβ gene coding region, with the proviso that the isolated nucleic acid molecule is not the 5′ flanking sequence of the Vβ gene for BV6S1, BV6S7, BV6S10, BV17S1, BV21S1 or BV21S4; or a portion of a 5′ flanking sequence greater than 200 bp upstream of a Vβ gene coding region, with the proviso that the isolated nucleic acid molecule is not the 5′ flanking sequence of the Vβ gene for BV3S1, BV5S6, BV6S1, BV6S3, BV6S7, BV6S10, BV8S1, BV8S3, BV8S4, BV8S5, BV13S2, BV13S3, BV13S4, BV13S5, BV13S9, BV17S1, BV21S1 or BV21 S4. Within other aspects, isolated nucleic acid molecules are provided comprising: a portion of a 3′ flanking sequence greater than 200 bp downstream of a V β gene coding region, with the proviso that the isolated nucleic acid molecule is not the 3′ flanking sequence of the Vβ gene for BV21S1; a portion of a 3′ flanking sequence greater than 100 bp downstream of a Vβ gene coding region, with the proviso that the isolated nucleic acid molecule is not the 3′ flanking sequence of the Vβ gene for BV3S1, BV15S1, BV16S1, BV17S1, BV20S1, BV21S1 or BV21S4. As utilized herein, a “portion” should be understood to include at least 14 nucleotides, preferably 16, 20, 24 or 30 nucleotides and possibly as much as the entire indicated flanking region (less one or more nucleotides).

Within another aspect of the present invention, nucleic acid probes are provided which are capable of specifically hybridizing to an isolated nucleic acid molecule as described above. Within one embodiment, the probe is between 16 and 24 nucleotides in length.

Within other aspects of the present invention, recombinant expression vectors are provided that comprise a promoter operably linked to any of the nucleic acid molecules described above.

Within another aspect of the present invention, a kit is provided comprising a panel of nucleic acid primers capable of specifically priming and allowing amplification of each and every Vβ gene, or each and every VβRNA or cDNA. Within a related embodiment, pairs of nucleic acid primers are provided which are capable of specifically priming and allowing amplification of Vβ genomic DNA, or VβRNA or cDNA. Within preferred embodiments, such primers are capable of specifically priming and allowing amplification of TCRBV1S1, TCRBV2S1, TCRBV2S2, TCRBV3S1, TCRBV4S1, TCRBV4S2, TCRBV5S1, TCRBV5S2, TCRBV5S3, TCRBV5S5, TCRBV5S6, TCRBV5S7, TCRBV5S8, TCRBV5S9, TCRBV6S1, TCRBV6S3, TCRBV6S4, TCRBV6S5, TCRBV6S7, TCRBV6S10, TCRBV6S11, TCRBV6S12, TCRBV6S14, TCRBV7S1, TCRBV7S2, TCRBV7S3, TCRBV8S1, TCRBV8S2, TCRBV8S3, TCRBV8S4, TCRBV8S5, TCRBV9S1, TCRBV9S2, TCRBV10S1, TCRBV10S2, TCRBV11S1, TCRBV12S2, TCRBV12S3, TCRBV12S4, TCRBV13S1, TCRBV13S2, TCRBV13S3, TCRBV13S4, TCRBV13S6, TCRBV13S7, TCRBV13S8, TCRBV14S1, TCRBV15S1, TCRBV16S1, TCRBV17S1, TCRBV18S1, TCRBV19S1, TCRBV20S1, TCRBV21S1, TCRBV21S3, TCRBV21S4 TCRBV22S1, TCRBV23S1, TCRBV24S1, TCRBV25S1, TCRBV26S1, TCRBV27S1, TCRBV28S1, TCRBV29S1, TCRBV30S1, TCRBV31S1, TCRBV32S1 and TCRBV33S1. Within related aspects of the present invention, nucleic acid primers are provided which are capable of specifically priming and allowing amplification of any one of the polymorphic sequences set forth in Figure Nos. 89-100.

Within another aspect, isolated T cell receptors are provided having a β chain that contains Vβ26, Vβ27, Vβ28, Vβ29, Vβ30, Vβ31, Vβ32, Vβ33, or Vβ34.

Within other aspects of the present invention, antibodies which are capable of specifically binding to cells that express a T cell receptor are provided. Within one embodiment, the antibody is a monoclonal antibody. Within other embodiments, the antibody is selected from the group consisting of Fab fragments and Fv fragments. Within a related aspect, a kit is provided, comprising a panel of antibodies which are capable of specifically binding to each and every unique β chain of a T cell receptor.

Within another aspect of the present invention, methods are provided for diagnosing organ transplant rejection in a patient following organ transplantation, comprising the steps of: (a) obtaining a biological sample containing T cells from a patient pre- and post-organ transplantation, (b) contacting the biological sample under conditions and for a time sufficient with a panel of antibodies capable of specifically binding to each and every unique β chain of a T cell receptor, and (c) detecting an increase of antibody binding in the post-organ transplantation biological sample relative to the level of antibody binding in the pre-organ transplantation sample, such that organ transplant rejection may be diagnosed in a patient following organ transplantation. Within one embodiment, the antibodies are labeled with a marker selected from the group consisting of enzymes, fluorophores, chromophores, and radionuclides.

Within a related aspect, methods are provided for diagnosing organ transplant rejection in a patient following organ transplantation, comprising the steps of: (a) obtaining a biological sample containing T cells from a patient pre- and post-organ transplantation, (b) extracting nucleic acids from the cells, (c) contacting the extracted nucleic acids with a panel of nucleic acid probes capable of specifically binding to each and every nucleic acid molecule encoding a β chain of a T cell receptor, and (d) detecting an increase of probe binding in the post-organ transplantation biological sample relative to the level of probe binding in the pre-organ transplantation sample, such that organ transplant rejection may be diagnosed in a patient following organ transplantation. Within one embodiment, such methods may further comprise, subsequent to the step of extracting nucleic acids, amplifying nucleic acids encoding Vβ regions.

Within yet another aspect, methods are provided for diagnosing organ transplant rejection in a patient following organ transplantation, comprising the steps of: (a) obtaining a biological sample containing T cells from a patient pre- and post-organ transplantation, (b) extracting nucleic acids from the cells, (c) amplifying nucleic acid molecules which encode Vβ regions, and (d) detecting an increase in the presence of amplified nucleic acid molecules which encode Vβ regions in the post-organ transplantation biological sample relative to the level of amplified molecules in the post-organ transplantation sample, such that organ transplant rejection may be diagnosed in a patient following organ transplantation.

Within certain embodiments of the above-described diagnostic methods, the extracted nucleic acids are ribonucleic acids. Within others, the nucleic acid probe is labeled with a marker selected from the group consisting of enzymes, fluorophores, chromophores, and radionuclides. Such methods may be readily applied to a wide variety of organ transplant patients, including, for example, those which have had an organ transplantation selected from the group consisting of heart, lung, kidney, spleen, liver, bone marrow, pancreas, thymus. lymph nodes, pineal glands, adrenal glands and skin.

Within other aspects of the present invention, methods are provided for determining a correlation between a disease or disease susceptibility and a selected polymorphism, comprising the steps of: (a) obtaining biological samples containing nucleated cells from a population. the population having individuals with a selected disease or disease susceptibility and individuals without the disease or disease susceptibility or individuals who are in remission from the selected disease, (b) extracting nucleic acids from the cells, (c) contacting the extracted nucleic acids with primers capable of specifically priming and allowing amplification of a selected polymorphism, (d) amplifying the selected polymorphism, and (e) detecting the presence of the polymorphism, and thereby determining a correlation between the disease or disease susceptibility and the selected polymorphism.

Within a related aspect, methods are provided for determining a correlation between a disease and a selected polymorphism, comprising the steps of (a) obtaining biological samples containing nucleated cells from a population, the population having individuals with a selected disease and individuals without the disease, (b) extracting ribonucleic acids from the cells, (c) reverse transcribing cDNA from the ribonucleic acids, (d) contacting the cDNA with primers capable of specifically priming and allowing amplification of a selected polymorphism, (e) amplifying the selected polymorphism, and (f) detecting the presence of the polymorphism, and thereby determining a correlation between the disease and the selected polymorphism.

Within yet another related aspect, methods are provided for determining a correlation between a disease and a selected polymorphism, comprising the steps of: (a) obtaining biological samples containing nucleated cells from a population, the population having individuals with a selected disease and individuals without the disease, (b) extracting nucleic acids from the cells, and (c) detecting the presence of the polymorphism, and thereby determining a correlation between the disease or disease susceptibility and the selected polymorphism.

Within certain embodiments of the above-described methods, the polymorphism may be a restriction fragment length polymorphism, a length difference of a simple repeat sequence, or a specific nucleotide substitution, deletion or insertion. Within other embodiments, the disease or disease susceptibility is selected from the group consisting of Addison's disease, atrophic gastritis, autoimmune hemolytic anemia, autoimmune neutropenia, bullous pemphigoid, Crohn's disease, coeliac disease, demyelinating neuropathies, dermatomyositis, Goodpasture's syndrome, Graves' disease, hemolytic anemia, idiopathic thrombocytopenia purpura, inflammatory bowel disease, insulin-dependent diabetes mellitus, juvenile diabetes, multiple sclerosis, myasthenia gravis, myocarditis, myositis, myxedema, pemphigus vulgaris, pernicious anaemia, primary glomerulonephritis, rheumatoid arthritis, scleritis, scleroderma, Sjogren's syndrome, systemic lupus ervthematosus, and type I diabetes.

Within other aspects of the present invention, methods are provided for determining a correlation between a disease resistance or disease susceptibility and a genetic marker, comprising the steps of: (a) obtaining biological samples containing nucleated cells from a population, the population having individuals with a selected disease resistance or disease susceptibility and individuals without the disease resistance or disease susceptibility, (b) extracting nucleic acids from the cells, (c) contacting the extracted nucleic acids with primers which are capable of specifically priming and allowing amplification of a series of selected genetic markers in the T cell receptor β gene region, the markers being selected such that they are in linkage disequilibrium with each other, (d) amplifying the genetic markers, and (e) determining the length of the amplified material, and thereby determining the correlation between a disease resistance or disease susceptibility and a genetic marker. Within certain embodiments, the series of genetic markers are at least 5 to 35 kb apart, and more preferably, at least 10 to 20 kb apart.

Within other aspects of the present invention, kits are provided which comprise a battery of primer pairs capable of specifically priming and allowing amplification of a series of selected markers in the T cell receptor β gene region, the markers being selected such that they are in linkage disequilibrium with each other. Within certain embodiments, the genetic markers are at least 5 to 35 kb apart, and more preferably, at least 10 to 20 kb apart.

These and other aspects of the present invention will become evident upon reference to the following detailed description and attached drawings. In addition, various references are set forth below which describe in more detail certain procedures or compositions (e.g., plasmids, etc.), and are therefore hereby incorporated by reference in their entirety as if each reference were individually noted for incorporation.

### BRIEF DESCRIPTION OF THE DRAWINGS

FIG. 1 is a diagram of the rearrangement process of individual gene segments, and the subsequent RNA splicing to generate a functional T cell receptor mRNA.

FIG. 2 is a representational map of the T cell receptor genes. The orientation of V, D, J, and C gene segments is shown for the α/δ,β, and γ gene families. The approximate chromosomal distance that encompasses each gene family is given for human and for mouse.

FIG. 3 is a schematic illustration of a representative cloning strategy.

FIG. 4 is a map of the TCR β locus. Below the map, cosmid clones used to generate the DNA sequence of this region are shown.

FIG. 5 is a table which provides estimates as to the precision of DNA sequence determination and the frequency of polymorphisms are presented.

FIG. 6 is a schematic illustration of the human β T cell receptor region. The relative positions of the TCR gene elements and trypsinogen genes are presented.

FIG. 7 is a schematic illustration of repeat structures present in the TCR β gene region. The number of repeat units is indicated and the % sequence divergence of the repeat units are also presented.

FIG. 8 is a schematic illustration of certain polymorphisms which are present in a 12.7 kb fragment. Nine polymorphisms are described and their location shown.

FIG. 9 is a table which presents the predicted amino acid translation of exon 2 of a member of each of the Vβ gene subfamilies, except for Vβ30 and Vβ32-34. The one-letter amino acid code is used. A dot (•) indicates a gap which is introduced to preserve maximum sequence identities.

FIG. 10 is a table which presents the DNA sequences of the exon/intron boundaries and recombination signal for each Vβ gene. The gene family is given and the genes are numbered in their relative chromosomal position.

FIG. 11 is a table which presents the recombination signal of each Vβ gene arranged by family.

FIG. 12 is a dot-matrix analysis of the human TCR β locus plotted against itself. Each dot represents 92% identity over 50 bases.

FIG. 13 is the genomic sequence of TCRBV1S1. The map position refers to the first base (A) of the initiator methionine codon in exon 1.

FIG. 14 is the genomic sequence of TCRBV2S1. The map position refers to the first base (A) of the initiator methionine codon in exon 1.

FIG. 15 is the genomic sequence of TCRBV3S1. The map position refers to the first base (A) of the initiator methionine codon in exon 1.

FIG. 16 is the genomic sequence of TCRBV4S1. The map position refers to the first base (A) of the initiator methionine codon in exon 1.

FIG. 17 is the genomic sequence of TCRBV5S1. The map position refers to the first base (A) of the initiator methionine codon in exon 1.

FIG. 18 is the genomic sequence of TCRBV5S2. The map position refers to the first base (A) of the initiator methionine codon in exon 1.

FIG. 19 is the genomic sequence of TCRBV5S3. The map position refers to the first base (A) of the initiator methionine codon in exon 1.

FIG. 20 of the genomic sequence of TCRBV5S5. The map position refers to the first base (A) of the initiator methionine codon in exon 1.

FIG. 21 is the genomic sequence of TCRBV5S6. The map position refers to the first base (A) of the initiator methionine codon in exon 1.

FIG. 22 is the genomic sequence of TCRBV5S7. The map position refers to the first base (A) of the initiator methionine codon in exon 1.

FIG. 23 is the genomic sequence of TCRBV5S8. The map position refers to the first base (A) of the initiator methionine codon in exon 1.

FIG. 24 is the genomic sequence of TCRBV6S1. The map position refers to the first base (A) of the initiator methionine codon in exon 1.

FIG. 25 is the genomic sequence of TCRBV6S3. The map position refers to the first base (A) of the initiator methionine codon in exon 1.

FIG. 26 is the genomic sequence of TCRBV6S4. The map position refers to the first base (A) of the initiator methionine codon in exon 1.

FIG. 27 is the genomic sequence of TCRBV6S5. The map position refers to the first base (A) of the initiator methionine codon in exon 1.

FIG. 28 is the genomic sequence of TCRBV6S7. The map position refers to the first base (A) of the initiator methionine codon in exon 1.

FIG. 29 is the genomic sequence of TCRBV6S10. The map position refers to the first base (A) of the initiator methionine codon in exon 1.

FIG. 30 is the genomic sequence of TCRBV6S11. The map position refers to the first base (A) of the initiator methionine codon in exon 1.

FIG. 31 is the genomic sequence of TCRBV6S12. The map position refers to the first base (A) of the initiator methionine codon in exon 1.

FIG. 32 is the genomic sequence of TCRBV6S14. The map position refers to the first base (A) of the initiator methionine codon in exon 1.

FIG. 33 is the genomic sequence of TCRBV7S1. The map position refers to the first base (A) of the initiator methionine codon in exon 1.

FIG. 34 is the genomic sequence of TCRBV7S2. The map position refers to the first base (A) of the initiator methionine codon in exon 1.

FIG. 35 is the genomic sequence of TCRBV7S3. The map position refers to the first base (A) of the initiator methionine codon in exon 1.

FIG. 36 is the genomic sequence of TCRBV8S1. The map position refers to the first base (A) of the initiator methionine codon in exon 1.

FIG. 37 is the genomic sequence of TCRBV8S2. The map position refers to the first base (A) of the initiator methionine codon in exon 1.

FIG. 38 is the genomic sequence of TCRBV8S3. The map position refers to the first base (A) of the initiator methionine codon in exon 1.

FIG. 39 is the genomic sequence of TCRBV8S4. The map position refers to the first base (A) of the initiator methionine codon in exon 1.

FIG. 40 is the genomic sequence of TCRBV8S5. The map position refers to the first base (A) of the initiator methionine codon in exon 1.

FIG. 41 is the genomic sequence of TCRBV9S1. The map position refers to the first base (A) of the initiator methionine codon in exon 1.

FIG. 42 is the genomic sequence of TCRBV9S2. The map position refers to the first base (A) of the initiator methionine codon in exon 1.

FIG. 43 is the genomic sequence of TCRBV10S1. The map position refers to the first base (A) of the initiator methionine codon in exon 1.

FIG. 44 is the genomic sequence of TCRBV11S1. The map position refers to the first base (A) of the initiator methionine codon in exon 1.

FIG. 45 is the genomic sequence of TCRBV12S2. The map position refers to the first base (A) of the initiator methionine codon in exon 1.

FIG. 46 is the genomic sequence of TCRBV12S3. The map position refers to the first base (A) of the initiator methionine codon in exon 1.

FIG. 47 is the genomic sequence of TCRBV12S4. The map position refers to the first base (A) of the initiator methionine codon in exon 1.

FIG. 48 is the genomic sequence of TCRBV13S1. The map position refers to the first base (A) of the initiator methionine codon in exon 1.

FIG. 49 is the genomic sequence of TCRBV13S2. The map position refers to the first base (A) of the initiator methionine codon in exon 1.

FIG. 50 is the genomic sequence of TCRBV13S3. The map position refers to the first base (A) of the initiator methionine codon in exon 1.

FIG. 51 is the genomic sequence of TCRBV13S4. The map position refers to the first base (A) of the initiator methionine codon in exon 1.

FIG. 52 is the genomic sequence of TCRBV13S5. The map position refers to the first base (A) of the initiator methionine codon in exon 1.

FIG. 53 is the genomic sequence of TCRBVI3S6. The map position refers to the first base (A) of the initiator methionine codon in exon 1.

FIG. 54 is the genomic sequence of TCRBVI3S7. The map position refers to the first base (A) of the initiator methionine codon in exon 1.

FIG. 55 is the genomic sequence of TCRBV13S8. The map position refers to the first base (A) of the initiator methionine codon in exon 1.

FIG. 56 is the genomic sequence of TCRBV13S9. The map position refers to the first base (A) of the initiator methionine codon in exon 1.

FIG. 57 is the genomic sequence of TCRBV14S1. The map position refers to the first base (A) of the initiator methionine codon in exon 1.

FIG. 58 is the genomic sequence of TCRBV15S1. The map position refers to the first base (A) of the initiator methionine codon in exon 1.

FIG. 59 is the genomic sequence of TCRBV16S1. The map position refers to the first base (A) of the initiator methionine codon in exon 1.

FIG. 60 is the genomic sequence of TCRBV17S1. The map position refers to the first base (A) of the initiator methionine codon in exon 1.

FIG. 61 is the genomic sequence of TCRBV18S1. The map position refers to the first base (A) of the initiator methionine codon in exon 1.

FIG. 62 is the genomic sequence of TCRBV19S1. The map position refers to the first base (A) of the initiator methionine codon in exon 1.

FIG. 63 is the genomic sequence of TCRBV20S1. The map position refers to the first base (A) of the initiator methionine codon in exon 1.

FIG. 64 is the genomic sequence of TCRBV21S1. The map position refers to the first base (A) of the initiator methionine codon in exon 1.

FIG. 65 is the genomic sequence of TCRBV2 1S3. The map position refers to the first base (A) of the initiator methionine codon in exon 1.

FIG. 66 is the genomic sequence of TCRBV21S4. The map position refers to the first base (A) of the initiator methionine codon in exon 1.

FIG. 67 is the genomic sequence of TCRBV22S1. The map position refers to the first base (A) of the initiator methionine codon in exon 1.

FIG. 68 is the genomic sequence of TCRBV23S1. The map position refers to the first base (A) of the initiator methionine codon in exon 1.

FIG. 69 is the genomic sequence of TCRBV24S1. The map position refers to the first base (A) of the initiator methionine codon in exon 1.

FIG. 70 is the genomic sequence of TCRBV25S1. The map position refers to the first base (A) of the initiator methionine codon in exon 1.

FIG. 71 is the genomic sequence of TCRBV26S1. The map position refers to the first base (A) of the initiator methionine codon in exon 1.

FIG. 72 is the genomic sequence of TCRBV27S1. The map position refers to the first base (A) of the initiator methionine codon in exon 1.

FIG. 73 is the genomic sequence of TCRBV28S1. The map position refers to the first base (A) of the initiator methionine codon in exon 1.

FIG. 74 is the genomic sequence of TCRBV29S1. The map position refers to the first base (A) of the initiator methionine codon in exon 1.

FIG. 75 is the genomic sequence of TCRBV30S1. The map position refers to the first base (A) of the initiator methionine codon in exon 1.

FIG. 76 is the genomic sequence of TCRBV31S1. The map position refers to the first base (A) of the initiator methionine codon in exon 1.

FIG. 77 is the genomic sequence of TCRBV32S1. The map position refers to the first base (A) of the initiator methionine codon in exon 1.

FIG. 78 is the genomic sequence of TCRBV33S1. The map position refers to the first base (A) of the initiator methionine codon in exoz 1.

FIG. 79 is the genomic sequence of TCRBV34S1. The map position refers to the first base (A) of the initiator methionine codon in exon 1.

FIG. 80A is the translated sequence of TCRBV1S1 presented in one-letter amino acid code. A “n” represents a frameshift and an “x” represents a stop codon. Frameshifts and stop codons are accommodated to preserve a similar amino acid sequence.

FIG. 80B is the translated sequence of TCRBV2S1 presented in one-letter amino acid code. A “n” represents a frameshift and an “x” represents a stop codon. Frameshifts and stop codons are accommodated to preserve a similar amino acid sequence.

FIG. 80C is the translated sequence of TCRBV3S1 presented in one-letter amino acid code. A “n” represents a frameshift and an “x” represents a stop codon. Frameshifts and stop codons are accommodated to preserve a similar amino acid sequence.

FIG. 80D is the translated sequence of TCRBV4S1 presented in one-letter amino acid code. A “n” represents a frameshift and an “x” represents a stop codon. Frameshifts and stop codons are accommodated to preserve a similar amino acid sequence.

FIG. 80E is the translated sequence of TCRBV5S1 presented in one-letter amino acid code. A “n” represents a frameshift and an “x” represents a stop codon. Frameshifts and stop codons are accommodated to preserve a similar amino acid sequence.

FIG. 80F is the translated sequence of TCRBV5S2 presented in one-letter amino acid code. A “n” represents a frameshift and an “x” represents a stop codon. Frameshifts and stop codons are accommodated to preserve a similar amino acid sequence.

FIG. 80G is the translated sequence of TCRBV5S3 presented in one-letter amino acid code. A “n” represents a frameshift and an “x” represents a stop codon. Frameshifts and stop codons are accommodated to preserve a similar amino acid sequence.

FIG. 81A is the translated sequence of TCRBV5S5 presented in one-letter amino acid code. A “n” represents a frameshift and an “x” represents a stop codon. Frameshifts and stop codons are accommodated to preserve a similar amino acid sequence.

FIG. 81B is the translated sequence of TCRBV5S6 presented in one-letter amino acid code. A “n” represents a frameshift and an “x” represents a stop codon. Frameshifts and stop codons are accommodated to preserve a similar amino acid sequence.

FIG. 81C is the translated sequence of TCRBV5S7 presented in one-letter amino acid code. A “n” represents a frameshift and an “x” represents a stop codon. Frameshifts and stop codons are accommodated to preserve a similar amino acid sequence.

FIG. 81D is the translated sequence of TCRBV5S8 presented in one-letter amino acid code. A “n” represents a frameshift and an “x” represents a stop codon. Frameshifts and stop codons are accommodated to preserve a similar amino acid sequence.

FIG. 81E is the translated sequence of TCRBV6S1 presented in one-letter amino acid code. A “n” represents a frameshift and an “x” represents a stop codon. Frameshifts and stop codons are accommodated to preserve a similar amino acid sequence.

FIG. 81F is the translated sequence of TCRBV6S3 presented in one-letter amino acid code. A “n” represents a frameshift and an “x” represents a stop codon. Frameshifts and stop codons are accommodated to preserve a similar amino acid sequence.

FIG. 81G is the translated sequence of TCRBV6S4 presented in one-letter amino acid code. A “n” represents a frameshift and an “x” represents a stop codon. Frameshifts and stop codons are accommodated to preserve a similar amino acid sequence.

FIG. 82A is the translated sequence of TCRBV6S5 presented in one-letter amino acid code. A “n” represents a frameshift and an “x” represents a stop codon. Frameshifts and stop codons are accommodated to preserve a similar amino acid sequence.

FIG. 82B is the translated sequence of TCRBV6S7 presented in one-letter amino acid code. A “n” represents a frameshift and an “x” represents a stop codon. Frameshifts and stop codons are accommodated to preserve a similar amino acid sequence.

FIG. 82C is the translated sequence of TCRBV6S10 presented in one-letter amino acid code. A “n” represents a frameshift and an “x” represents a stop codon. Frameshifts and stop codons are accommodated to preserve a similar amino acid sequence.

FIG. 82D is the translated sequence of TCRBV6S11 presented in one-letter amino acid code. A “n” represents a frameshift and an “x” represents a stop codon. Frameshifts and stop codons are accommodated to preserve a similar amino acid sequence.

FIG. 82E is the translated sequence of TCRBV6S12 presented in one-letter amino acid code. A “n” represents a frameshift and an “x” represents a stop codon. Frameshifts and stop codons are accommodated to preserve a similar amino acid sequence.

FIG. 82F is the translated sequence of TCRBV6S14 presented in one-letter amino acid code. A “n” represents a frameshift and an “x” represents a stop codon. Frameshifts and stop codons are accommodated to preserve a similar amino acid sequence.

FIG. 82G is the translated sequence of TCRBV7S1 presented in one-letter amino acid code. A “n” represents a frameshift and an “x” represents a stop codon. Frameshifts and stop codons are accommodated to preserve a similar amino acid sequence.

FIG. 83A is the translated sequence of TCRBV7S2 presented in one-letter amino acid code. A “n” represents a frameshift and an “x” represents a stop codon. Frameshifts and stop codons are accommodated to preserve a similar amino acid sequence.

FIG. 83B is the translated sequence of TCRBV7S3 presented in one-letter amino acid code. A “n” represents a frameshift and an “x” represents a stop codon. Frameshifts and stop codons are accommodated to preserve a similar amino acid sequence.

FIG. 83C is the translated sequence of TCRBV8S1 presented in one-letter amino acid code. A “n” represents a frameshift and an “x” represents a stop codon. Frameshifts and stop codons are accommodated to preserve a similar amino acid sequence.

FIG. 83D is the translated sequence of TCRBV8S2 presented in one-letter amino acid code. A “n” represents a frameshift and an “x” represents a stop codon. Frameshifts and stop codons are accommodated to preserve a similar amino acid sequence.

FIG. 83E is the translated sequence of TCRBV8S3 presented in one-letter amino acid code. A “n” represents a frameshift and an “x” represents a stop codon. Frameshifts and stop codons are accommodated to preserve a similar amino acid sequence.

FIG. 83F is the translated sequence of TCRBV8S4 presented in one-letter amino acid code. A “n” represents a frameshift and an “x” represents a stop codon. Frameshifts and stop codons are accommodated to preserve a similar amino acid sequence.

FIG. 83G is the translated sequence of TCRBV8S5 presented in one-letter amino acid code. A “n” represents a frameshift and an “x” represents a stop codon. Frameshifts and stop codons are accommodated to preserve a similar amino acid sequence.

FIG. 84A is the translated sequence of TCRBV9S1 presented in one-letter amino acid code. A “n” represents a frameshift and an “x” represents a stop codon. Frameshifts and stop codons are accommodated to preserve a similar amino acid sequence.

FIG. 84B is the translated sequence of TCRBV9S2 presented in one-letter amino acid code. A “n” represents a frameshift and an “x” represents a stop codon. Frameshifts and stop codons are accommodated to preserve a similar amino acid sequence.

FIG. 84C is the translated sequence of TCRBV10S1 presented in one-letter amino acid code. A “n” represents a frameshift and an “x” represents a stop codon. Frameshifts and stop codons are accommodated to preserve a similar amino acid sequence.

FIG. 84D is the translated sequence of TCRBV11S1 presented in one-letter amino acid code. A “n” represents a frameshift and an “x” represents a stop codon. Frameshifts and stop codons are accommodated to preserve a similar amino acid sequence.

FIG. 84E is the translated sequence of TCRBV12S2 presented in one-letter amino acid code. A “n” represents a frameshift and an “x” represents a stop codon. Frameshifts and stop codons are accommodated to preserve a similar amino acid sequence.

FIG. 84F is the translated sequence of TCRBV12S3 presented in one-letter amino acid code. A “n” represents a frameshift and an “x” represents a stop codon. Frameshifts and stop codons are accommodated to preserve a similar amino acid sequence.

FIG. 84G is the translated sequence of TCRBV12S4 presented in one-letter amino acid code. A “n” represents a frameshift and an “x” represents a stop codon. Frameshifts and stop codons are accommodated to preserve a similar amino acid sequence.

FIG. 85A is the translated sequence of TCRBV13S1 presented in one-letter amino acid code. A “n” represents a frameshift and an “x” represents a stop codon. Frameshifts and stop codons are accommodated to preserve a similar amino acid sequence.

FIG. 85B is the translated sequence of TCRBV13S2 presented in one-letter amino acid code. A “n” represents a frameshift and an “x” represents a stop codon. Frameshifts and stop codons are accommodated to preserve a similar amino acid sequence.

FIG. 85C is the translated sequence of TCRBV13S3 presented in one-letter amino acid code. A “n” represents a frameshift and an “x” represents a stop codon. Frameshifts and stop codons are accommodated to preserve a similar amino acid sequence.

FIG. 85D is the translated sequence of TCRBV13S4 presented in one-letter amino acid code. A “n” represents a frameshift and an “x” represents a stop codon. Frameshifts and stop codons are accommodated to preserve a similar amino acid sequence.

FIG. 85E is the translated sequence of TCRBV13S5 presented in one-letter amino acid code. A “n” represents a frameshift and an “x” represents a stop codon. Frameshifts and stop codons are accommodated to preserve a similar amino acid sequence.

FIG. 85F is the translated sequence of TCRBV13S6 presented in one-letter amino acid code. A “n” represents a frameshift and an “x” represents a stop codon. Frameshifts and stop codons are accommodated to preserve a similar amino acid sequence.

FIG. 85G is the translated sequence of TCRBV13S7 presented in one-letter amino acid code. A “n” represents a frameshift and an “x” represents a stop codon. Frameshifts and stop codons are accommodated to preserve a similar amino acid sequence.

FIG. 86A is the translated sequence of TCRBV13S8 presented in one-letter amino acid code. A “n” represents a frameshift and an “x” represents a stop codon. Frameshifts and stop codons are accommodated to preserve a similar amino acid sequence.

FIG. 86B is the translated sequence of TCRBV13S9 presented in one-letter amino acid code. A “n” represents a frameshift and an “x” represents a stop codon. Frameshifts and stop codons are accommodated to preserve a similar amino acid sequence.

FIG. 86C is the translated sequence of TCRBV14S1 presented in one-letter amino acid code. A “n” represents a frameshift and an “x” represents a stop codon. Frameshifts and stop codons are accommodated to preserve a similar amino acid sequence.

FIG. 86D is the translated sequence of TCRBV15S1 presented in one-letter amino acid code. A “n” represents a frameshift and an “x” represents a stop codon. Frameshifts and stop codons are accommodated to preserve a similar amino acid sequence.

FIG. 86E is the translated sequence of TCRBV16S1 presented in one-letter amino acid code. A “n” represents a frameshift and an “x” represents a stop codon. Frameshifts and stop codons are accommodated to preserve a similar amino acid sequence.

FIG. 86F is the translated sequence of TCRBV7S1 presented in one-letter amino acid code. A “n” represents a frameshift and an “x” represents a stop codon. Frameshifts and stop codons are accommodated to preserve a similar amino acid sequence.

FIG. 86G is the translated sequence of TCRBV18S1 presented in one-letter amino acid code. A “n” represents a frameshift and an “x” represents a stop codon. Frameshifts and stop codons are accommodated to preserve a similar amino acid sequence.

FIG. 87A is the translated sequence of TCRBV19S1 presented in one-letter amino acid code. A “n” represents a frameshift and an “x” represents a stop codon. Frameshifts and stop codons are accommodated to preserve a similar amino acid sequence.

FIG. 87B is the translated sequence of TCRBV20S1 presented in one-letter amino acid code. A “n” represents a frameshift and an “x” represents a stop codon. Frameshifts and stop codons are accommodated to preserve a similar amino acid sequence.

FIG. 87C is the translated sequence of TCRBV21S1 presented in one-letter amino acid code. A “n” represents a frameshift and an “x” represents a stop codon. Frameshifts and stop codons are accommodated to preserve a similar amino acid sequence.

FIG. 87D is the translated sequence of TCRBV21S3 presented in one-letter amino acid code. A “n” represents a frameshift and an “x” represents a stop codon. Frameshifts and stop codons are accommodated to preserve a similar amino acid sequence.

FIG. 87E is the translated sequence of TCRBV21S4 presented in one-letter amino acid code. A “n” represents a frameshift and an “x” represents a stop codon. Frameshifts and stop codons are accommodated to preserve a similar amino acid sequence.

FIG. 87F is the translated sequence of TCRBV22S1 presented in one-letter amino acid code. A “n” represents a frameshift and an “x” represents a stop codon. Frameshifts and stop codons are accommodated to preserve a similar amino acid sequence.

FIG. 87G is the translated sequence of TCRBV23S1 presented in one-letter amino acid code. A “n” represents a frameshift and an “x” represents a stop codon. Frameshifts and stop codons are accommodated to preserve a similar amino acid sequence.

FIG. 88A is the translated sequence of TCRBV24S1 presented in one-letter amino acid code. A “n” represents a frameshift and an “x” represents a stop codon. Frameshifts and stop codons are accommodated to preserve a similar amino acid sequence.

FIG. 88B is the translated sequence of TCRBV25S1 presented in one-letter amino acid code. A “n” represents a frameshift and an “x” represents a stop codon. Frameshifts and stop codons are accommodated to preserve a similar amino acid sequence.

FIG. 88C is the translated sequence of TCRBV26S1 presented in one-letter amino acid code. A “n” represents a frameshift and an “x” represents a stop codon. Frameshifts and stop codons are accommodated to preserve a similar amino acid sequence.

FIG. 88D is the translated sequence of TCRBV27S1 presented in one-letter amino acid code. A “n” represents a frameshift and an “x” represents a stop codon. Frameshifts and stop codons are accommodated to preserve a similar amino acid sequence.

FIG. 88E is the translated sequence of TCRBV28S1 presented in one-letter amino acid code. A “n” represents a frameshift and an “x” represents a stop codon. Frameshifts and stop codons are accommodated to preserve a similar amino acid sequence.

FIG. 88F is the translated sequence of TCRBV29S1 presented in one-letter amino acid code. A “n” represents a frameshift and an “x” represents a stop codon. Frameshifts and stop codons are accommodated to preserve a similar amino acid sequence.

FIG. 89 is the genomic sequence of a microsatellite and flanking sequence. The microsatellite is underlined. The map position refers to the first base of the microsatellite.

FIG. 90 is the genomic sequence of a microsatellite and flanking sequence. The microsatellite is underlined. The map position refers to the first base of the microsatellite.

FIG. 91 is the genomic sequence of a microsatellite and flanking sequence. The microsatellite is underlined. The map position refers to the first base of the microsatellite.

FIG. 92 is the genomic sequence of a microsatellite and flanking sequence. The microsatellite is underlined. The map position refers to the first base of the microsatellite.

FIG. 93 is the genomic sequence of a microsatellite and flanking sequence. The microsatellite is underlined. The map position refers to the first base of the microsatellite.

FIG. 94 is the genomic sequence of a microsatellite and flanking sequence. The microsatellite is underlined. The map position refers to the first base of the microsatellite.

FIG. 95 is the genomic sequence of a microsatellite and flanking sequence. The microsatellite is underlined. The map position refers to the first base of the microsatellite.

FIG. 96 is the genomic sequence of a microsatellite and flanking sequence. The microsatellite is underlined. The map position refers to the first base of the microsatellite.

FIG. 97 is the genomic sequence of a microsatellite and flanking sequence. The microsatellite is underlined. The map position refers to the first base of the microsatellite.

FIG. 98 is the genomic sequence of a microsatellite and flanking sequence. The microsatellite is underlined. The map position refers to the first base of the microsatellite.

FIG. 99 is the genomic sequence of a microsatellite and flanking sequence. The microsatellite is underlined. The map position refers to the first base of the microsatellite.

FIG. 100 is a presentation of di-, tri-, tetra-, and pentanucleotide repeat sequences in the TCR β gene region. The map positions of the first and last nucleotide of each repeat are given, as well as the length of the repeat sequence. The nucleotide sequence of each repeat is also presented.

FIG. 101 is a table which sets forth a series of primers for amplification of Vβ cDNA or RNA, when paired with a Cβ primer such as: TGTGGGAGATCTCTGCTTCT (Sequence I.D. No. 1181).

FIG. 102 is a table which sets forth a series of primers for amplification of Vβ genes.

FIG. 103 is a table which sets forth a series of primers for amplification of Vβ genes.

FIG. 104 is a table which sets forth a series of primers for amplification and analysis of several selected point mutations.

FIG. 105 is a graph which depicts the relative location and number of di-, tri-, tetra-, and pentanucleotide repeats.

FIGS. 106A, 106B, 106C, and 106D are a table which set forth the amplification conditions and allele distribution of general novel TCRβ microsatellites.

FIG. 107 is a graph which depicts the association of microsatellites with disease susceptibility alleles.

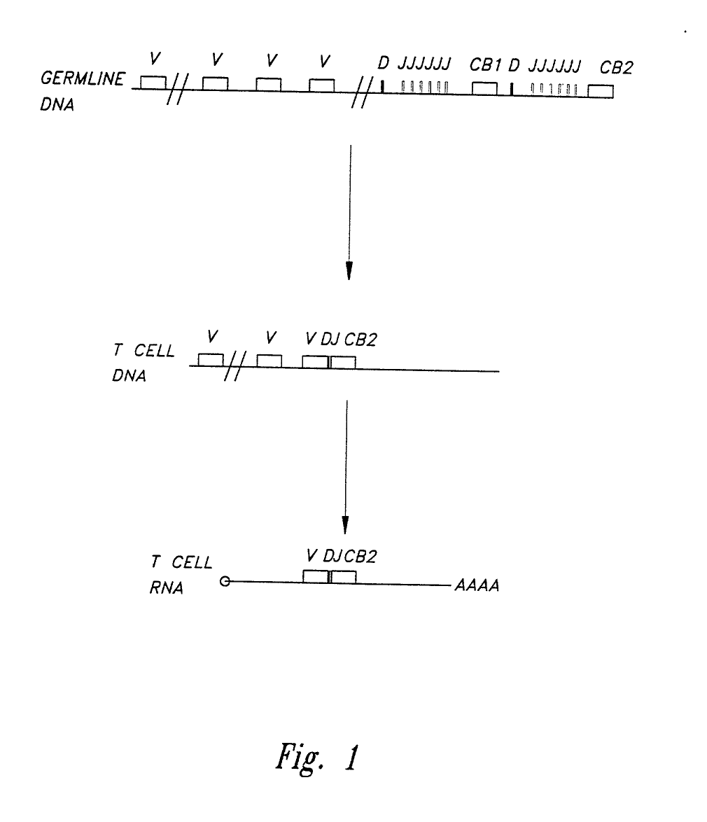

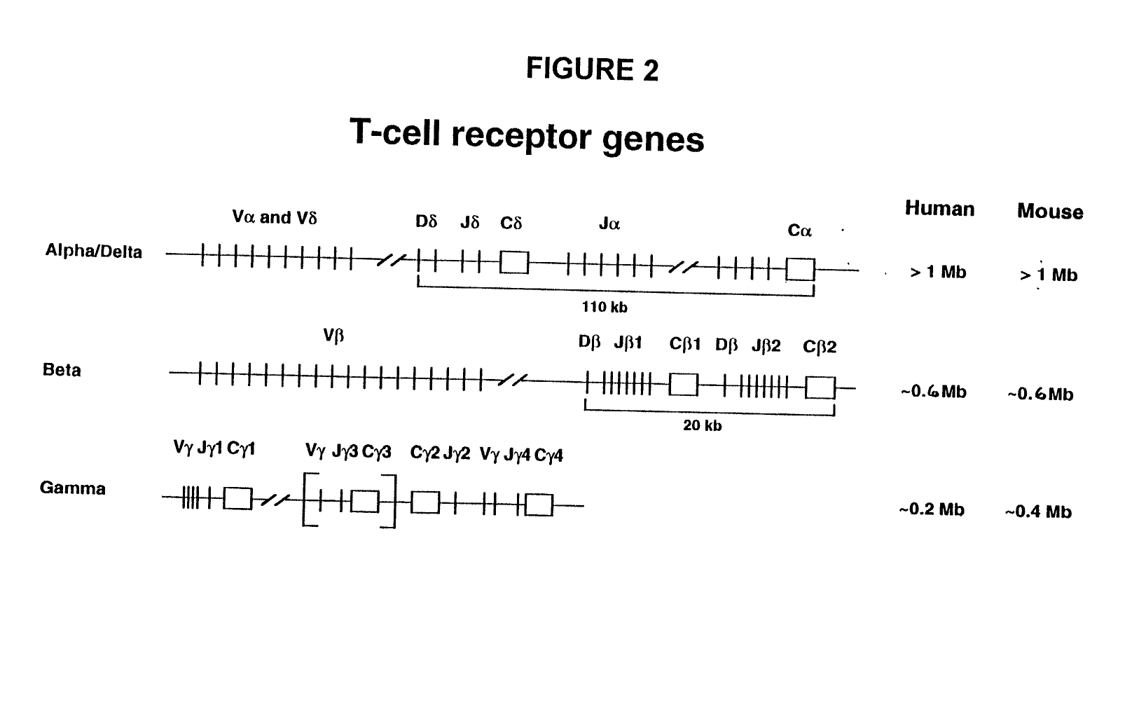

### DETAILED DESCRIPTION OF THE INVENTION

Prior to setting forth the invention, it may be helpful to an understanding thereof to first set forth definitions of certain terms that will be used hereinafter.

“Polymorphism” refers to a site or trait encoded by a chromosome that shows variation within a given population. Representative examples of polymorphisms include Restriction Fragment Length Polymorphisms (RFLPs); nucleotide substitutions, deletions, insertions, and variation in the number of repeat units of either simple repeats (e.g., di-, tri-, tetra- or penta-nucleotide repeats); or more complex repeats such as Variable Number of Tandem Repeats (“VNTR”), Short Interspersed Nuclear Elements (“SINES”), and Long Interspersed Nuclear Elements (“LINES”).

“Nucleic acid molecule” refers to a nucleic acid polymer or nucleic acid sequence, which exists in the form of a separate fragment or as a component of a larger nucleic acid construct. The nucleic acid molecule must have been derived from nucleic acids isolated at least once in substantially pure form, (i.e., substantially free of contaminating endogenous materials), and in a quantity or concentration enabling identification and recovery. Within certain embodiments, such sequences may be provided in the form of an open reading frame uninterrupted by internal nontranslated sequences, or introns. As utilized herein, nucleic acid molecules should be understood to include deoxyribonucleic acid (“DNA”) molecules (including genomic and cDNA molecules), ribonucleic acid (“RNA”) molecules, hybrid or chimeric nucleic acid molecules (e.g., DNA-RNA hybrids), and where appropriate, nucleic acid molecule analogs and derivatives (e.g. peptide nucleic acids (“PNA”)). Nucleic acid molecules of the present invention may also comprise sequences of non-translated nucleic acids.

“Recombinant expression vector” refers to a replicable nucleic acid construct used either to amplify or to express nucleic acid sequences which encode T cell or soluble T cell receptors. This construct comprises an assembly of (1) a genetic element or elements having a regulatory role in gene expression, for example, promoters, and (2) the structural or coding sequence of interest. The recombinant expression vector may also comprise appropriate transcription and translation initiation and termination sequences.

As noted above, the present invention provides a complete sequence of the β gene family. Previous to the present invention 4% or less of this sequence was known, thereby impeding the development of both diagnostics and therapeutics for T cell receptor related diseases. Through use of the entire sequence, both diagnostic and therapeutic compositions and methods may now be prepared and implemented. For example, as discussed in more detail below, knowledge of the entire Vβ gene region allows unambiguous identification and interrogation of each and every Vβ gene, as well as the development of diagnostics which are capable of specifically detecting these genes and the proteins encoded by these genes. Moreover, knowledge of the entire Vβ gene sequence allows the development of detailed genetic maps, as well as provides a basis for determining disease association with particular coding sequences (i. e., polymorphisms).

### The TCRβ Gene Region

Prior to sequencing the human T cell receptor locus, it was known that most of the V segment subfamilies had multiple members. The exact number of members in each of the multigene subfamilies was unknown for the larger families, e.g., Vβ6, Vβ5, and Vβ3. This was so because multiple, and often indistinguishable, bands were obtained from Southern blot hybridizations using probes from the multigene subfamilies. (The criterion for membership within a subfamily is based on sequence homology greater than or equal to 75%.) To complicate matters even further, some of the subfamilies are evolutionarily related (e.g., Vβ6 and Vβ8), sufficiently so as to result in cross-hybridization under typical experimental conditions. Hence, the sequencing of the β locus represented an enormous new technical challenge because of all the closely related sequences.

Because the initial cosmid libraries were made from genomic cell lines (YACs were not available in the late 1980s when the mapping of the T cell receptor locus was begun), hybridization screening was required for identifying cosmids from the TCR locus. However, the results of this screening were too ambiguous to be useful for generating a map, because of all the closely related sequences.

To solve this problem, restriction digest clustering was employed as a method for mapping the cosmids. Unfortunately, this method also gave ambiguous results for significant portions of the locus. This was so because, unbeknownst at the time, the locus contains long (7-25 kb) homologous internal repeats, several of which are in the 90%-94% similarity range. Because of the high degree of homology, restriction digest patterns of several cosmids from different parts of the locus looked alike. (From the sequence provided herein, ˜44% of the locus is repeated internally, or ˜65% if the chromosome 9 translocation is counted.) Hence, not only were the V gene segments similar, but so were large homology units as blocks of sequence within the locus.

The third complication faced came from two large (22 kb; 15 kb) insertion/deletion polymorphisms, both of which are located within the repeat clusters referred to above. In addition, each diploid human cell line had two distinct β loci (one maternal and the other paternal) which differed on average by 1/600 nucleotides. Thus. base and size polymorphisms had to be separated into the distinct maternal and paternal chromosome constellations or haplotypes.

Thus, to map the multitude of similar V genes, the very similar homology units, and to identify the two distinct haplotypes in each library, it was necessary to carry out a preliminary restriction map analyses and then determine the 3′ and 5′ sequences on ambiguous cosmids. From this end sequence data, STSs (sequence tagged sites) were developed from unique sequences and used to determine which cosmids overlapped. This approach allowed the development of a complete physical map of this complex locus.

In all, it was necessary to use a map-sequence-map-sequence bootstrap strategy to complete this cosmid contig map. Without having a sequence (where the repeat units were sorted out) to base a map on, the map would have been difficult to impossible to complete. This integration of mapping and sequencing to finish the project has been unique among large-scale DNA sequencing projects undertaken to date.

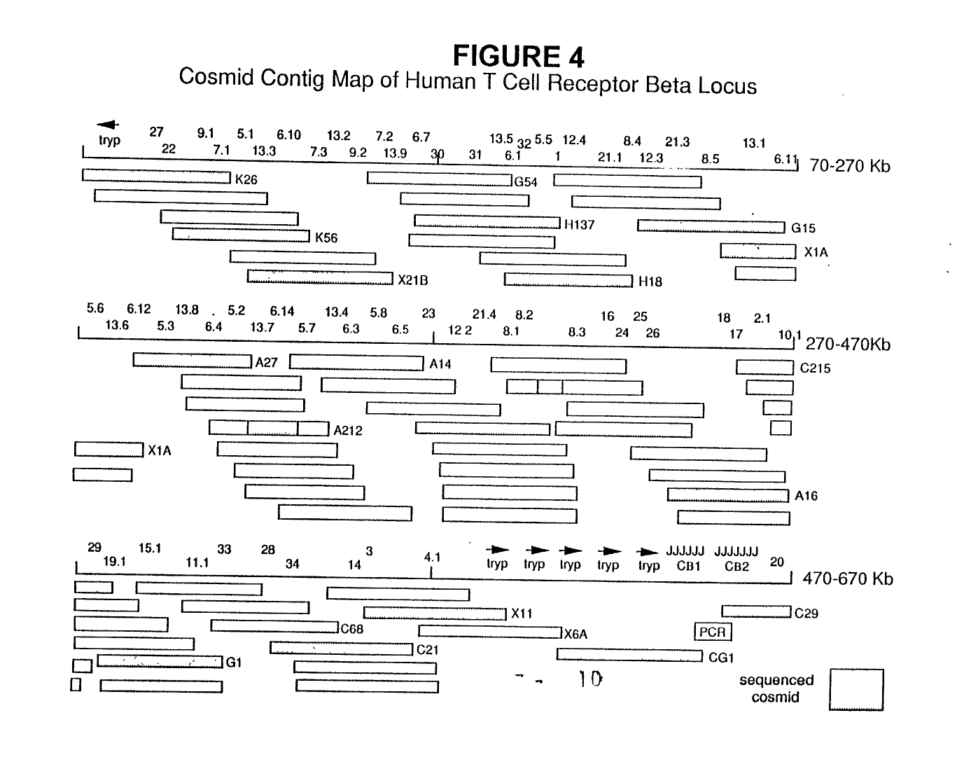

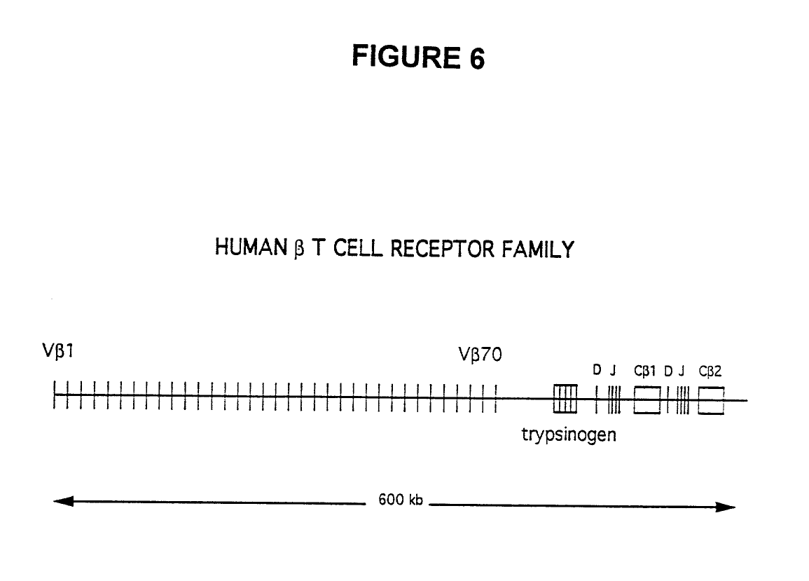

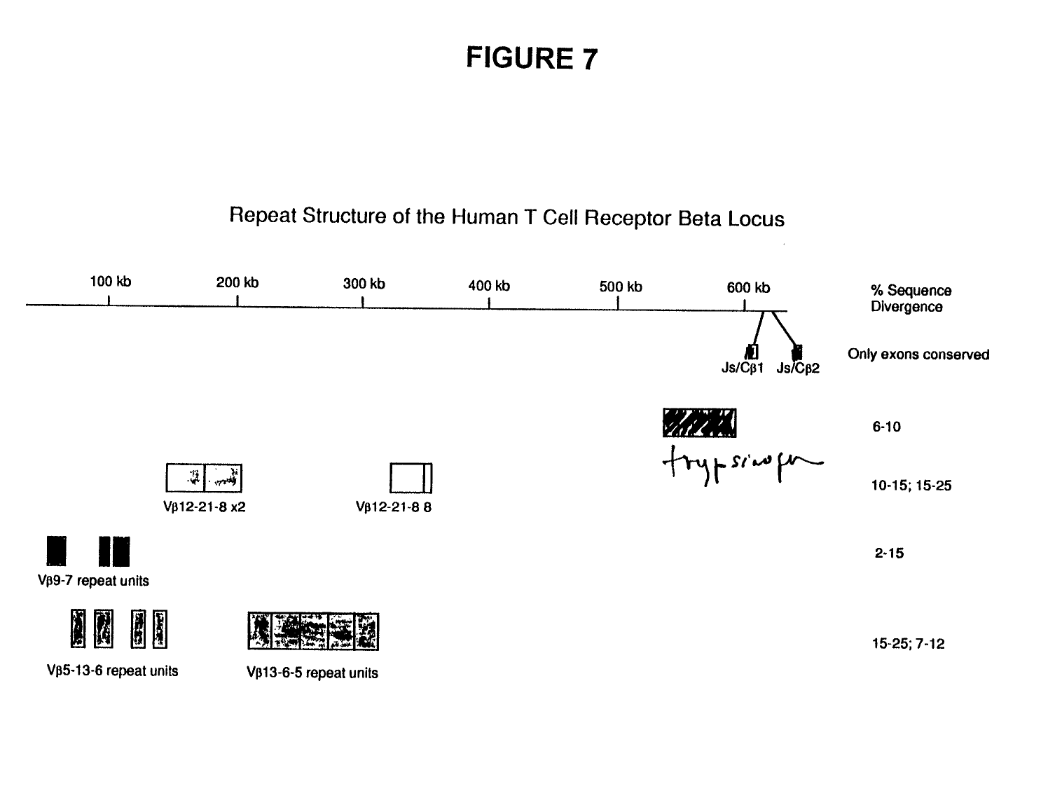

The T cell receptor Vβ gene locus is composed of approximately 685 kb of DNA, and contains multiple gene segments that encode variable (V), diversity (D), joining (J), and constant (C) regions. The general location of the genes is shown in FIG. 6. In particular, as is shown in FIG. 4, the gene locus is composed of 67 V gene segments, 2 D gene segments, 13 functional J gene segments, and 2 C gene segments. The C genes are called Cβ1 and Cβ2.

The V gene segments are segregated into 34 subfamilies. A V gene segment which has 75% or greater nucleotide sequence identity to another V gene is placed in the same subfamily. As an example, TCRBV6S3 is a gene name for the third gene segment of subfamily 6. Most of the subfamilies have a single gene segment member. Subfamily 9 has two members, subfamilies 7, 12, and 21 each have three gene segment numbers, subfamily 8 has five gene segment members, subfamily 5 has seven gene segment members, and subfamilies 6 and 13 each have nine gene segment members.

The order of the V gene segments on the chromosome is shown in FIG. 4, which presents a map of the Vβ locus. Briefly, the 5′ most V gene segment is TCRBV27S1 and the 3′ most gene segment is TCRBV20S1. TCRBV20S1 is located 3′ of the C regions and in the opposite transcriptional orientation. All the other V gene segments are in the same transcriptional orientation. One notable feature of the order of the V gene segments is the random interspersion of subfamily members. Thus, the nine members of the TCRBV6 and TCRBV13 gene families are dispersed across the locus. In addition to the TCR gene segments, six trypsinogen genes are located within this locus as well.

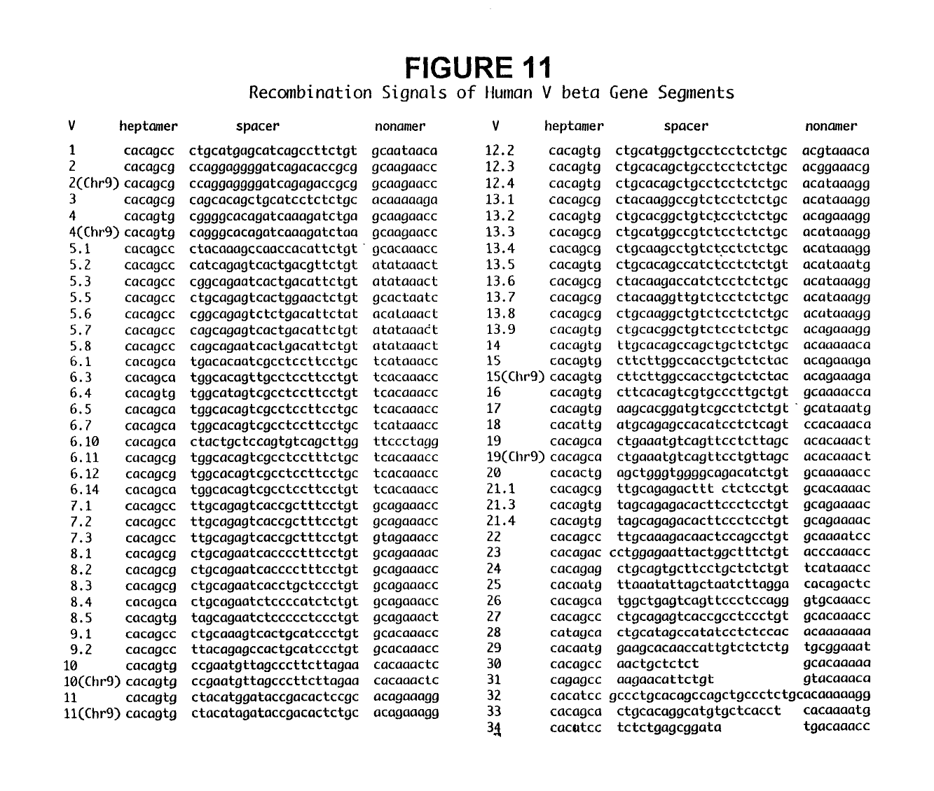

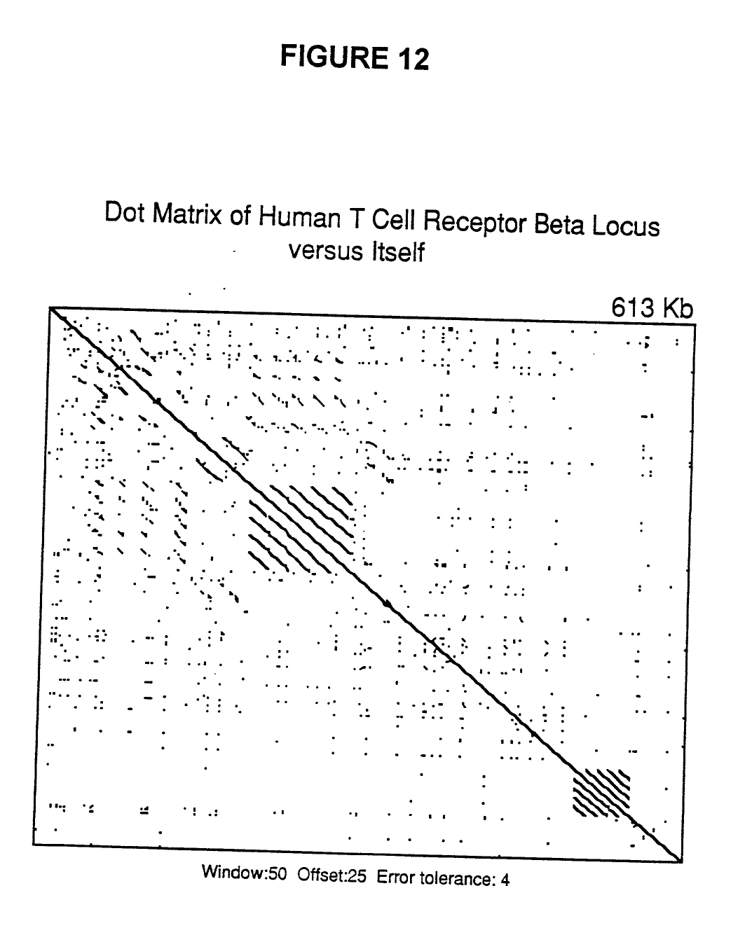

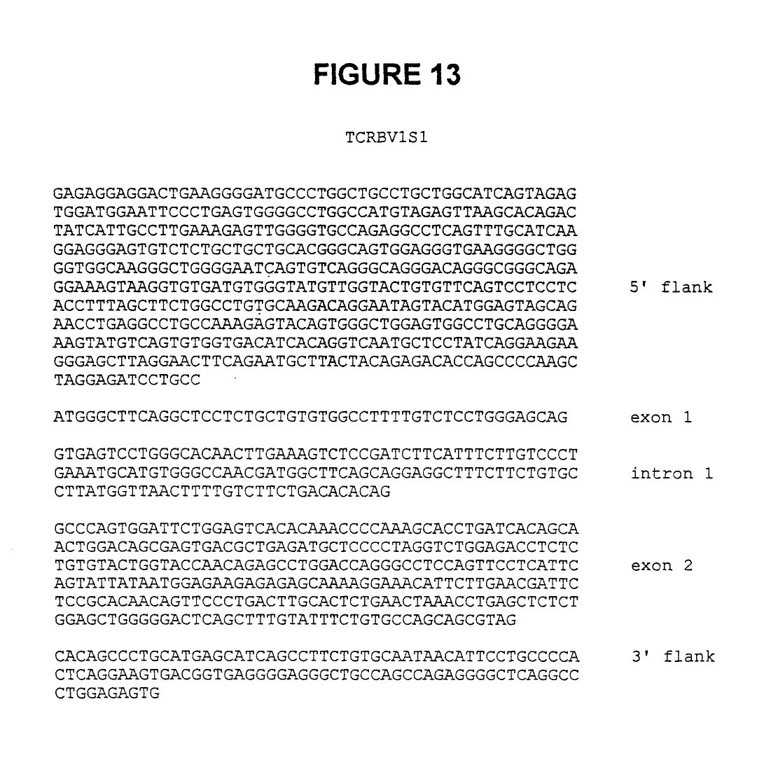

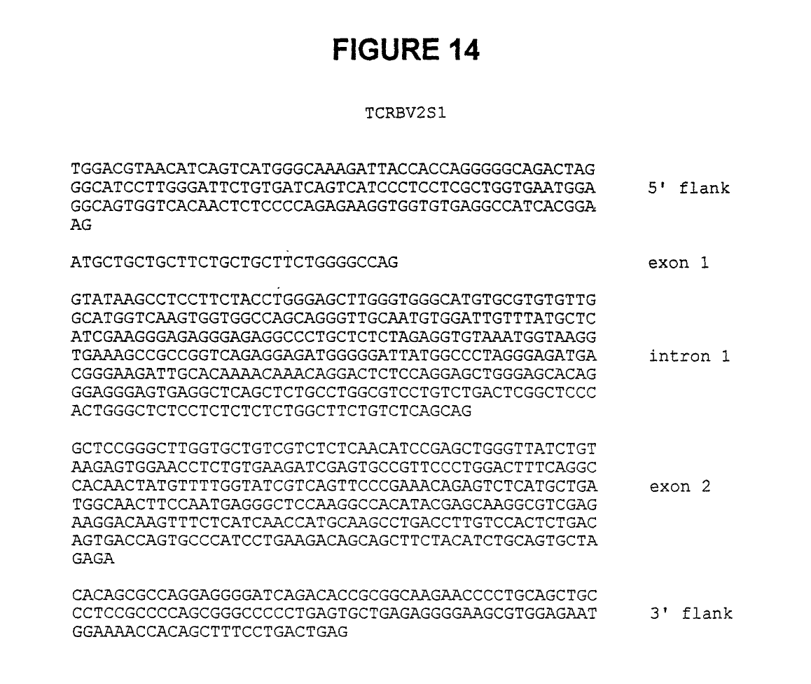

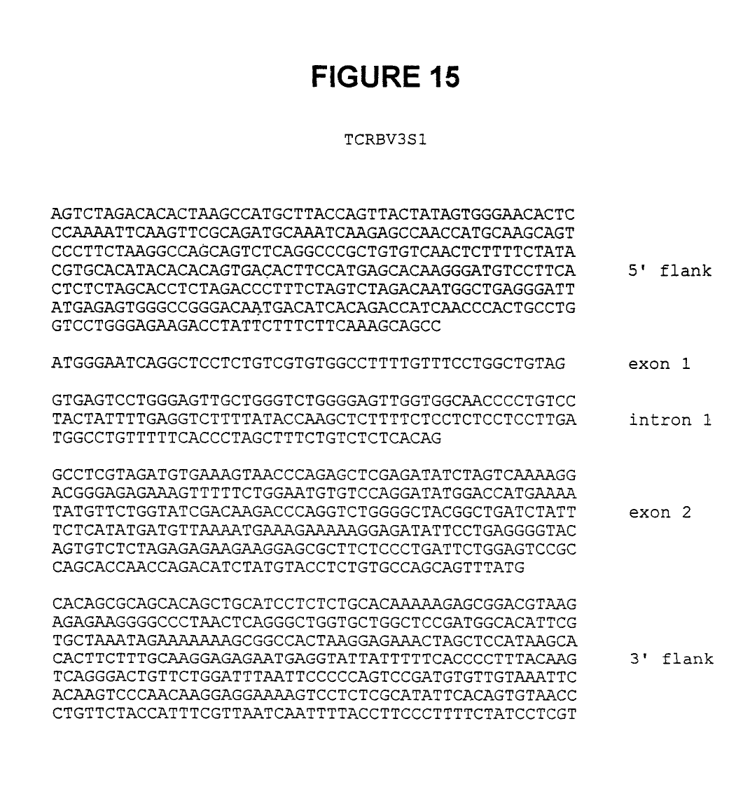

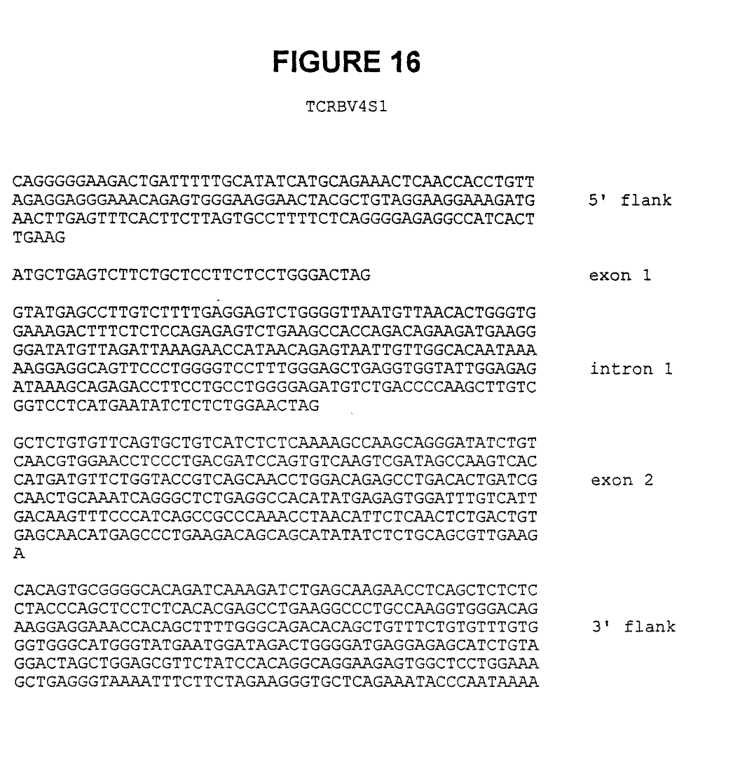

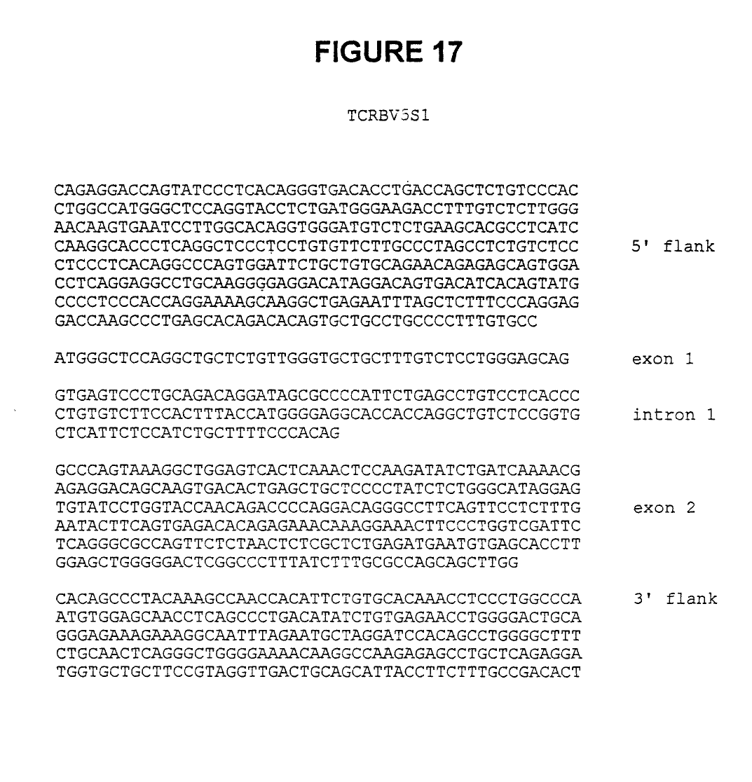

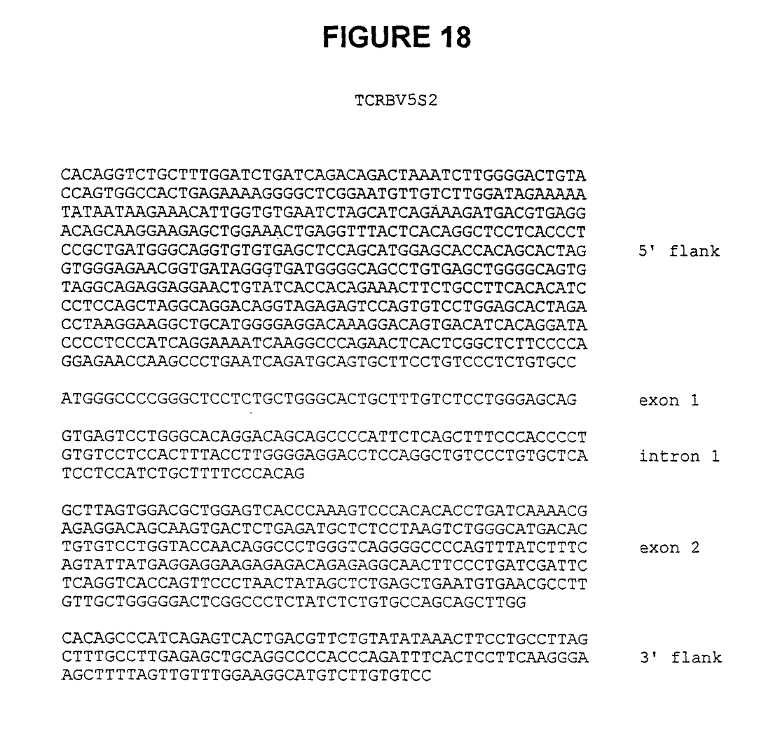

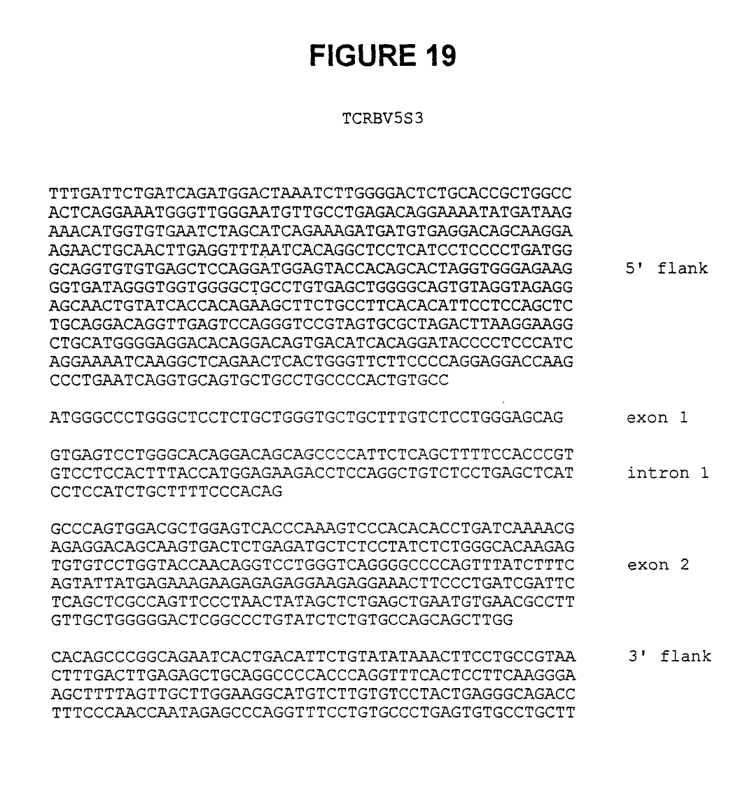

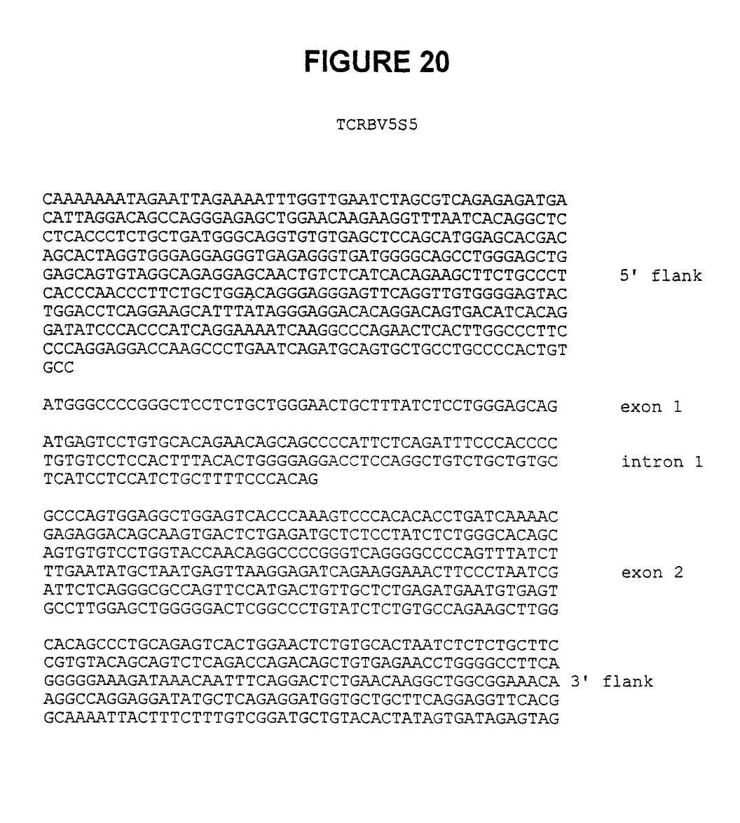

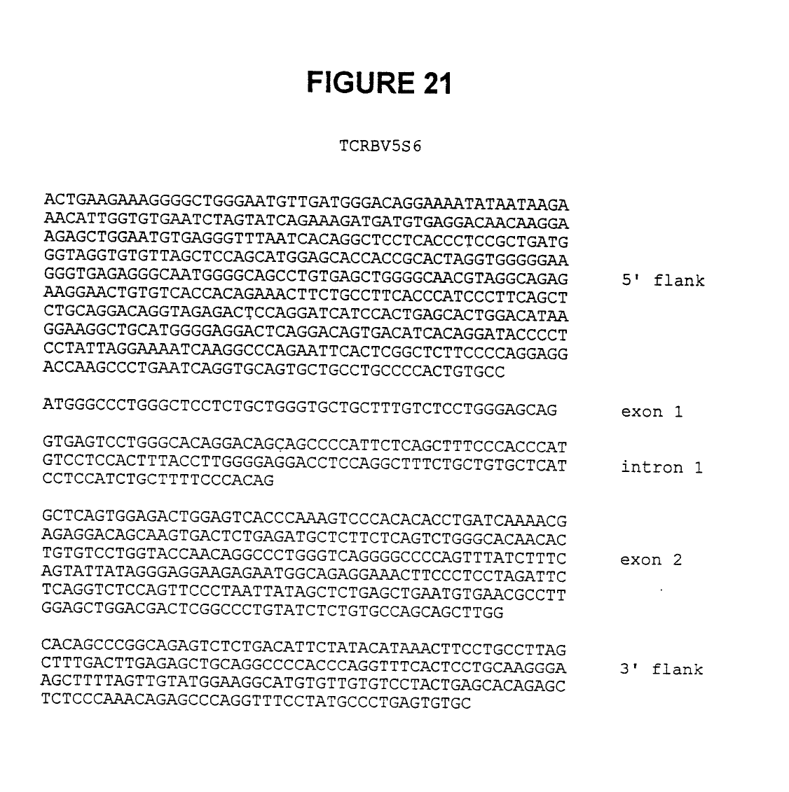

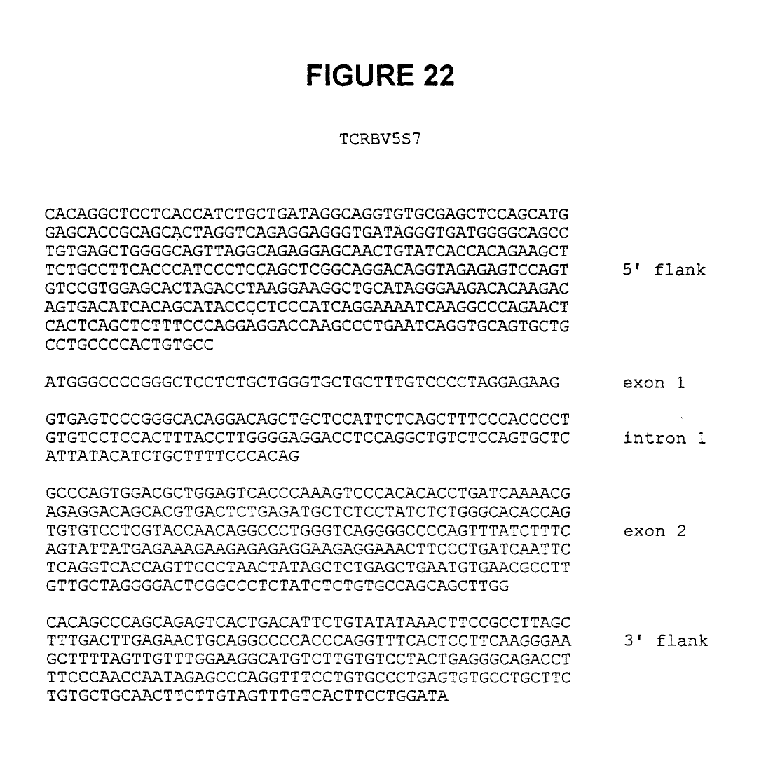

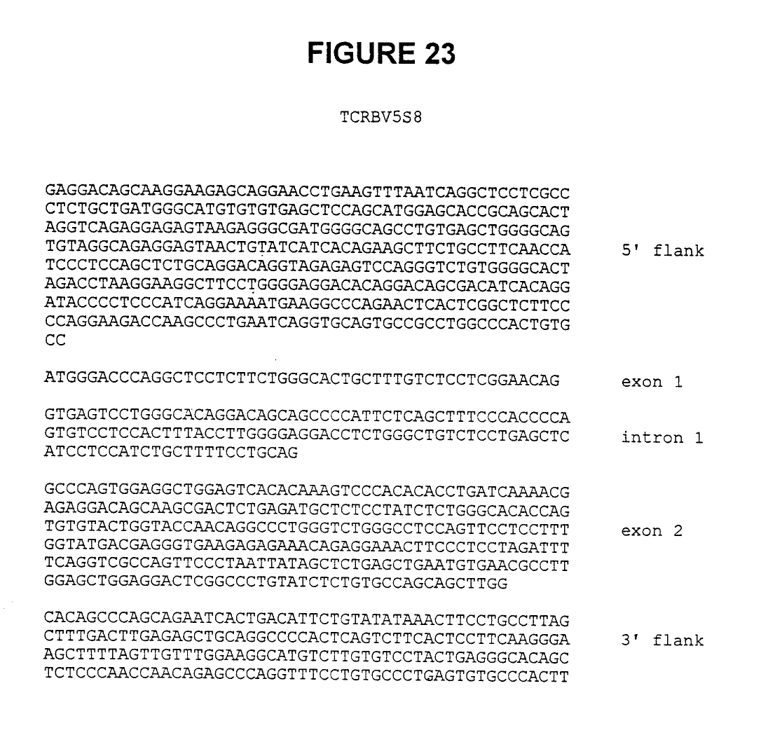

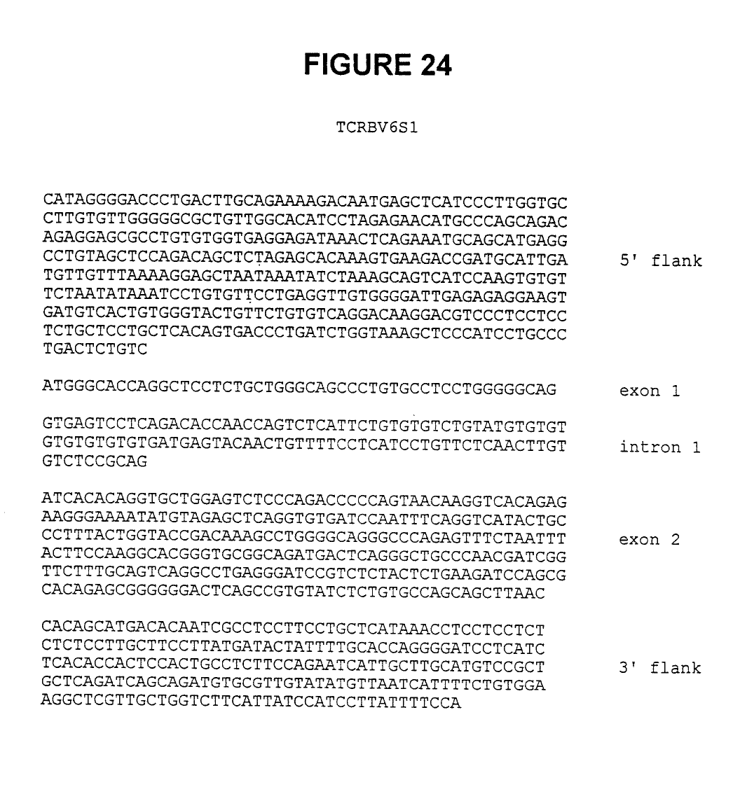

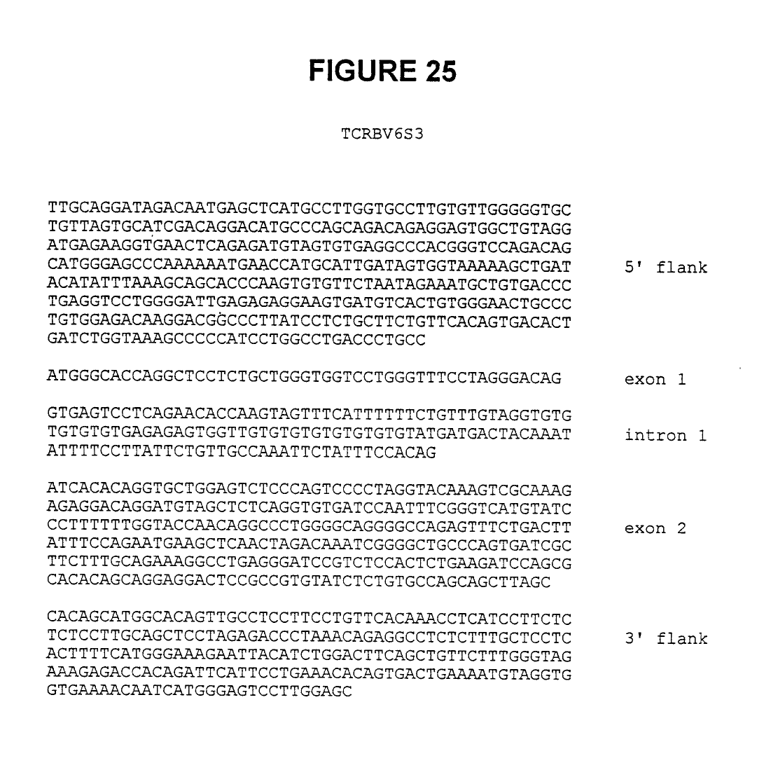

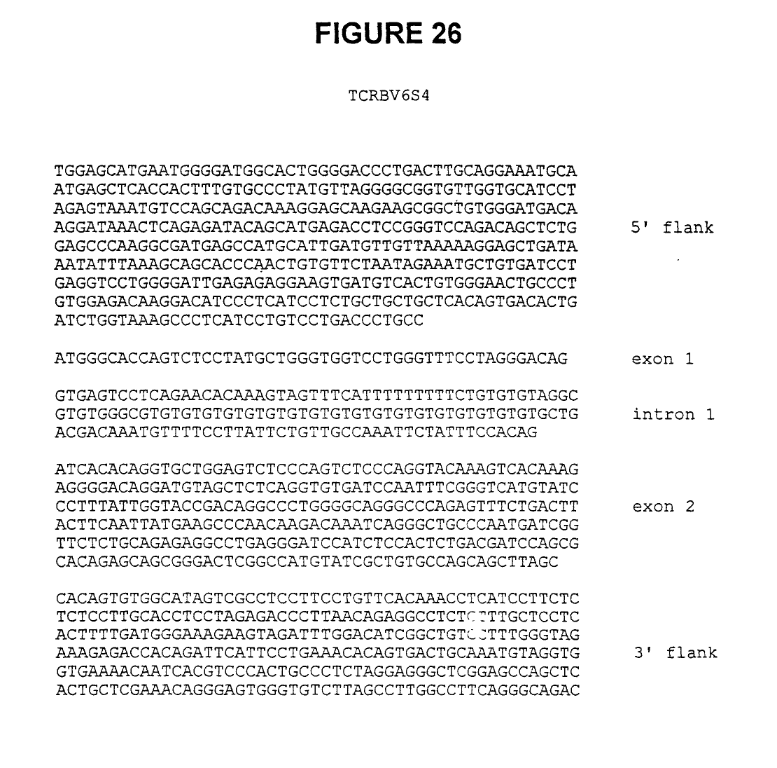

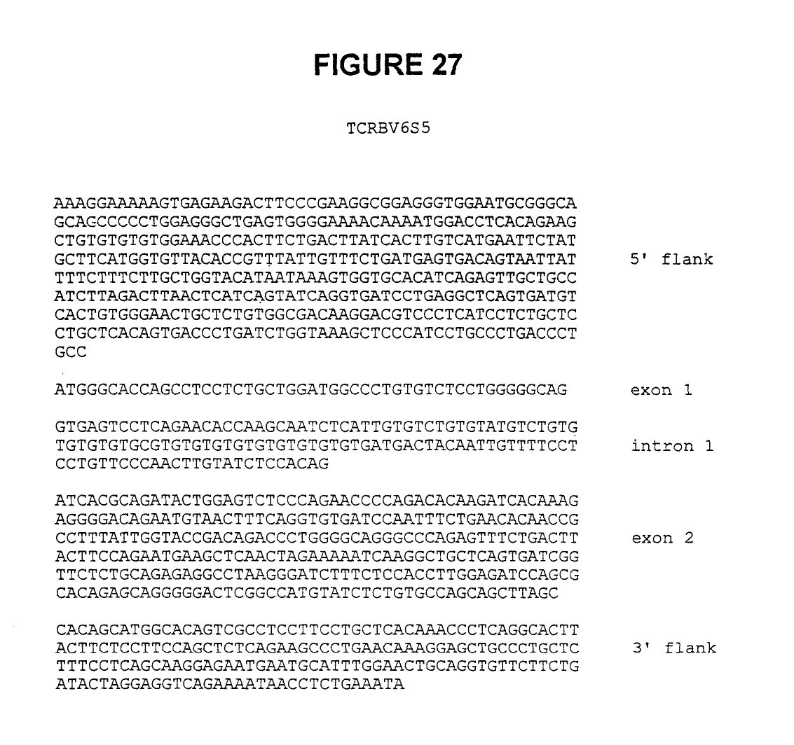

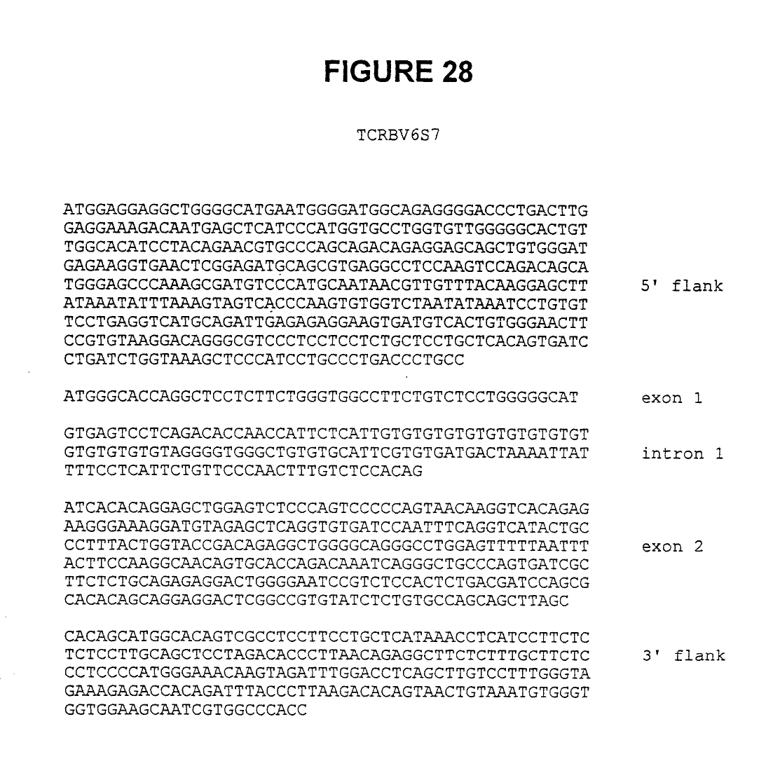

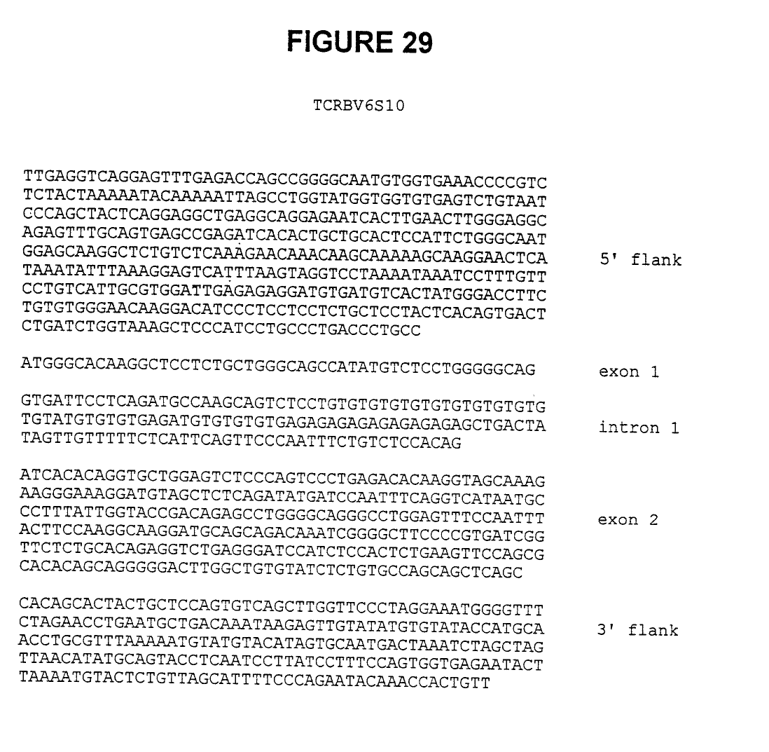

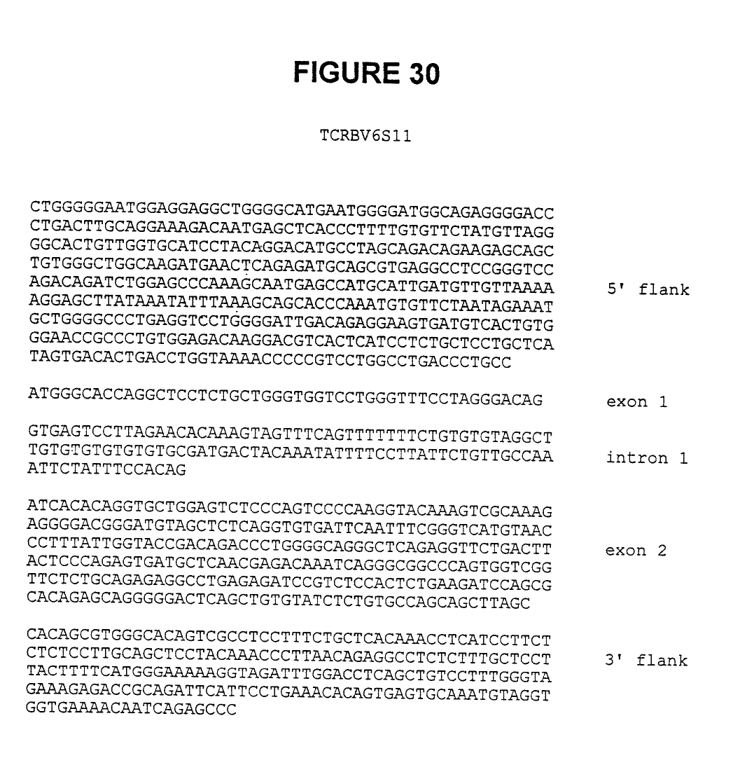

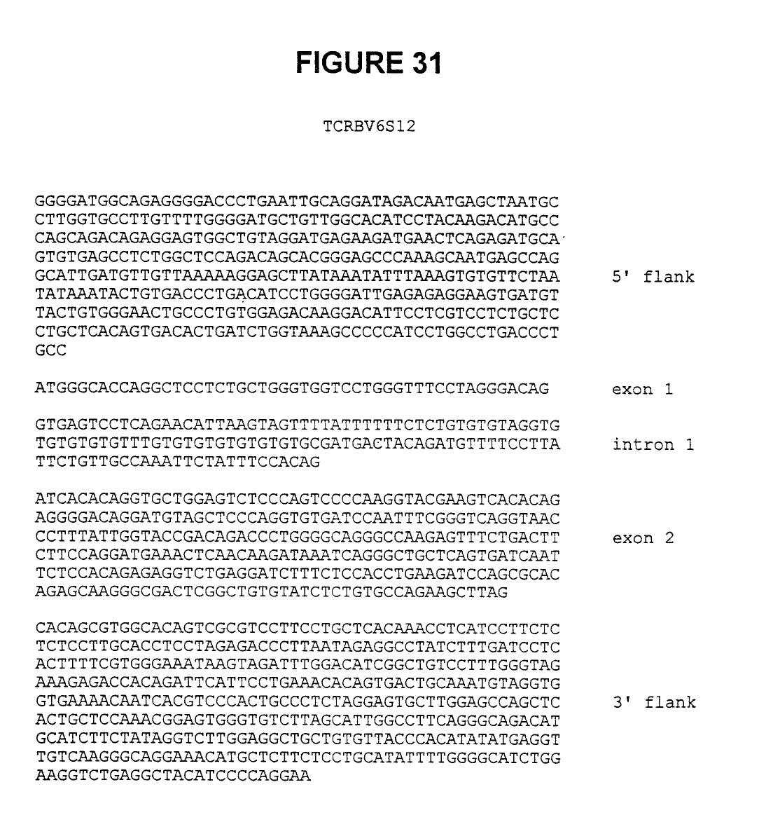

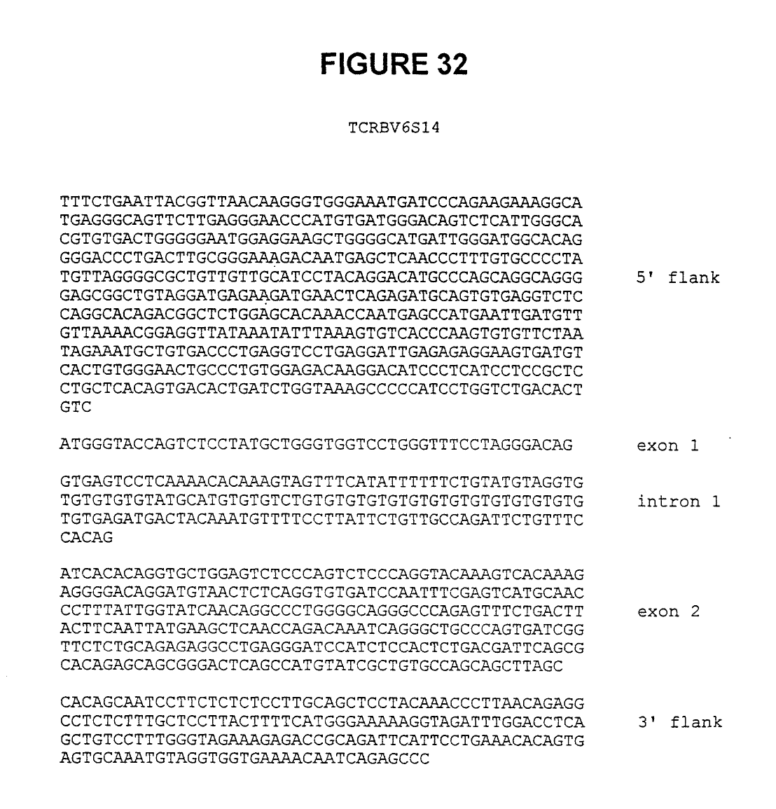

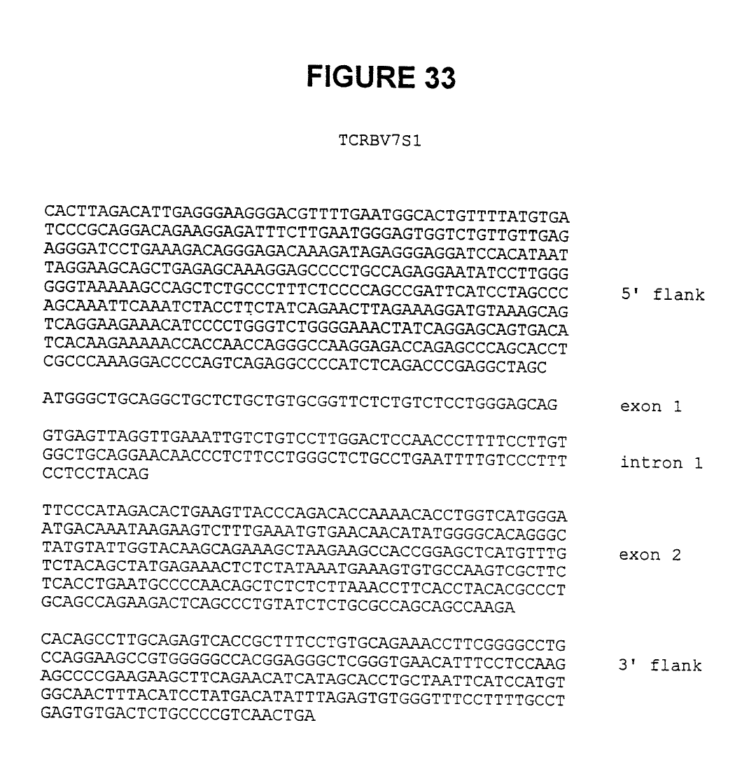

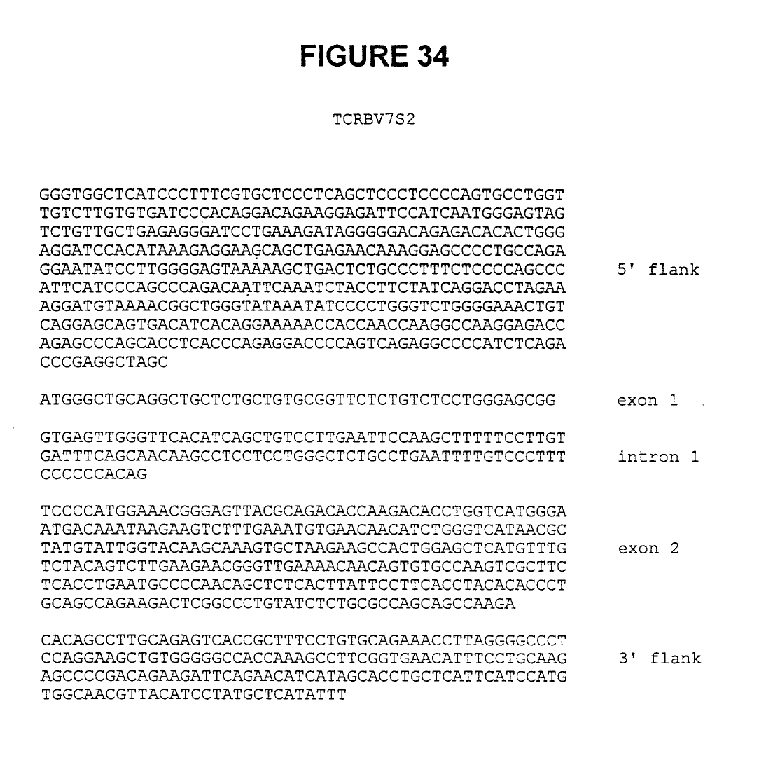

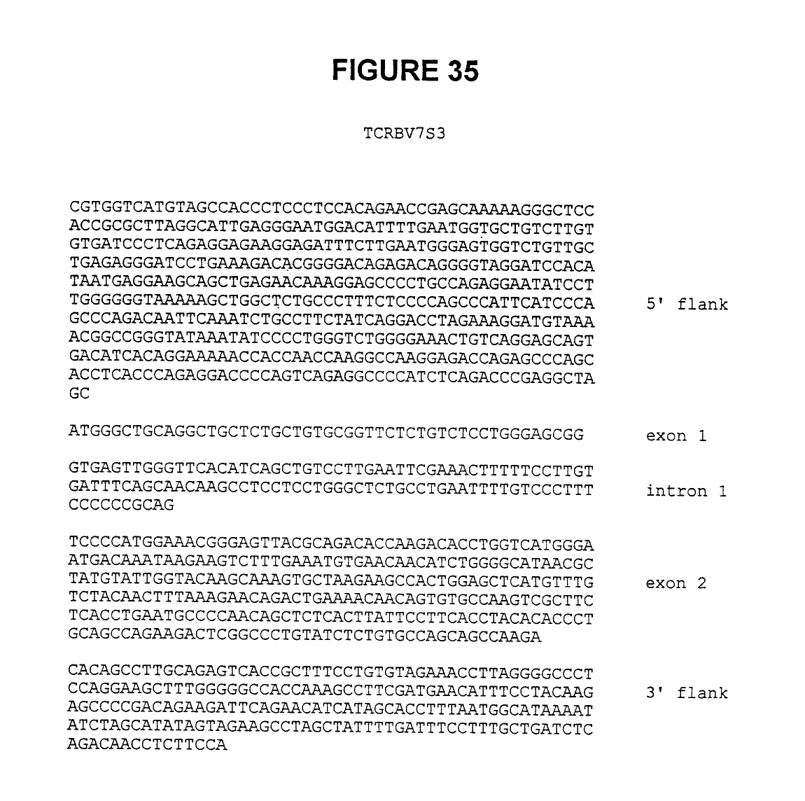

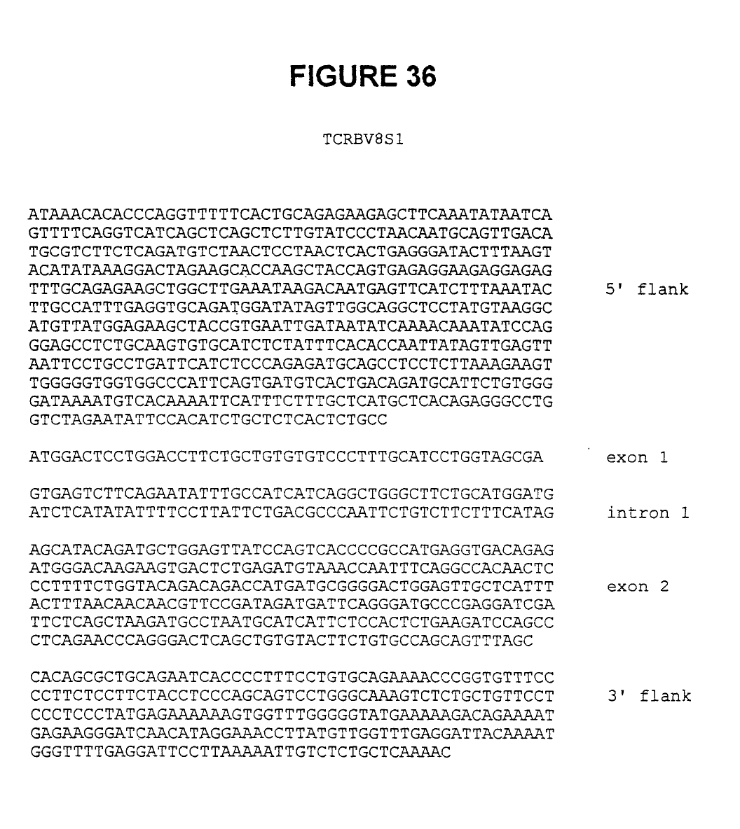

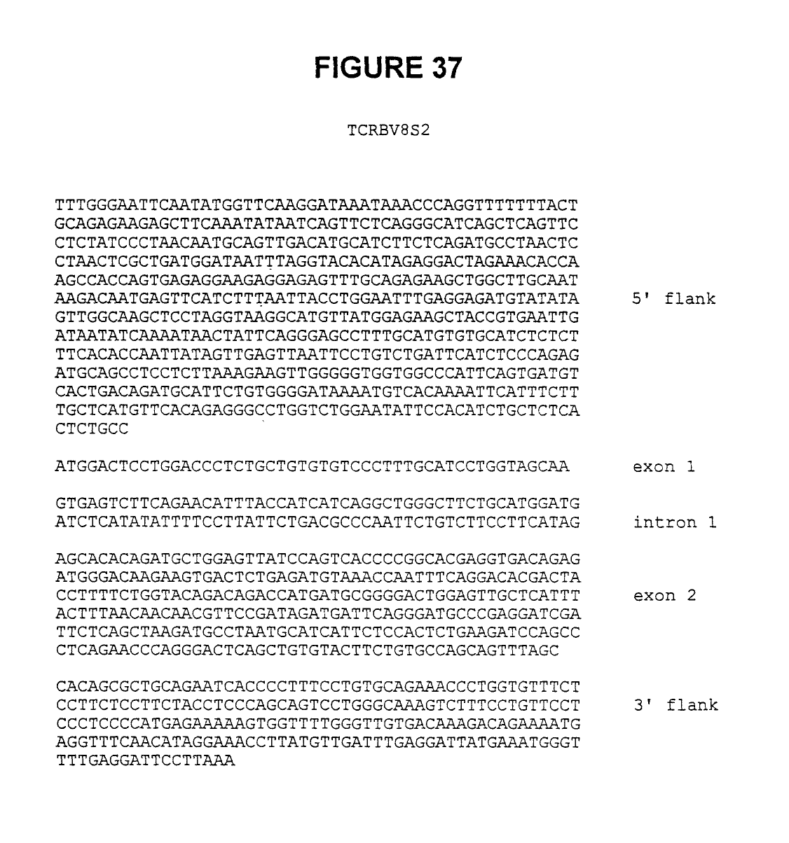

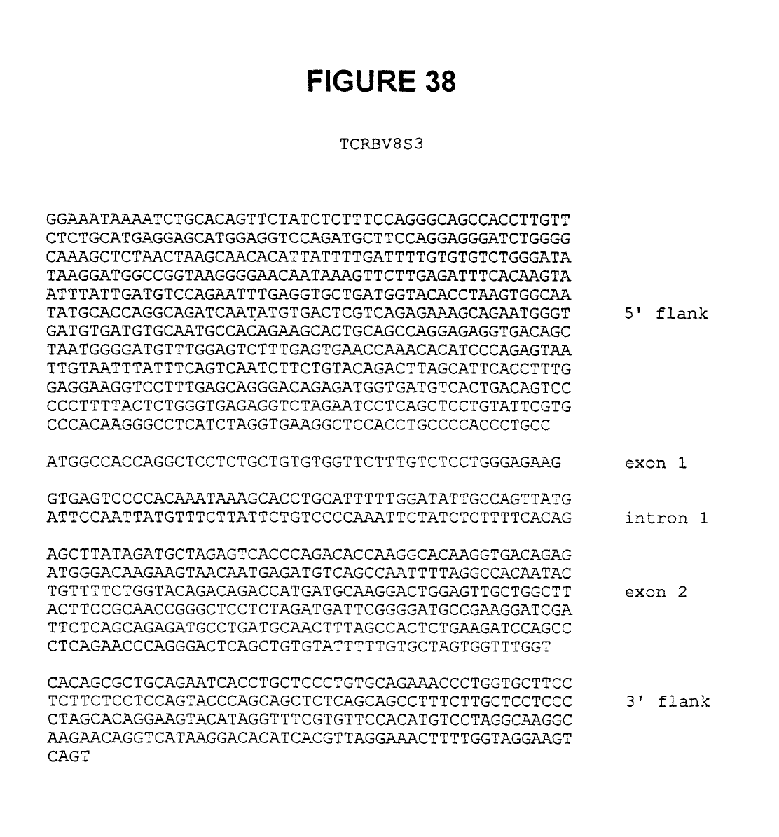

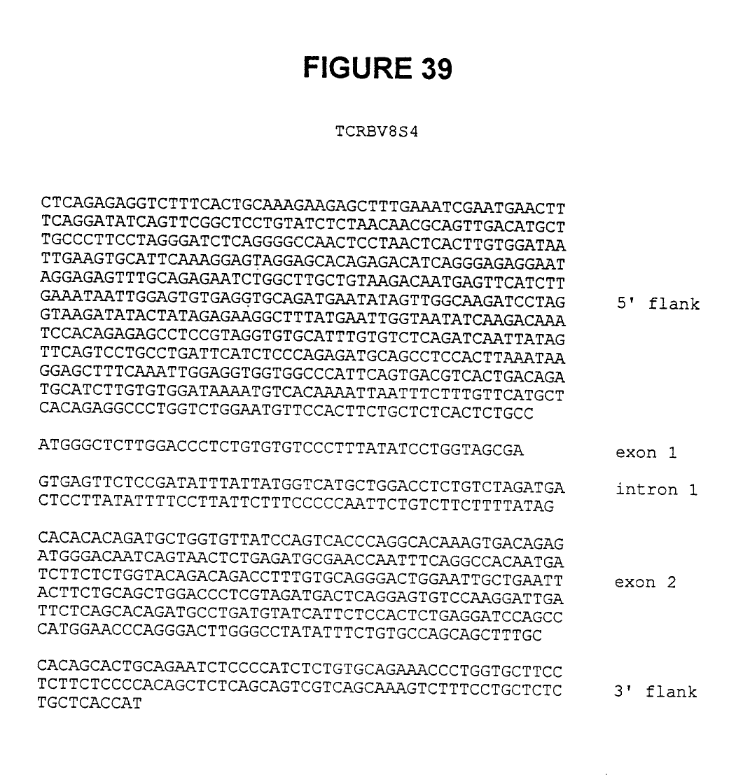

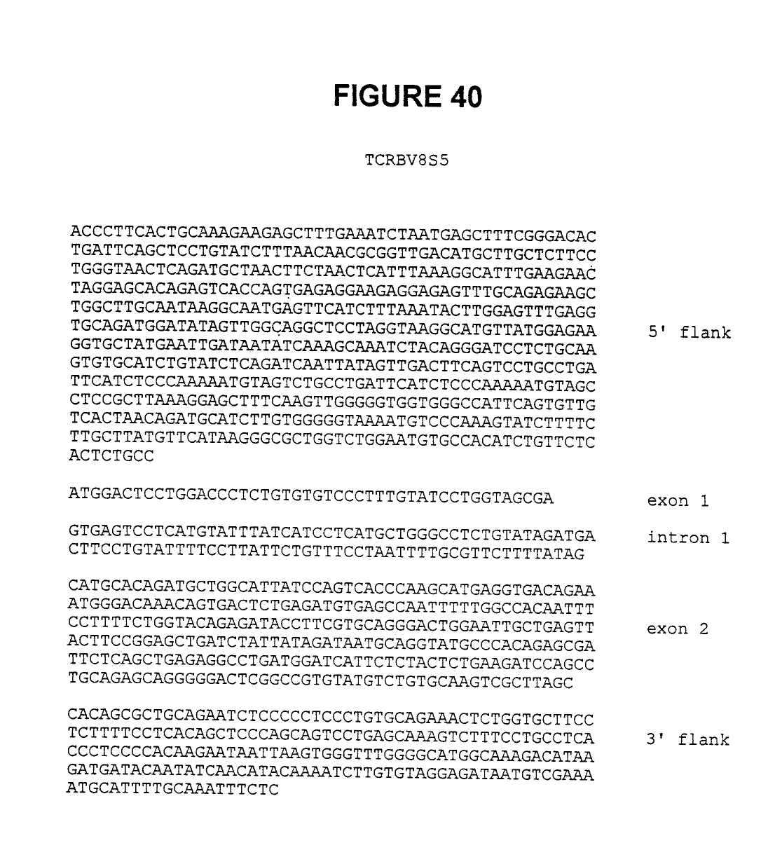

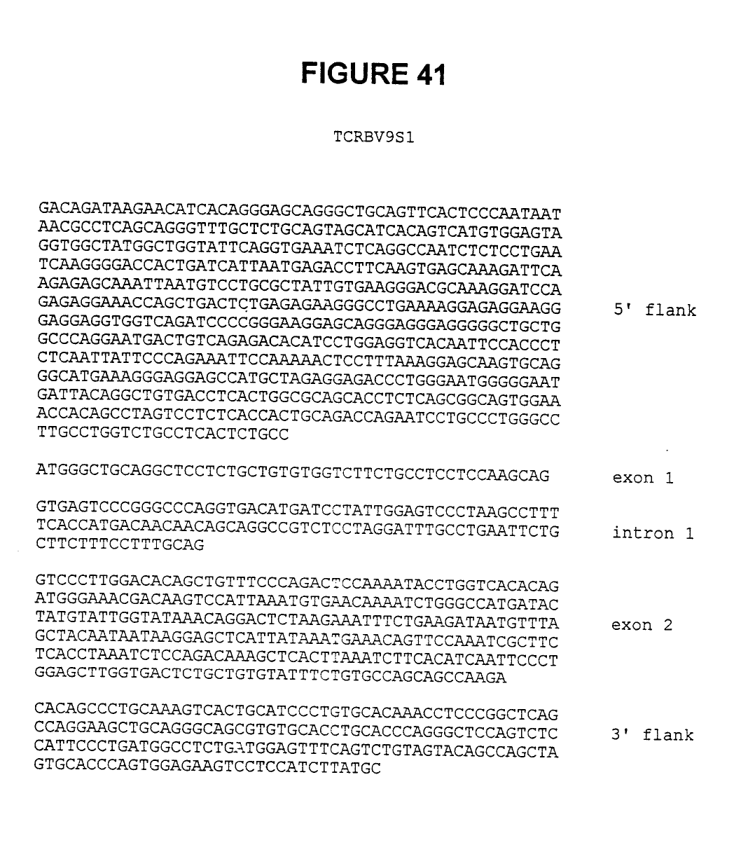

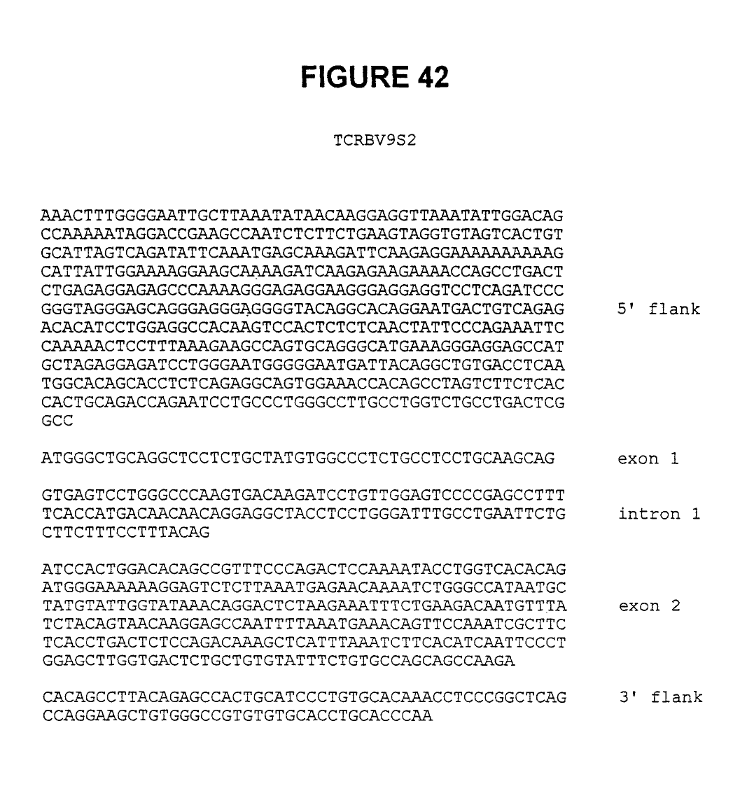

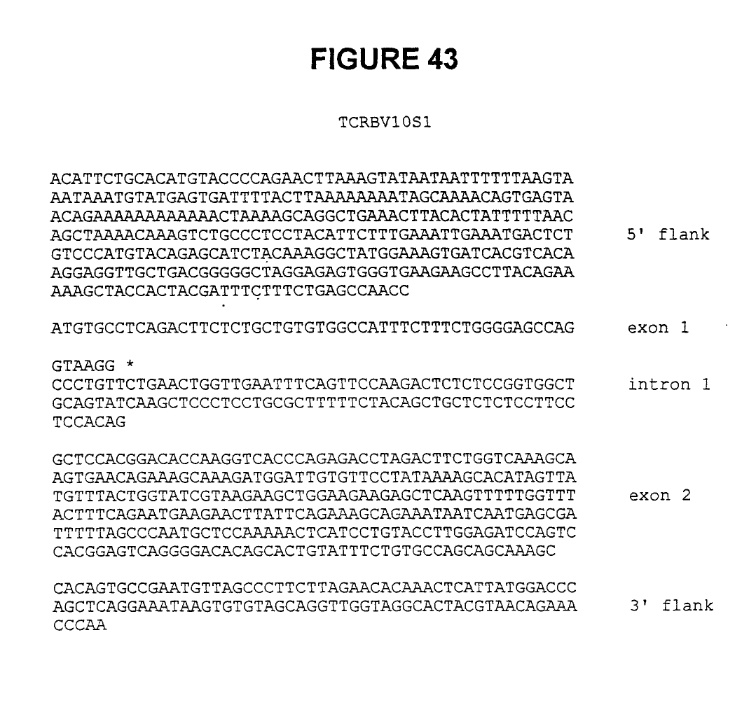

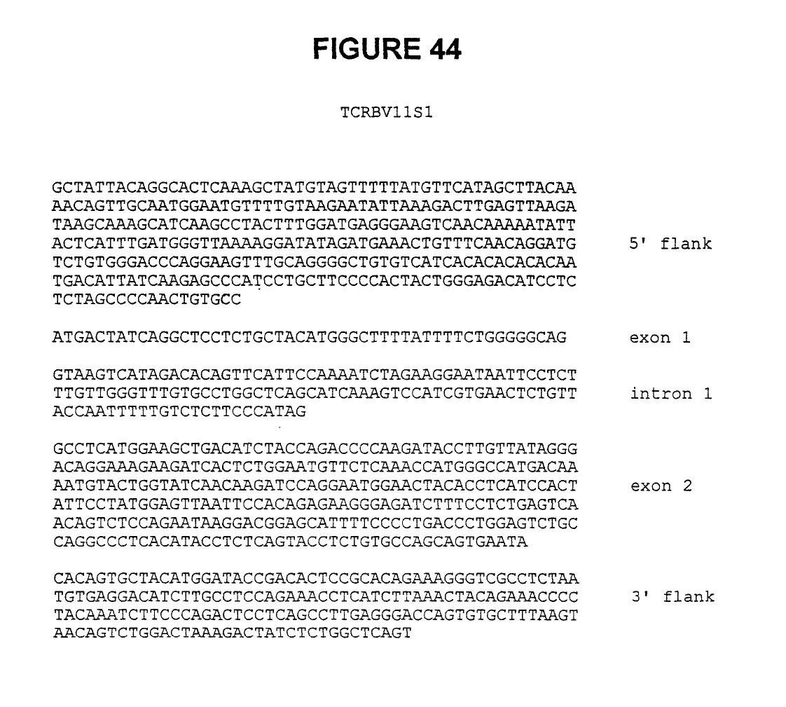

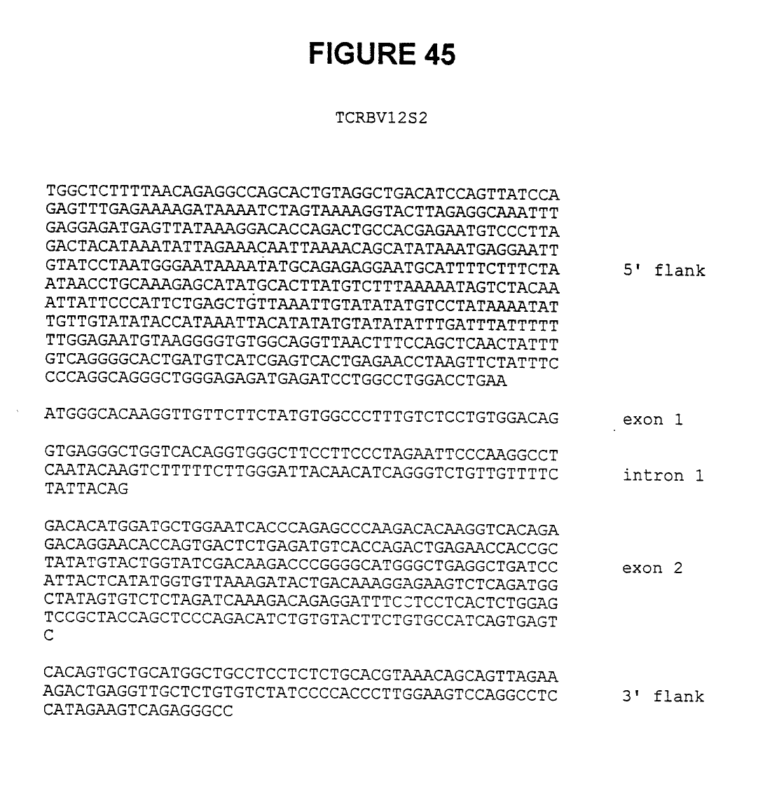

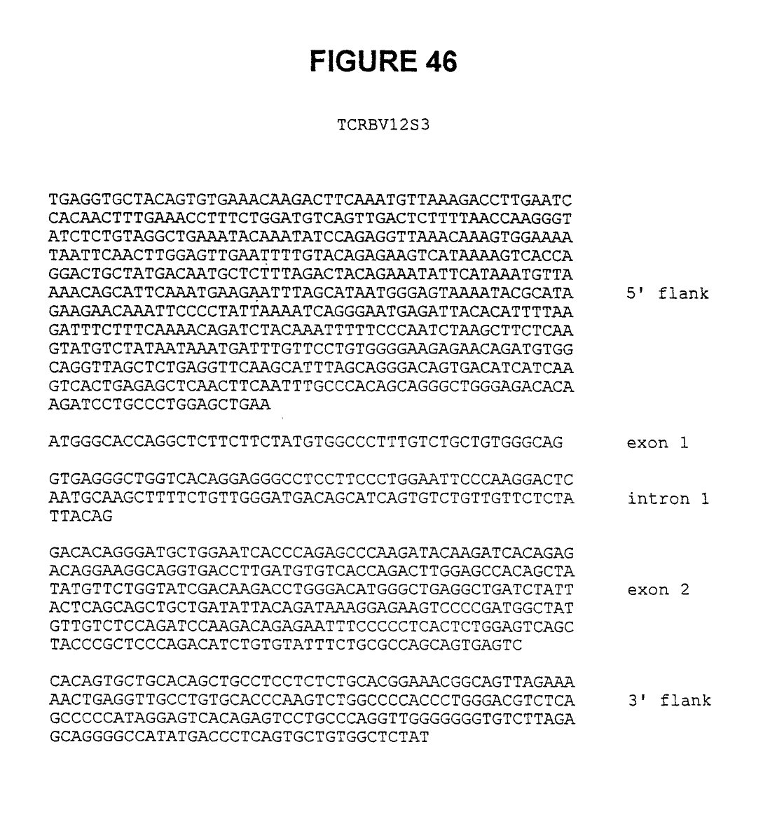

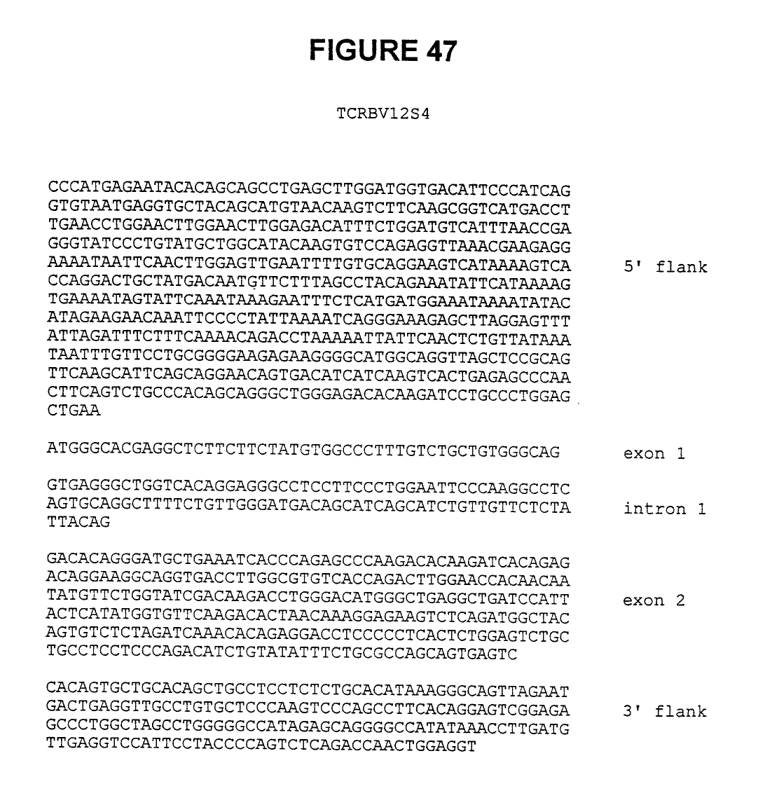

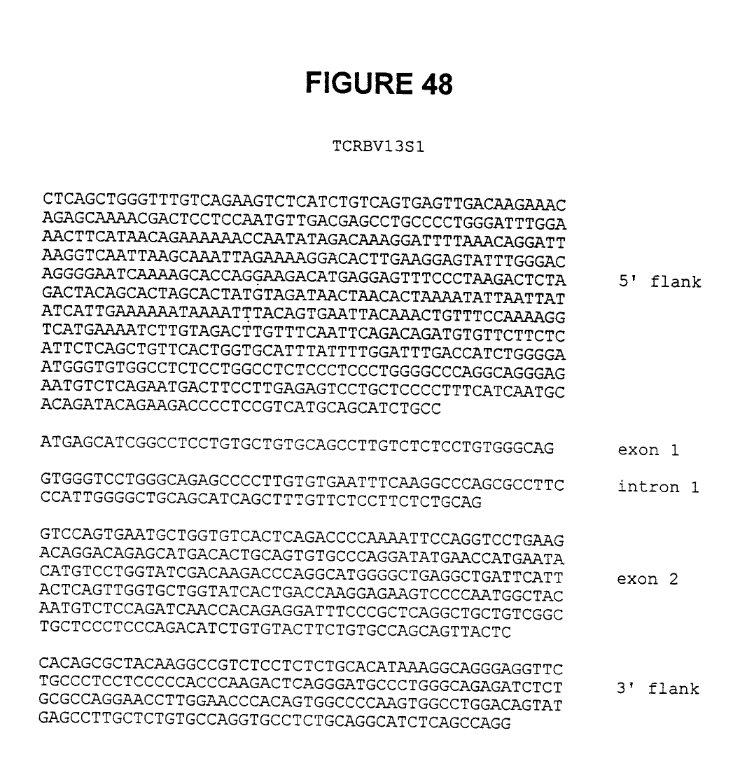

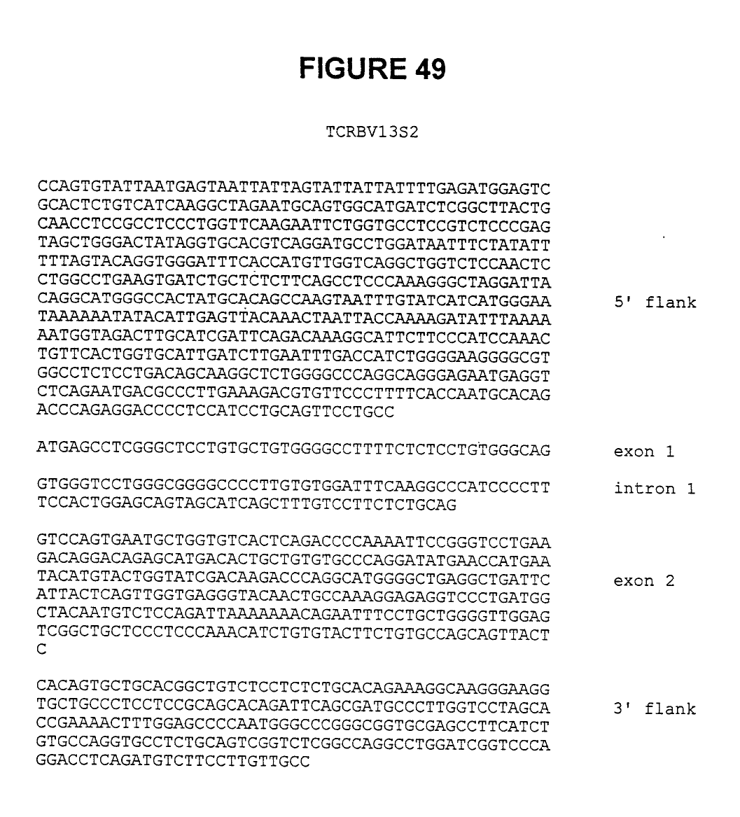

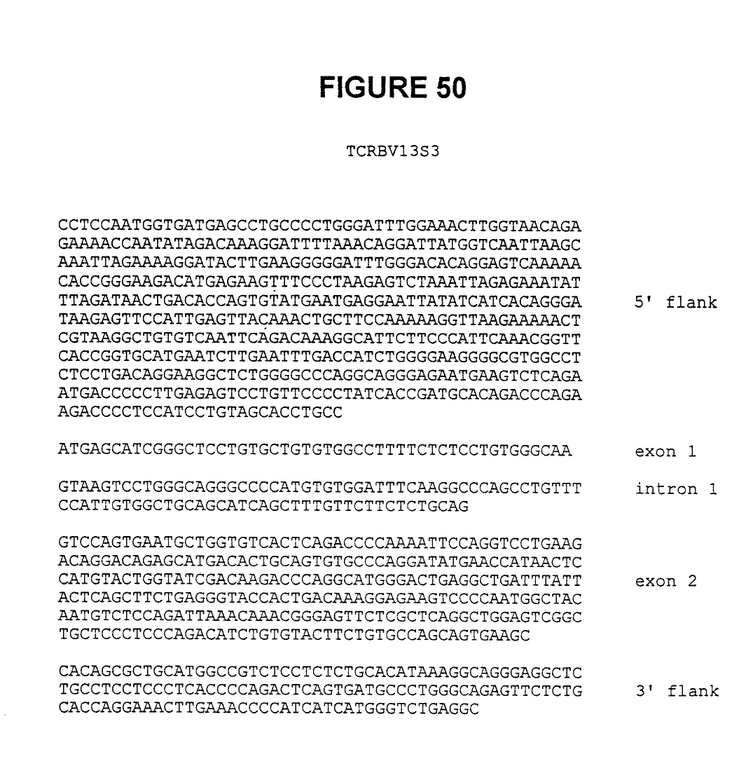

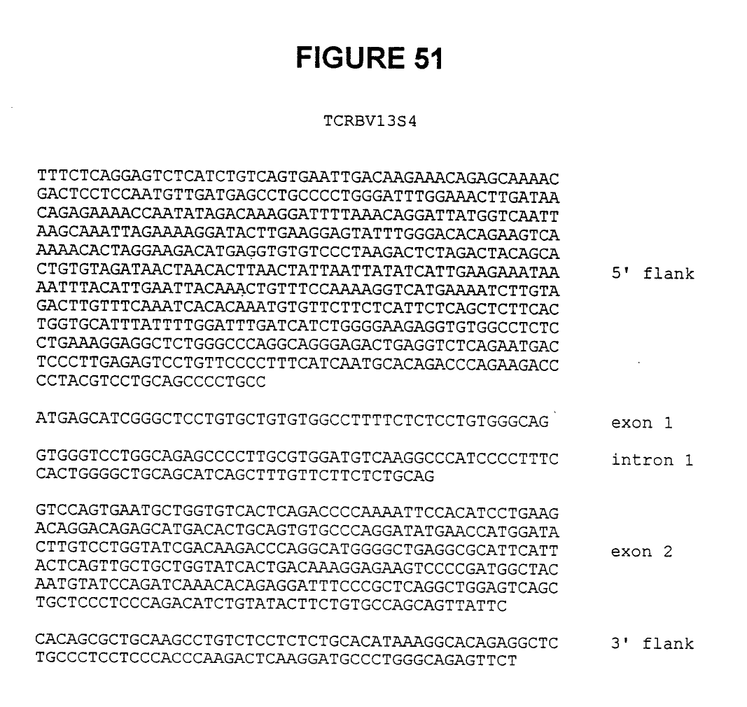

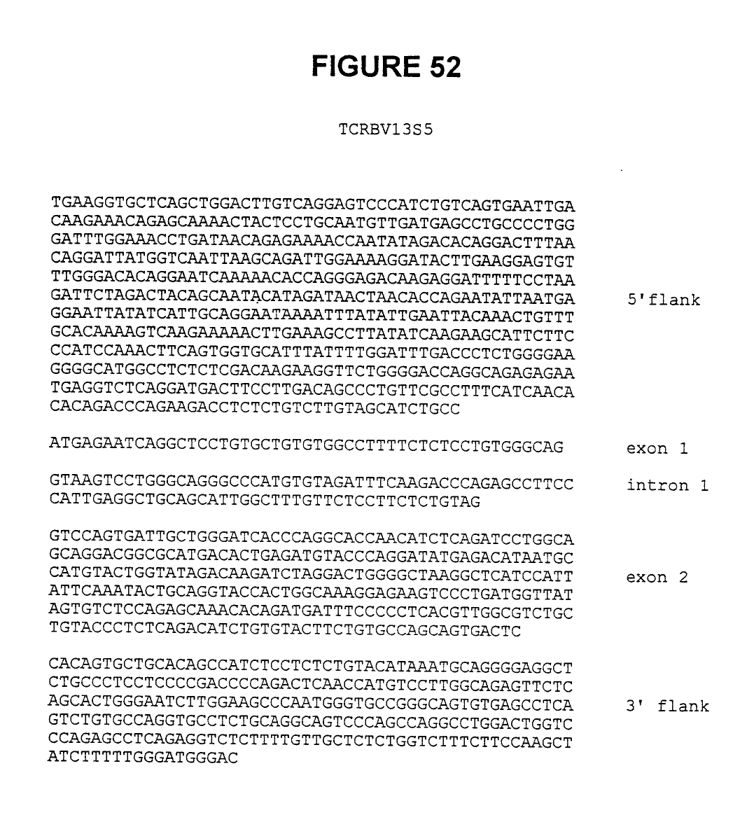

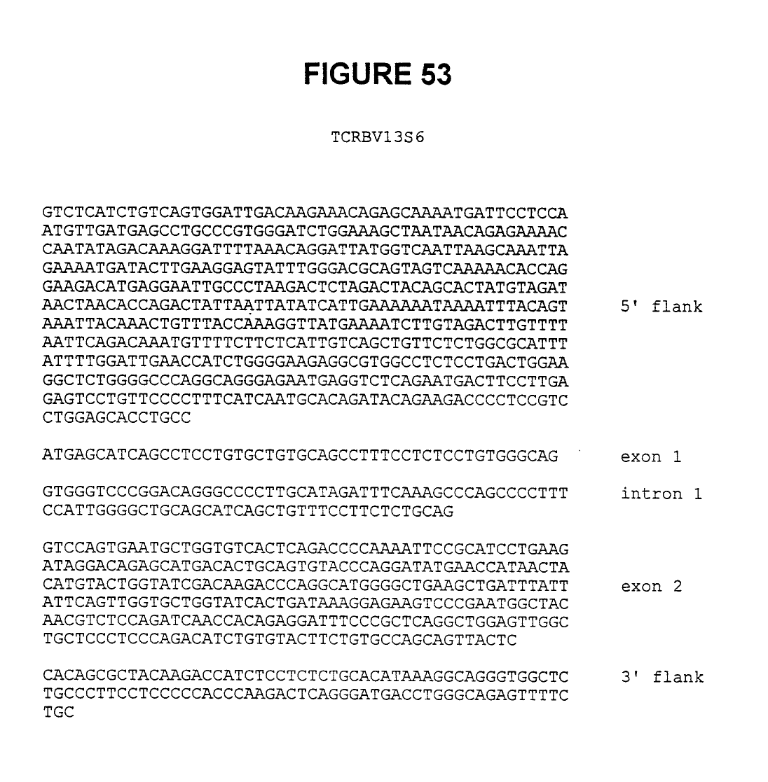

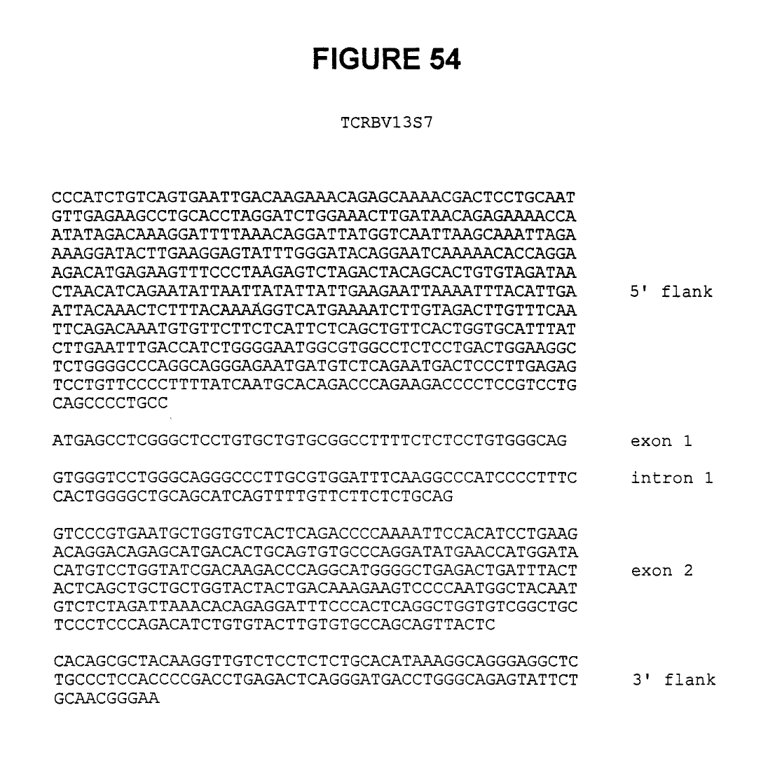

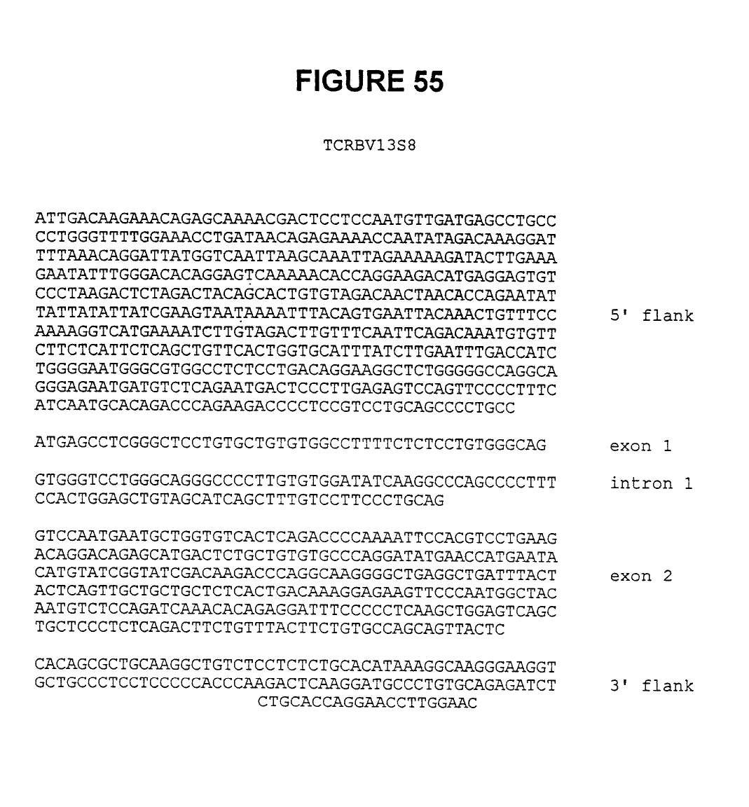

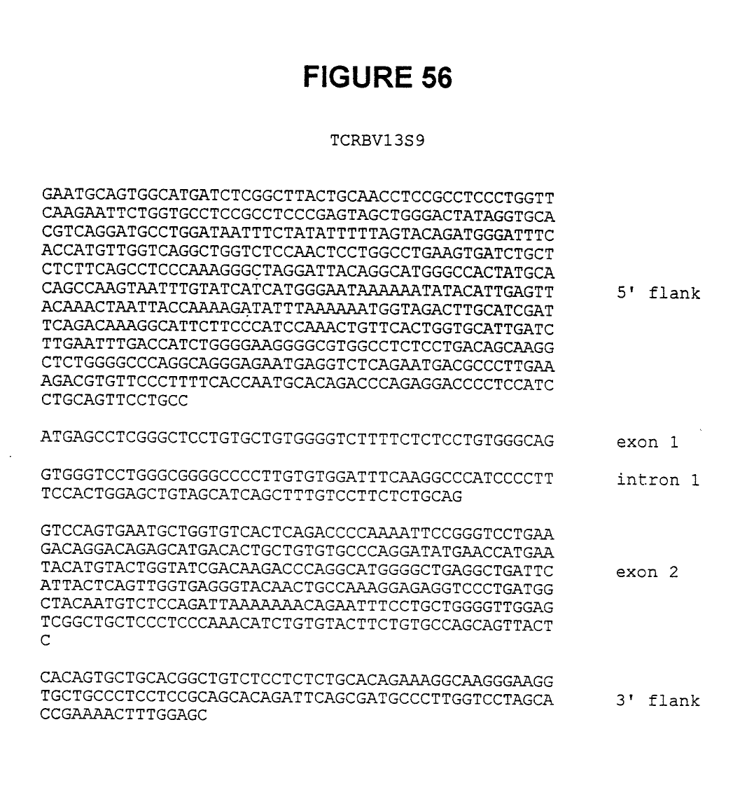

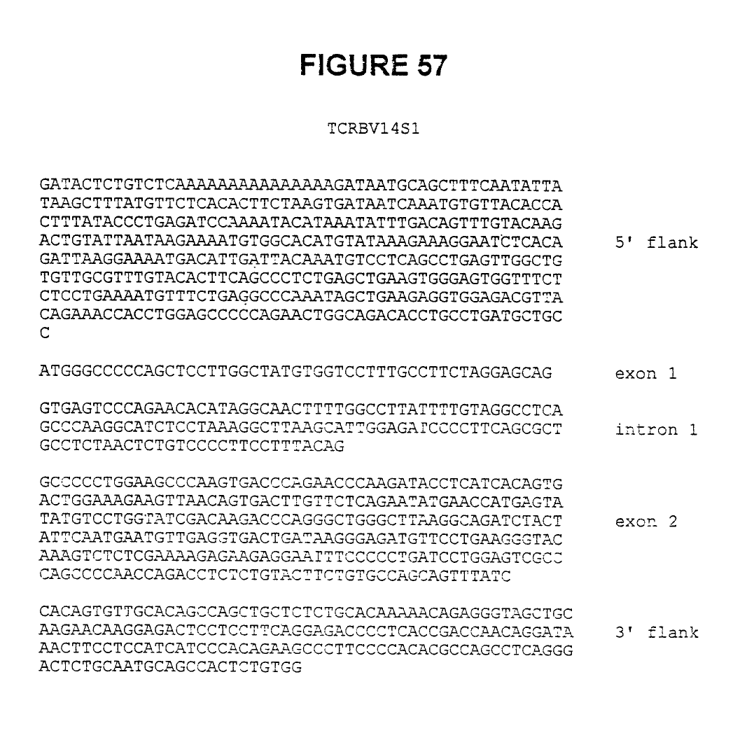

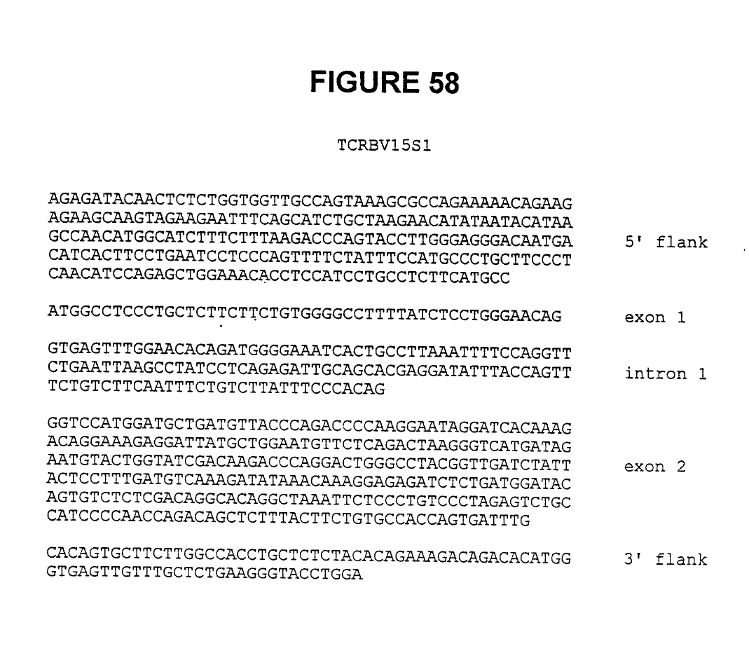

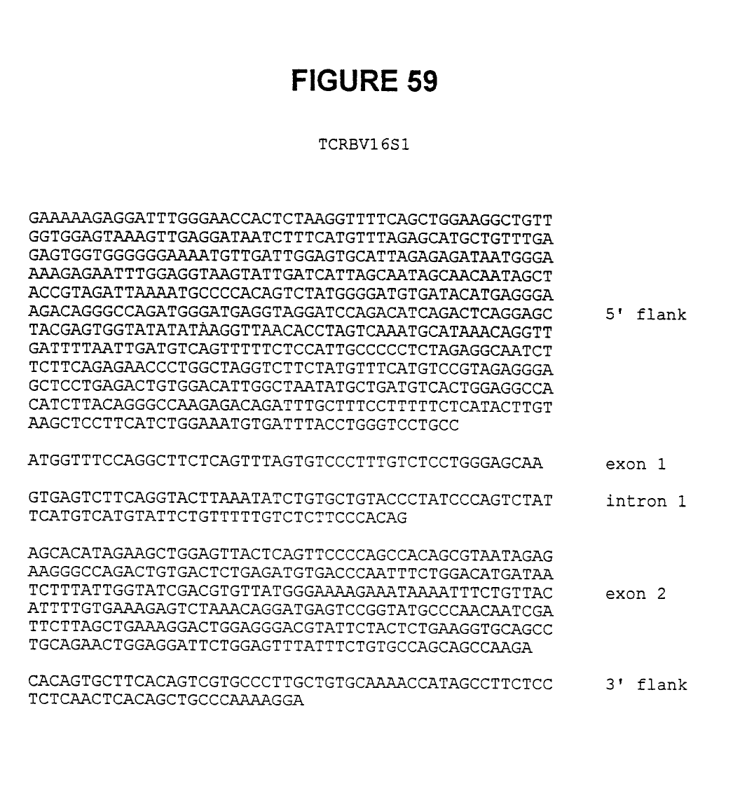

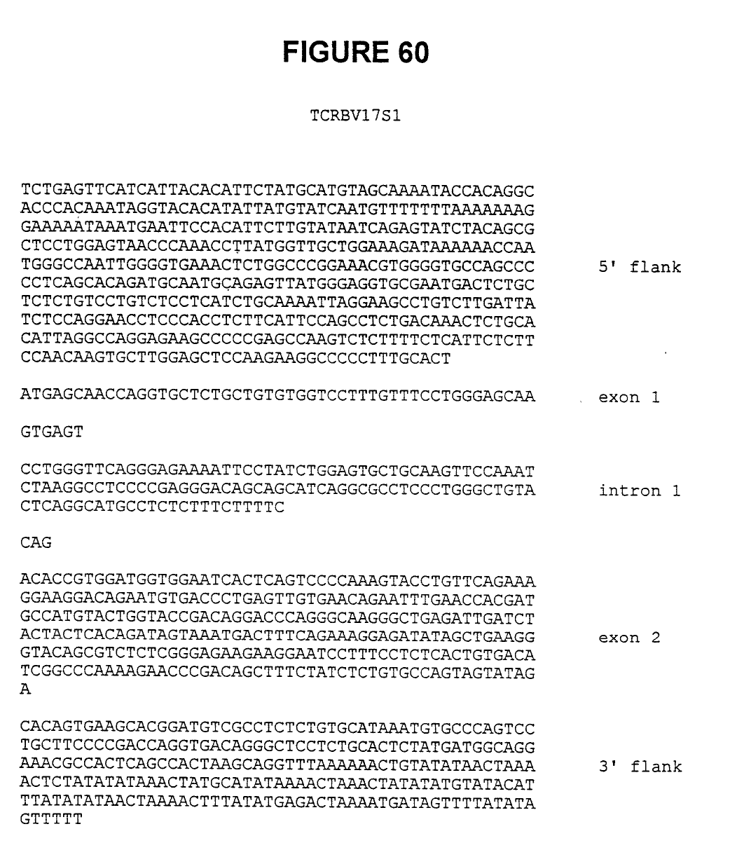

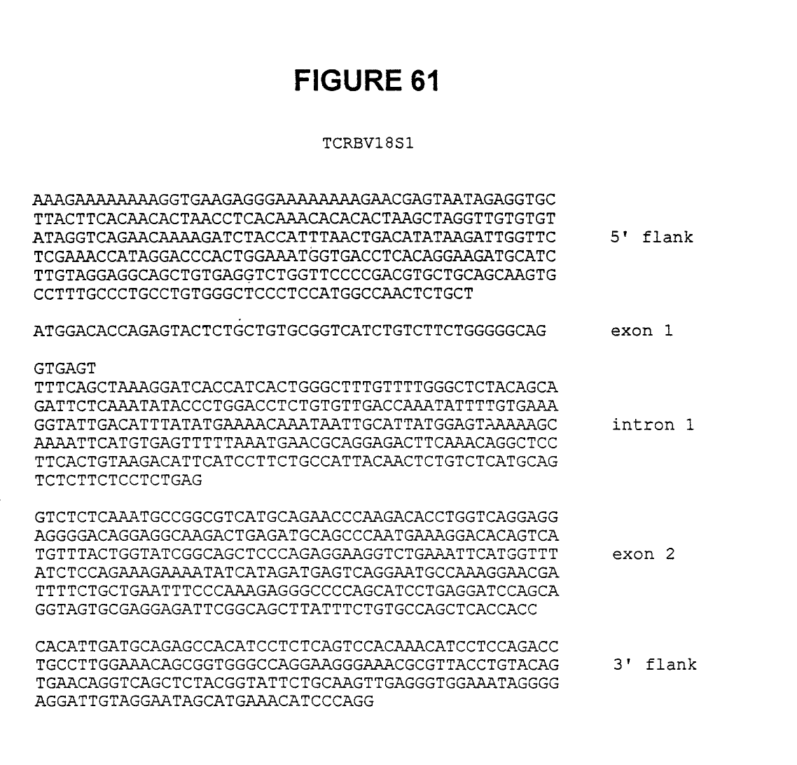

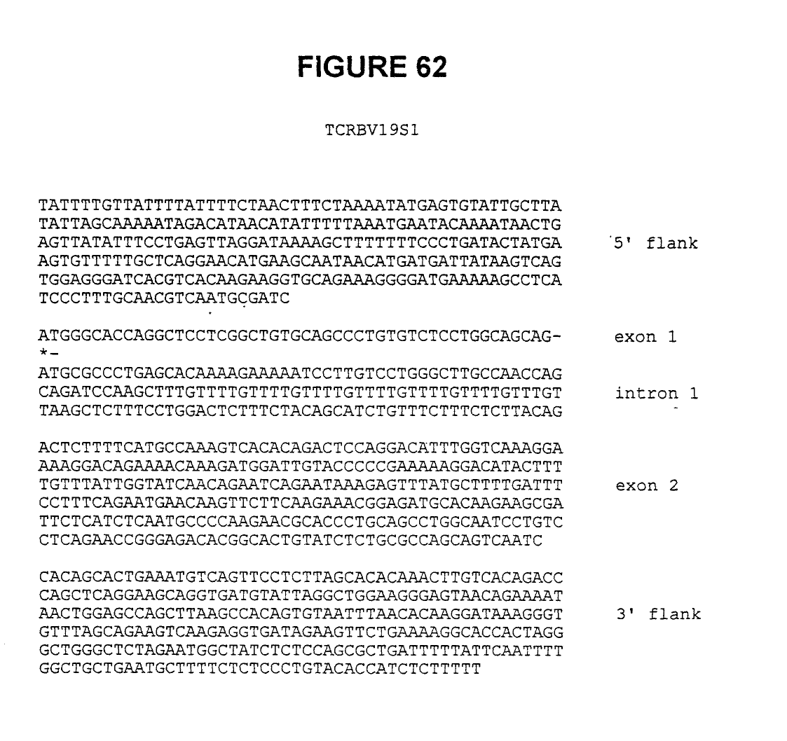

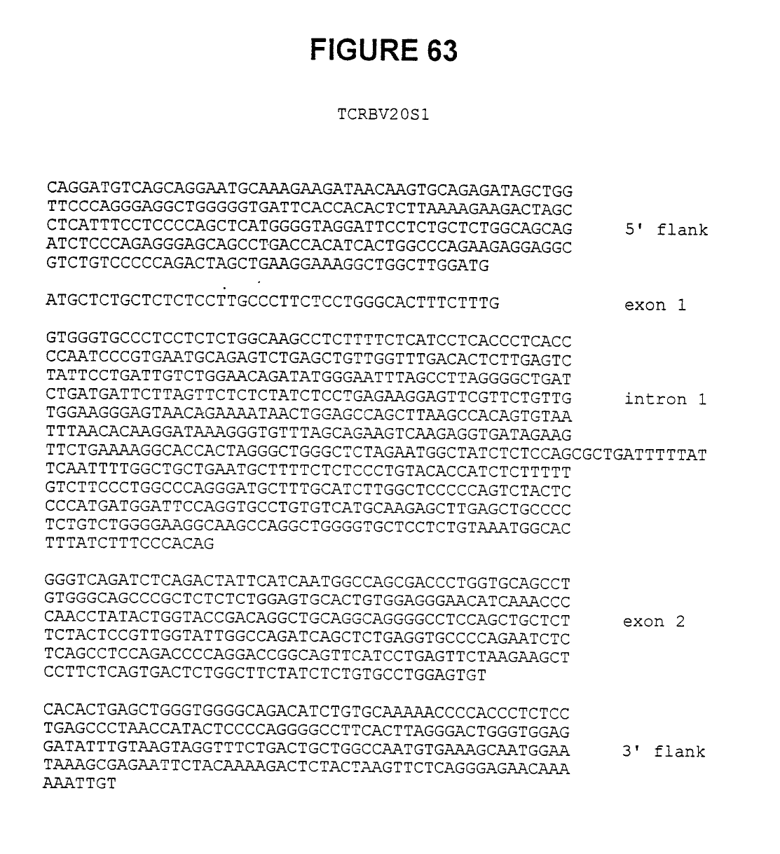

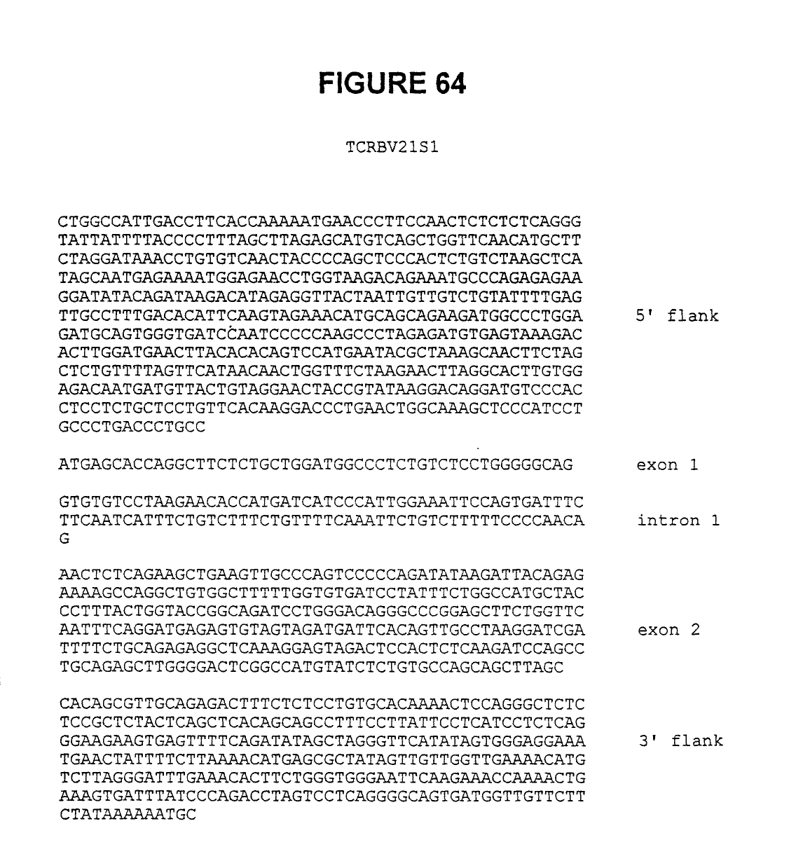

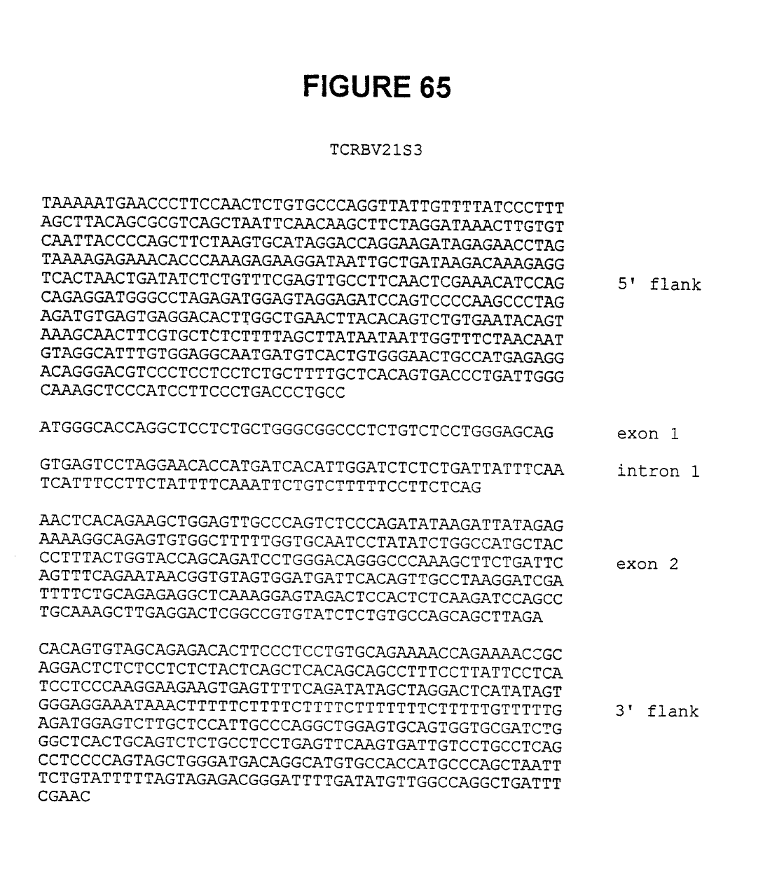

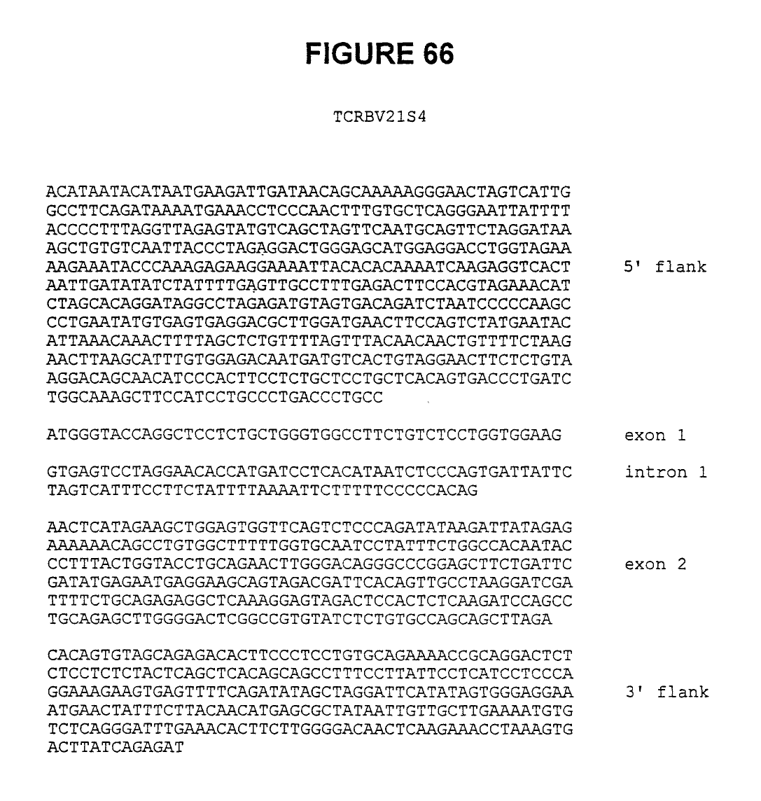

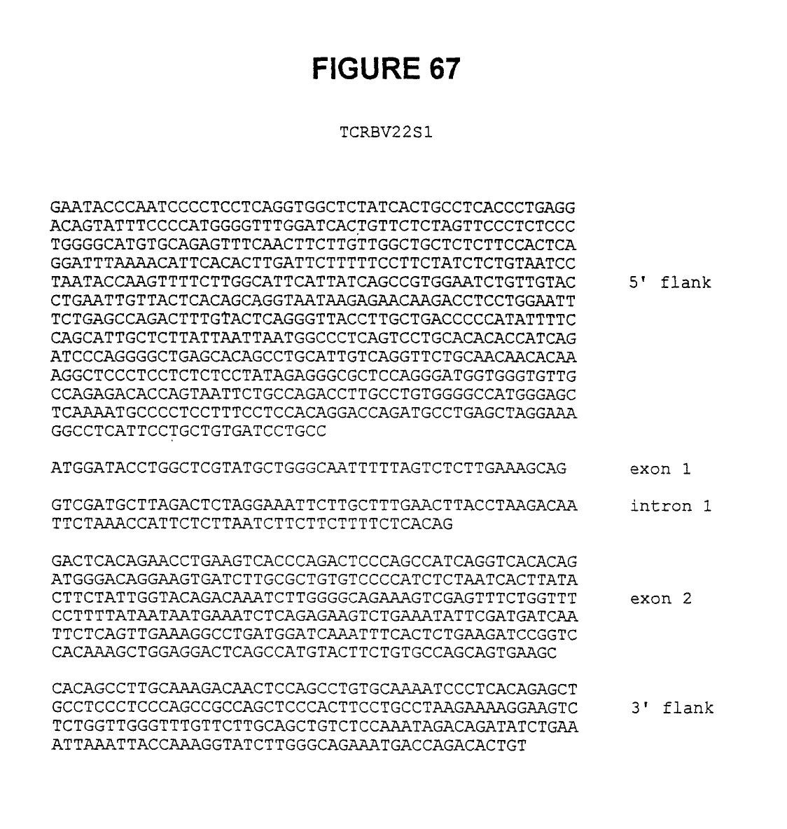

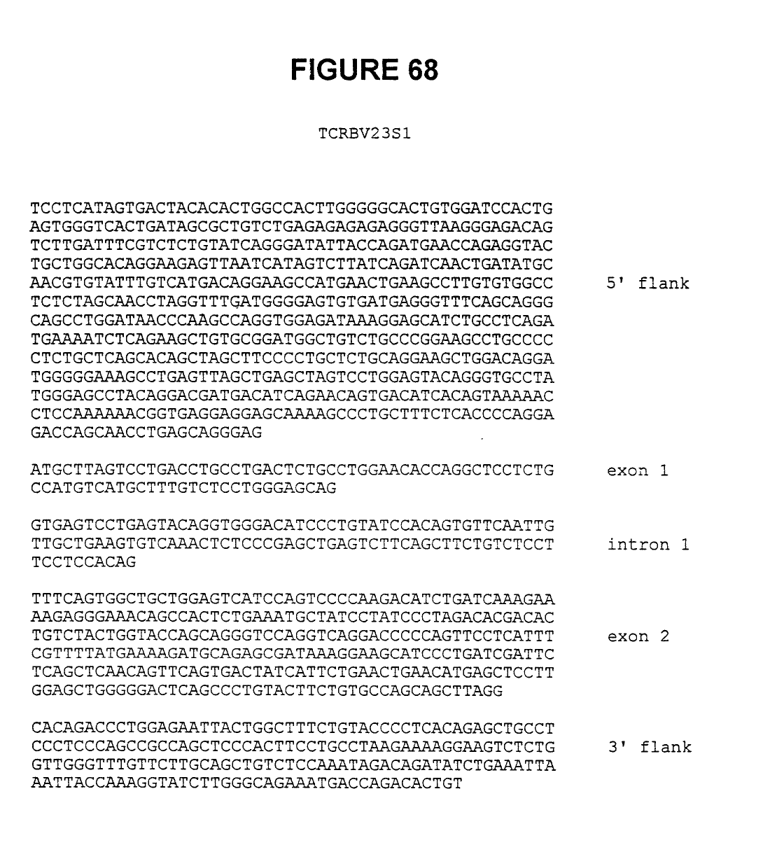

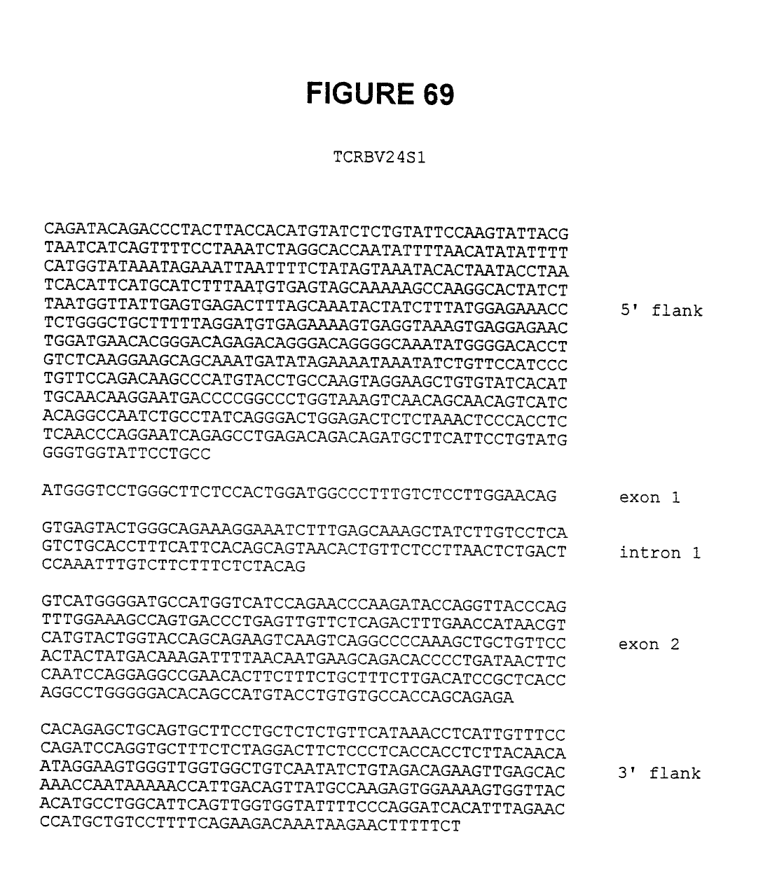

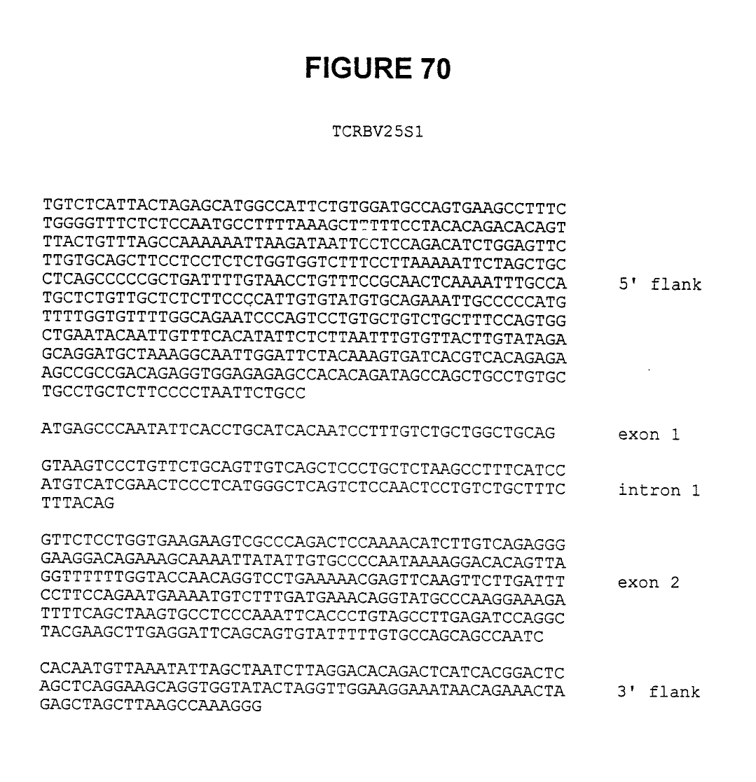

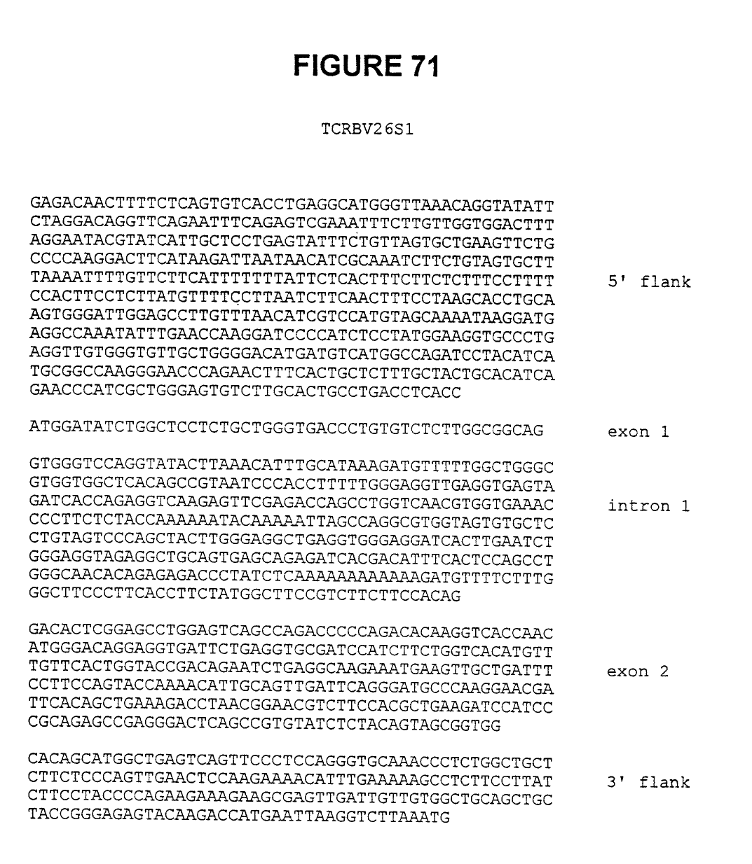

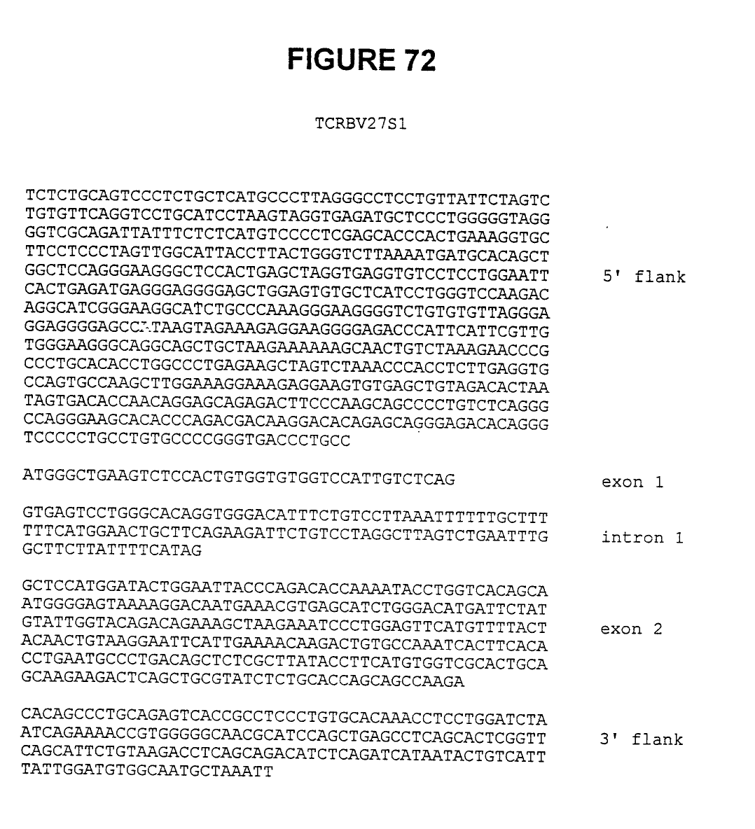

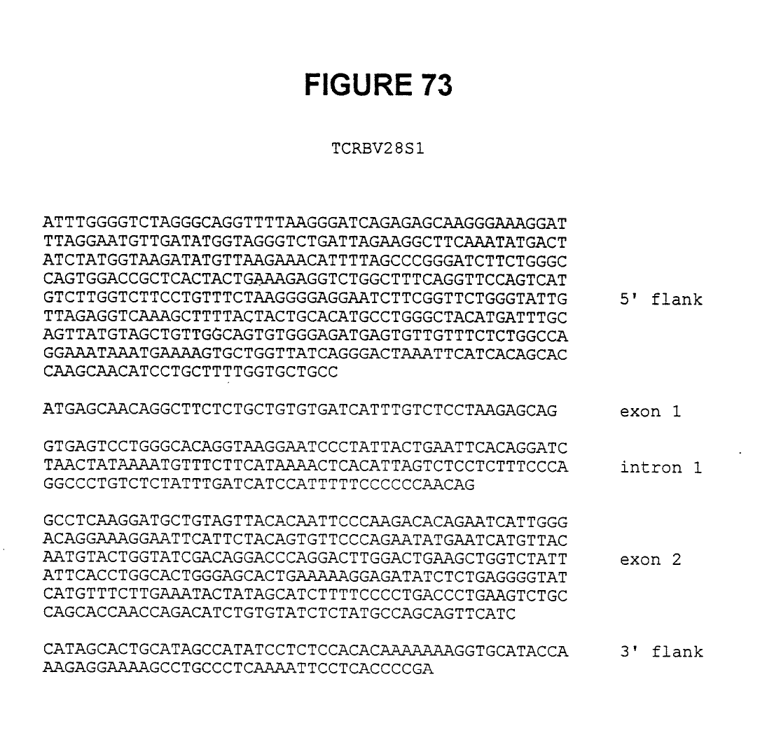

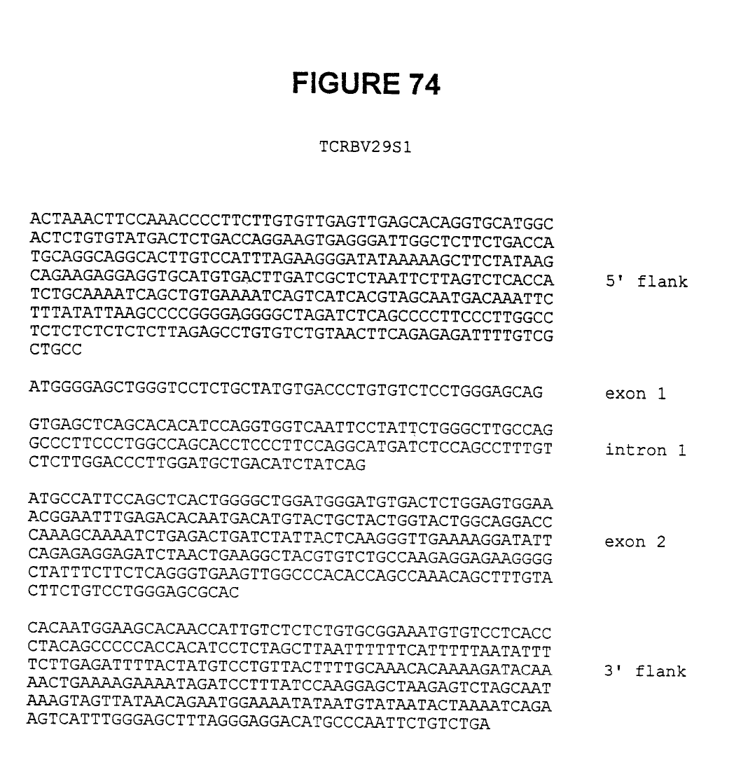

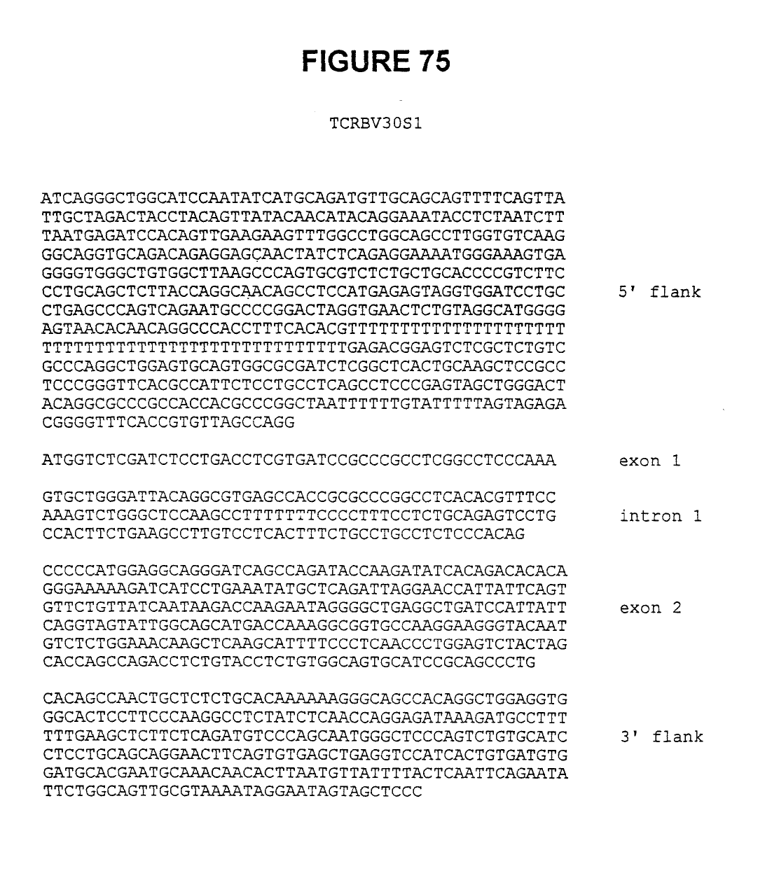

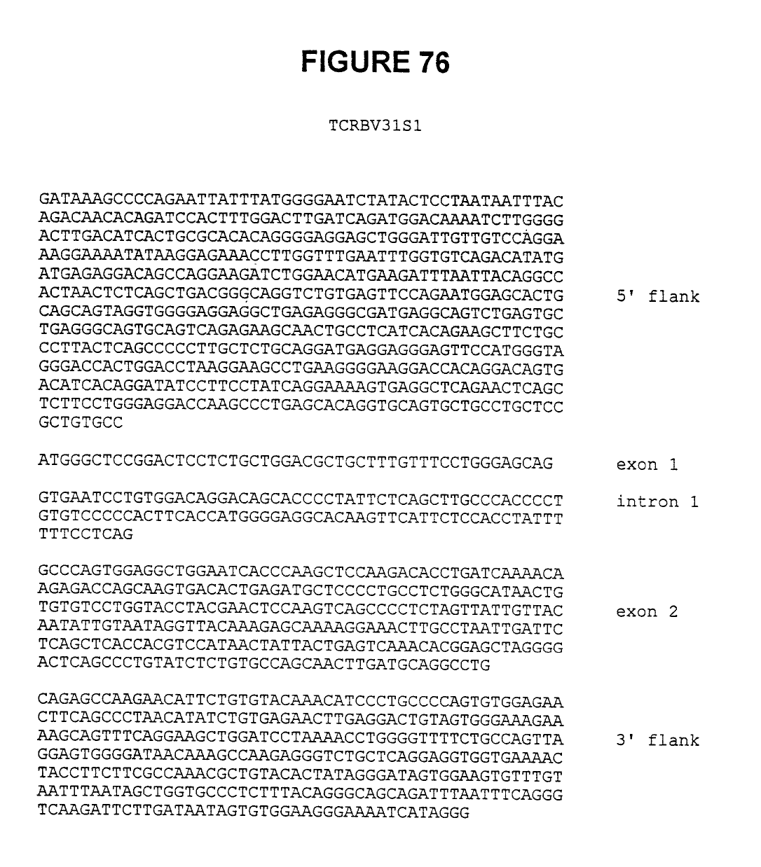

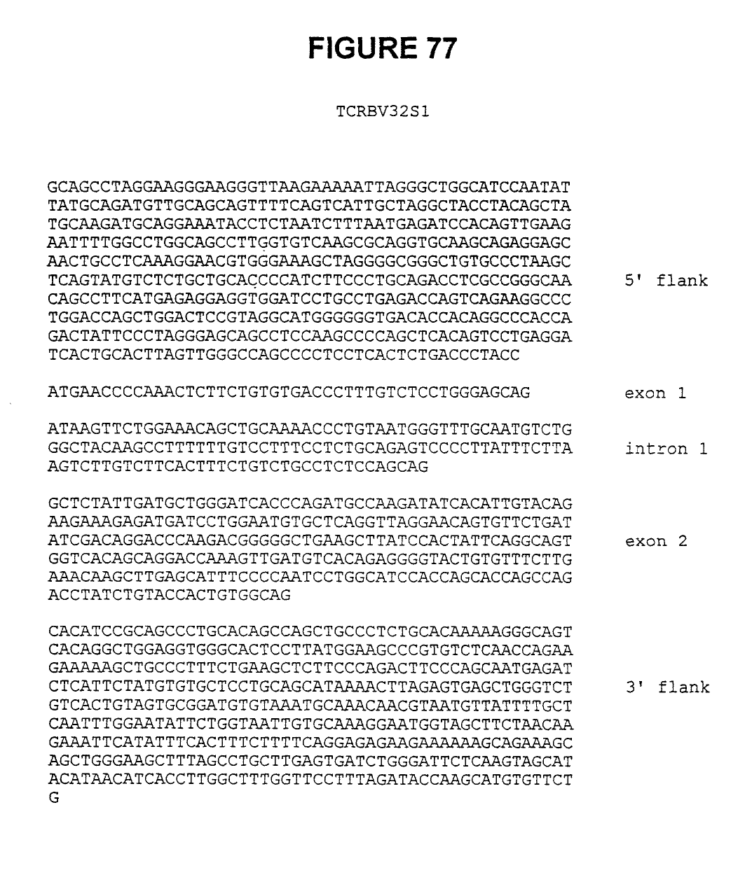

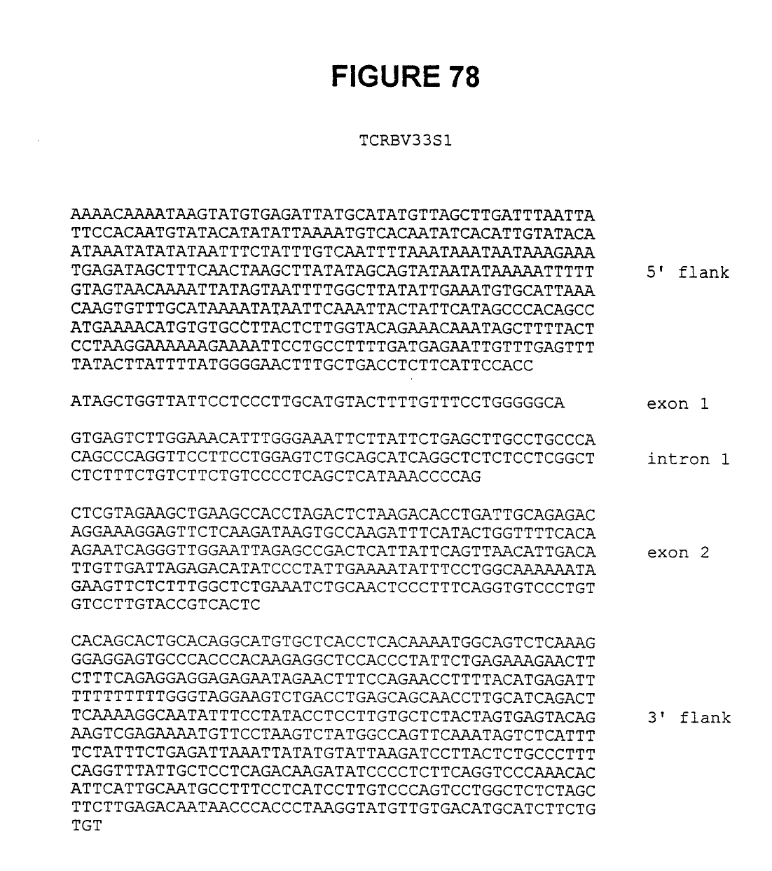

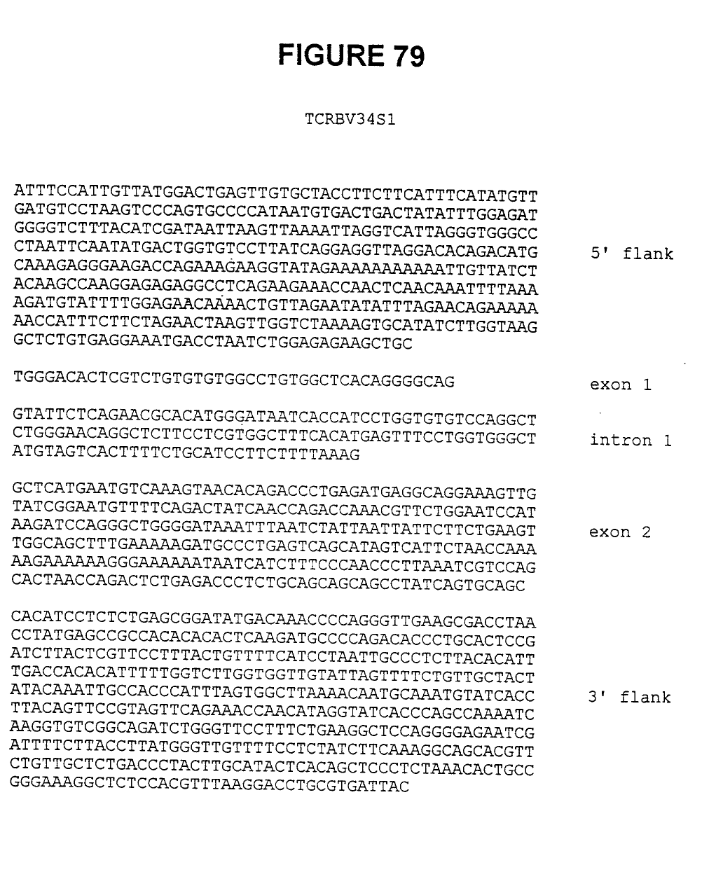

The nucleotide sequence for each of these 67 V gene segments are shown in FIGS. 13-79. Briefly, for purposes of illustration each V gene sequence is sectioned into: (a) a 5′ flanking sequence, (b) a first exon which contains most of the leader sequence, (c) the first intron, (d) the second exon, which contains both the remainder of the leader sequence, and the sequence encoding the mature polypeptide, and (e) the 3′ flanking sequence, which contains the recombination signals necessary for the joining of the V and D gene segments. A table which contains the recombination signals of each of the Vβ gene segments is presented in FIG. 11. In this figure, each sequence is broken down into three component parts, a heptamer, an approximately 22 base spacer, and a nonamer.

Of the 67 V gene segments, as many as 20 may be pseudogenes. Briefly, these pseudogenes can result from a variety of causes, including for example, frame shifts (e.g., TCRBV29S1 and TCRBV30S1), stop codons (e.g., TCRBV27S1 and TCRBV8S4), deletion of conserved cystine residues (e.g., TCRBV28S1), and lack of a consensus splice donor site (e.g., TCRBV5S5). The status of these genes as pseudogenes is of course tentatively made as some of these problems may be polymorphic, or may not be inhibitory to transcription and translation.

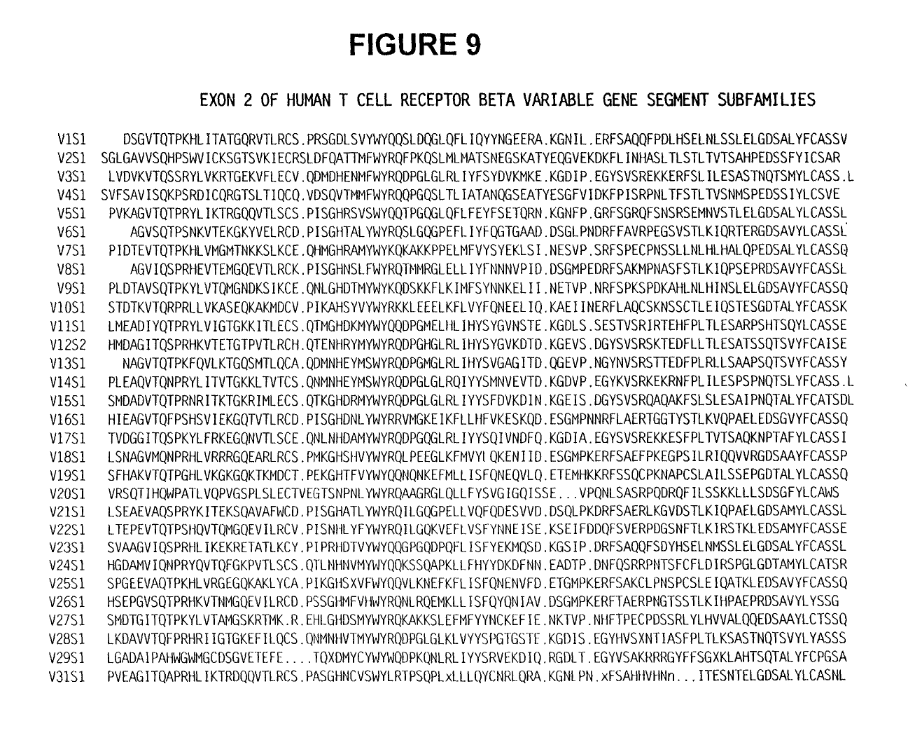

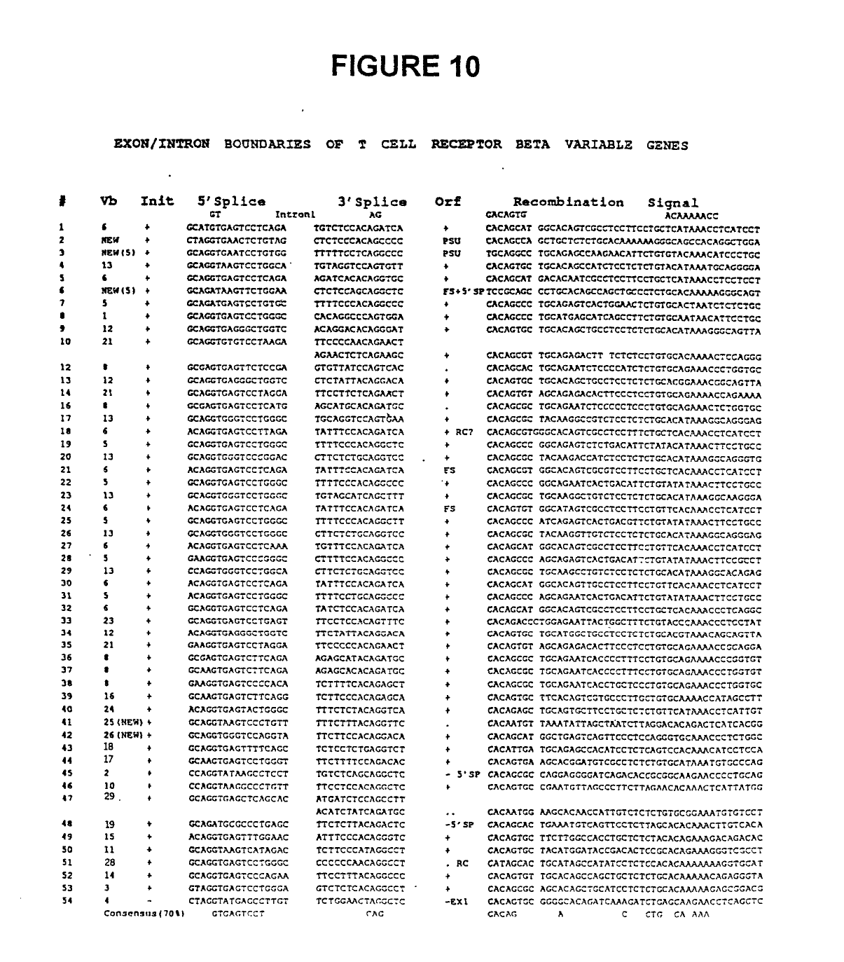

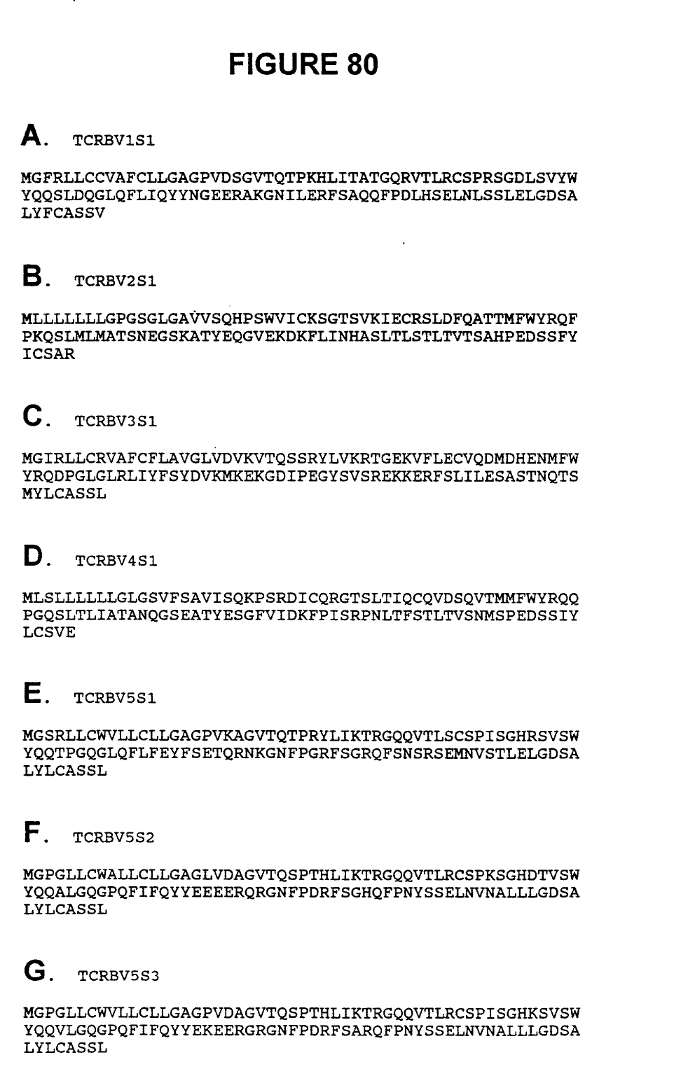

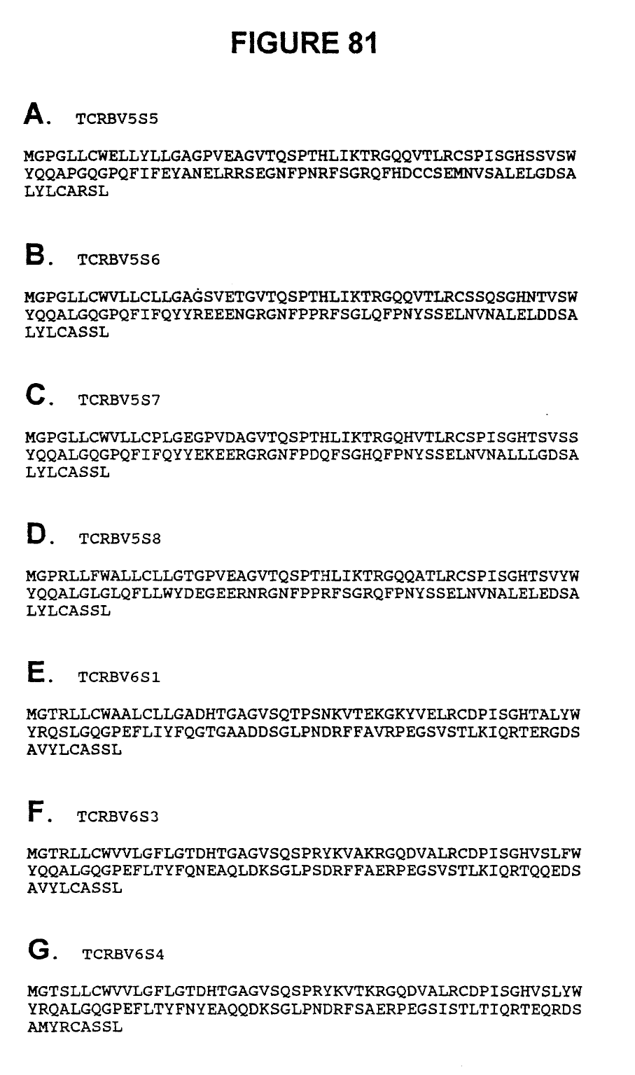

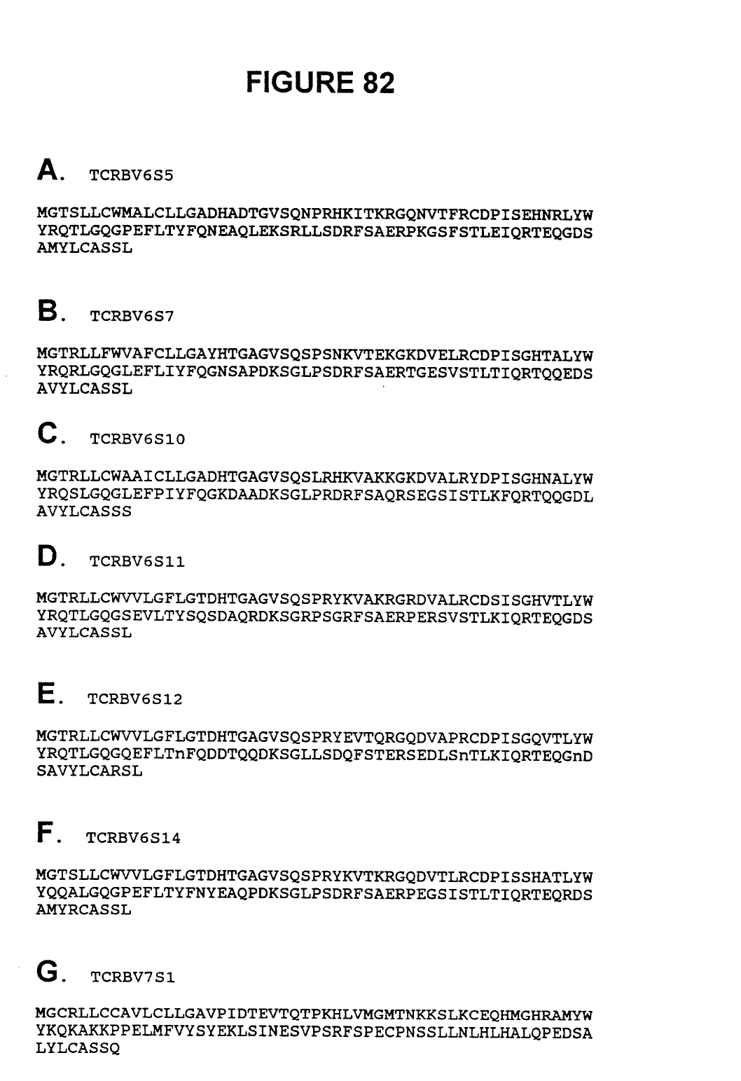

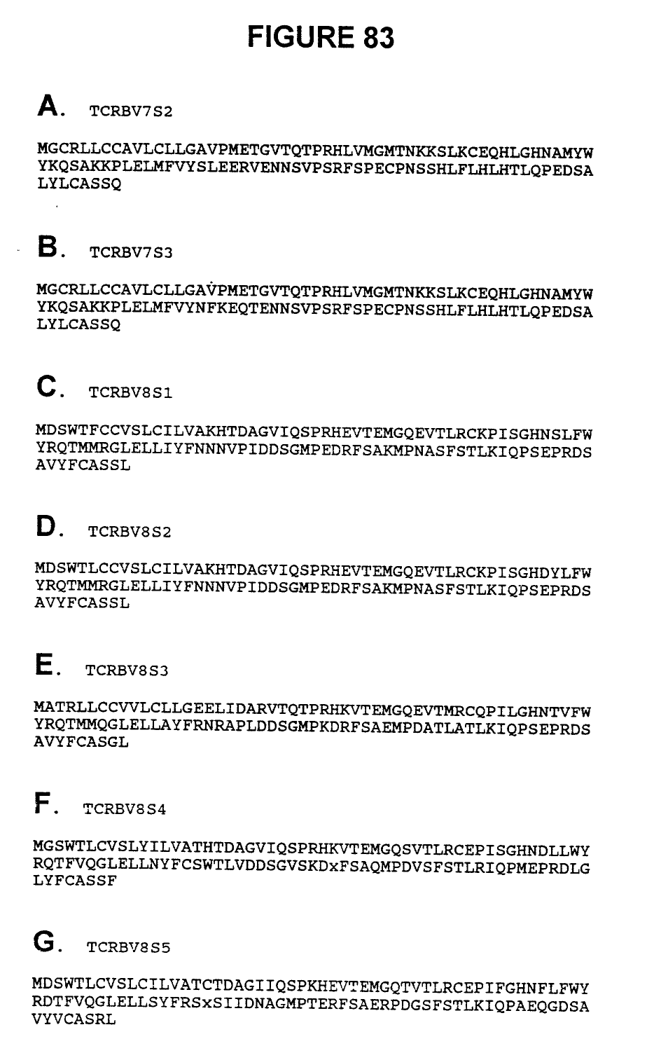

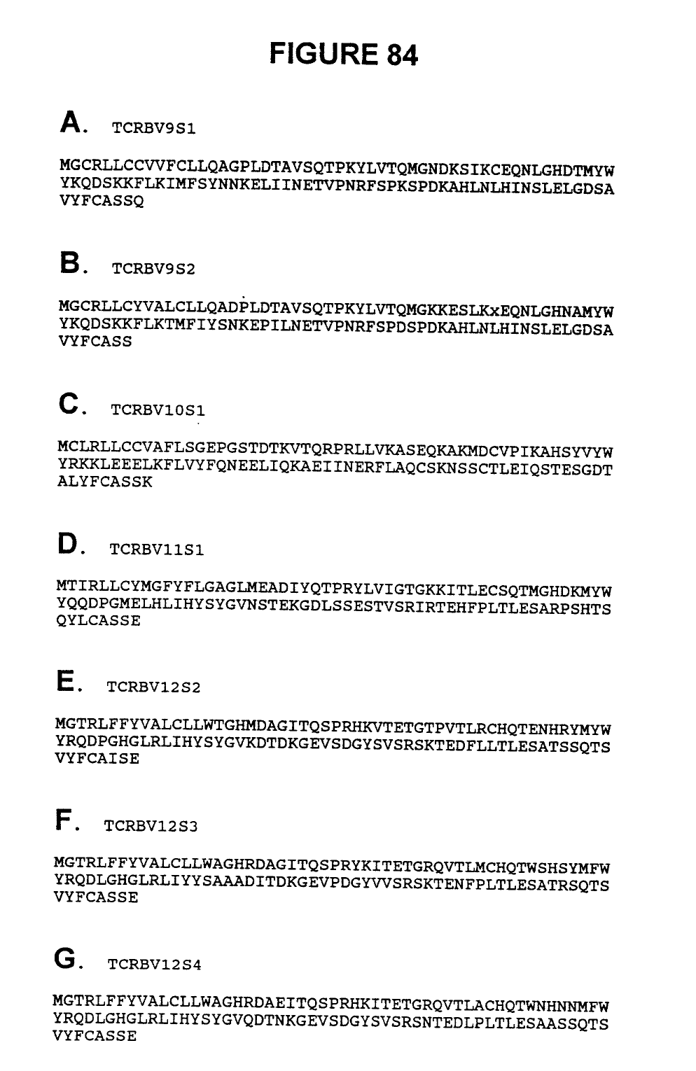

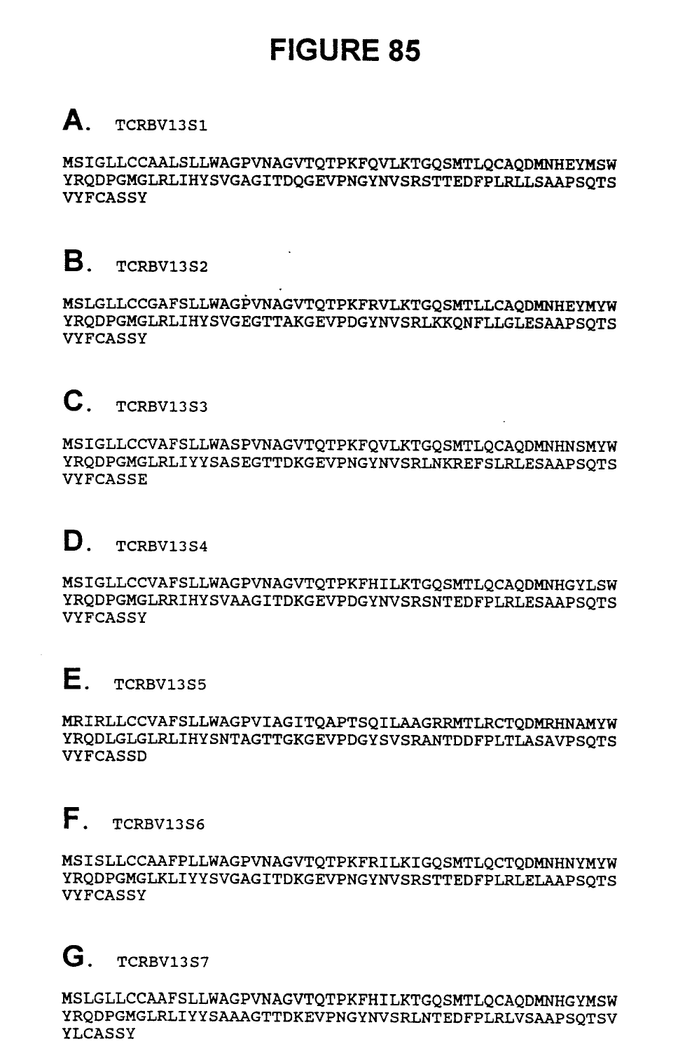

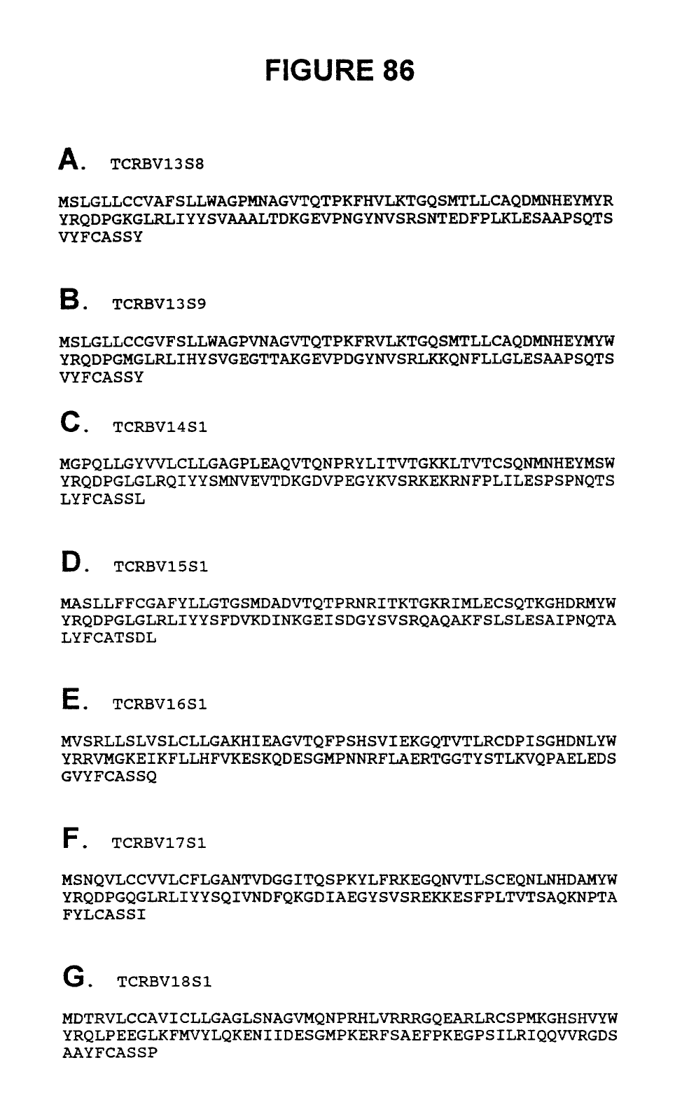

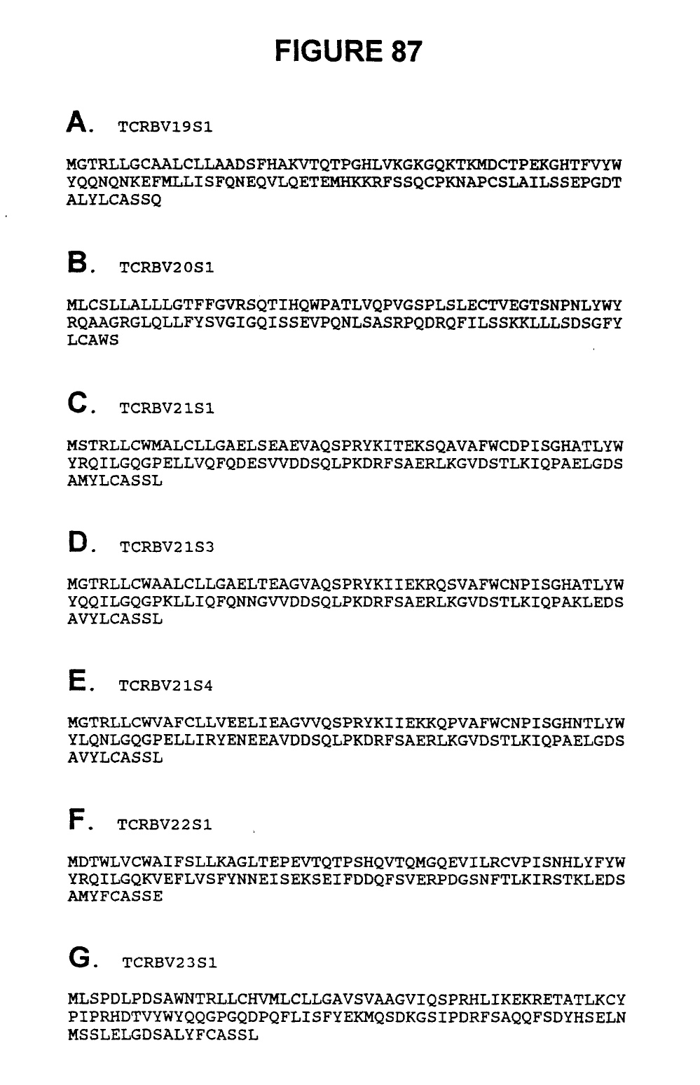

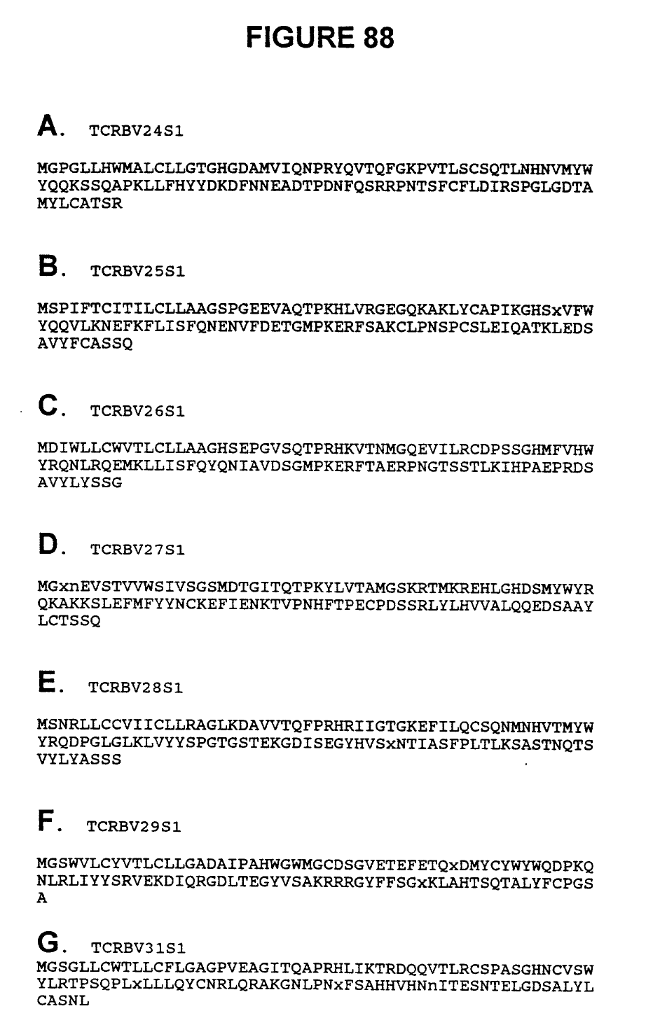

Predicted amino acid translations of each V gene is presented in FIGS. 80 to 88, in the one-letter amino acid code. Accommodations for stop codons and frame shifts are made to maintain homologous sequences. TCRBV25-TCRBV34 are previously unreported. An alignment of the amino acid sequences of exon 2 for one member of each of the subfamilies, except for Vβ30 and β32-β34, is presented in FIG. 9. In this figure, a dot represents a gap introduced to maintain an alignment of common motifs, and the sequences are presented in a numerical order, and not in their order of relatedness.

### TCR Nucleic Acid Molecules

Although the above T cell receptors have been provided for purposes of illustration, the present invention should not be so limited. In particular, “TCR” and “sTCR” (soluble T cell receptor) as utilized herein should be understood to include a wide variety of T cell receptors which are encoded by nucleic acid molecules that have substantial similarity to the sequences disclosed herein. As utilized within the context of the present invention, nucleic acid molecules which encode T cell receptors are deemed to be substantially similar to those disclosed herein if: (a) the nucleic acid sequence is derived from the coding region of a native T cell receptor gene (including, for example, allelic variations of the sequences disclosed herein); (b) the nucleic acid sequence is capable of hybridization to nucleic acid sequences of the present invention under conditions of high stringency (e.g., 50% formamide, 5×SSPE, 5× Denhardt's, 0.1% SDS, 100 ug/ml Salmon Sperm DNA, and a temperature of 42° C.; see also Sambrook et al., Molecular Cloning: A Laboratory Manual, 2d Ed., Cold Spring Harbor Laboratory Press, NY, 1989); or (c) nucleic acid sequences are degenerate as a result of the genetic code to the nucleic acid sequences defined in (a) or (b). Furthermore, as noted above, although DNA molecules are primarily referred to herein, as should be evident to one of skill in the art given the disclosure provided herein, a wide variety of related nucleic acid molecules may also be utilized in various embodiments described herein, including for example, RNA, nucleic acid analogues, as well as chimeric nucleic acid molecules which may be composed of more than one type of nucleic acid.

In addition, as noted above, within the context of the present invention “T cell receptors” and “soluble T cell receptors” should be understood to include derivatives and analogs of the T cell receptors described above. Such derivatives include allelic variants and genetically engineered variants that contain conservative amino acid substitutions and/or minor additions, substitutions or deletions of amino acids, the net effect of which does not substantially change the biological activity or function of the T cell receptor. Such derivatives are generally greater than about 74% to 80% identical, preferably greater than 85% to 90% identical, and most preferably greater than 92%, 95% or 98% identical. Homology may be determined, for example, by comparing sequence information using the GAP computer program, version 6.0, available from the University of Wisconsin Genetics Computer Group (UWGCG).

The primary amino acid structure of T cell receptors may also be modified by derivatizing amino acid side chains, and/or the amino or carboxy terminus with various functional groups, in order to allow for the formation of various conjugates (e.g., protein-TCR conjugates). Alternatively, conjugates of TCR (and sTCR) may be constructed by recombinantly producing fusion proteins. Such fusion proteins may comprise, for example, TCR-protein Z wherein protein Z is a lymphokine receptor; a binding portion of an antibody; a toxin (as discussed below); or a protein or peptide which facilitates purification or identification of TCR (e.g., poly-His). For example, a fusion protein such as TCR (His)ₙ or sTCR (His)ₙ may be constructed in order to allow purification of the protein via the poly-His residue, for example, on a NTA nickel-chelating column. The amino acid sequence of a T cell receptor may also be linked to a peptide such as Asp-Tyr-Lys-Asp-Asp-Asp-Asp-Lys (DYKDDDDK) (Sequence I.D. No. 985) (Hopp et al., Bio/Technology 6:1204, 1988) in order to facilitate purification of expressed recombinant protein.

The present invention also includes TCR (and sTCR) proteins which may be produced either with or without associated native-pattern glycosylation. For example, expression of TCR DNAs in bacteria such as E. coli provides non-glycosylated molecules. In contrast, TCR expressed in yeast or mammalian expression systems (as discussed below) may vary in both glycosylation pattern and molecular weight from native TCR, depending on the amino acid sequence and expression system which is utilized. In addition, functional mutants of mammalian TCR having inactivated glycosylation sites may also be produced in a homogeneous, reduced-carbohydrate form, utilizing oligonucleotide synthesis, site-directed mutagenesis, or random mutagenesis techniques. Briefly, N-glycosylation sites in eukaryotic proteins are generally characterized by the amino acid triplet Asn-A₁-Z, where A₁ is any amino acid except Pro, and Z is Ser or Thr. In this triplet, asparagine provides a side chain amino group for covalent attachment of carbohydrate. Such sites may be eliminated by deleting Asn or Z, substituting another amino acid for Asn or for residue Z, or inserting a non-Z amino acid between A₁ and Z, or an amino acid other than Asn between Asn and A₁.

Proteins which are substantially similar to TCR proteins may also be constructed by, for example, substituting or deleting various amino acid residues which are not required for biological activity. For example, cysteine residues may be deleted or replaced with other amino acids to prevent formation of incorrect intramolecular disulfide bridges upon renaturation. Similarly, adjacent dibasic amino acid residues may be modified for expression in yeast systems in which KEX2 protease activity is present.

Not all mutations in the nucleotide sequence which encodes TCR will be expressed in the final product. For example, nucleotide substitutions may be made in order to avoid secondary structure loops in the transcribed mRNA, or to provide codons that are more readily translated by the selected host, and thereby enhance expression within a selected host.

Generally, substitutions at the amino acid level should be made conservatively, i.e., the most preferred substitute amino acids are those which have characteristics resembling those of the residue to be replaced. When a substitution, deletion, or insertion strategy is adopted, the potential effect of the deletion or insertion on biological activity should be considered utilizing, for example, the signaling assay disclosed within the Examples.

Mutations which are made to the sequence of the nucleic acid molecules of the present invention should generally preserve the reading frame phase of the coding sequences. Furthermore, the mutations should preferably not create complementary regions that could hybridize to produce secondary mRNA structures, such as loops or hairpins, which would adversely affect translation of the receptor mRNA. Although a mutation site may be predetermined, it is not necessary that the nature of the mutation per se be predetermined. For example, in order to select for optimum characteristics of mutants at a given site, random mutagenesis may be conducted at the target codon, and the expressed TCR mutants screened for the biological activity. Representative methods for random mutagenesis include those described by Ladner et al. in U.S. Pat. Nos. 5,096,815; 5,198,346; and 5,223,409.

As noted above, mutations may be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, site-directed mutagenesis procedures may be employed to provide an altered gene having particular codons altered according to the substitution, deletion, or insertion required. Exemplary methods of making the alterations set forth above are disclosed by Walder et al. (Gene 42:133, 1986); Bauer et al. (Gene 37:73, 1985); Craik, Bio Techniques, January 1985, 12-19); Smith et al. (Genetic Engineering: Principles and Methods, Plenum Press, 1981); Sambrook et al. (Molecular Cloning: A Laboratory Manual, 2d Ed., Cold Spring Harbor Laboratory Press, 1989); and U.S. Pat. Nos. 4,518,584 and 4,737,462, which are incorporated by reference herein.

T cell receptors (including the whole TCR, the β chain above, peptide fragments, or synthetic peptides containing Vβ amino acids), as well as substantially similar derivatives or analogs may be used as therapeutic reagents, immunogens, reagents in receptor-based immunoassays, or as binding agents for affinity purification procedures. Moreover, T cell receptors of the present invention may be utilized to screen compounds for T cell receptor agonist or antagonistic activity. T cell receptor proteins may also be covalently bound through reactive side groups to various insoluble substrates, such as cyanogen bromide-activated, bisoxirane-activated, carbonyldiirnidazole-activated, or tosyl-activated, agarose structures, or by adsorbing to polyolefin surfaces (with or without gluteraldehyde cross-linking). Once bound to a substrate, T cell receptors may be used to selectively bind (for purposes of assay or purification) anti-TCR antibodies.

### Expression of TCR Nucleic Acid Molecules

As noted above, the present invention provides recombinant expression vectors capable of directing the expression of the above described nucleic acid molecules. Briefly, in order to express nucleic acid molecules of the present invention, a nucleic acid molecule which encodes a T cell receptor sequence (or portion thereof) is inserted into a suitable expression vector, which in turn is used to transform or transfect appropriate host cells for expression. Host cells for use in practicing the present invention include mammalian, avian, plant, insect, bacterial and fungal cells. Preferred eukaryotic cells include cultured mammalian cell lines (e.g., rodent or human cell lines) and fungal cells, including species of yeast (e.g., Saccharomyces spp., particularly S. cerevisiae, Schizosaccharomyces spp., or Kluyveromyces spp.). Methods for producing recombinant proteins in a variety of prokaryotic and eukaryotic host cells are generally known in the art (see “Gene Expression Technology,” Methods in Enzymology, Vol. 185, Goeddel (ed.), Academic Press, San Diego, Calif., 1990; see also, “Guide to Yeast Genetics and Molecular Biology,” Methods in Enzymology, Guthrie and Fink (eds.), Academic Press, San Diego, Calif., 1991). In general, a host cell will be selected on the basis of its ability to produce the protein of interest at a high level or its ability to carry out at least some of the processing steps necessary for the biological activity of the protein. In this way, the number of cloned DNA sequences which must be transfected into the host cell may be minimized and overall yield of biologically active protein may be maximized.

Suitable yeast vectors for use in the present invention include YRp7 (Struhl et al., Proc. Natl. Acad. Sci. USA 76:1035-1039, 1978), YEp13 (Broach et al., Gene 8:121-133, 1979), POT vectors (Kawasaki et al., U.S. Pat. No. 4,931,373, which is incorporated by reference herein), pJDB249 and pJDB219 (Beggs, Nature 275:104-108, 1978) and derivatives thereof. Such vectors will generally include a selectable marker, which may be one of any number of genes that exhibit a dominant phenotype for which a phenotypic assay exists to enable transfornants to be selected. Preferred selectable markers are those that complement host cell auxotrophy, provide antibiotic resistance or enable a cell to utilize specific carbon sources, and include LEU2 (Broach et al., ibid.), URA3 (Botstein et al., Gene 8:17, 1979), HIS3 (Struhl et al., ibid.) or POT1 (Kawasaki et al., ibid.). Another suitable selectable marker is the CAT gene, which confers chloramphenicol resistance on yeast cells.

Preferred promoters for use in yeast include promoters from yeast glycolytic genes (Hitzeman et al., J. Biol. Chem. 255:12073-12080, 1980; Alber and Kawasaki, J. Mol. Appl. Genet. 1:419-434, 1982; Kawasaki, U.S. Pat. No. 4,599,311) or alcohol dehydrogenase genes (Young et al., in Genetic Engineering of Microorganisms for Chemicals, Hollaender et al. (eds.), p. 355, Plenum, New York, 1982; Ammerer, Meth. Enzymol. 101:192-201, 1983). The expression units may also include a transcriptional terminator. A preferred transcriptional terminator is the TPII terminator (Alber and Kawasaki, ibid).

Techniques for transforming fungi are well known in the literature, and have been described, for instance, by Beggs (ibid.), Hinnen et al. (Proc. Natl. Acad. Sci. USA 75:1929-1933, 1978), Yelton et al. (Proc. Natl. Acad. Sci. USA 81:1740-1747, 1984), and Russell (Nature 301:167-169, 1983). The genotype of the host cell will generally contain a genetic defect that is complemented by the selectable marker present on the expression vector. Choice of a particular host and selectable marker is well within the level of ordinary skill in the art. To optimize production of the heterologous proteins in yeast, for example, it is preferred that the host strain carries a mutation, such as the yeast pep4 mutation (Jones, Genetics 85:23-33, 1977), which results in reduced proteolytic activity.

In addition to fungal cells, cultured mammalian cells may be used as host cells within the present invention. Preferred cultured mammalian cells for use in the present invention include the COS-1 (ATCC No. CRL 1650), COS-7 (ATCC No. CRL 1651), BHK (ATCC No. CRL 1632), and 293 (ATCC No. CRL 1573; Graham et al., J. Gen. Virol. 36:59-72, 1977) cell lines. A preferred BHK cell line is the BHK 570 cell line (deposited with the American Type Culture Collection under accession number CRL 10314). In addition, a number of other mammalian cell lines may be used within the present invention, including Rat Hep I (ATCC No. CRL 1600), Rat Hep II (ATCC No. CRL 1548), TCMK (ATCC No. CCL 139), Human lung (ATCC No. CCL 75.1), Human hepatoma (ATCC No. HTB-52), Hep G2 (ATCC No. HB 8065), Mouse liver (ATCC No. CCL 29.1), NCTC 1469 (ATCC No. CCL 9.1), SP2/0-Ag14 (ATCC No. 1581), HIT-T15 (ATCC No. CRL 1777), and RINm 5AHT₂B (Orskov and Nielson, FEBS 229(1):175-178, 1988).

Mammalian expression vectors for use in carrying out the present invention should include a promoter capable of directing the transcription of a cloned gene or cDNA. Preferred promoters include viral promoters and cellular promoters. Viral promoters include the immediate early cytomegalovirus promoter (Boshart et al., Cell 41:521-530, 1985) and the SV40 promoter (Subrarnani et al., Mol. Cell. Biol. 1:854-864, 1981). Cellular promoters include the mouse metallothionein-1 promoter (Palmiter et al., U.S. Pat. No. 4,579,821), a mouse Vⱼ promoter (Bergman et al., Proc. Natl. Acad. Sci. USA 81:7041-7045, 1983; Grant et al., Nuc. Acids Res. 15:5496, 1987) and a mouse V_{H} promoter (Loh et al., Cell 33:85-93, 1983). A particularly preferred promoter is the major late promoter from Adenovirus 2 (Kaufnan and Sharp, Mol. Cell. Biol. 2:1304-13199, 1982). Such expression vectors may also contain a set of RNA splice sites located downstream from the promoter and upstream from the DNA sequence encoding the peptide or protein of interest. Preferred RNA splice sites may be obtained from adenovirus and/or immunoglobulin genes. Also contained in the expression vectors is a polyadenylation signal located downstream of the coding sequence of interest. Suitable polyadenylation signals include the early or late polyadenylation signals from SV40 (Kaufman and Sharp, ibid.), the polyadenylation signal from the Adenovirus 5 E1B region and the human growth hormone gene terminator (DeNoto et al., Nuc. Acids Res. 9:3719-3730, 1981). The expression vectors may include a noncoding viral leader sequence, such as the Adenovirus 2 tripartite leader, located between the promoter and the RNA splice sites. Preferred vectors may also include enhancer sequences, such as the SV40 enhancer and the mouse 1 enhancer (Gillies, Cell 33:717-728, 1983). Expression vectors may also include sequences encoding the adenovirus VA RNAs. Suitable vectors can be obtained from commercial sources (e.g., Stratagene, La Jolla, Calif.).

Cloned DNA sequences may be introduced into cultured mammalian cells by, for example, calcium phosphate-mediated transfection (Wigler et al., Cell 14:725, 1978; Corsaro and Pearson, Somatic Cell Genetics 7:603, 1981; Graham and Van der Eb, Virology 52:456, 1973), electroporation (Neumann et al., EMBO J. 1:841-845, 1982), or DEAE-dextran mediated transfection (Ausubel et al. (eds.), Current Protocols in Molecular Biology, John Wiley and Sons, Inc., NY, 1987). To identify cells that have stably integrated the cloned DNA, a selectable marker is generally introduced into the cells along with the gene or cDNA of interest. Preferred selectable markers for use in cultured mammalian cells include genes that confer resistance to drugs, such as neomycin, hygromycin, and methotrexate. The selectable marker may be an amplifiable selectable marker. Preferred amplifiable selectable markers are the DHFR gene and the neomycin resistance gene. Selectable markers are reviewed by Thilly (Mammalian Cell Technology, Butterworth Publishers, Stoneham, Mass.). The choice of selectable markers is well within the level of ordinary skill in the art.

Selectable markers may be introduced into the cell on a separate vector at the same time as the T cell receptor sequence, or they may be introduced on the same vector. If on the same vector, the selectable marker and the T cell receptor sequence may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA” to the mixture which is introduced into the cells.

Transfected mammalian cells are allowed to grow for a period of time, typically 1-2 days, to begin expressing the DNA sequencers) of interest. Drug selection is then applied to select for growth of cells that are expressing the selectable marker in a stable fashion. For cells that have been transfected with an amplifiable selectable marker the drug concentration may be increased in a stepwise manner to select for increased copy number of the cloned sequences, thereby increasing expression levels. Cells expressing the introduced sequences are selected and screened for production of the protein of interest in the desired form or at the desired level. Cells which satisfy these criteria may then be cloned and scaled up for production.

Preferred prokaryotic host cells for use in carrying out the present invention are strains of the bacteria Escherichia coli, although Bacillus and other genera are also useful. Techniques for transforming these hosts and expressing foreign DNA sequences cloned therein are well known in the art (see, e.g., Maniatis et al., Molecular Cloning: A Laboratory Manual, Cold Spring Harbor Laboratory, 1982; or Sambrook et al., supra). Vectors used for expressing cloned DNA sequences in bacterial hosts will generally contain a selectable marker, such as a gene for antibiotic resistance, and a promoter that functions in the host cell. Appropriate promoters include the trp (Nichols and Yanofsky, Meth. Enzymol. 101:155-164, 1983), lac (Casadaban et al., J. Bacteriol. 143:971-980, 1980), and phage k (Queen, J. Mol. Appl. Genet. 2:1-10, 1983) promoter systems. Plasmids useful for transforming bacteria include pBR322 (Bolivar et al., Gene 2:95-113, 1977), the pUC plasmids (Messing, Meth. Enzymol 101:20-78, 1983; Vieira and Messing, Gene 19:259-268, 1982), pCQV2 (Queen, ibid.), and derivatives thereof. Plasmids may contain both viral and bacterial elements.

Given the teachings provided herein, promoters, terminators and methods for introducing expression vectors encoding T cell receptor sequences of the present invention into avian and insect cells would be evident to those of skill in the art. The use of baculoviruses, for example, as vectors for expressing heterologous DNA sequences in insect cells has been reviewed by Atkinson et al. (Pestic. Sci. 28:215-224,1990).

Host cells containing DNA molecules of the present invention are then cultured to express a DNA molecule encoding a T cell receptor sequence. The cells are cultured according to standard methods in a culture medium containing nutrients required for growth of the chosen host cells. A variety of suitable media are known in the art and generally include a carbon source, a nitrogen source, essential amino acids, vitamins and minerals, as well as other components (e.g., growth factors or serum) that may be required by the particular host cells. The growth medium will generally select for cells containing the DNA molecules by, for example, drug selection or deficiency in an essential nutrient which is complemented by the selectable marker on the DNA construct or co-transfected with the DNA construct.

Suitable growth conditions for yeast cells, for example, include culturing in a chemically defied medium, comprising a nitrogen source, which may be a non-amino acid nitrogen source or a yeast extract, inorganic salts, vitamins and essential amino acid supplements at a temperature between 4° C. and 37° C., with 30° C. being particularly preferred. The pH of the medium is preferably maintained at a pH greater than 2 and less than 8, more preferably pH 5-6. Methods for maintaining a stable pH include buffering and constant pH control. Preferred agents for pH control include sodium hydroxide. Preferred buffering agents include succinic acid and Bis-Tris (Sigma Chemical Co., St. Louis, Mo.). Due to the tendency of yeast host cells to hyperglycosylate heterologous proteins, it may be preferable to express the T cell receptors of the present invention in yeast cells having a defect in a gene required for asparagine-linked glycosylation. Such cells are preferably grown in a medium containing an osmotic stabilizer. A preferred osmotic stabilizer is sorbitol supplemented into the medium at a concentration between 0.1 M and 1.5 M, preferably at 0.5 M or 1.0 M. Cultured mammalian cells are generally cultured in commercially available serum-containing or serum-free media. Selection of a medium and growth conditions appropriate for the particular cell line used is within the level of ordinary skill in the art.

T cell receptor sequences may also be expressed in non-human transgenic animals, particularly transgenic warm-blooded animals. Methods for producing transgenic animals, including mice, rats, rabbits, sheep and pigs, are known in the art and are disclosed, for example, by Hammer et al. (Nature 315:680-683, 1985), Palmiter et al. (Science 222:809-814. 1983), Brinster et al. (Proc. Natl. Acad. Sci. USA 82:4438-4442, 1985), Palmiter and Brinster (Cell 41:343-345, 1985) and U.S. Pat. No. 4,736,866. Briefly, an expression unit, including a DNA sequence to be expressed together with appropriately positioned expression control sequences, is introduced into pronuclei of fertilized eggs. Introduction of DNA is commonly done by microinjection. Integration of the injected DNA is detected by blot analysis of DNA from tissue samples, typically samples of tail tissue. It is generally preferred that the introduced DNA be incorporated into the germ line of the animal so that it is passed on to the animal's progeny.

Within a preferred embodiment of the invention, a transgenic animal, such as a mouse, is developed by targeting a mutation to disrupt a T cell receptor sequence (see Mansour et al., “Disruption of the proto-oncogene int-2 in mouse embryo-derived stem cells: a general strategy for targeting mutations to non-selectable genes,” Nature 336:348-352, 1988). Such animals may readily be utilized as a model to study the immunological role of the T cell receptor.

### Purification of TCR and Soluble TCR

As noted above, the present invention also provides soluble T cell receptors and receptor peptides. Within the context of the present invention, TCR peptides should be understood to include portions of a T cell receptor (and, more preferably, portions of one of the β chains described herein) or derivatives thereof discussed above, which do not contain transmembrane domains, and which are at least 8, and more preferably 10 or greater, amino acids in length. Briefly, the structure of the T cell receptors as well as the putative transmembrane domain may be predicted from the primary translation products using the hydrophopicity plot function of, for example, PROTEAN (DNA STAR, Madison, Wis.), or according to the methods described by Kyte and Doolittle (J. Mol. Biol. 157:105-132, 1982).

Soluble T cell receptors and receptor peptides may be prepared by, among other methods, culturing suitable host/vector systems as described above in order to produce the recombinant translation products of the present invention. Supernatants from such cell lines may then be treated by a variety of purification procedures in order to isolate the soluble T cell receptor or receptor peptides. For example, the supernatant may be first concentrated using commercially available protein concentration filters, such as an Amicon or Millipore Pellicon ultrafiltration unit. Following concentration, the concentrate may be applied to a suitable purification matrix such as, for example, an anti-TCR antibody bound to a suitable support. Alternatively, anion or cation exchange resins may be employed in order to purify the receptor or peptide. Finally, one or more reversed-phase high performance liquid chromatography (RP-HPLC) steps may be employed to further purify the T cell receptor peptide.

Alternatively, T cell receptor peptides may also be prepared utilizing standard polypeptide synthesis protocols, and purified utilizing the above-described procedures.

A T cell receptor peptide is deemed to be “isolated” or purified within the context of the present invention, if only a single band is detected subsequent to SDS-polyacrylamide gel analysis followed by staining with Coomassie Brilliant Blue.

### Nucleic Acid Probes and Primers

As noted above, the present invention provides nucleic acid probes and primers which are capable of specifically hybridizing to the isolated nucleic acid molecules described herein and, in the case of primers, which are capable of specifically priming and allowing or otherwise assisting in the amplification of a desired sequence. Briefly, previous to the present disclosure it was impossible to specifically interrogate each and every Vβ gene, since the genes can be related by as much as 98% and the sequence of all Vβ genes was not known. Therefore, based upon the disclosure provided herein, nucleic acid probes and primers may, for the first time, be readily designed and synthesized for a variety of applications, including for example, diagnostic assays and therapeutic use.

Within one aspect of the present invention, nucleic acid probes are provided which are capable of specifically hybridizing to an isolated nucleic acid molecule encoding a Vβ gene (including, for example, a 5′ flanking sequence, introns, coding region, or 3′ flanking sequence). Within other aspects, probes are provided which are capable of specifically hybridizing to the polymorphisms described herein, and primers are provided which are capable of allowing or assisting in the amplification of a desired polymorphism. As utilized within the context of the present invention, probes and primers are considered to be “capable of specifically hybridizing” to T cell receptor nucleic acids if they hybridize under conditions of high stringency to a particular or selected Vβ gene sequence (see Sambrook et al., supra); but not to the sequences of other Vβ gene regions (including, for example, closely related Vβ genes). Within one embodiment, probes can be specifically hybridized to a target T cell receptor Vβ gene sequence if they hybridize in the presence of 50% formamide, 5×SSPE, 5× Denhardt's, 0.1% SDS and 100 ug/ml salmon sperm DNA at 42° C., followed by a first wash with 2×SSC and 0.1% SDS at 42° C., and a second wash with 0.2×SSC and 01.% SDS at 55° C. to 60° C.

Within one aspect of the present invention, the nucleic acid probes may be composed of either deoxyribonucleic acids (DNA) ribonucleic acids (RNA), nucleic acid analogues, peptide nucleotide acids (“PNA”) or any combination of these (e.g., chimeric nucleic acid molecules), and may be as few as about 12 nucleotides in length, usually about 14 to 24 nucleotides in length, and possibly much larger. Within certain embodiments of the invention, probes may be either single stranded or double stranded, and may form a duplex, triplex or quadruplex with a given target nucleic acid molecule (see U.S. Pat. No. 5,176,996, entitled “Method for Masking Synthetic Oligonucleotides which Bind Specifically to Target Sites on Duplex DNA Molecules, by Forming a Colinear Triplex, the Synthetic Oligonucleotides and methods of Use”). Selection of probe size is somewhat dependent upon the use of the probe and the method of detection. For example, in order to determine the presence of various polymorphic forms of a T cell receptor within an individual, a shorter probe may be preferred.

Probes and primers may be constructed and labeled using techniques which are well known in the art. Shorter probes of, for example, 12 or 14 bases may be generated synthetically. Longer probes of about 75 bases to less than 1.5 kb are preferably generated by, for example, PCR amplification in the presence of labeled precursors such as ³²P-dCTP, digoxigenin-dUTP, or biotin-dATP. Probes of more than 1.5 kb are generally most easily amplified by transfecting a cell with a plasmid containing the relevant probe, growing the transfected cell into large quantities, and purifying the relevant sequence from the transfected cells (see Sambrook et al., supra).

Both probes may be labeled by a variety of markers, including, for example, radioactive markers, fluorescent markers, enzymatic markers, and chromogenic markers. The use of ³²p is particularly preferred for marking or labeling a particular probe.

As noted above, probes of the present invention may also be utilized to detect the presence of a T cell receptor mRNA or DNA within a sample. However, if nucleic acid molecules containing the T cell receptor of interest are present in only a limited number, or if it is desired to detect a selected mutant sequence which is present in only a limited number, then it may be beneficial to amplify the relevant sequence such that it may be more readily detected or obtained.

Therefore, as noted above, within other aspects of the present invention primers are provided which are capable of specifically priming, and allowing or other assisting in the amplification of a desired sequence. As utilized within the context of the present invention, primers are considered to be “specifically priming” if they prime the amplification of only one selected Vβ gene sequence. For example, a primer “specifically primes” TCRBV6S10 if it primes only the amplification of this Vβ, and not other related Vβs.

Preferably, primers should be selected such that they are highly specific and form stable duplexes with the target sequence. The primers should also be non-complementary, especially at the 3′ end, should not form dimers with themselves or other primers, and should not form secondary structures or duplexes with other regions of DNA. Within one embodiment, primers are first selected by eye based upon an alignment of related Vβ gene families. Potential primer sites are then selected in order to maximize the specificity of the individual primers. The primers may be further evaluated utilizing a computer program such as PRIMER, in order to evaluate a potential primer for characteristics such as length and melting point.

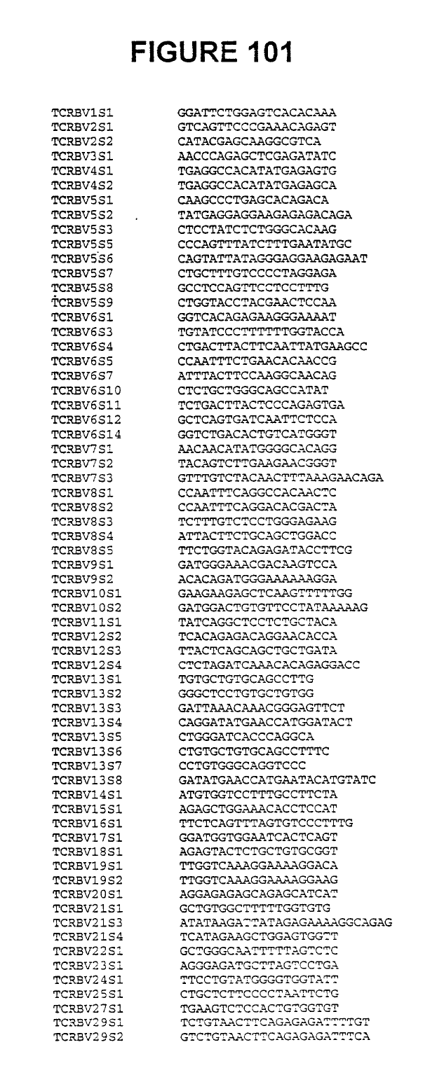

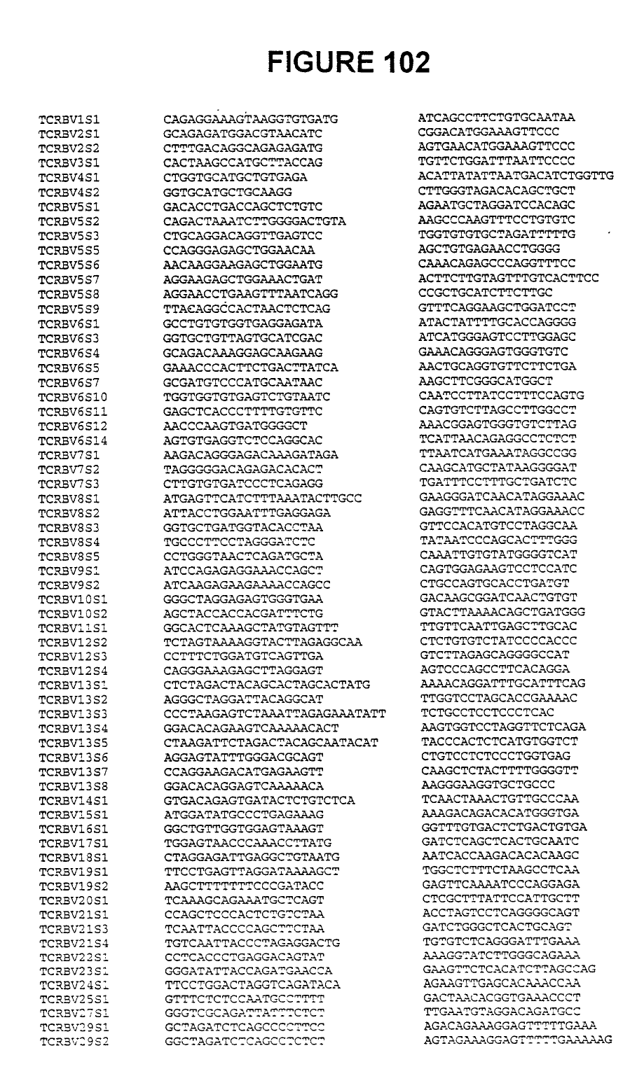

One set of particularly preferred primers are set forth below in FIGS. 101 and 102. Briefly, FIG. 102 provides a representative list of suitable 5′ and 3′ genomic primers, and FIG. 101 provides a representative list of suitable cDNA or RNA primers. Where the primer matches more than one sequence, or where it matches a sequence other than that described in the name, that match is noted in parentheses. These primers were selected such that they have a predicted melting point of between 54° C. and 62° C. (most are between 56° C. and 58° C.). In addition, primers were selected such that they had a length of between 18 and 30 bases (with an optimum of 20), a GC content of between 40% and 60%, a maximum self complementarity of 10 and a maximum 3′ complementarity of 6 (as defined in the program Primer).

A variety of methods may be utilized in order to amplify a selected sequence, including, for example, RNA amplification (see Lizardi et al., Bio/Technology 6:1197-1202, 1988; Kramer et al., Nature 339:401-402, 1989; Lomeli et al., Clinical Chem. 35(9):1826-1831, 1989; Cahill et al., Clin. Chem. 37:1482, 1991; Lizardi et al., Biotechnol. 6:1197, 1988; U.S. Pat. No. 4,786,600), and DNA amplification utilizing Polymerase Chain Reaction (“PCR”) (see U.S. Pat. Nos. 4,683,195, 4,683,202, and 4,800,159). Within other embodiments, alternative detection/amplification systems may also be utilized, including for example, the Cycling Probe Reaction (“CPR”) (see also, U.S. Pat. Nos. 4,876,187, and 5,011,769); Ligase Chain Reaction (“LCR”) or Ligase Amplification Reaction (“LAR”) (Barany, PNAS 88:189, 1991; Barringer et al., Gene 89:117, 1990; Wu and Wallace, Genomics 4:560, 1989); Transcription-Based Amplification System (“TAS”) (Kwoh et al., PNAS 86:1173, 1989); Self-Sustained Sequence Replication (“3SR”) (Guatelli et al., PNAS 87:1874, 1990 (UCSD and Salk Institute)); and Strand Displacement Amplification (“SDA”) (Walker et al., Nucleic Ac. Res. 20:1691, 1992; Walker et al., PNAS 89:392, 1992).

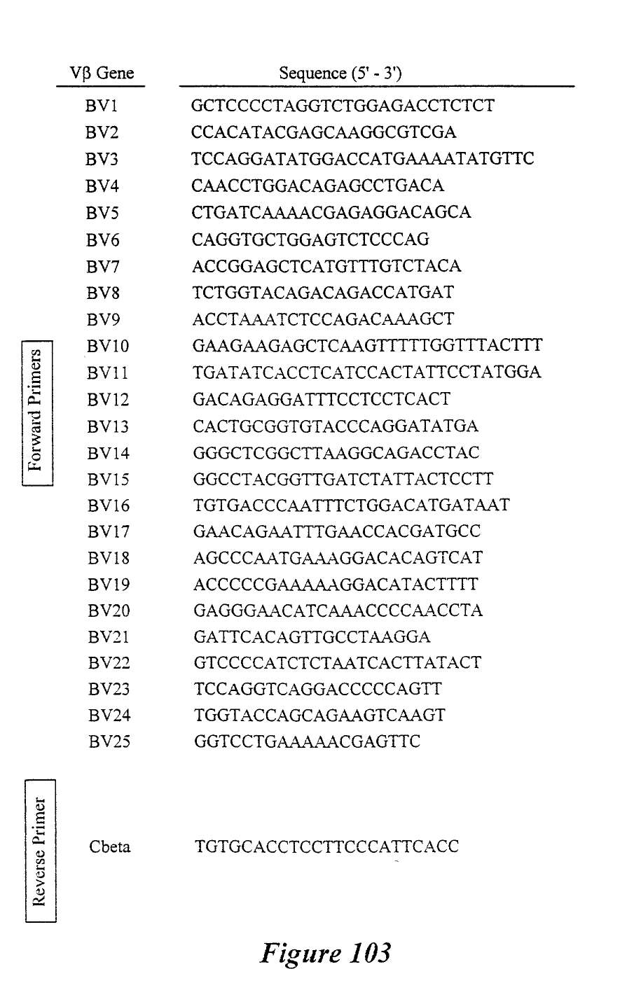

Within one embodiment, PCR amplification is utilized in order to obtain a T cell receptor nucleic acid. Briefly, within one embodiment of the present invention PCR reactions are carried out in a Perkin-Elmer 9600, utilizing a final sample volume of 10 microliters. The reaction mixture should include 25 ng of DNA, 10 pMoles of each primer, 2.5 mM Magnesium chloride, 200 uM of dATP, dTTP, dCTP and dGTP, 50 mM potassium chloride, 20 mM TRIS, pH8.3 and 0.25 units of Taq DNA polymerase. The cycling conditions are 90 seconds at 94° C., 30 cycles of 15 seconds at 94° C., 20 seconds at 54° C. and 30 seconds at 72° C., followed by 3.5 minutes at 72° C. In a particularly preferred embodiment, the primer pairs consist of one member of the list in FIG. 101 and the Cβ primer: TGTGGGAGATCTCTGCTTCT (Sequence I.D. No. 1181). In another particularly preferred embodiment, the primer pairs are those shown in FIG. 102. Within yet another embodiment, the primer pairs are those shown in FIG. 103, and the cycling conditions are: 33 cycles of 20 seconds at 94° C., 45 seconds at 60° C., and 90 seconds at 72° C.

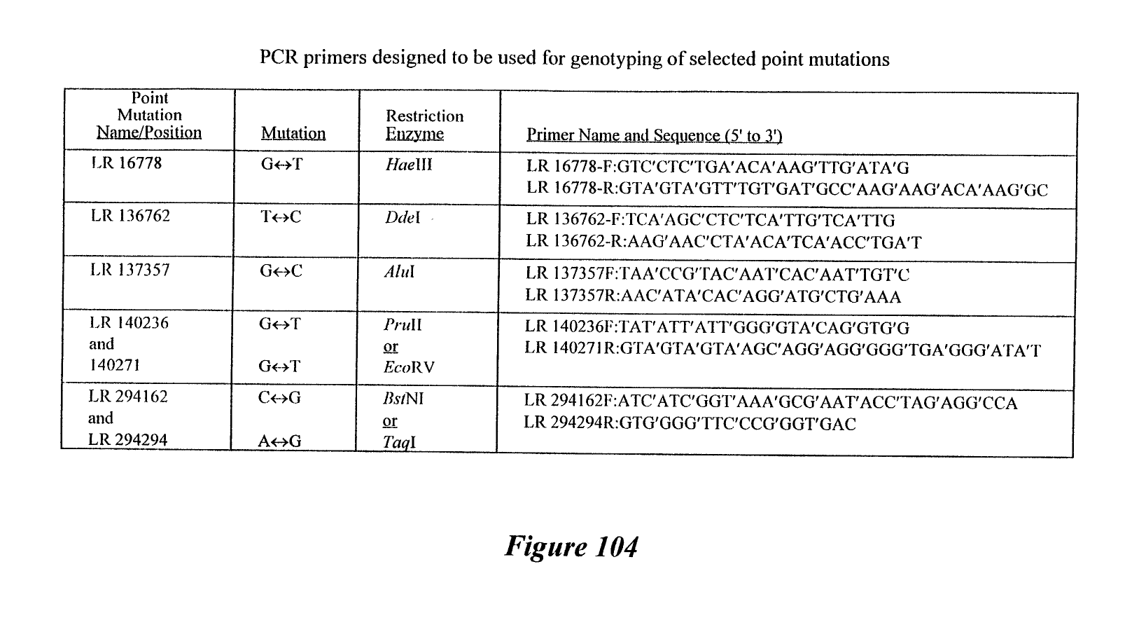

Within related embodiments of the invention, PCR primers may be designed to amplify, and thus allow interrogation of, any selected region, including for example, the genotyping of point mutations. For example, utilizing the PCR primer pairs set forth in FIG. 104 a variety of different point mutations may be amplified under standard PCR conditions (e.g., 35 cycles with an annealing temperature of 60° C.), in order to allow detection of the point mutation. Moreover, the sequence of the amplified fragment may be analyzed in order to allow determination of other polymorphisms.

Within other aspects of the present invention, probes may be designed and synthesized for a variety of therapeutic uses. Briefly, once a sequence-specific probe is designed and verified, the sequence may be incorporated into other molecules. For example, antisense molecules may be prepared based upon a probe sequence, and utilized in order to inhibit expression of a particular V gene which may be involved in disease progression (U.S. Pat. Nos. 5,135,917; 5,248,671). Various gene therapy techniques as described below may be utilized in order to introduce the antisense molecule into all T cells or target it to a subset of T cells. Within other embodiments of the invention, the probe sequence may be incorporated into a ribozyme sequence (U.S. Pat. Nos. 5,116,742; 5,225,337; 5,246,921). Briefly, ribozymes are used to cleave specific RNAs, and are designed such that it can only affect one specific RNA. The substrate binding sequence is between 10 to 20 nucleotides long. The length of this sequence is sufficient to allow a hybridization event with the target RNA and dissociation of the ribozyme from the cleaved RNA molecule. Ribozymes may be delivered to T cells by a variety of different methods, including those which are discussed in more detail below. (See pharmaceutical compositions described below.)

### Antibodies to TCR Vβ

As noted above, the present invention also provides antibodies which are capable of specifically binding to either whole TCR (e.g., an αβ dimer), β chain alone, peptide fragments, or synthetic peptides containing Vβ amino acids. Within the context of the present invention the term “antibodies” includes polyclonal antibodies, monoclonal antibodies, fragments thereof such as F(ab′)₂ and Fab fragments, as well as recombinantly produced binding partners. These binding partners incorporate the variable regions from a gene which encodes a specifically binding monoclonal antibody. Antibodies are defined to be specifically binding if they bind to a particular T cell receptor (or β chain) if it binds to the receptor with an affity of greater than about Kₐ 10⁸ M⁻¹ (see Scatchard, Ann. N.Y. Acad. Sci. 51:660-672, 1949). Within particularly preferred embodiments of the invention, antibodies are provided which are capable of specifically binding to any of TCRBV1S1, TCRBV2S1, TCRBV2S2, TCRBV3S1, TCRBV4S1, TCRBV4S2, TCRBV5S1, TCRBV5S2, TCRBV5S3, TCRBV5S5, TCRBV5S6, TCRBV5S7, TCRBV5S8, TCRBV5S9, TCRBV6S1, TCRBV6S3, TCRBV6S4, TCRBV6S5, TCRBV6S7, TCRBV6S10, TCRBV6S11, TCRBV6S12, TCRBV6S14, TCRBV7S1, TCRBV7S2, TCRBV7S3, TCRBV8S1, TCRBV8S2, TCRBV8S3, TCRBV8S4, TCRBV8S5, TCRBV9S1, TCRBV9S2, TCRBV10S1, TCRBV10S2, TCRBV11S1, TCRBV12S2, TCRBV12S3, TCRBV12S4, TCRBV13S1, TCRBV13S2, TCRBV13S3, TCRBV13S4, TCRBV13S5, TCRBV13S6, TCRBV13S7, TCRBV13S8, TCRBV14S1, TCRBV15S1, TCRBV16S1, TCRBV17S1, TCRBV18S1, TCRBV19S1, TCRBV20S1, TCRBV21S1, TCRBV21S3, TCRBV21S4 TCRBV22S1, TCRBV23S1, TCRBV24S1, TCRBV25S1, TCRBV26S1, TCRBV27S1, TCRBV28S1, TCRBV29S1, TCRBV30S1, TCRBV31S1, TCRBV32S1 TCRBV33S1 and TCRBV34S1.

Briefly, polyclonal antibodies may be readily generated by one of ordinary skill in the art from a variety of animals, such as rabbits, mice, and rats. Briefly, animals are immunized with Vβ protein, either alone or with an adjuvant, such as Freund's complete adjuvant, or one of a number of commercial adjuvants, by intraperitoneal, intramuscular, subcutaneous, or intravenous injections. The Vβ protein may be part of a complete TCR molecule, an isolated chain, a peptide fragment, a synthetic peptide, or a cell which expresses TCR naturally or by transformation with a vector containing the Vβ gene construct of interest. Several injections may be required to generate sufficient antibody concentration in serum. Small samples of serum are collected and tested for reactivity to the Vβ immunogen by any of a number of methods, including ELISA. Particularly preferred polyclonal antisera will give a signal that is at least three times greater than background. After the titer has reached a desirable level or a plateau in terms of its concentration in serum, larger quantities of antisera may be obtained by weekly bleedings or by exsanguination of the animal.

Monoclonal antibodies may be readily generated by one of ordinary skill in the art from conventional techniques (see U.S. Pat. Nos. RE32,011, 4,902,614, 4,543,439, and 4,411,993; see also Antibodies: A Laboratory Manual, Harlow and Lane (eds.), Cold Spring Harbor Laboratory Press, 1988). Briefly, an animal, typically a mouse or a rat, is immunized with a Vβ protein as described above for generating a polyclonal antibody. The animal may be tested for production of specific antibodies. After an animal displays immunoreactivity, a final injection of the immunogen is administered and three to four days later the spleen or lymph nodes of the animal are removed. Cells from these organs are released after disruption of the organ by physical or enzymatic manipulation. Cells are collected, washed, and red cells may be lysed by the addition of hypotonic solution, followed immediately by a wash in physiological buffer. Immune cells may also be generated by in vitro immunization (see Harlow and Lane, supra).

Immune cells are then immortalized by fusion with a myeloma cell or by transfection with a virus, such as the Epstein-Barr virus or an oncogenic virus. A preferred method is to fuse the immune cells with a suitable non-producing myeloma cell line to create a hybridoma which secretes the anti-Vβ antibody in a monoclonal fashion. Many myeloma cell lines have been developed for fusion partners and are well known in the art. They may be obtained from sources such as the American Type Culture Collection (ATCC), Rockville, Md. (see Catalogue of Cell Lines and Hybridomas, 7th ed., ATCC, 1992). Representative myeloma lines include: for humans SKO-007 (ATCC No. CRL 8033; for mice SP2/0-Ag14 (ATCC No. CRL 1581), NS-1 (ATCC No. TIB 18), and P3X63Ag8 (ATCC No. TIB 9), arid for rats Y3-Ag1.2.3 (ATCC No. CRL 1631) and YB2/0 (ATCC No. CRL 1662). Fusion between the myeloma cell line and the immune cells is preferably accomplished by polyethylene glycol (PEG), but may also be accomplished by other methods known in the art.

Following fusion, cells are cultured in a suitable medium, such as RPMI 1640 or DMEM, supplemented with a protein source, such as fetal bovine serum (e.g. HYCLONE, Logan, Utah) or synthetic formulation. Additionally, feeder cells, typically thymocytes or irradiated splenocytes, may be added. Hybridomas are growth selected by the addition of a reagent that inhibits growth of or is toxic to the myeloma cell due to a mutation in a gene that is required for utilization of the reagent. Lymphocytes alone do not grow in the medium because they are generally non-dividing cells, but a fused cell grows because the wild-type gene supplied by the lymphocyte complements the deficiency of the fusion partner.

Hybridomas are then screened for production of the desired specificity, and hybridomas meeting the criteria may be cloned. Representative assays for screening antibodies include the ELISA assay as noted above, as well as various other related “sandwich” type assays (e.g., dot blots, etc.). Within particularly preferred embodiments, the specificity and affinity of antibodies may be determined by flow cytometry. Briefly, the affinity of binding of each antibody may be determined, for example, in a fluorescence activated cell sorter, and the binding of the antibody to a T cell receptor bearing cell determined by a shift of the flow histogram in the rightward direction. Utilizing such procedures, antibodies may be developed that are specific for a particular Vβ, family-specific (e.g., specific for all members of the Vβ13 family, or specific for some, but not all members of a given family.

Antibodies from the culture supernatants can be purified according to conventional techniques (see Antibodies: A Laboratory Manual, supra). Suitable techniques include salt precipitation, peptide or protein affinity columns, HPLC, protein A or protein G columns, or a combination of these techniques.

Other techniques may also be utilized to construct monoclonal antibodies (Huse et al., Science 246:1275, 1989; Sastry et al., Proc. Natl. Acad. Sci. USA 86:5728, 1989). Techniques include construction of a antibody library in an expression vector. Antibodies may be expressed as single chains, Fab fragments or on the surface of bacteriophages. Clones expressing antibodies of the appropriate specificity are purified and high level expression of the monoclonal antibody fragments are induced.

Similarly, binding partners may also be constructed utilizing recombinant DNA techniques to incorporate the variable regions of a gene which encodes a specifically binding antibody. The construction of these proteins may be readily accomplished by one of ordinary skill in the art (see Larrick et al., “Polymerase Chain Reaction Using Mixed Primers: Cloning of Human Monoclonal Antibody Variable Region Genes From Single Hybridoma Cells,” Biotechnology 7:934-938, September 1989; Riechmann et al., “Reshaping Human Antibodies for Therapy,” Nature 332:323-327, 1988; Roberts et al., “Generation of an Antibody with Enhanced Affinity and Specificity for its Antigen by Protein Engineering,” Nature 328:731-734, 1987; Verhoeyen et al., “Reshaping Human Antibodies: Grafting an Antilysozyme Activity,” Science 239:1534-1536, 1988; Chaudhary etal., “A Recombinant Immunotoxin Consisting of Two Antibody Variable Domains Fused to Pseudomonas Exotoxin,” Nature 339:394-397, 1989; see also, U.S. Pat. No. 5,132,405 entitled “Biosynthetic Antibody Binding Sites”), given the disclosure provided herein. Briefly, within one embodiment, DNA molecules encoding T cell receptor-specific antigen binding domains are amplified from hybridomas which produce a specifically binding monoclonal antibody, and inserted directly into the genome of a cell which produces human antibodies (see Verhoeyen et al., supra; see also Reichmann et al., supra). This technique allows the antigen-binding site of a specifically binding mouse or rat monoclonal antibody to be transferred into a human antibody. Such antibodies are preferable for therapeutic use in humans because they are not as antigenic as rat or mouse antibodies.

Alternatively, the antigen-binding sites (variable region) may be either linked to, or inserted into, another completely different protein (see Chaudhary et al., supra), resulting in a new protein with antigen-binding sites of the antibody as well as the functional activity of the completely different protein. As one of ordinary skill in the art will recognize, the antigen-binding sites of the antibody may be found in the variable region of the antibody. Furthermore, DNA sequences which encode smaller portions of the antibody or variable regions which specifically bind to mammalian TCR may also be utilized within the context of the present invention. These portions may be readily tested for binding specificity to the T cell receptor utilizing assays described below.

Within a preferred embodiment, genes which encode the variable region from a hybridoma producing a monoclonal antibody of interest are amplified using oligonucleotide primers for the variable region. These primers may be synthesized by one of ordinary skill in the art, or may be purchased from commercially available sources. Stratacyte (La Jolla, Calif.) sells primers for mouse and human variable regions including, among others, primers for V_{Ha}, V_{Hb}, V_{Hc}, V_{Hd}, C_{Hl}, V_{L} and C_{L} regions. These primers may be utilized to amplify heavy or light chain variable regions, which may then be inserted into vectors such as IMMUNOZAP™(H) or IMMUNOZAP™(L) (Stratacyte), respectively. These vectors may then be introduced into E. coli for expression. Utilizing these techniques, large amounts of a single-chain protein containing a fusion of the V_{H} and V_{L} domains may be produced (see Bird et al., Science 242:423-426, 1988).

Other “antibodies” which may also be prepared utilizing the disclosure provided herein, and thus which are also deemed to fall within the scope of the present invention include humanized antibodies (e.g., U.S. Pat. No. 4,816,567 and WO 94/10332), micobodies (e.g., WO 94/09817) and transgenic antibodies (e.g., GB 2 272 440).

For example, within one embodiment of the invention, the genes encoding the heavy and light chain variable regions are cloned and the DNA sequence is determined. Amino acid translation is predicted from the sequence. Residues characteristic of human antibodies are introduced by site-directed mutagenesis or by PCR amplification from primers incorporating the residues to be altered. Clones with the new sequence are isolated and verified by determining the DNA sequence. The humanized variable regions are cloned into a vector containing the constant regions of human heavy and light chains, so that the resulting protein contains human framework residues, non-human CDR regions and human constant regions.

Once suitable antibodies have been obtained, they may be isolated or purified by many techniques well known to those of ordinary skill in the art (see Antibodies: A Laboratory Manual, supra). Suitable techniques include peptide or protein affinity columns, HPLC or RP-HPLC, purification on protein A or protein G columns, or any combination of these techniques. Within the context of the present invention, the term “isolated” as used to define antibodies or binding partners means “substantially free of other blood components.”

Antibodies of the present invention may be provided in a variety of forms, including for example as a battery or panel of antibodies with differing reactivities which are contained (either separately or together) within a kit or package. In particular, within one aspect of the present invention, a kit is provided comprising a panel of antibodies which are capable of specifically binding to each and every unique β chain of a T cell receptor. As utilized herein, a panel of antibodies which specifically bind to each and every “unique” β chain of a T cell receptor should not be understood within all embodiments to refer to a panel of antibodies which can specifically bind to each and every Vβ gene product, but rather, to that panel of antibodies which can maximally distinguish the entire complement of Vβ gene products.

Antibodies of the present invention have many uses. For example, antibodies may be utilized in flow cytometry to sort T cell receptor-bearing cells, or to histochemically stain T cell receptor-bearing tissues. Briefly, in order to detect T cell receptors on cells, the cells (or tissue) are incubated with a labeled antibody which specifically binds to a T cell receptor, followed by detection of the presence of bound antibody. These steps may also be accomplished with additional steps such as washings to remove unbound antibody. Representative examples of suitable labels, as well as methods for conjugating or coupling antibodies to such labels are described in more detail below.

In addition, purified antibodies may also be utilized therapeutically to block the binding of T cell receptor substrate to the T cell receptor in vitro or in vivo. As noted above, a variety of assays may be utilized to detect antibodies which block or inhibit the binding of a ligand to a T cell receptor, including inter alia, inhibition and competition assays noted above. Within one embodiment, monoclonal antibodies (prepared as described above) are assayed for binding to the T cell receptor in the absence of a putative ligand, as well as in the presence of varying concentrations of the ligand. Blocking antibodies are identified as those which, for example, bind to a T cell receptor and, in the presence of a ligand, block or inhibit the binding of the ligand to the T cell receptor.

Antibodies of the present invention may also be coupled or conjugated to a variety of other compounds (or labels) for either diagnostic or therapeutic use. Such compounds include, for example, toxic molecules, molecules which are nontoxic but which become toxic upon exposure to a second compound, and radionuclides. Representative examples of such molecules are described in more detail below.

Antibodies which are to be utilized therapeutically are preferably provided in a therapeutic composition comprising the antibody or binding partner and a physiologically acceptable carrier or diluent. Suitable carriers or diluents include, among others, neutral buffered saline or saline, and may also include additional excipients or stabilizers such as buffers, sugars such as glucose. sucrose, or dextrose, chelating agents such as EDTA, and various preservatives.

### Labels

The nucleic acid molecules, antibodies, and T cell receptors (including sTCR) of the present invention may be labeled or conjugated (either through covalent or non-covalent means) to a variety of labels or other molecules, including for example, fluorescent markers, enzyme markers, toxic molecules, molecules which are nontoxic but which become toxic upon exposure to a second compound, and radionuclides.

Representative examples of fluorescent labels suitable for use within the present invention include, for example, Fluorescein Isothiocyanate (FITC), Rhodamine, Texas Red, Luciferase and Phycoerythrin (PE). Particularly preferred for use in flow cytometry is FITC which may be conjugated to purified antibody according to the method of Keltkarnp in “Conjugation of Fluorescein Isothiocyanate to Antibodies. I. Experiments on the Conditions of Conjugation,” Immunology 18:865-873, 1970. (See also Keltkamp, “Conjugation of Fluorescein Isothiocyanate to Antibodies. II. A Reproducible Method,” Immunology 18:875-881, 1970; and Goding, “Conjugation of Antibodies with Fluorochromes: Modification to the Standard Methods,” J. Immunol. Methods 13:215-226, 1970.) For histochemical staining, HRP, which is preferred, may be conjugated to the purified antibody according to the method of Nakane and Kawaoi (“Peroxidase-Labeled Antibody: A New Method of Conjugation,” J. Histochem. Cytochem. 22:1084-1091, 1974; see also, Tijssen and Kurstak, “Highly Efficient and Simple Methods for Preparation of Peroxidase and Active Peroxidase Antibody Conjugates for Enzyme Immunoassays,” Anal. Biochem. 136:451-457, 1984).

Representative examples of enzyme markers or labels include alkaline phosphatase, horse radish peroxidase, and β-galactosidase. Representative examples of toxic molecules include ricin, abrin. diphtheria toxin, cholera toxin, gelonin, pokeweed antiviral protein, tritin, Shigella toxin, and Pseudomonas exotoxin A. Representative examples of molecules which are nontoxic, but which become toxic upon exposure to a second compound include thymidine kinases such as HSVTK and VZVTK. Representative examples of radionuclides include Cu-64, Ga-67, Ga-68, Zr-89, Ru-97, Tc-99m, Rh-105, Pd-109, In-111, I-123, I-125, I-131, Re-186, Re-188, Au-198, Au-199, Pb-203, At-211, Pb-212 and Bi-212.

As will be evident to one of skill in the art given the disclosure provided herein, the above described nucleic acid molecules, antibodies, and T cell receptors may also be labeled with other molecules such as colloidal gold, as well either member of a high affinity binding pair (e.g., avidin-biotin).

### Pharmaceutical Compositions and Therapeutic Uses

As noted above, the present invention provides pharmaceutical compositions, as well as methods for using the same (for either prophylactic or therapeutic use). Briefly, pharmaceutical compositions of the present invention may comprise TCR (including, for example, an entire TCR complex such as αβ, the β chain above, or portions of the β chain above), sTCR, antibody which is capable of specifically binding TCR, TCR antagonists or agonists, antisense sequences and ribozymes, in combination with a pharmaceutically acceptable carrier, diluent, or excipient. Such compositions may comprise buffers such as neutral buffered saline, phosphate buffered saline and the like, carbohydrates such as glucose, mannose, sucrose or dextrose, proteins, polypeptides or amino acids, antioxidants, chelating agents such as EDTA or glutathione, and preservatives.

Compositions of the present invention may be formulated for the manner of administration indicated, including for example, for oral, nasal, venous, vaginal or rectal administration. Within other embodiments, the compositions may be administered as part of a sustained release implant (e.g., intra-articularly). Within yet other embodiments, the compositions may be formulated as a lyophilizate, utilizing appropriate excipients which provide stability as a lyophilizate, and subsequent to rehydration.

Pharmaceutical compositions of the present invention may be utilized in order to treat a wide variety of diseases including, for example, T-cell associated diseases. As utilized herein, “T-cell associated diseases” refers to diseases which are mediated at least in part by T cells, or a subpopulation of T cells. Generally, such diseases include, for example, the general classes of autoimmune diseases, degenerative nervous system diseases, graft-versus-host disease, hypersensitivity diseases, infectious diseases, and neoplastic diseases. Representative examples of autoimmune diseases include Addison's disease, atrophic gastritis, autoimmune hemolytic anemia, autoimmune neutropenia, bullous pemphigoid, Crohn's disease, coeliac disease, demyelinating neuropathies, dermatomyositis, Goodpasture's syndrome, Graves' disease, hemolytic anemia, idiopathic thrombocytopenia purpura, inflammatory bowel disease, insulin-dependent diabetes mellitus, juvenile diabetes, multiple sclerosis, myasthenia gravis, myocarditis, myositis, myxedema, pemphigus vulgaris, pernicious anaemia, primary glomerulonephritis, rheumatoid arthritis, scleritis, scleroderma, Sjogren's syndrome, systemic lupus erythematosus, and type I diabetes. Representative examples of degenerative nervous system diseases include multiple sclerosis and Alzheimer's disease. Representative examples of hypersensitivity diseases include Type I hypersensitivities such as contact with allergens that lead to allergies, Type II hypersensitivities such as those present in Goodpasture's syndrome, myasthenia gravis, and autoimmune hemolytic anemia, and Type IV hypersensitivities such as those manifested in leprosy, tuberculosis, sarcoidosis and schistosomiasis. Representative examples of infectious diseases include viral infections caused by viruses such as HIV, HBV (e.g., A, B or C), HSV, HPV, EBV, CMV, influenza; fungal infections such as those caused by the yeast genus Candida; parasitic infections such as those caused by schistosomes, filaria, nematodes, trichinosis, or protozoa such as trypanosomes causing sleeping sickness, plasmodium causing malaria or leishmania which cause leischmaniasis; and bacterial infections such as those caused by mycobacterium, corynebacteriurn, streptococcus, or staphylococcus. Representative examples of neoplastic diseases include lymphoproliferative diseases such as leukemias, lymphomas, Non-Hodgkin's lymphoma, and Hodgkin's lymphoma, and cancers such as cancer of the brain, breast, colon, lung, liver, pancreas, and prostate.

Pharmaceutical compositions of the present invention may be administered in a manner appropriate to the disease to be treated (or prevented). Although appropriate dosages may be determined by clinical trials, the quantity and frequency of administration will be determined by such factors as the condition of the patient, and the type and severity of the patient's disease.

Within other aspects of the present invention, viral vectors are provided which may be utilized to treat diseases wherein either the T cell receptor (or a mutant T cell receptor) is over-expressed, or where no T cell receptor is expressed. Briefly, within one embodiment of the invention, viral vectors are provided which direct the production of antisense T cell receptor RNA, in order to prohibit the over-expression of T cell receptors, or the expression of mutant T cell receptors. Within another embodiment, viral vectors are provided which direct the expression of T cell receptor cDNA. Viral vectors suitable for use in the present invention include, among others, recombinant vaccinia vectors (U.S. Pat. Nos. 4,603,112 and 4,769,330), recombinant pox virus vectors (PCT Publication No. WO 89/01973), and preferably, recombinant retroviral vectors (“Recombinant Retroviruses with Amphotropic and Ecoptropic Host Ranges,” PCT Publication No. WO 90/02806; “Retroviral Packaging Cell Lines and Processes of Using Same,” PCT Publication No. WO 89/07150; and “Antisense RNA for Treatment of Retroviral Disease States,” PCT Publication No. WO/03451), and herpesvirus vectors (Kit, Adv. Exp. Med. Biol. 215:219-236, 1989; U.S. Pat. No. 5,288,641).

Within various embodiments of the invention, the above-described compositions may be administered in vivo, or ex vivo. Representative routes for in vivo administration include intradermally (“i.d.”), intracranially (“i.c.”), intraperitoneally (“i.p.”), intrathecally (“i.t.”), intravenously (“i.v.”), subcutaneously (“s.c.”) or intramuscularly (“i.m.”).

Within other embodiments of the invention, the vectors which contain or express nucleic acid molecules of the present invention, or even the nucleic acid molecules themselves, may be administered by a variety of alternative techniques, including for example direct DNA injection (Acsadi et al., Nature 352:815-818, 1991); microprojectile bombardment (Williams et al., PNAS 88:2726-2730, 1991); liposomes (Pickering et al., Circ. 89(1):13-21, 1994; and Wang et al., PNAS 84:7851-7855, 1987); lipofection (Felgner et al., Proc. Natl. Acad. Sci. USA 84:7413-7417, 1989); DNA ligand (Wu et al., J. of Biol. Chem. 264:16985-16987, 1989); administration of DNA linked to killed adenovirus (Michael et al., J. Biol. Chem. 268(10):6866-6869, 1993; and Curiel et al., Hum. Gene Ther. 3(2):147-154, 1992), retrotransposons, cytofectin-mediated introduction (DMRIE-DOPE, Vical, Calif.) and transferrin-DNA complexes (Zenke).

### Magnetic, Electronic and Optical Storage, Transmission and Use of Vβ Sequence Information

The present invention also provides devices wherein the entire T cell receptor β gene locus (or portions thereof) may be placed or contained on storage media (e.g., magnetic, electronic or optical forms), and further, transmitted or utilized in a variety of applications. For example, within one embodiment of the invention the T cell receptor sequence disclosed in Seq. I.D. No. 1 may be stored on magnetic storage media either entirely, or in portions. For example, the portions may be of greater than about 25 to 50 kb, preferably greater than 100 to 150 kb, more preferably greater than 200 to 250 kb, and most preferably greater than 300, 350, 400, 450, 500, 550, 600 or 650 kb. Representative examples of suitable magnetic storage media include 5¼- and 3½-inch floppy disks of various densities and manufacturers (e.g., single-sided or double-sided disks from manufacturers such as Memorex, Verbatim, Maxell, and 3M) and magnetic tape (e.g., 0.5 inch with a density ranging from 1600 to 6,250 bits per inch, 9 track). Alternatively, such sequence information may be stored within the hard drive or on the electronic storage memory (e.g, RAM or ROM) of a computer. Within other embodiments of the invention, the T cell receptor sequences disclosed herein may be contained within optical storage media or utilized within optical matrices. Representative examples of such optical devices include CD-ROM disk and magnetio-optical disks.

The present invention also provides methods for transmitting the data from one location to another, including for example, by modem transfer utilizing any of a variety of file transfer protocols (e.g., Kermit, X-Modem, etc.).

Within yet another aspect of the present invention, methods are provided for utilizing the sequence information provided herein. For example, within one aspect of the present invention, methods are provided in a computer system for storing sequence information about a T cell receptor β gene, such methods comprising the steps of, for each base within a portion of a T cell receptor β gene sequence, inputting an indication of the type of the base, representing the type of the base in a format suitable for storage, and storing the representation of the base type so that both the type of the base and the position of the base within the T cell receptor β gene sequence can be later retrieved. Complementary methods are provided in this aspect of the invention for retrieving stored sequence information about a T cell receptor β gene, such methods comprising the steps of, for each base within a portion of a T cell receptor β gene sequence, retrieving a representation of the type of the base from the stored sequence information, and, when necessary, converting the retrieved representation into a different format. Within a related embodiment, the stored sequence information is compressed by one or more data compression techniques such as run length encoding, LZ77-LZ78 compression, and Huffman encoding. Within still another aspect of the present invention, methods are provided for searching sequence information about a portion of a T cell receptor β gene, the methods comprising the steps of receiving a criterion for the search, identifying a subset of bases within the T cell receptor β gene sequence that meets the criterion, and selecting the identified subset of bases from the T cell receptor β gene sequence. In the preferred embodiments of the present invention, the portions of the T cell receptor β gene sequence stored, retrieved, and searched include portions of lengths greater than about 25 to 50 kb, preferably greater than 100 to 150 kb, more preferably greater than 200 to 250 kb, and most preferably greater than 300, 350, 400, 450, 500, 550, 600, or 650 kb.

Briefly, numerous programs are commercial available and suitable for analyzing the Vβ sequence. For example, many of the basic analytical tools for the analysis of DNA are available in the GCG package (Genetics Computer Corp., Madison, Wis., 608-231-5200.). Other programs which may also be readily obtained and utilized include restriction site mapping programs, such as Map, MapPlot and MapSort (GCG). (Restriction maps are used to subclone fragments and to design and interpret hybridization experiments.) Primers for hybridization, sequencing or PCR amplification may also be selected utilizing programs such as Primer (contact primer@genome.wi.edu), which has been discussed in more detail above.

Other programs that may also be utilized to store and manipulate data include Perl (a unix text processing language), which can be used to manipulate and extract DNA sequences from a string (available at ftp.uu.net). Briefly, Perl can be used to change file formats, digest the results of other programs and extract subsequences from the whole sequence. It can also be used to extract subsequences for synthesis of the Vβ genes or variants or mutants of the genes, and to design anti-sense RNA or DNA to block expression of particular genes.

Within other embodiments, sequence similarity searches can be carried out with programs such as BLAST (ftp from ncbi.nlm.nih.gov) or fasta or tfasta (GCG). Sequence comparisons can also be carried out with Compare, DotPlotBestFit or Gap (GCG). Multiple alignments of related proteins can be performed with PileUp (GCG). More distant relatives of the T-cell receptors could be recognized by application of ProfileMaker and ProfileSearch (GCG) to identify conserved patterns and use these to search the databases. Such alignment and profiling tools may also be used to recognize regulatory sequences which control expression of the genes.

Sequence searching and alignment techniques may also be used to identify the most closely related sequences for which a three-dimensional structure is available. In particular, having the amino acid sequence encoded by the entire set of Vβ genes provides an unprecedented opportunity to model the structure of the variable region of the T cell receptor. Briefly, based upon the techniques of homology modeling (see Lee and Levitt, Nature 352:448-451, 1991; Levitt, J. Mol. Biol. 226:507-533, 1992; Lee, J. Mol. Biol. 236:918-939, 1994; and U.S. Pat. No. 5,241,470), consensus sequences of the Vβ gene family may be determined, and the general family structure determined based upon the known structure of several well-known high resolution structures (i.e., the immunoglobulin family variable region). Having the entire set of V β gene products makes it much easier to model the consensus structure of the protein because it identifies with higher reliability than with fewer examples the common amino acid residues in the sequences. Suitable programs for carrying out the above analysis include, for example, the program Look (Molecular Applications Group, Palo Alto, Calif.), which will model the three-dimensional structure of an unknown protein based on an alignment to a known structure, yielding a model with optimal side-chain packing.

One related advantage provided by the above modeling is that the structural differences among the T cell receptor variable regions should also be predictable. In particular, differences among the Vβ sequences that result in structural features that can be recognized by another molecule (e.g., a feature on the accessible surface of the protein) can be identified if all Vβ sequences are known. Such information allows the assessment of a particular structural feature upon the biological impact of a particular T cell receptor.

### Methods for Identification of Vβ Genes

The present invention also provides methods for identifying and interrogating particular Vβ genes and groups of Vβ genes. Briefly, as noted above, previous to the present invention less than 4% of the sequence for the Vβ gene was known. Therefore, it was difficult to construct probes and primers suitable for amplifying and uniquely identifying each Vβ gene. Given the sequence information disclosed within the present application, novel primers and probes can now be developed which are suitable for amplifying and interrogating each Vβ gene. In particular, given the complete DNA sequences of each Vβ gene and flanking regions, DNA sequence regions of similarity and uniqueness may be identified by computer analysis. Generally, regions of unique sequence which are optimally 15 to 30 bases long are suitable for oligonucleotide probes and primers. Especially preferred are regions with multiple differences to reduce annealing of the primer to a non-identical sequence and regions with differences concentrated in an area such that the 3′ most bases of the primer will not cross-anneal to a non-identical sequence. With these criteria in rnind, a set of oligonucleotide primers for each Vβ gene may be designed. A preferred length of the primers is from 15 to 30 bases.

In order to identify or interrogate a particular Vβ gene, appropriate primers and/or probes are identified and prepared as discussed above. Once such probes and/or primers have been identified, each unique Vβ gene may be readily interrogated. For example, within one aspect of the present invention, target DNA is first prepared from a T cell population or a clone. Target DNA may be either genomic DNA, or cDNA generated from RNA. Within this embodiment of the present invention, oligonucleotide primers are selected to anneal within coding regions of the Vβ gene of interest. PCR amplification may be conducted by thermal cycling, preferably in an automated fashion utilizing, for example, a Perkin Elmer 9600 Thermal Cycler. Conditions for thermal cycling are optimized using each set of oligonucleotide primers on a homogeneous source of DNA containing the target region. Parameters to be determined include incubation times for annealing, extension, and denaturation, incubation temperatures for each part of the cycle, number of cycles, and cation concentration. Once conditions are established, amplifications may be performed.

Amplified products may be detected by techniques well known to one skilled in the art. For example, products may be visualized by intercalation of ethidium bromide concomitant with gel electrophoresis, or by transfer of the amplification reaction to a solid support, such as a nylon membrane, and subsequent hybridization with a sequence-specific probe. The probe may be detected from a radiolabel attached to the probe or by non-radioactive detection methods. Radiolabeling of an oligomer is preferably accomplished by the transfer of ³²pO₄ to the 5′-OH group of the oligomer by polynucleotide kinase. Alternatively, either a small molecule, such as digoxigenin or biotin, is incorporated into the DNA probe. A protein which binds to the small molecule, such as an antibody or avidin, is coupled to an enzyme capable of cleaving a chemiluminescent substrate, or the enzyme is coupled directly to the probe. In all cases, the probe is hybridized to the amplification products. Preferred hybridization conditions vary according to whether the probe is an oligomer or a longer piece of DNA. Typical hybridization conditions for oligomers of various lengths can be found in (5M TMACl hybridization conditions). Typical hybridization conditions for longer pieces of DNA are well known in the art (see Maniatis; Greene). After hybridization and washing off any unhybridized probe, hybrids are detected either by direct exposure to film, phosphor imaging (for radioactive probes), or subsequent application of a chemiluminescent substrate and exposure to film.

Within another aspect of the present invention, amplification primers are selected to anneal to sequences flanking a particular Vβ gene. In this case, the primers are designed to amplify one specific Vβ gene. Two general criteria may be utilized in selecting oligonucleotide. primer sequences. The first criteria is to find a region of unique sequence for a Vβ gene. The second criteria is to identify, among the unique sequences, a sequence which has low identity to regions flanking the remaining Vβ genes and, if possible, has a cluster of low identity at the 3′ end of the primer.

One such strategy to identify primer pairs is as follows. First, sequences of all the Vβ genes and flanking regions need to be determined. It is important to also have determined the sequences of Vβ genes which are pseudogenes and other non-expressed Vβ genes in order to design a truly specific primer. The sequences of each V β and flanking regions are aligned and regions of uniqueness and low homology identified. Identification can be made by composing the alignment to highlight non-identical bases. Regions of low homology will then be readily apparent. Alternatively, a computer program can be used to identify these regions.

When candidate primer pairs are identified for each Vβ gene, testing of these pairs is done to confirm their specificity. Each primer pair is used in amplification of either genomic DNA or a set of clones that contains each Vβ gene. Optimal conditions for amplification are determined as above. If amplification is performed on genomic DNA, identity of the amplified Vβ gene can be made by hybridization with sequence-specific probes or by determining the DNA sequence of the amplified product. Any primer pairs that do not specifically amplify a single Vβ gene can be discarded and a new set chosen based on the criteria outlined above.

Analysis of the presence of a particular Vβ gene in an individual is made by amplification of genomic DNA. Cells are isolated from an individual. Peripheral blood cells are one readily obtainable source of cells from a human. Other cell sources can also be used, such as skin or sperm. DNA is isolated from the cells by any one of a number of methods known to one skilled in the art (see Sambrook et al., supra). Amplification is performed under either a standard set of conditions (see PCR Protocols, supra) or under a set of optimally determined conditions. Detection of amplified product is made visually after gel electrophoresis, or by hybridization with a radioactive or non-radioactive probe. The preferred probe is sequence specific for the particular Vβ gene in question, but a general probe for Vβ genes can also be used. Another method of detection of the amplified product is to use radioactive-labeled primers or radioactive-labeled nucleotides in the amplification. Detection of amplified product is then made by autoradiography or phosphor imaging following removal of unincorporated labeled material by gel electrophoresis, gel filtration, or other separation techniques.

Within another aspect of the present invention, Vβ PCR products are cloned into a vector such as, for example, PGem-7zf (promega). The plasmids are then linearized, and their in vitro transcription products are tested in pools against populations of T cell mRNA isolated from a variety of individuals in an RNAse protection assay (Baccala et al., PNAS 88:2908-2912, 1991).

### Analysis of Polymorphisms

Within particularly preferred aspects of the present invention, utilizing the above-described principles, one can readily determine a correlation between a disease or disease susceptibility and a selected polymorphism. For example, within one embodiment, methods are provided comprising the steps of: (a) obtaining biological samples containing nucleated cells from a population, the population having individuals with a selected disease or disease susceptibility and individuals without the disease or disease susceptibility or individuals who are in remission from the selected disease, (b) extracting nucleic acids from the cells, (c) contacting the extracted nucleic acids with primers capable of specifically priming and allowing amplification of a selected polymorphism, (d) amplifying the selected polymorphism, and (e) detecting the presence of the polymorphism, and thereby determining a correlation between the disease or disease susceptibility and the selected polymorphism. Within another embodiment, methods are provided for determining a correlation between a disease and a selected polymorphism, comprising the steps of: (a) obtaining biological samples containing nucleated cells from a population, the population having individuals with a selected disease and individuals without the disease, (b) extracting ribonucleic acids from the cells, (c) reverse transcribing cDNA from the ribonucleic acids, (d) contacting the cDNA with primers capable of specifically priming and allowing amplification of a selected polymorphism, (e) amplifying the selected polymorphism, and (f) detecting the presence of the polymorphism, and thereby determining a correlation between the disease and the selected polymorphism.

Within yet another related aspect, methods are provided for determining a correlation between a disease and a selected polymorphism, comprising the steps of: (a) obtaining biological samples containing nucleated cells from a population, the population having individuals with a selected disease and individuals without the disease, (b) extracting nucleic acids from the cells, and (c) detecting the presence of the polymorphism, and thereby determining a correlation between the disease or disease susceptibility and the selected polymorphism.

As noted above, a variety of polymorphisms may be readily detected, including, for example, restriction fragment length polymorphisms, length differences of a simple repeat sequence, and specific nucleotide substitution, deletion or insertion.

Such polymorphisms may be correlated with a wide variety of T cell associated diseases Within other embodiments, the disease or disease susceptibility may be selected from the group consisting of Addison's disease, atrophic gastritis, autoimmune hemolytic anemia, autoimmune neutropenia, bullous pemphigoid, Crohn's disease, coeliac disease, demyelinating neuropathies, dernatomyositis, Goodpasture's syndrome, Graves' disease, hemolytic anemia, idiopathic thrombocytopenia purpura, inflammatory bowel disease, insulin-dependent diabetes mellitus, juvenile diabetes, multiple sclerosis, myasthenia gravis, myocarditis, myositis, myxedema, pemphigus vulgaris, pernicious anaemia, primary glomerulonephritis, rheumatoid arthritis, scleritis, scleroderma, Sjogren's syndrome, systemic lupus erythematosus, and type I diabetes.

Within other aspects of the present invention, methods are provided for determining a correlation between a disease resistance or disease susceptibility and a genetic marker, comprising the steps of: (a) obtaining biological samples containing nucleated cells from a population, the population having individuals with a selected disease resistance or disease susceptibility and individuals without the disease resistance or disease susceptibility, (b) extracting nucleic acids from the cells, (c) contacting the extracted nucleic acids with primers which are capable of specifically priming and allowing amplification of a series of selected genetic markers in the T cell receptor β gene region, the markers being selected such that they are in linkage disequilibrium with each other, (d) amplifying the genetic markers, and (e) determining the length of the amplified material, and thereby determining the correlation between a disease resistance or disease susceptibility and a genetic marker. As utilized within the context of the present invention, genetic markers are deemed to be in linkage disequilibrium with each other if there is a statistically significant correlation between the genetic markers. Within certain embodiments, the series of genetic markers are at least 5 to 35 kb apart, and more preferably, at least 10 to 20 kb apart.

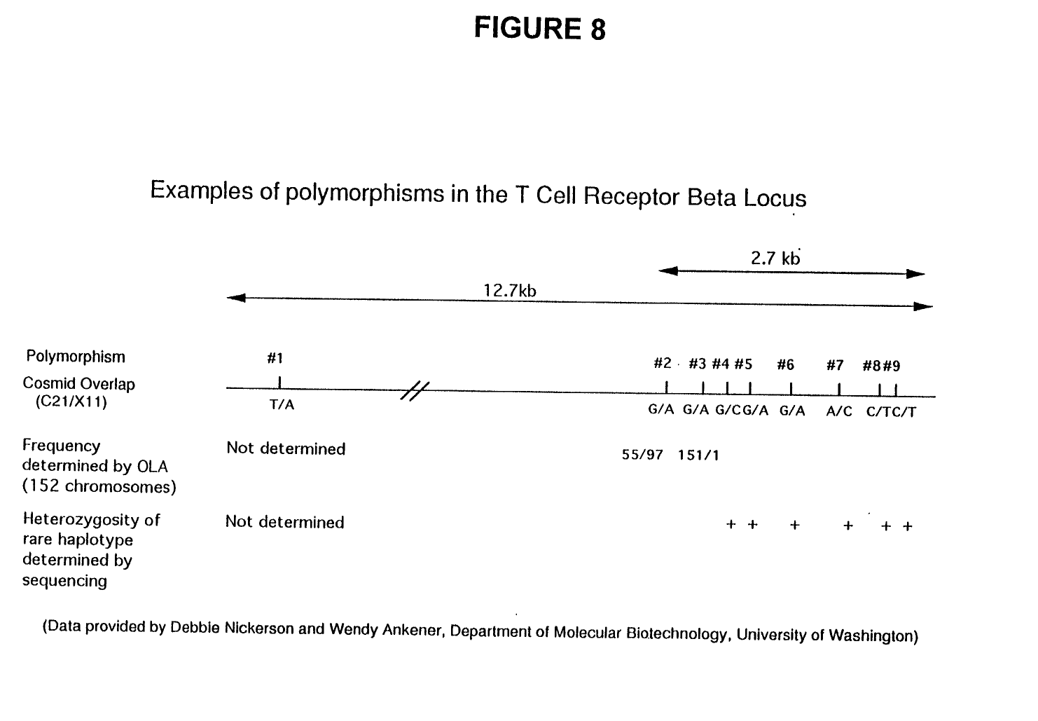

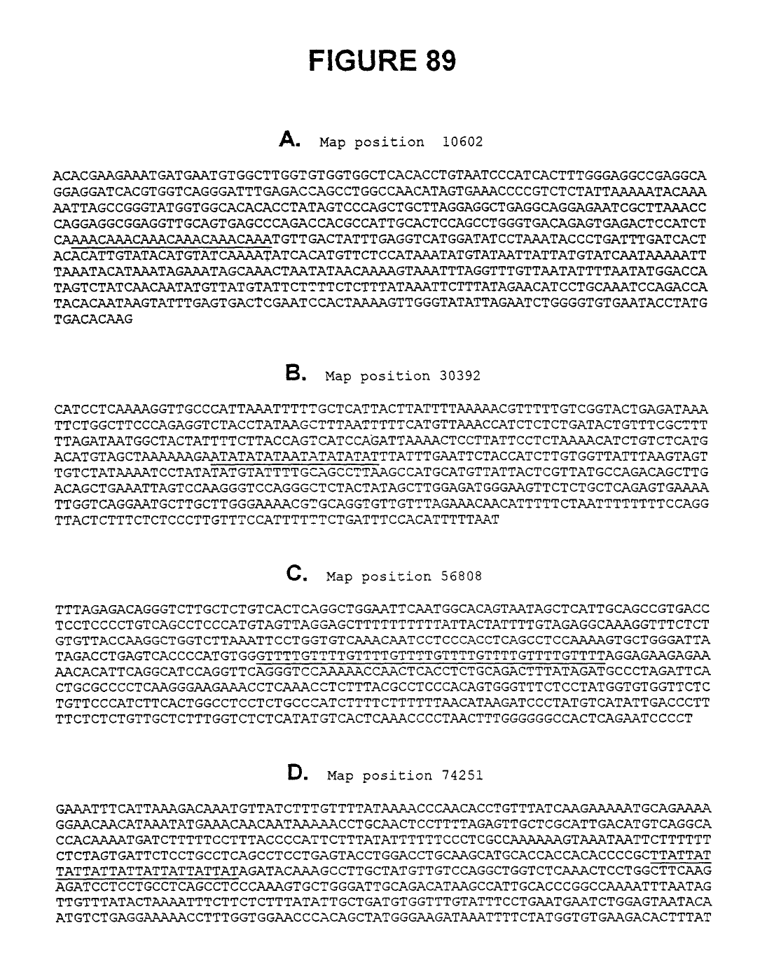

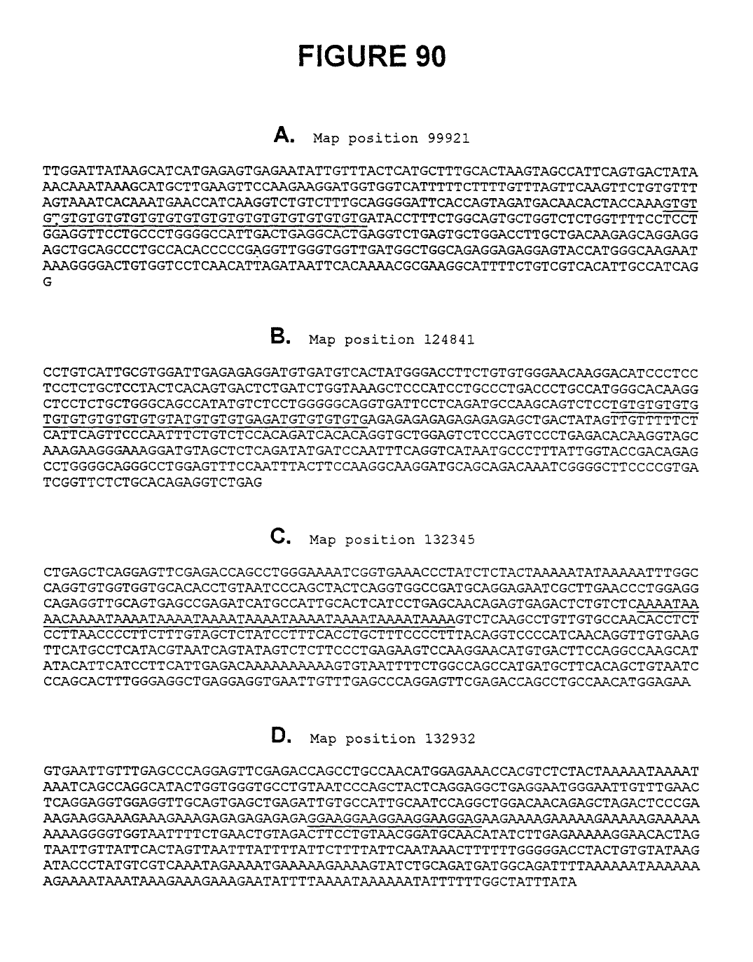

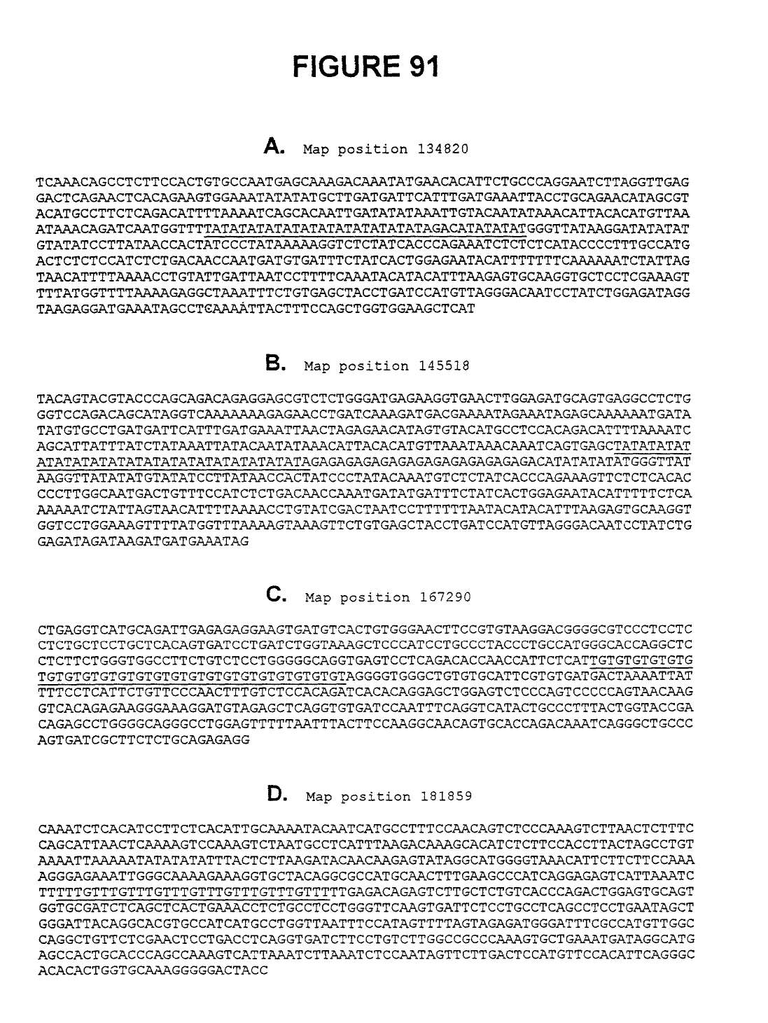

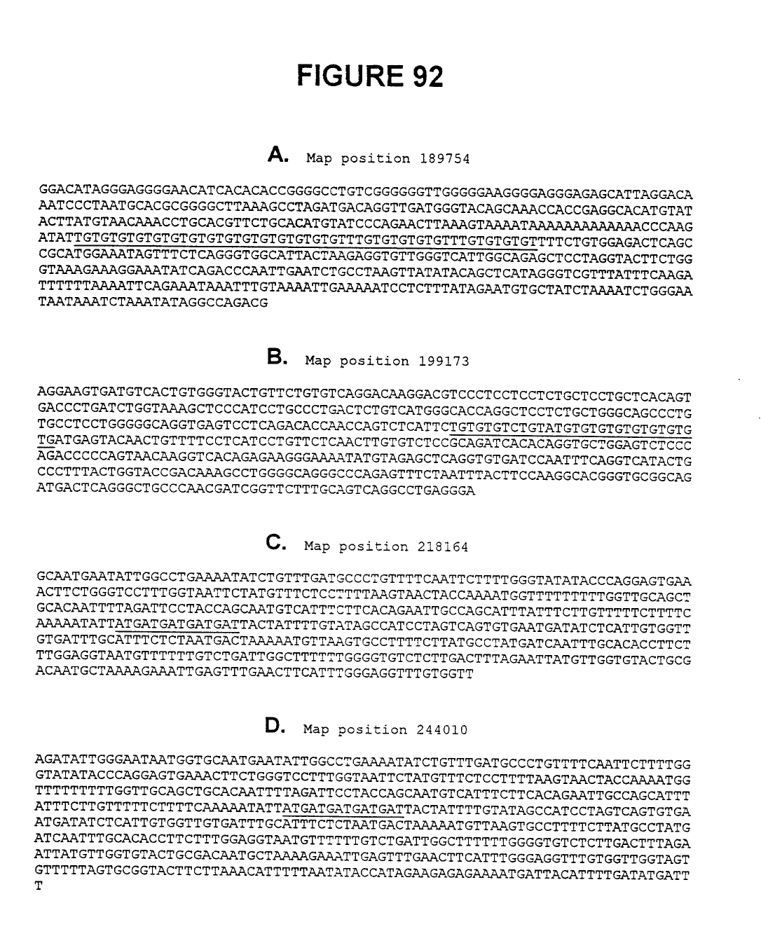

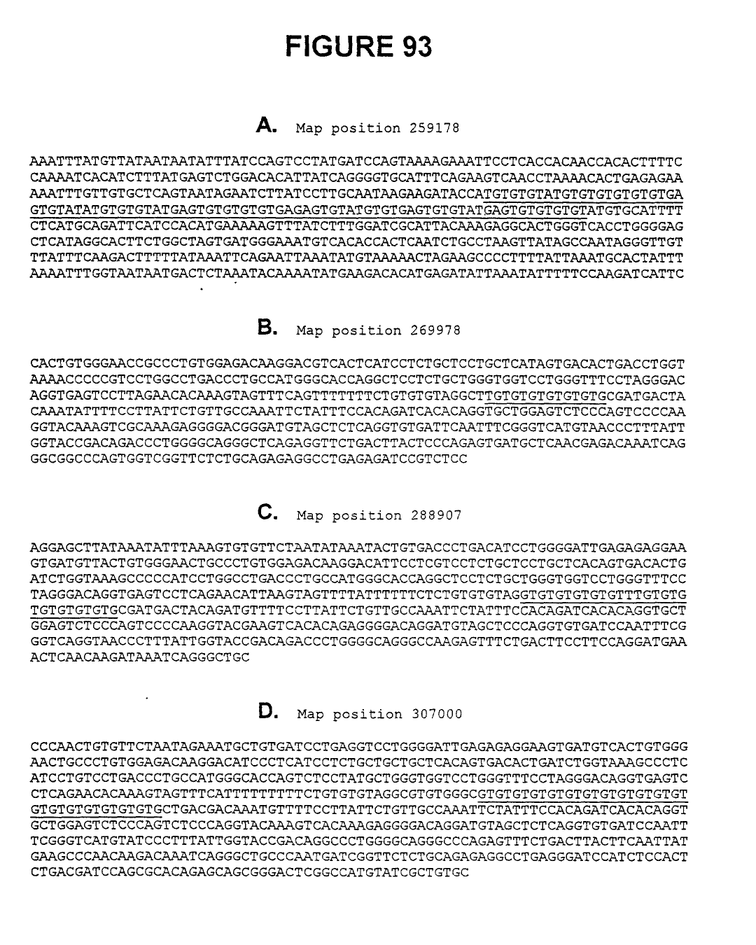

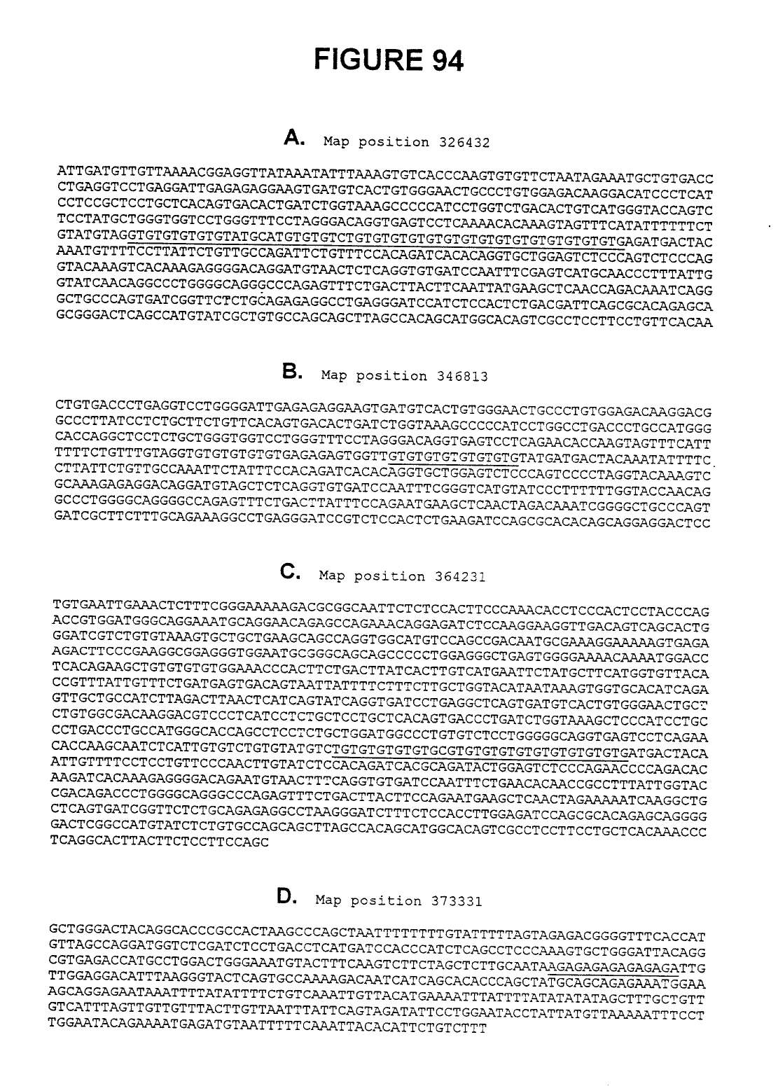

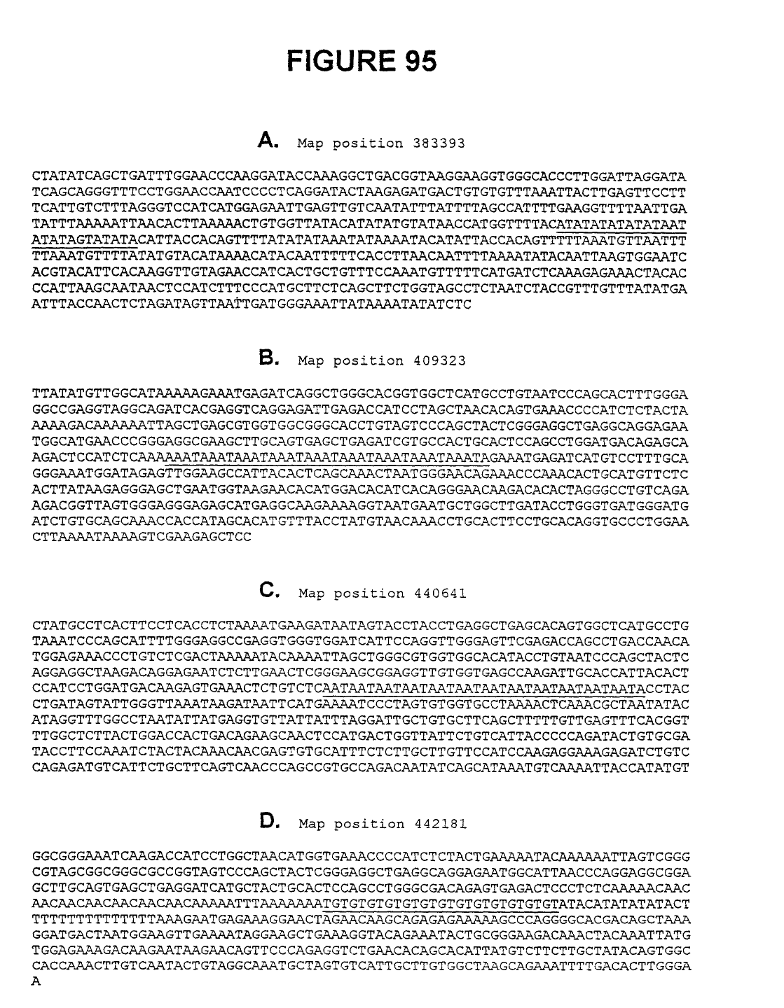

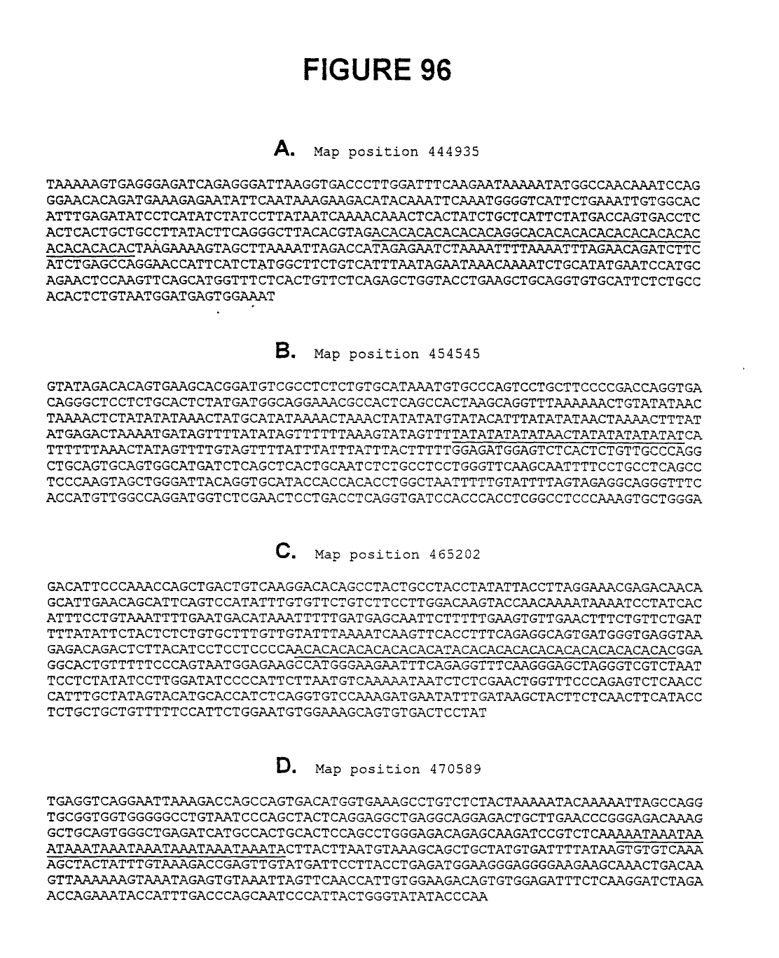

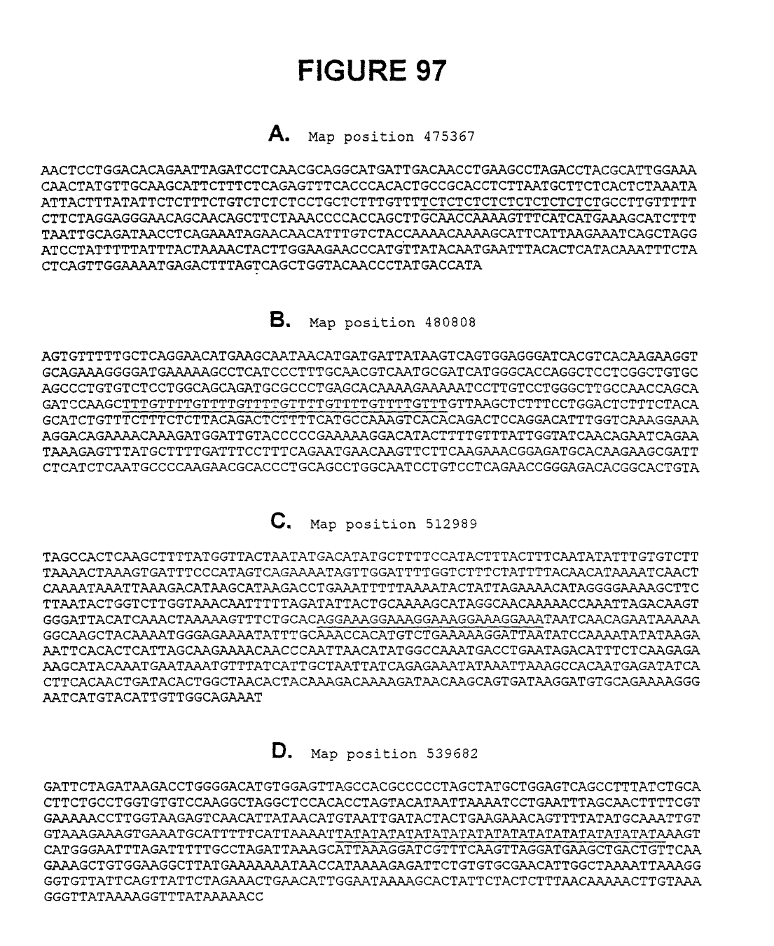

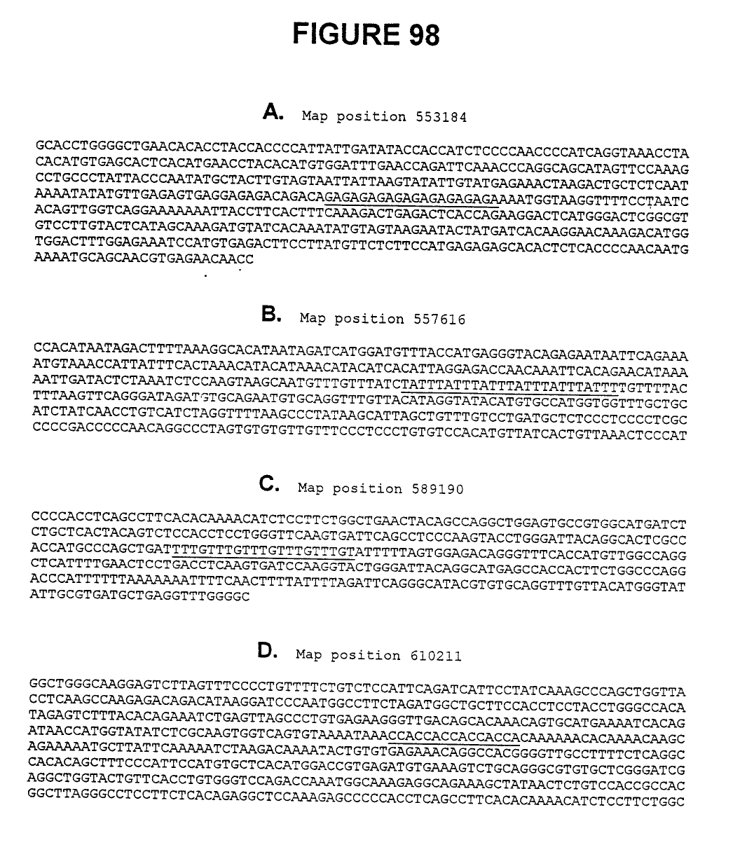

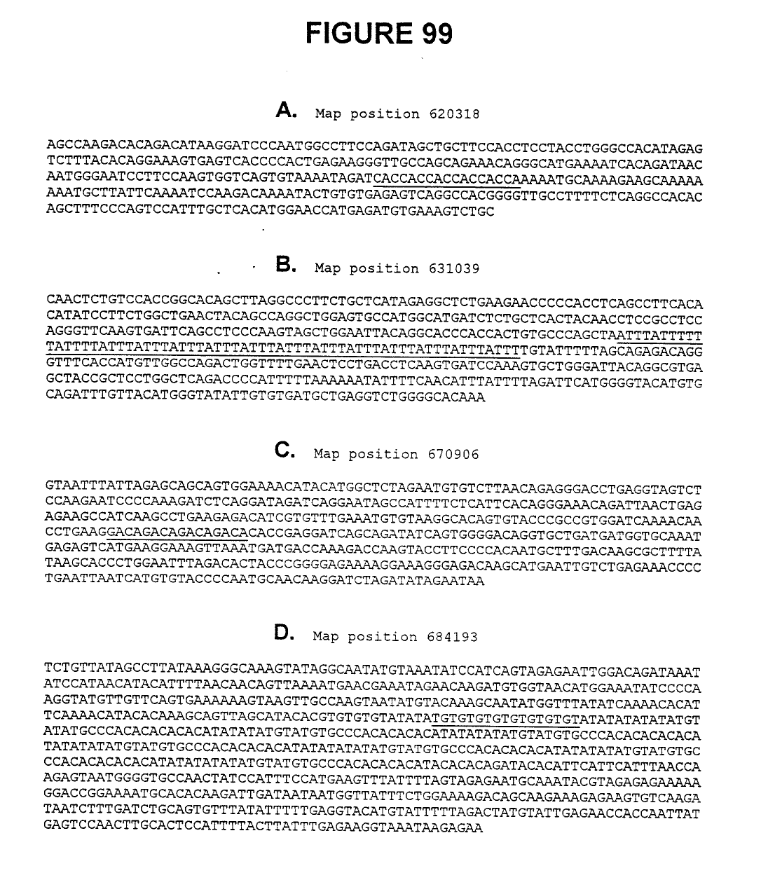

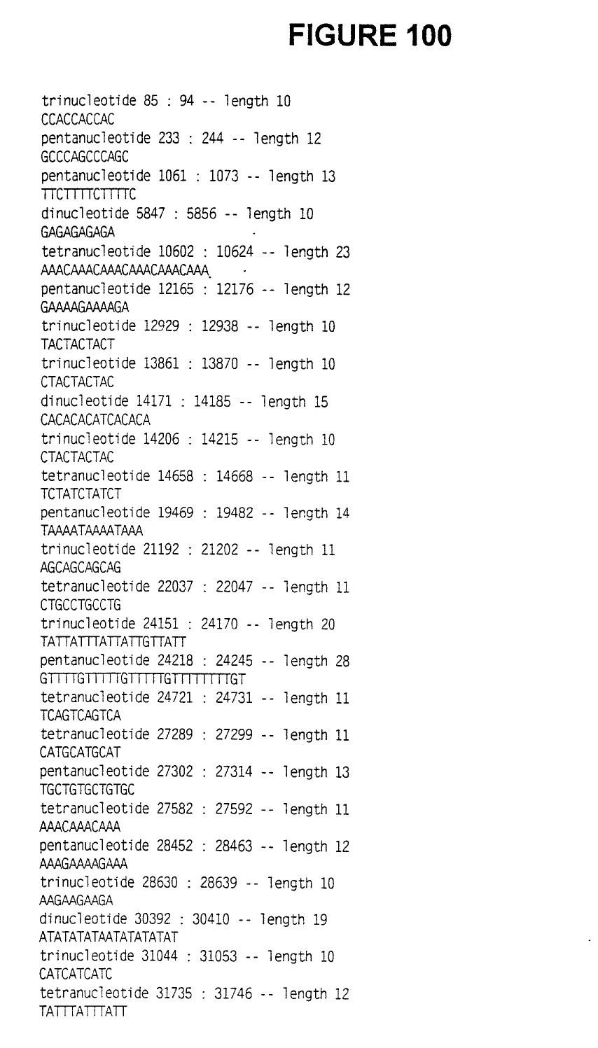

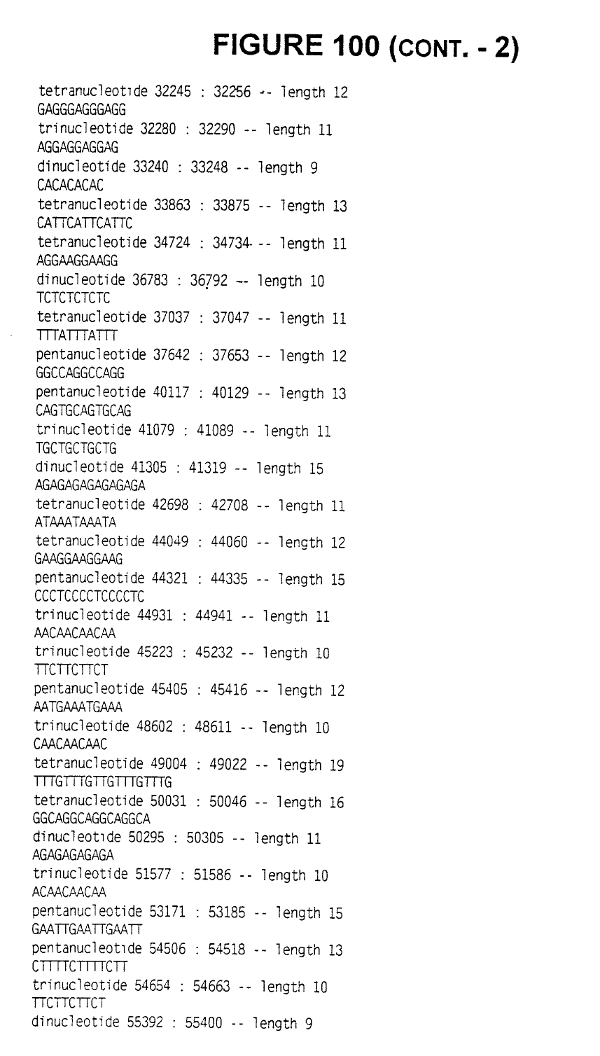

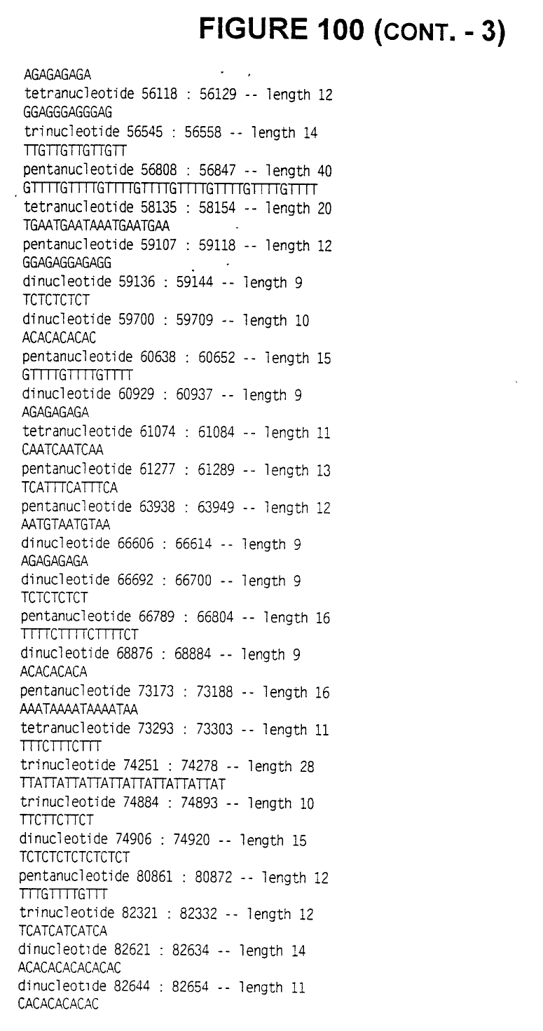

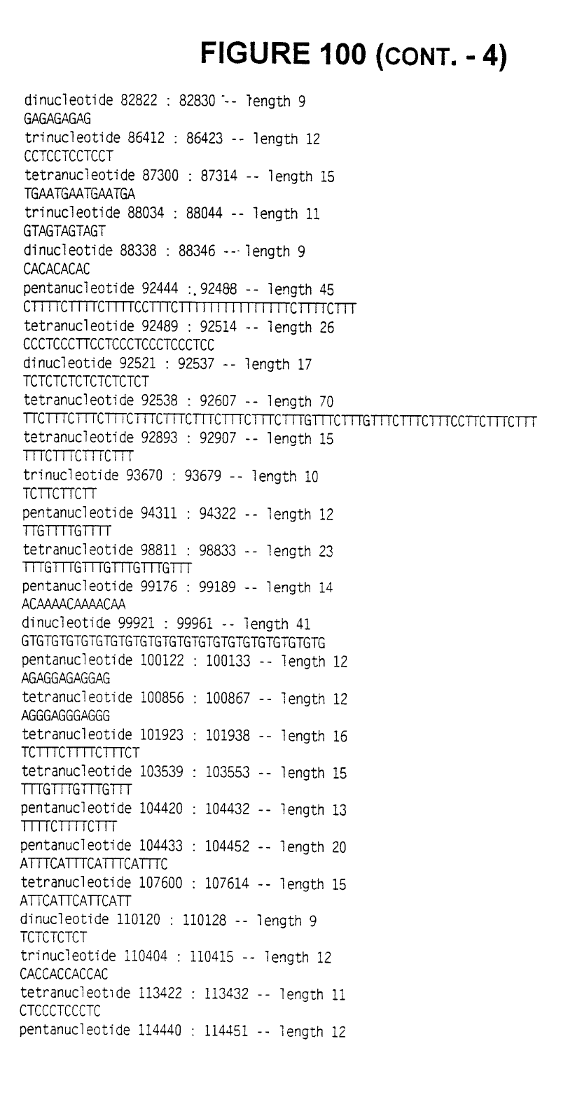

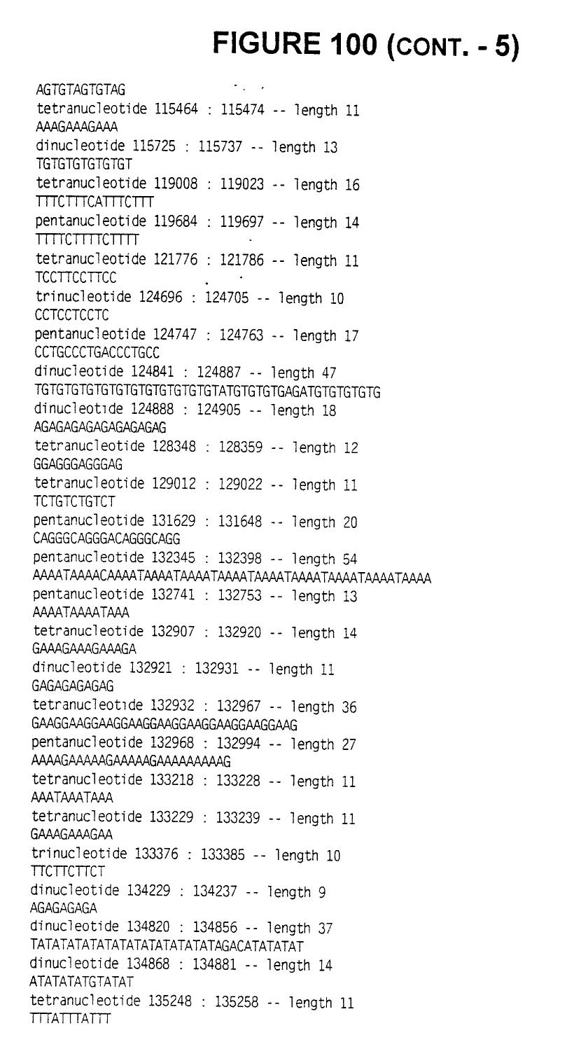

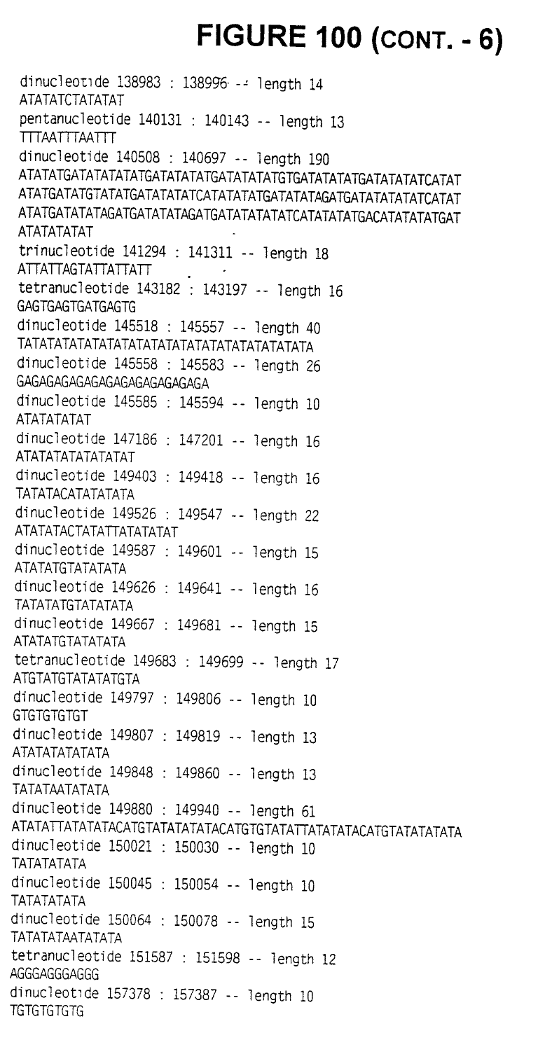

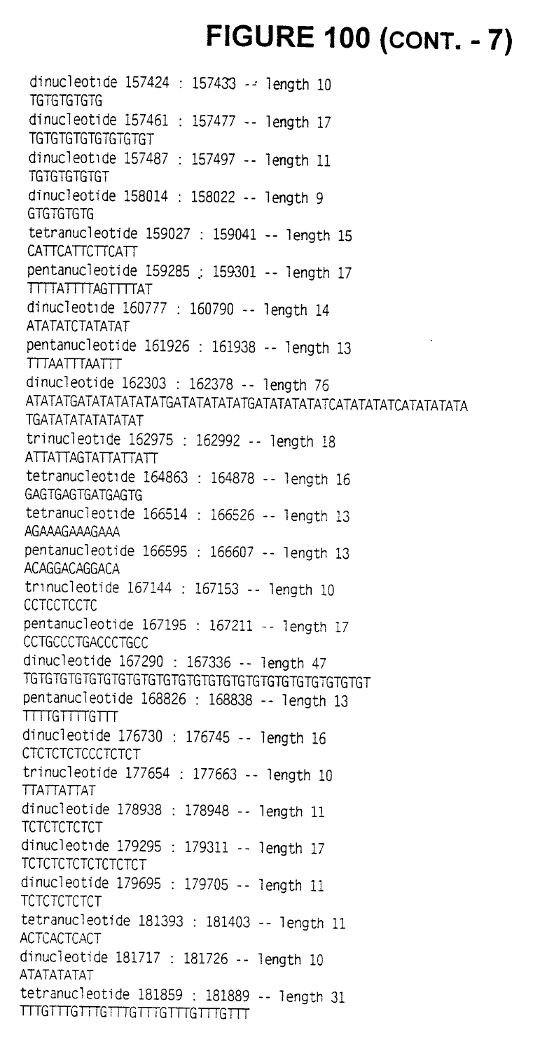

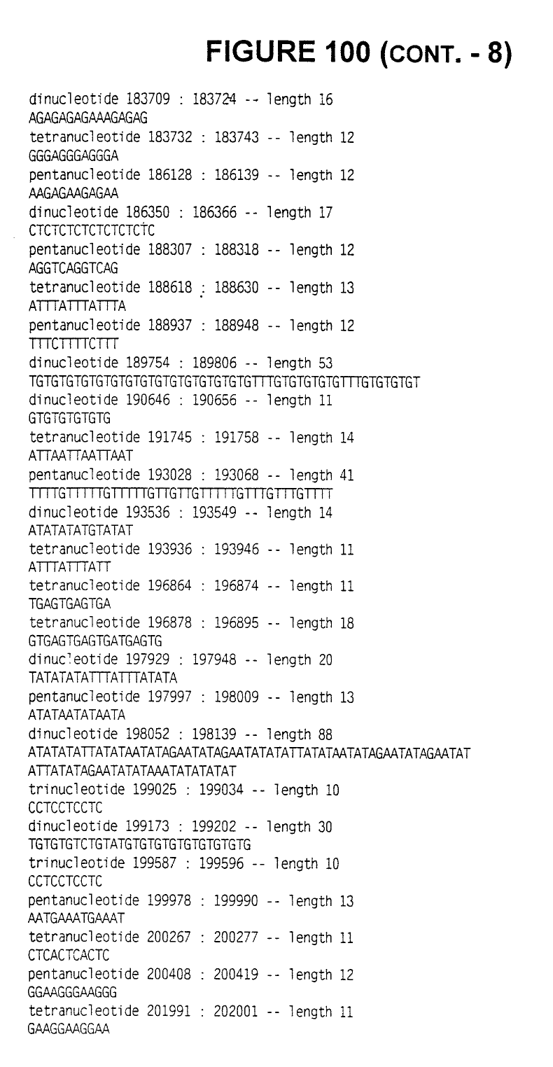

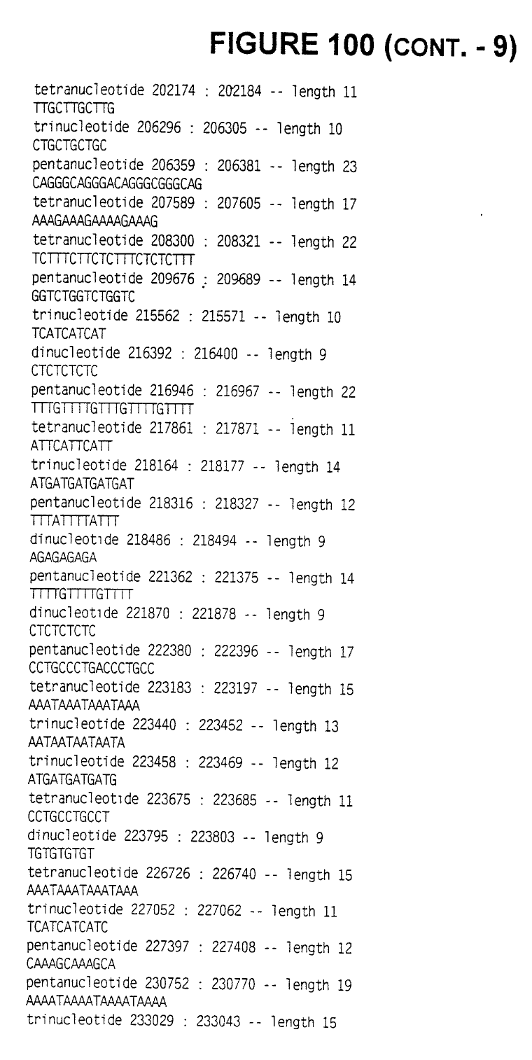

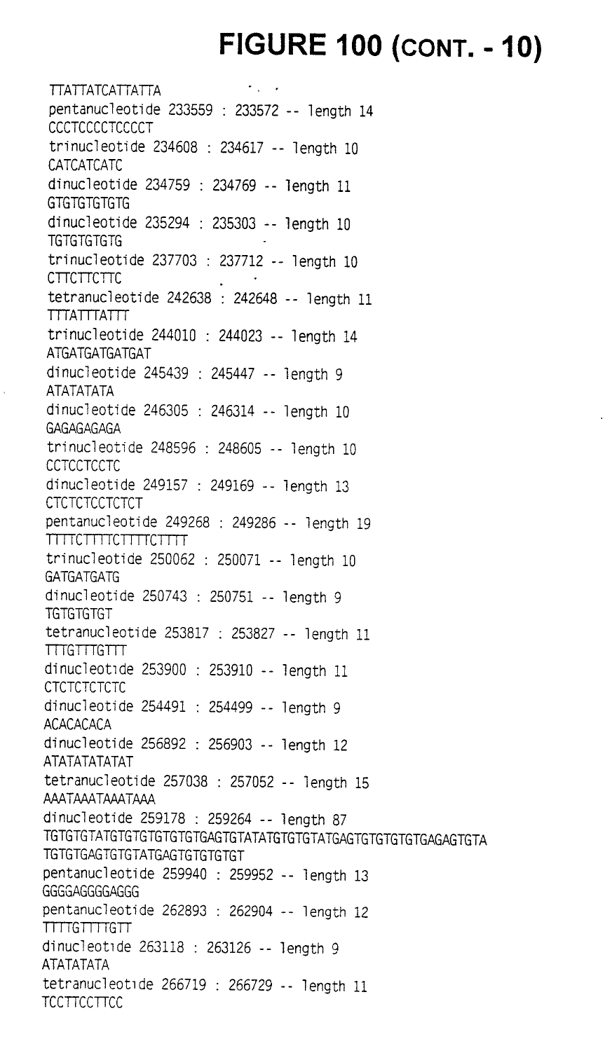

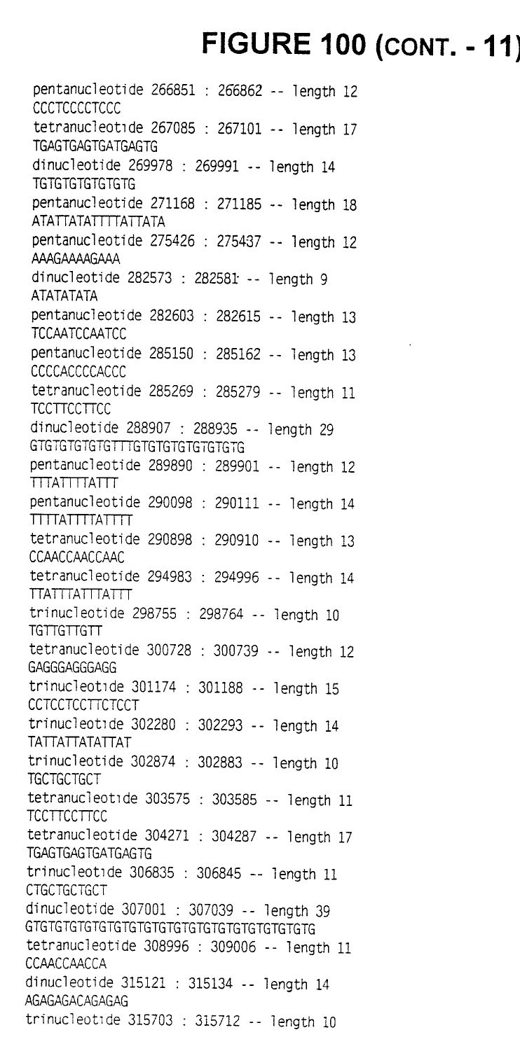

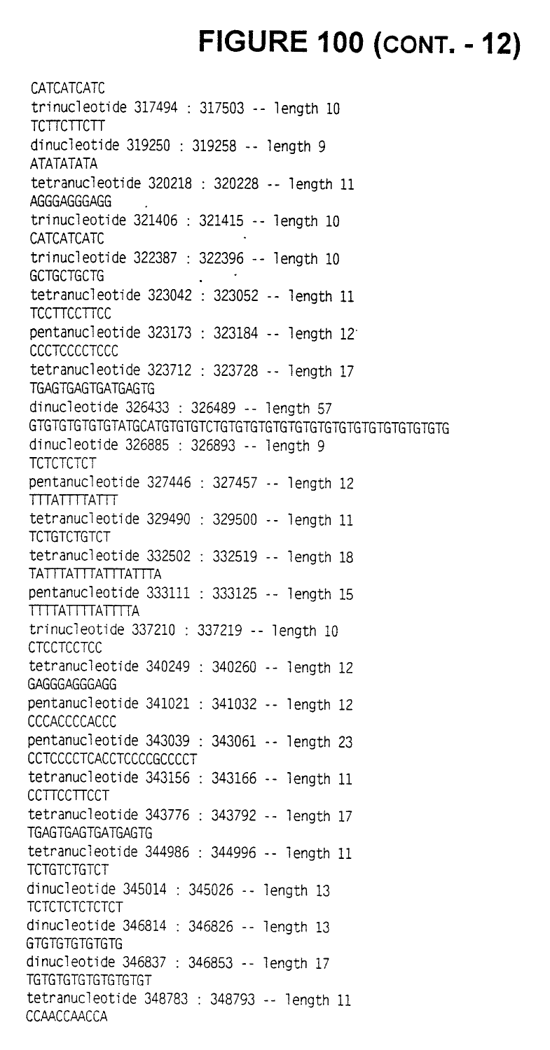

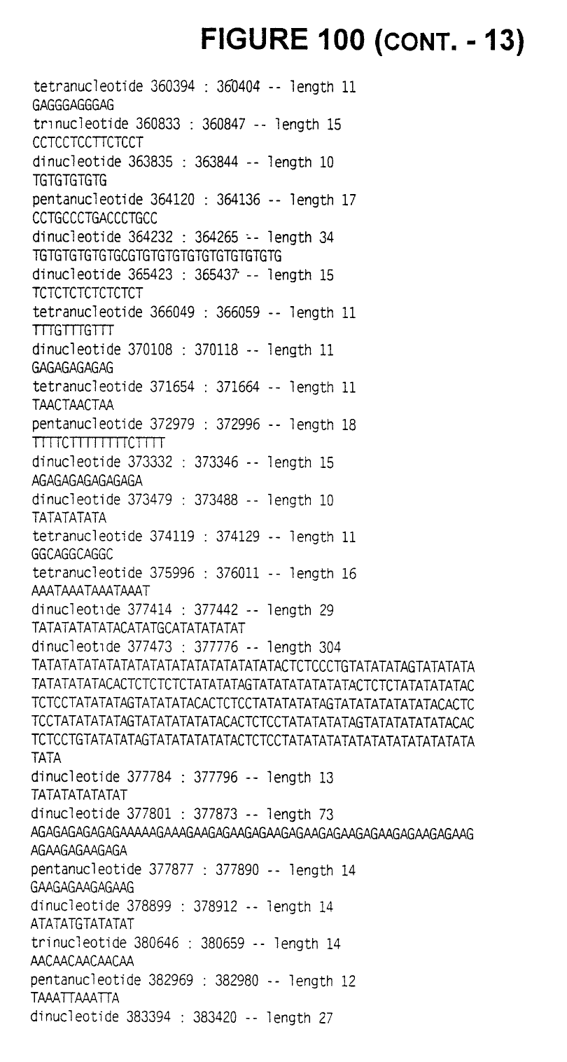

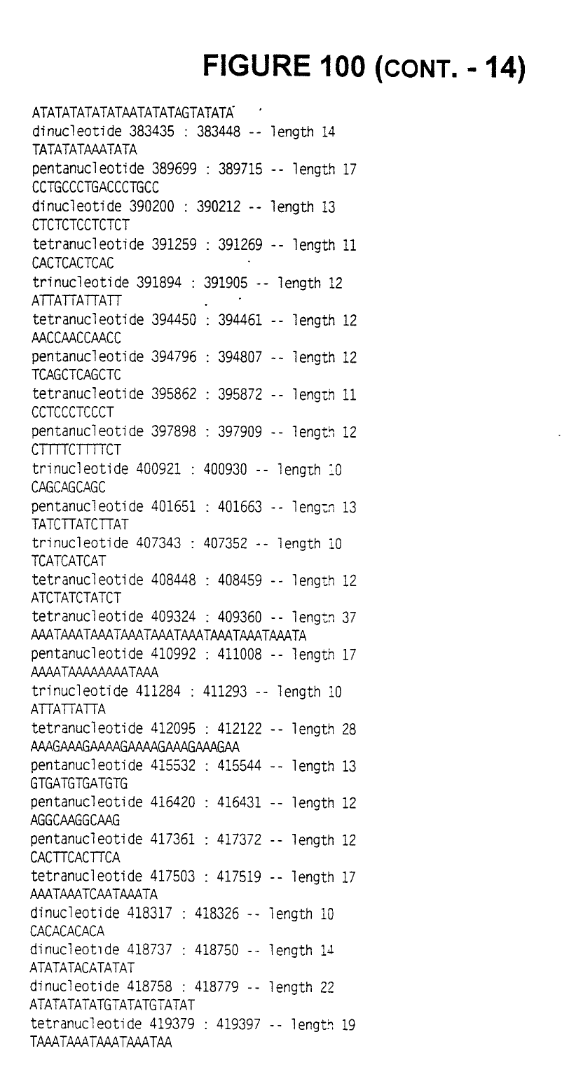

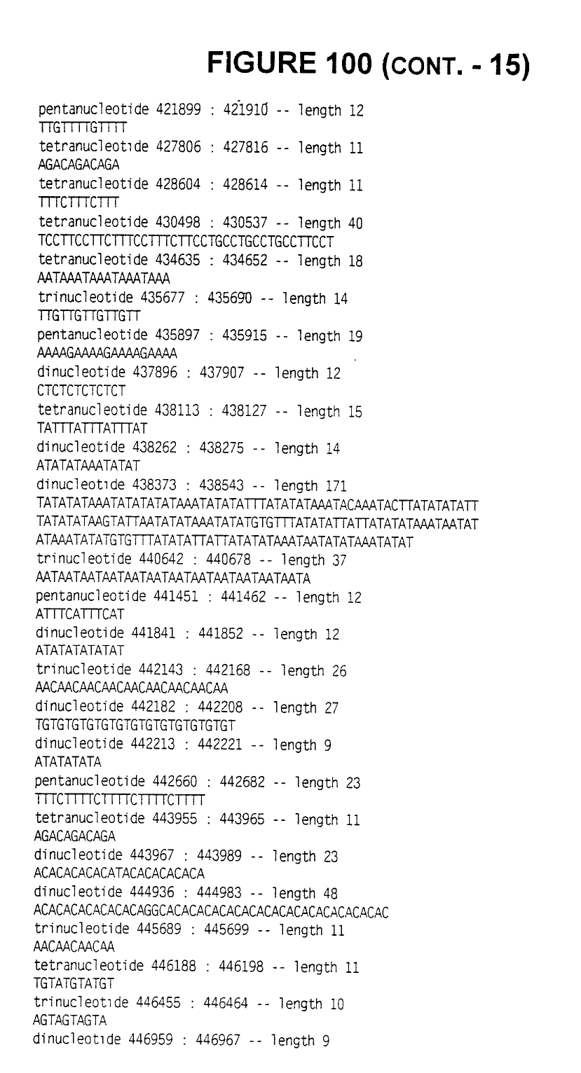

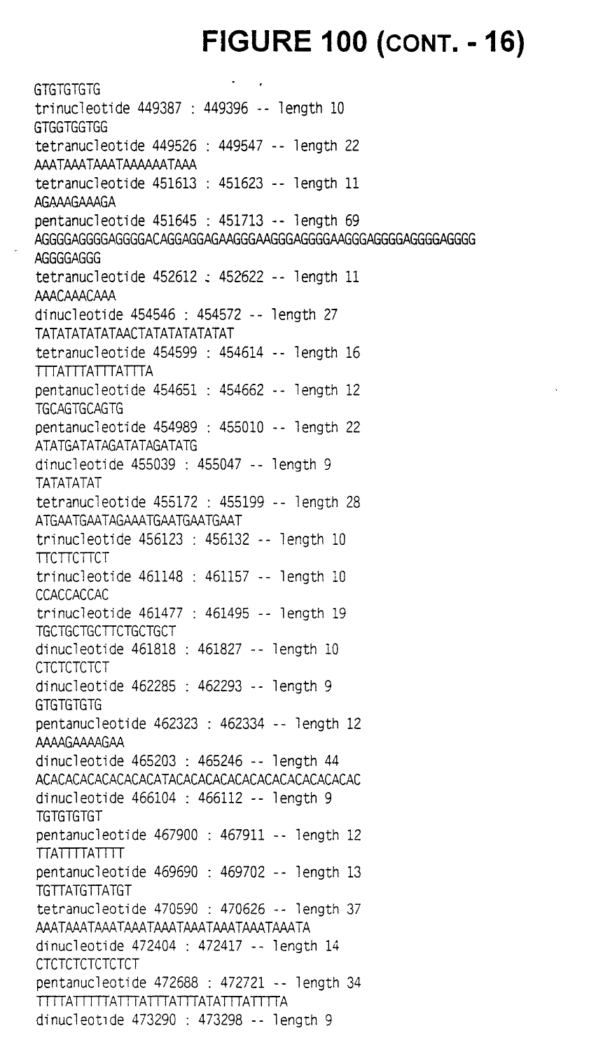

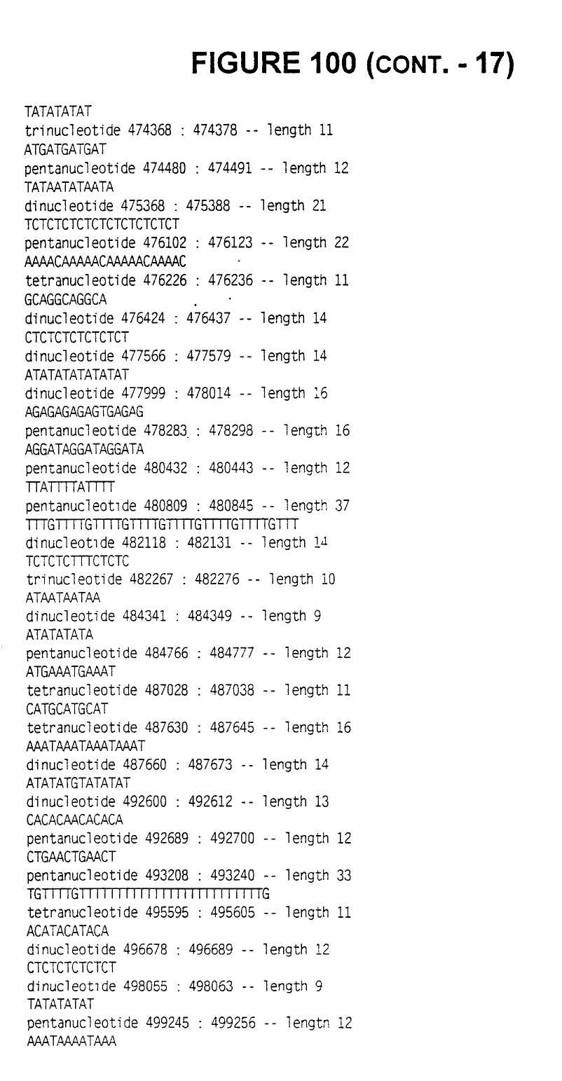

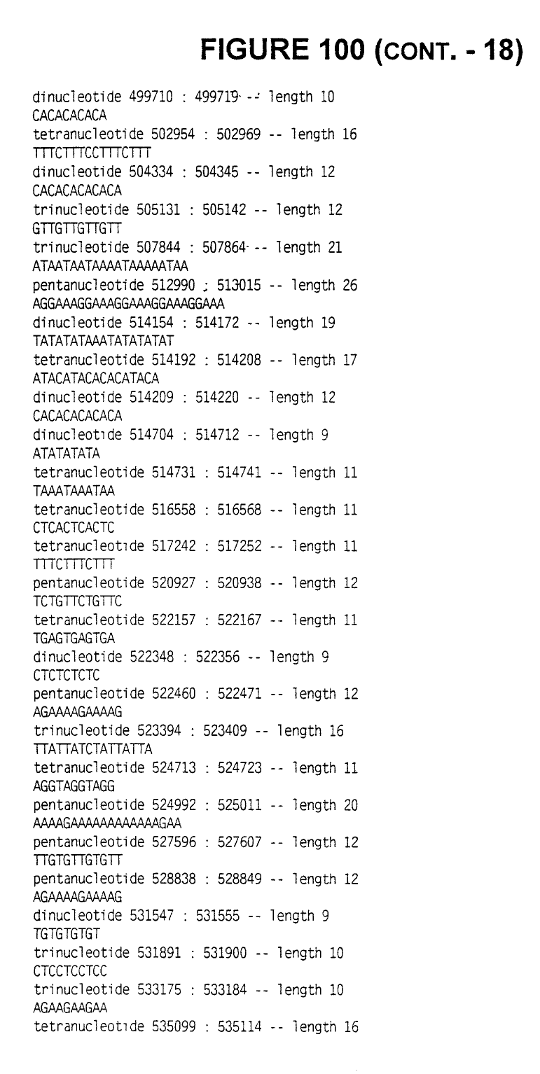

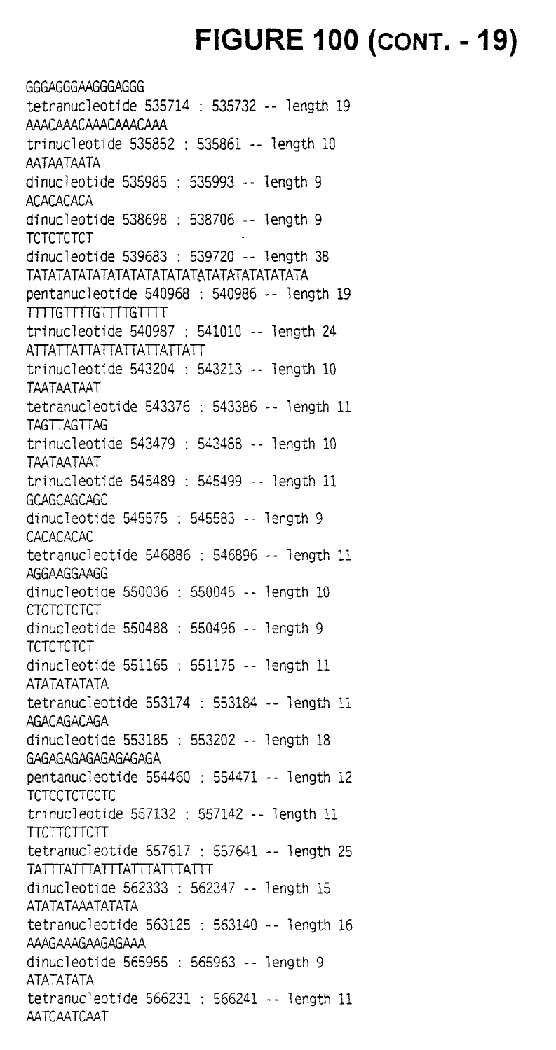

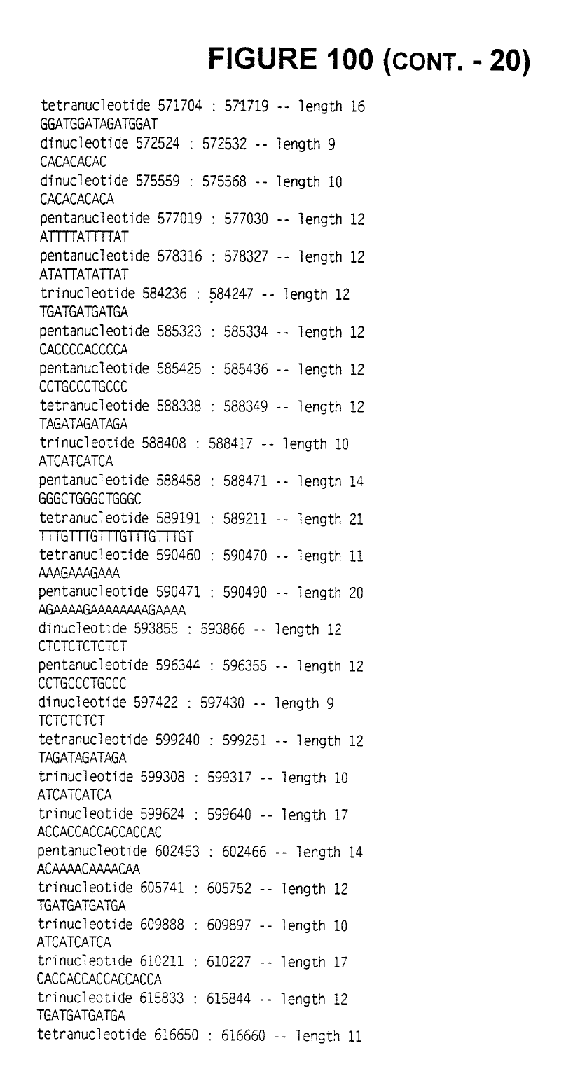

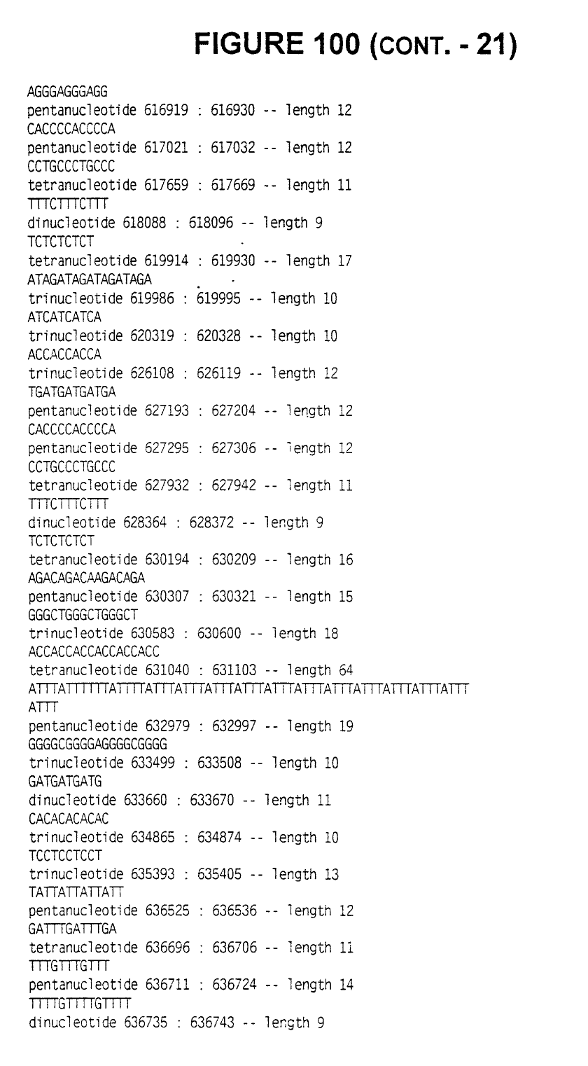

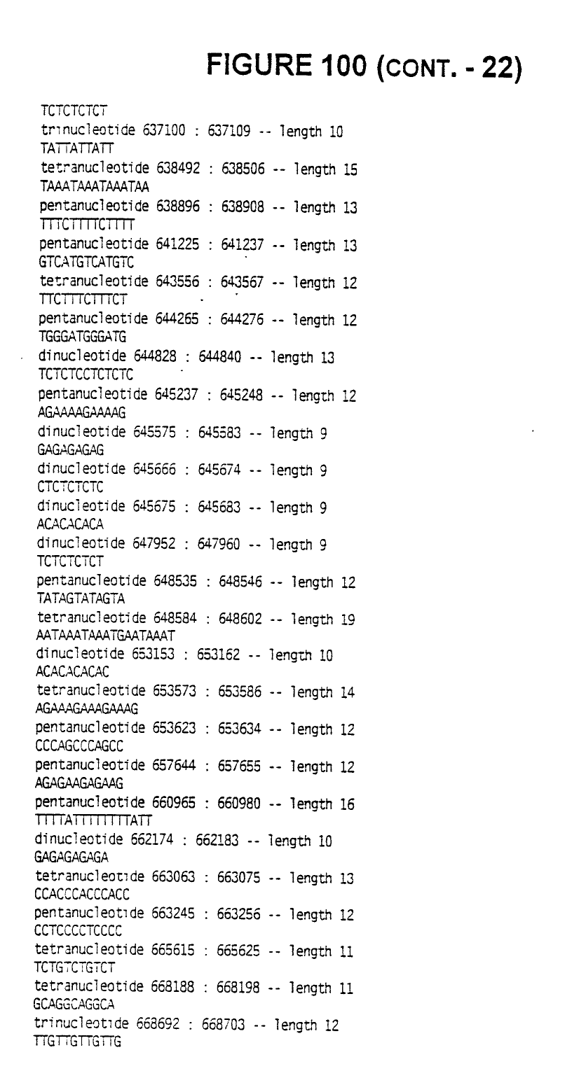

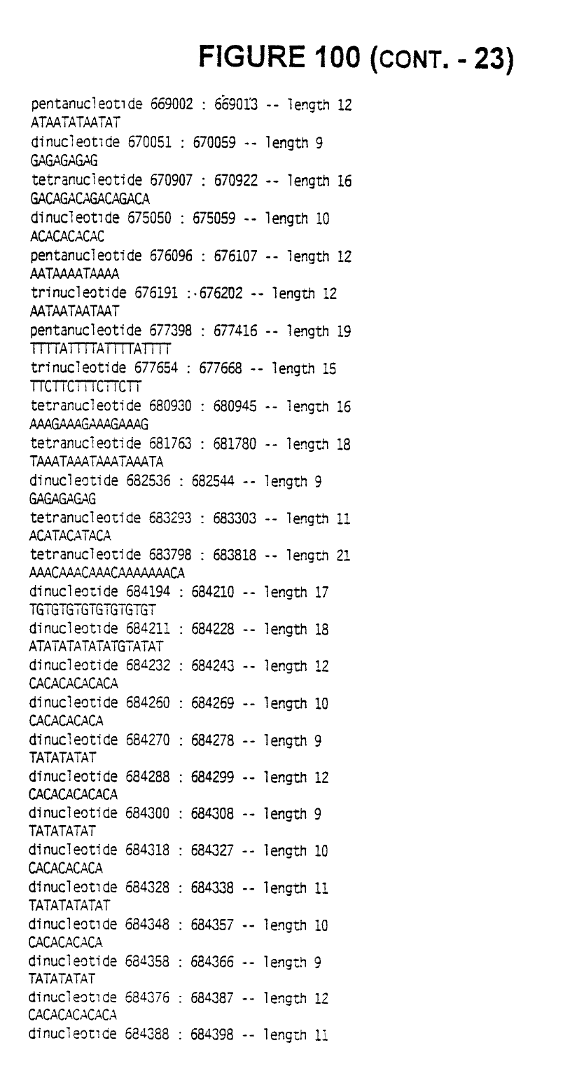

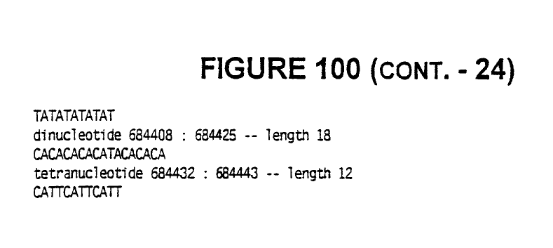

Within other aspects of the present invention, the above-described amplification/detection methods may also be utilized in order to amplify polymorphic repeat sequences or polymorphic base changes. Briefly, in this case, regions of polymorphisms can be first identified from the complete genomic sequence (FIGS. 8 and 100). In particular, identification may be readily accomplished by computer analysis searching for known repeat sequences, such as Alu repeats, SINES, LINES, and the like, as well as simple repeats, such as (CA)ₙ. Surrounding non-repeat sequences are identified as candidate regions for primer pairs (see FIGS. 89-99). Amplification on genomic DNA of different individuals using these primer pairs will reveal the extent of polymorphism of any of these repeats. Because these repeats tend to be highly polymorphic in different members of the same species, these repeats serve as useful genetic markers.

Single nucleotide changes which are polymorphic may also be detected by the amplification/detection methods described above. Briefly, such polymorphisms were previously analyzed by restriction mapping, which limits the usefulness to those changes which create or destroy a restriction site, by heteroduplex formation followed by S1 digestion, heteroduplex formation followed by cleavage at mismatched nucleotides by RNase A (Myers et al., Proc. Natl. Acad. Sci. USA 82:7575, 1985), allele-specific oligonucleotide hybridization (Conner et al., Proc. Natl. Acad. Sci. USA 80:278, 1983), or by denaturing gradient gel electrophoresis (Myers et al., Nature 313:495, 1985). PCR analysis is simpler to perform than any of these other techniques, and it is more likely to detect the polymorphism. Therefore, within one embodiment of the invention, a primer pair flanking the polymorphic nucleotide is first selected utilizing the principles described above. PCR is then performed using genomic DNA as a template. The polymorphism is detected by hybridization of an oligomer probe spanning the polymorphism. A single base difference between the probe and the target sequence will inhibit hybridization. Alternatively, one of the primers can contain as its 3′ most base the polymorphic nucleotide. Polymerization from the primer can only occur if the 3′ most base anneals to the target DNA. Thus, if the target contains a different sequence, no amplification occurs. Amplified products can be directly visualized following gel electrophoresis and in the presence of ethidium bromide. As an alternative to PCR, single nucleotide polymorphisms can be detected by a ligase-mediated technique (Landegren et al., Science 241:1077, 1988; U.S. Pat. No. 4,988,617). In this assay, two oligonucleotides are designed to anneal immediately adjacent to each other on a target DNA molecule. The two oligomers are joined covalently by DNA ligase, provided that the nucleotides at the junction are correctly base-paired. If a heat stable ligase is used, amplification of the signal can be accomplished. The ligation product is detected by incorporation of a radioactive label on one of the oligomers or by incorporation of a biotin label and subsequent nonradioactive detection system as described above.

### Detection of Organ Transplant Rejection and Other T-Cell Associated Diseases

Within another aspect of the present invention, methods are provided for diagnosing organ transplant rejection in a patient following organ transplantation, comprising the steps of: (a) obtaining a biological sample containing T cells from a patient pre- and post-organ transplantation, (b) contacting the biological sample under conditions and for a time sufficient with a panel of antibodies capable of specifically binding to each and every unique β chain of a T cell receptor, and (c) detecting an increase of antibody binding in the post-organ transplantation biological sample relative to the level of antibody binding in the pre-organ transplantation sample, such that organ transplant rejection may be diagnosed in a patient following organ transplantation.

Within a related aspect, methods are provided for diagnosing organ transplant rejection in a patient following organ transplantation, comprising the steps of: (a) obtaining a biological sample containing T cells from a patient pre- and post-organ transplantation, (b) extracting nucleic acids from the cells, (c) contacting the extracted nucleic acids with a panel of nucleic acid probes capable of specifically binding to each and every nucleic acid molecule encoding a β chain of a T cell receptor, and (d) detecting an increase of probe binding in the post-organ transplantation biological sample relative to the level of probe binding in the pre-organ transplantation sample, such that organ transplant rejection may be diagnosed in a patient following organ transplantation. Within one embodiment, such methods may further comprise, subsequent to the step of extracting nucleic acids, amplifying nucleic acids encoding Vβ regions.

Within yet another aspect, methods are provided for diagnosing organ transplant rejection in a patient following organ transplantation, comprising the steps of: (a) obtaining a biological sample containing T cells from a patient pre- and post-organ transplantation, (b) extracting nucleic acids from the cells, (c) amplifying nucleic acid molecules which encode Vβ regions, and (d) detecting an increase in the presence of amplified nucleic acid molecules which encode Vβ regions in the post-organ transplantation biological sample relative to the level of amplified molecules in the post-organ transplantation sample, such that organ transplant rejection may be diagnosed in a patient following organ transplantation.

Briefly, in accordance with any of the above-described methods, biological samples are first obtained from patients at intervals both prior to, and following organ transplantation. Samples may be obtained from peripheral blood, the site of organ transplant, or accumulated fluids in or near the transplanted organ. Cells in the sample may be collected by centrifugation from whole sample, if the sample is a fluid, or following disruption of the sample if the sample is a solid.

Antibodies to TCR are incubated with cells under standard staining conditions. A preferred method of staining is when the antibody is present in saturating amount. The antibodies are directed. to a Vβ polypeptide established to be present in an αβ TCR and is present on a higher frequency of T cells in the biological sample isolated from a transplant patient than from a normal individual. In a preferred embodiment, antibodies are monoclonal. Antibody binding to T cells is quantified. Antibody can be directly or indirectly labeled with a detecting agent and assayed by flow cytometry, confocal microscopy, electron microscopy, light microscopy, or other available method. In one embodiment, antibody is labeled with an enzyme, a fluorophore, a chromophore, or radionuclides. A preferred embodiment is labeling with a fluorophore and detection by flow cytometry or confocal microscopy.

A typical protocol involves lysing the cells in a Tris buffer containing 0.5% SDS, 50 mM EDTA, and 150 μg/mL proteinase K and incubating the reaction at 50° C. for 30 min. Subsequently, the nucleic acids can be precipitated by the addition of ethanol. For PCR analysis, the mRNA must be transcribed into cDNA using reverse transcriptase. cDNA is added to the PCR reaction mixture. To avoid amplification of the genomic Vβ gene, the primer pair consists of an upstream primer complementary to the Vβ gene and a downstream primer complementary to the Cβ gene. Genomic Vβ genes are either unrearranged (in T cells expressing a different Vβ ) or rearranged to a Dβ and a Jβ but are located at a sufficient distance from the Cβ gene that amplification does not occur. To detect an increase in the frequency of T cells expressing the particular Vβ is the amplification must be done in a quantitative fashion and compared to a control (non-diseased) tissue. Methods for quantitative PCR are known in the art (see PCR Protocols, supra). Detection methods for the amplified products are as described above. Quantitation may be determined using a phosphor imager (Molecular Dynamics) if a radioactive label is used for detection. Non-radioactive labels can be quantified by densitometry readings of gels or film images.

The following examples are offered by way of illustration, and not by way of limitation.

### EXAMPLES

### Example 1

### Construction of YAC Libraries

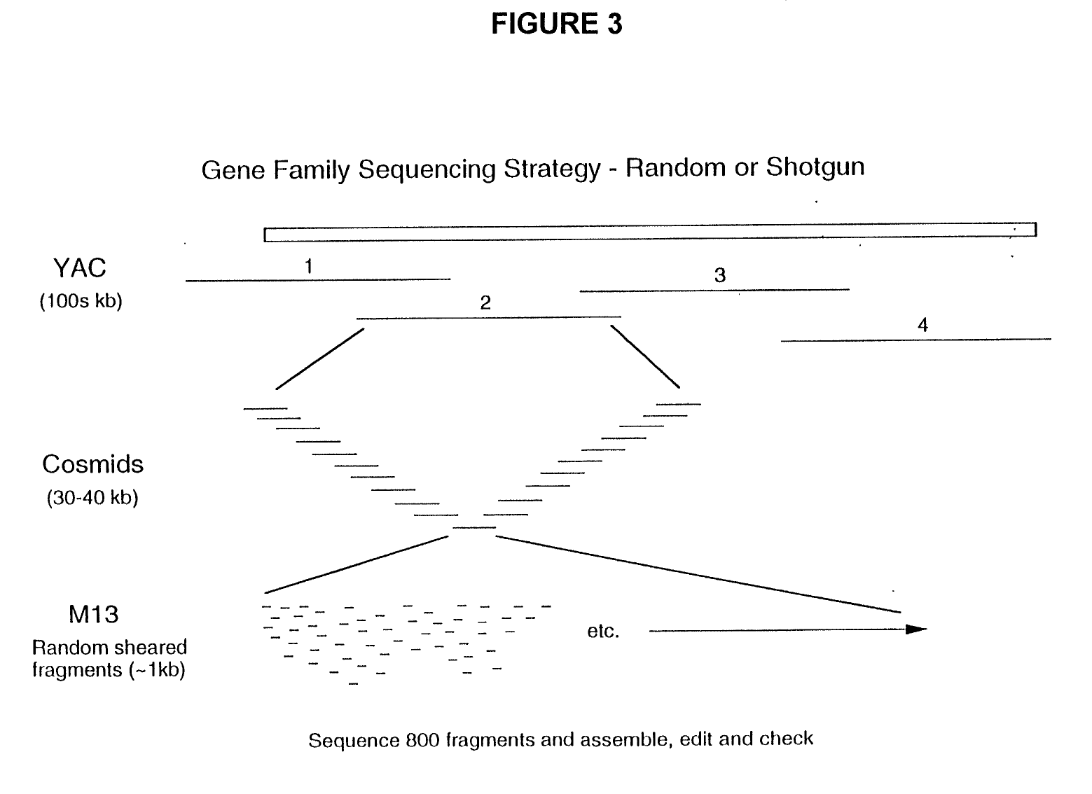

The general strategy of YAC library construction and subsequent subcloning into cosmids and M13 to prepare for DNA sequencing is presented in FIG. 3. Briefly, a YAC—human library containing inserts of human DNA that range in size up to several hundred kb was constructed according to the protocol of Burke et al., Science 236:806, 1987, in a vector (such as YAC4) containing an EcoRI cloning site, and selectable markers for growth in yeast hosts. In particular, the vector was prepared by double digestion with EcoRI and BamHI which yields three fragments: a left chromosome arm containing the centromere, a right chromosome arm, and a stuffer sequence that separates the two TEL sequences present in the plasmid. The reaction mixture was then treated with an excess of calf intestinal alkaline phosphatase (CIAP, Boehringer Mannheim, molecular biology grade) to inhibit religation. Essentially, 50 μg of vector arm DNA is treated with CIAP according to manufacturer's instructions. Following incubation, the reaction mixture is extracted with phenol, chloroform, and then precipitated with ethanol. The stuffer insert is not separated from the other two vector fragments. Arms are resuspended in 10 mM Tris, 1 mM EDTA.

Human DNA is prepared by a limit digest with EcoRI. The amount of EcoRI to use is determined experimentally by digesting samples of genomic DNA with decreasingly smaller amounts of enzyme. Following digestion, samples are electrophoresed on 0.5% agarose gels in the presence of 0.5 μg/mL ethidium bromide. Visual inspection is used to determine an optimum amount of enzyme to yield fragments in the size range 20 to 300 kb. A scaled up digestion is then performed on at least 25 μg of human DNA. Fragments are predominantly in the size range 50 to 700 kb. Fifty μg of vector is ligated to 25 μg of human fragments. The ligation reaction is carried out for 12 hours at 15° C. with 50 U of T4 ligase (Boehringer Mannheim) in 200 μL of 50 mM Tris-Hcl, 10 mM MgCl₂, 1 mM ATP, pH 7.5. After ligation, the reaction mixture is extracted with phenol, followed by an extraction with chloroform, and dialyzed against 10 mM Tris, pH 8, 1 mM EDTA. Half the ligation mixture is transformed into 5×10⁷ AB1380 cells. These cells are converted to spheroplasts with lyticase and plated onto four 100-mm Petri dishes with the use of a synthetic spheroplast-regeneration medium lacking uracil (Sherman et al., in Methods in Yeast Genetics, Cold Spring Harbor Laboratory, Cold Spring Harbor, N.Y., 1979). The transformation protocol is performed according to Rose and Broach in Methods in Yeast Genetics, supra. Greater than 90% of Ura+ transformants are usable clones and contain human DNA ranging in size up to more than 400 kb.

### Example 2

### Construction of Cosmid Libraries

Cosmid libraries are constructed by ligation of either human genomic or YAC DNA, which has been digested with Sau3AI into 30-40 kb fragments, with pWE15A cosmid DNA, which has been linearized with BamHI and treated with calf intestinal alkaline phosphatase.

The cosmid vector, pWE15A, is a modification of the vector pWE15 (Wahl et al., Proc. Natl. Acad. Sci. USA, 84, 2160, 1987). The version pWE15A contains a polylinker with 15 infrequently cleaved restriction enzyme sites, which are asymmetrically centered around the BamHI cite. The BamHI site is the cloning site used for the insertion of genomic or YAC DNA. Cosmid vector DNA is digested with BamHI according to manufacturer's instructions. Complete digestion is verified by analyzing a small amount of DNA, 0.1 to 0.5 μg, in a 0.8% agarose gel. Upon complete digestion, plasmid DNA is extracted with an equal volume of phenol:chloroform:isoamyl alcohol, and subsequently with an equal volume of chloroform:isoamyl alcohol. For each extraction, the upper aqueous layer is removed and the organic phases are reextracted with a half volume of 10 mM Tris, pH8.0, 1 mM EDTA(TE). All aqueous layers are pooled together. The DNA solution is adjusted to 0.3 M sodium acetate, pH5.5, and the DNA is then precipitated with 2.5 volumes of ethanol. Following chilling, the sample is centrifuged in a microfuge for 15 minutes. The DNA pellet is washed with 70% ethanol and dried under vacuum. Vector DNA is resuspended in 10 mM Tris, 1 mM EDTA at a concentration of 1 mg/ml.

The linearized vector DNA is dephosphorylated with calf intestinal alkaline phosphatase (CIAP). For each 50 pmol of DNA termini, 2.5 units CIAP is added in 50 mM Tris, pH8.0, 10 mM MgCl₂, 1 mM ZnCl₂. The reaction is incubated at 37° C. for ten minutes. The DNA is cleaned up by extraction with phenol:chloroform, followed by an extraction with chloroform. The aqueous phases are pooled together and the DNA is precipitated by adjusting the solution to 0.3 M sodium acetate and adding 2.5 volumes of ethanol. The DNA pellet is collected by centrifugation, washed with 70% ethanol, and dried under vacuum. Cosmid vector DNA is then resuspended in 0.1×TE at 1 mg/ml.

High molecular weight genomic DNA and YAC DNA is prepared for ligation. The isolation method for these DNAs must be gentle enough so that very large fragments of DNA are purified (greater than 100 to 150 kb). Precautions are taken to minimize shearing of DNA. Thus, any mixing or sampling of the DNA solution should be done by gentle inversion or gentle pipetting through a wide tip pipette. Yeast containing the desired YACs and the human cell line ATCC 1521 are resuspended in 15 ml of 0.1 M NaCl, 50 mM Tris, pH7.5, 1 mM EDTA. The cells are lysed by the addition of SDS to 0.5% and proteinase K to 100 μg/ml. Yeast cells are made susceptible to lysis by treatment with lyticase. Lohr in Yeast, A Practical Approach, I. Campbell & J. H. Duffus ed. 5, p. 125, IRL Press, Oxford and Washington, D.C., 1988. The DNA solutions are mixed by gentle inversion and incubated for one hour at 50° C. DNA is extracted with an equal volume of phenol:chloroform:isoamyl alcohol. The aqueous layer was removed after centrifugation. DNA is precipitated following the addition of sodium acetate to 0.2 M and two volumes of ethanol. The precipitate is collected by centrifugation and washed with 70% ethanol. Without drying completely, the DNA is resuspended in TE. The quality of the DNA can be checked by electrophoresis in a 0.3% agarose gel. Good quality DNA suitable for cloning into cosmid vectors will co-migrate with a T4 DNA marker (160 kb).

The genomic or YAC DNA is prepared for insertion into the cosmid vector by digestion with Sau3AI to generate fragments of 30 to 40 kb in length. Samples of DNA are digested at increasing times with a set amount of enzyme. The optimum digestion time is determined analytically by digesting approximately 10 μg of DNA in a 100 μl reaction buffer (10 mM Tris, pH7.4, 10 mM MgCl₂, 15 mM NaCl). Approximately 0.5 to 1 units of Sau3AI are added to the DNA and aliquots are removed at intervals from zero up to 60 minutes. The enzyme in each aliquot is inactivated by adding EDTA to 20 mM. Samples are analyzed by electrophoresis in a 0.3% agarose gel. Digestion times yielding fragments of the appropriate size are thus determined. Once the optimal time is determined, 100 to 200 μg of DNA is digested in a scaled-up prep. Aliquots for optimal time points are removed and the enzyme inactivated as above. Samples are pooled and can be analyzed on an agarose gel to ensure that the appropriate size distribution has been attained. If so, the digested DNA is extracted as above with phenol:chloroform and precipitated. DNA is resuspended in TE and fractionated on a 10 to 60% linear sucrose gradient. Fractions are collected after centrifugation, diluted with TE, and the DNA is ethanol precipitated. DNA precipitates are collected by centrifugation and resuspended in TE. The molecular size of DNA in each fraction is checked on a 0.3% agarose gel. Fractions which have a molecular size of approximately 30 to 40 kb are chosen and pooled together. This DNA, which is suitable for cloning into the plasmid cosmid vector, is referred to as insert DNA.

A ligation reaction is performed for each library construction. Approximately 3 μg of vector DNA prepared as described above is mixed with approximately 1.5 μg of insert DNA in 20 μl of 10 mM Tris, pH7.5, 10 mM MgCl₂, 5 mM DTT, 1 mM ATP. Approximately 1 to 2 Weiss units of T4 DNA ligase is added to the reaction, and the reaction is incubated at 14° C. overnight. The ligation reaction is packaged into λ packaging extracts (Stratagene) according to manufacturer's instructions. Briefly, 2 μl of the ligation reaction is added to a 10 μl freeze-thaw lysate and a 15 μl sonic extract and incubated at room temperature for 30 minutes to 1 hour. The reaction is diluted with 1 ml of 10 mM Tris, 100 mM NaCl, 10 mM MgCl₂, 0.01% gelatin. The packagings are titrated by infection of an E. coli strain, such as C600, with ten-fold serial dilutions of the packaging. Titers usually range from 100,000 to 800,000 transformants per microgram of size-fractionated insert DNA.

Genomic libraries are screened by growing colonies on nitrocellulose membranes, lysing the colonies, and hybridizing the DNA with TCR Vβ and Cβ probes. YAC-cosmid libraries are screened with human repetitive sequences. Approximately 20,000 to 50,000 colony-forming units are incubated with an equal volume of E. coli, such as strains HB101 or C600, at room temperature for ten minutes. The cells are spread onto 137 mm nitrocellulose filters which have been placed on LB agar plates containing 50 μg/ml ampicillin. Plates are incubated at 37° C. until the colonies are about 0.2 ml in diameter (usually 8 to 10 hours). A second set of nitrocellulose filters are placed on LB agar plates containing 50 μg/ml ampicillin for replica plating. The master filter is replicated onto the wetted replica filter by placing the two filters together with the colony side down and pressing the filters together. Nitrocellulose filters are marked for orientation. The filters are then separated and placed back on the agar plates. After replica plating the master and replica filters are incubated until the colonies are approximately 0.5 mm in diameter. Replica filters are removed from the LB-amp plates and further incubated at 37° C. on LB agar plates containing 250 μg/ml chloramphenicol for 20 hours in order to amplify the cosmid DNA. The filters are removed from these plates, colonies are lysed, and the DNA is fixed on the filters by sequential 30-minute incubations on Whatman 3 MM paper which is saturated with 0.5 M NaOH, 1.5 M NaCl, followed by 1 M Tris, pH7.5, 1.5 M NaCl, followed by 6×SSC (20×SSC=3 M NaCl, 0.3 M Na citrate, pH 7.0). Between each incubation the filters are blotted on dry Whatman 3 MM filters. Filters are baked at 68° C. for four hours and stored at room temperature until ready for use.

Filters are prepared for hybridization by incubation in 6×SSC, 2× Denhardt's solution (100× Denhardts=2% bovine serum albumin, 2% Ficoll 2% polyvinylpyrrolidine) at 68° C. overnight. The filters are rinsed in 6×SSC and then hybridized in a solution of 6×SSC, 2× Denhardt's, 1 mM EDTA, 100 μg/ml salmon sperm DNA, 0.5% SDS, and 2-5×10⁶ CPM of ³²P-labeled probe/ml of solution. Hybridizations are carried out overnight in heat-sealable bags at 68° C. Alternatively, hybridizations can be carried out in the same solution but with the addition of 50% formamide at 42° C. Following hybridization the filters are washed three times in 2×SSC, 0.5% SDS at 68°. A final rinse of the filters is done at room temperature in 2×SSC. The filters are blotted dry and exposed to autoradiography film. The human genomnic DNA library is screened with human TCR Vβ and CVβ probes. The YAC-converted cosmid library is screened with human repetitive sequences, such as an Alu repeat to identify clones containing human DNA.

### Example 3

### Cosmid Growth, Fragmentation, and Subcloning

Cosmid DNA is prepared according to the procedure detailed in Molecular Cloning: A Laboratory Manual, 2d Edition, edited by J. Sambrook, E. F. Fritsch, and T. Maniatis (Cold Spring Harbor Laboratory Press, 1989). Cells containing the cosmid are grown in 150 ml of L broth with the appropriate antibiotic (ampicillin or tetracycline) for 16 to 20 hours at 37° C. Cells are pelleted then resuspended in 2.4 ml of 50 mM glucose, 25 mM Tris-HCl (pH 8.0), 10 mM EDTA (pH 8.0), 100 μg/mL lysozyme. After 5 min incubation at room temperature, 4.8 ml of 0.2 M NaOH, 1% SDS are added and the suspension is gently mixed by inversion. After 5 minutes, 4.0 ml of 5 M KoAc, 2 M HAC is added, the suspension is mixed by shaking and, after 15 minutes, is spun for 10 minutes. Seven ml (0.6 volume) of isopropanol is added to the supernatant, the solution is mixed, incubated at 15 minutes at room temperature, and centrifuged. After removing all of the supernatant, the pellet is resuspended in 500 μl of 10 mM Tris, pH. 7.5, 1 mM EDTA, containing 10 μg/ml RNAse A and incubated at 37° C. for 30 minutes. DNA is extracted with an equal volume of phenol:chloroform-isoamyl alcohol (24:1). DNA is precipitated by the addition of 125 μl of 5 M NaCl and 750 μl 13% polyethylene glycol. The precipitated material is spun for 15 minutes in a microfuge, washed twice with 200 ml 70% ethanol, dried, and resuspended in 150 μl of water or TE. This procedure yields roughly 100 μg to 1 mg of DNA.

Approximately 1 kb insert DNA fragments are randomly generated from a cosmid by sonicating 10-15 μg of DNA in 50 μl water using a Heat Systems-Ultrasonics Inc. cup horn sonicator with the following settings: output control, 4.5; duty cycle, 100%; pulse, continuous. Generally, 20 to 40 seconds of sonication is sufficient. After sonication, the fragment ends are repaired by T4 DNA polymerase by adding 12 μl H₂O, 7 μl of 10× T4 polymerase buffer (500 mM Tris, pH 8.8; 150 mM ammonium sulfate; 65 mM magnesium chloride; 1 mM EDTA; 500 m g/ml BSA; 100 mM 2-mercaptoethanol), 1 μl 10 mM dNTPs, and 1.5 units of T4 DNA polymerase (Boehringer-Mannheim Biochemicals) and incubating the reaction for 30 min at 37° C. Fragments are electrophoresed in a 1.5% agarose gel, and fragments approximately 800 to 1500 bp long are isolated from the gel onto DEAE 81 paper (Whatman), eluted with Tris-EDTA, 1M NaCl, ethanol precipitated, washed with 70% ethanol, and resuspended in 25 μl of Tris-EDTA, pH 7.5.

Fragments are cloned into an M13 vector. Approximately 5 to 50 ng of fragments is ligated to 10-50 ng M13 vector in a 20 μl reaction containing 10× ligase buffer (500 mM Tris-Cl pH 7.6 100 mM MgCl₂, 10 mM dithiothreitol, 1 mM ATP), and 1 unit of T4 DNA ligase (Boehringer Mannheim Biochemicals) overnight at room temperature. M13mp9 RF DNA (Boehringer Mannheim) is prepared by digestion with HincII, and treated with calf intestinal alkaline phosphatase. An aliquot of the ligation mixture is transformed into 85 μl of frozen competent DH5aF′ or DH5aF′IQ cells (BRL, Life Technologies, Inc.) in accordance with the manufacturers instructions. Approximately 50-150 clear plaques are obtained per transformation plate.

### Example 4

### DNA Template Preparation

DNA is prepared from 10 ml of phage cultures grown in 2×YT broth to which 1 ml of frozen DH5aF′IQ cells and 1 ml of 10 mg/ml kanamycin is added. Frozen cells are prepared by adding a scraping of DH5aF′IQ cells obtained from a frozen competent cell kit (see above) to 500 ml of L broth or 2×YT broth containing 10 μg/ml kanamycin and grown overnight at 37° C. A phage plaque or 4 μl of phage culture is used to inoculate the culture. The culture is incubated overnight at 37° C. with rotation. The cells are spun for 15 minutes at 4000 RPM in a Beckman J6 centrifuge. Supernatant is transferred to 15 ml polypropylene centrifuge tubes, and 2 ml of 20% polyethylene glycol (mol wt 8000), 2.5 M sodium chloride are added. The tubes are capped, mixed by inversion, and incubated at least 30 minutes at room temperature. Phage particles are collected by centrifugation for 30 minutes at 3500 RPM in a J6 centrifuge. The supernatant is poured off, and the remaining supernatant, after draining to the bottom of the tube, is removed by aspiration; it is important to remove as much PEG as possible. The phage pellets are resuspended in 250 μl of Tris-EDTA, pH 8, transferred to Eppendorf tubes and 250 μl of phenol equilibrated with Tris-EDTA is added. The solution is vortexed and the phases separated by centrifugation. The aqueous phase is further extracted with 220 μl of chloroform-isoamyl alcohol (24:1). The aqueous phase is transferred to a new Eppendorf tube, and the DNA is precipitated with 1/10 volume of 3 M sodium acetate pH 5.2 and 2 volumes of ethanol. After a 70% ethanol wash and vacuum drying, the DNA pellet is resuspended in 50-80 μl Tris-EDTA, pH 8. DNA concentration is determined by measuring the absorbance at 260 nm.

### Example 5

### DNA Sequence Reactions

The precision of a consensus sequence is improved by determining the sequence of a portion (10%-30%) of clones by Sequenase method and the remainder with Taq polymerase (cycle sequencing method) method of DNA sequencing. Cycle sequencing reactions are performed either by the Catalyst sequencing robot (Applied Biosystems, Inc.) or by a 96 well thermocycler (Perkin Elmer Gene Amp 9600) using the cycle sequencing kits and protocols developed by Applied Biosystems. The optimal DNA concentration for reactions run in the 96 well thermocycler is about 4-5 fold lower than it is for reactions run in the Catalyst.

For Sequenase reactions, the following stocks are used: 5× sequencing buffer (1 M Tris-Cl, pH 7.4; 1 M sodium chloride, 0.1 M dithiothreitol), Sequenase dilution buffer (2 M Tris-Cl pH 7.5, 10 mM 2-mercaptoethanol, 1 mg/ml BSA), 1 M MnCl₂, dNTP mix (2 mM each of dATP, dCTP, dGTP, 3 mM dGTP), 50 mM ddATP, 50 mM ddCTP, 100 mM ddGTP, 50 mM ddTTP, 0.4 pmole/μl fluorescent dye primers (Applied Biosystems), and Sequenase Version 1 (United States Biochemical Corporation). Four triphosphate mixes, one for each dideoxynucleoside, are prepared by combining the dNTP mix and a dideoxynucleoside stock in a 12:1 ratio. Twenty-four sets of sequencing reactions are performed at a time in round bottom 96 well plates. A microtiter plate is divided into three sets of four columns, designated “A,” “C,” “G,” and “T.” Three 3 μl of MnCl₂ stock is added to 197 μl 5× sequencing buffer and 6 μl is distributed to each of the “A” wells. Approximately 2.5 to 3 μg of template DNA and water is added to bring the total volume in the “A” wells to 25 μl. Then 1 μl of “C” primer is added to the “C” wells, 2 μl “G” primer is added to the “G” wells, and 2 μl “T” primer is added to the T wells. Four μl of the template mix is distributed to the “C” wells, and 8 μl is distributed to each of the “G” and “T” wells. 1 μl “A” primer is added to the remaining template in the “A” wells. The microtiter plate is covered and incubated at 55° C. for 5 minutes, then allowed to cool at room temperature for 15 minutes (annealing step). A premix of Sequenase enzyme is prepared by adding 28 μl of enzyme (13 U/ml) to 270 μl Sequenase dilution buffer. Four premixes are prepared by adding 48 μl of the diluted enzyme to 60 ml of the dNTP/ddATP brew and 60 μl of the dNTP/ddCTP brew, and 96 μl of diluted enzyme to 120 μl of the dNTP/ddGTP brew and 120 μl of the dNTP/ddTTP brew. After annealing, 3.5 μl of the appropriate enzyme/triphosphate premix is added to the “A” and “C” wells, and 7.0 μl of the enzyme/triphosphate premix is added to the “G” and “T” wells. The plate is again covered and incubated at 37° C. bath for 7 minutes. Reactions are terminated by adding 100 μl of sodium acetate/ethanol (150 μl 3 M sodium acetate, pH 5.2, 4.8 ml absolute ethanol) to the “A” wells. The “A,” “C,” “G,” and “T” reactions are pooled horizontally and transferred to Eppendorf tubes. After cooling for at least 15 minutes, the reactions are spun for 15 minutes in a microfuge. The pellets are washed with 200 μl 70% ethanol, dried, and resuspended in 4 μl of gel loading buffer as described in the Applied Biosystems 373A Sequencer manual. For all sequencing protocols, the gel conditions recommended by Applied Biosystems for the 373A automated sequencer are followed.

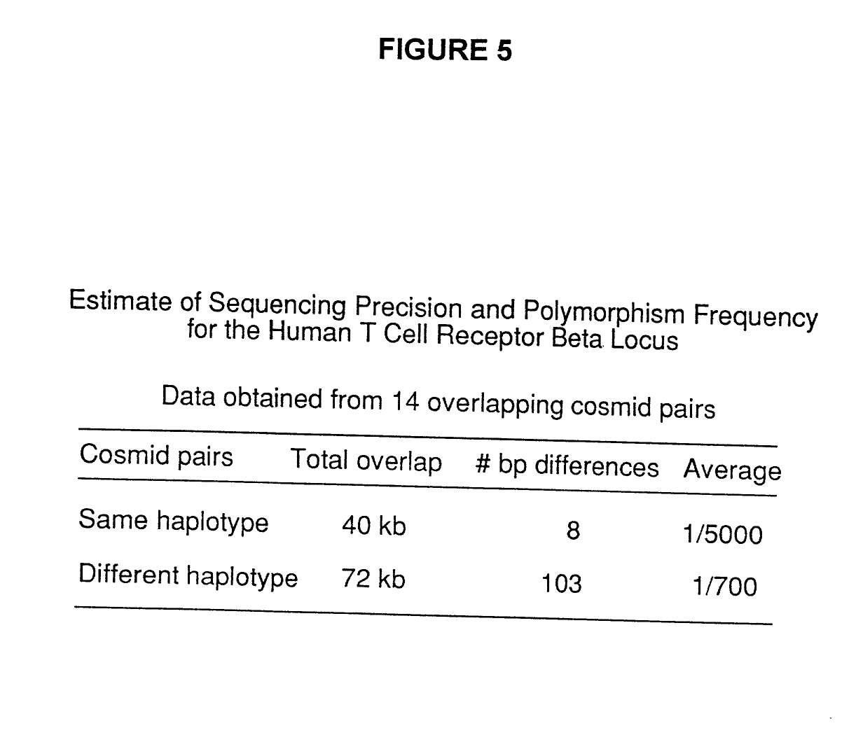

With this method, the DNA sequence is accurate to 1/5000 bases (FIG. 5).

### Example 6

### Analysis of Polymorphic Repeat Sequences

Polymorphic lengths of simple repeat sequences are detected by PCR analysis of genomic DNA using primers located in unique sequences flanking the repeat. Differences in the length of the repeat sequences are readily detected following gel electrophoresis of the amplified products.

Cosmid C215 has a repeat sequence, TAAA, which is repeated 8 times. Primer sequences are chosen in the unique sequences flanking this repeat region. The 5′ primer has the sequence 5′GCCTGGGAGACAGAGCAAGA-3′ and is located 33 bases upstream of the repeat sequence. The 3′ primer has the sequence 5′-CACATAGCAGCTGCTTTACA-3′ and is complementary to a sequence located 31 bases downstream of the repeat sequence. The predicted length of the amplified product synthesized from cosmid C215, which has an 8-fold repeat, is 96 bp. Oligonucleotides to be used as primers are synthesized on an automated DNA synthesizer, such as ABI Model.

Genomic DNA is isolated from peripheral blood cells or other tissue samples. Briefly, cells are lysed in 50 mM Tris, pH 8.0, 10 mM EDTA, 0.5% Triton-X100, and 200 μg/mL proteinase K, and incubated at 50° C. for 30 minutes. Proteinase K is heat inactivated by incubation at 95° C. for 10 minutes. The amplification reaction mixture contains approximately 50-250 ng of genomic DNA in 50 mM KCl, 10 mM Tris, pH 8.3, 2.5mM MgCl₂, 200 μM each of dATP, dCTP, dGTP, and dTTP, 1 μM of each oligonucleotide primer, and 1 unit of Taq DNA polymerase. The reaction mixture is overlaid with mineral oil to prevent evaporation. PCR reactions are carried out in a DNA thermocycler for an initial period at 95° C. for 4 minutes followed by 35 cycles of 94° C. for 1 minute, 60° C. for 1 minute, and 72° C. for 2 minutes.

The amplified products are analyzed by gel electrophoresis in one of two systems, agarose gel electrophoresis or polyacrylamide gel electrophoresis. For an agarose gel, bromphenol blue gel loading dye is added to each reaction tube. The sample is then loaded on a 4% Nu-Sieve agarose gel containing ethidium bromide. Following electrophoresis, amplified products are visualized by UV excitation. Alternatively, samples are ethanol precipitated, resuspended in formamide containing bromphenol blue and xylene cylanol, and loaded on an 8% polyacrylamide (19:1) gel. This gel is stained with ethidium bromide, or autoradiographed if radioactive-labeled primers or nucleotides are included in the reaction mixture. Appropriate size markers, as well as an amplified sample of cosmid C215 are used to aid analysis.

### Example 7

### Amplification and Analysis of TCRβ Microsatellites

A. Marker Detection

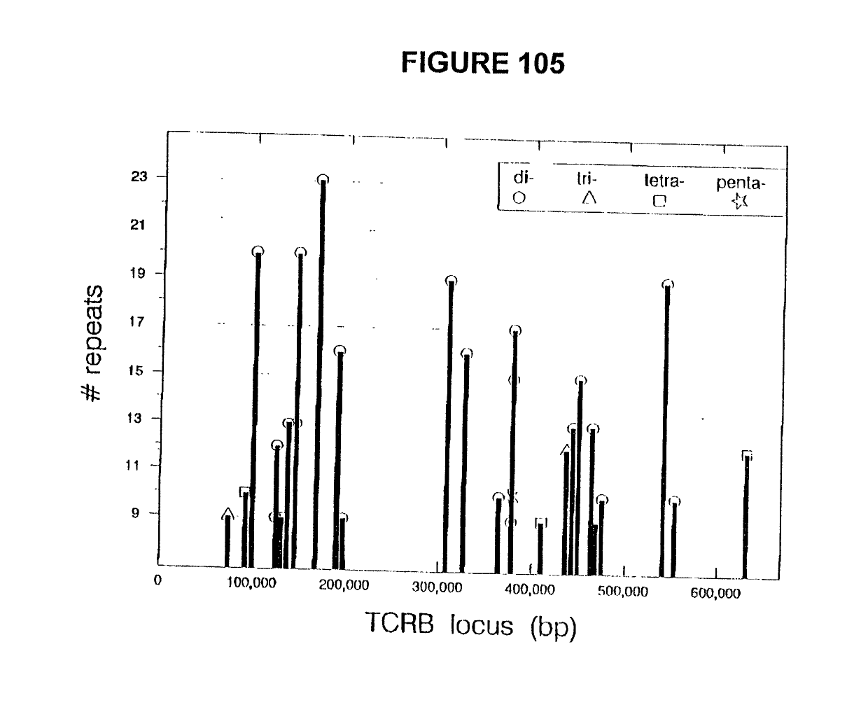

The 685 kb contig of human TCRβ DNA sequence (GenBank Accession No. L36092) is scanned by computer analysis for microsatellites involving di-, tri-, tetra-, or pentanucleotide repeats. Using a minimum length of n≧9, there were 21 dinucleotide repeats, two trinucleotide repeats, five tetranucleotide repeats, and one pentanucleotide repeat (FIG. 105). As expected, the microsatellites with the greatest number of repeat units were dinucleotide repeats. Based on all 21 dinucleotide repeats, eleven were of the core sequence AC, six were AT, and four were AG. None of the repeats occurred within the coding sequence of known genes in this region.

In order to examine each microsatellite for length polymorphism, fifteen unrelated Caucasian CEPH parents are used as the source of template DNAs for the PCR amplifications. One locus extending from basepair location 377472-377872 contained multiple dinucleotide repeats and a pentanucleotide repeat (n≧9) but was refractory to amplification attempts, likely because of its highly complex repetitive nature.

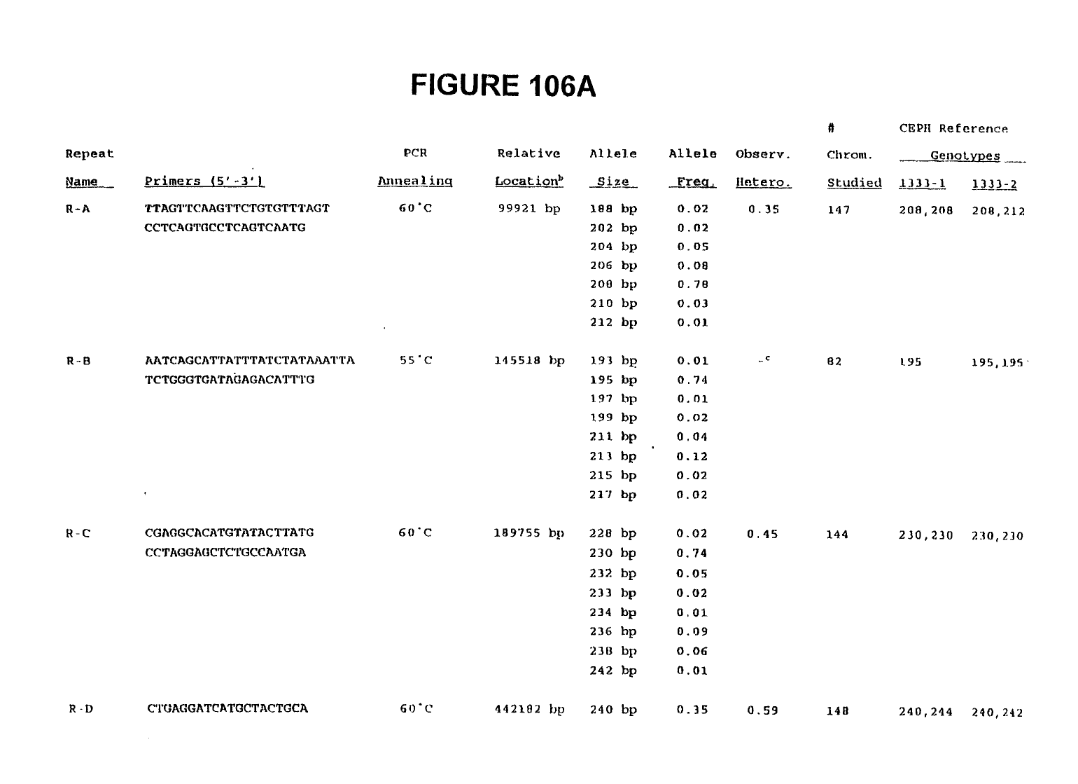

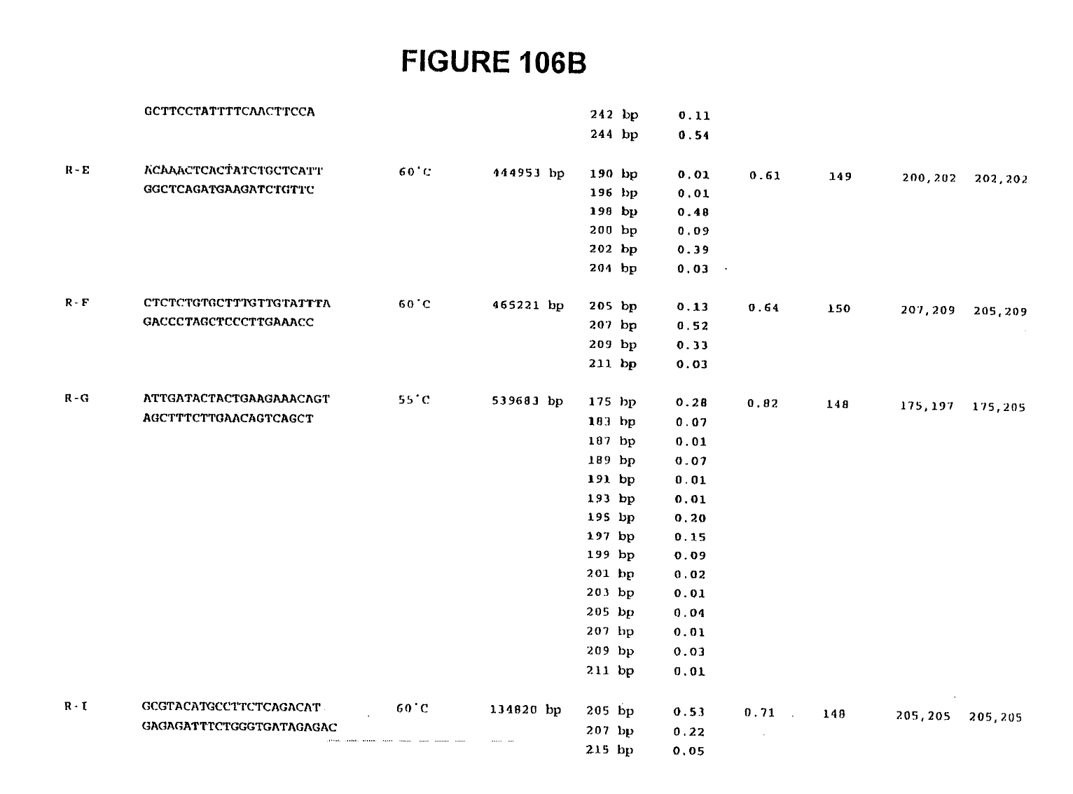

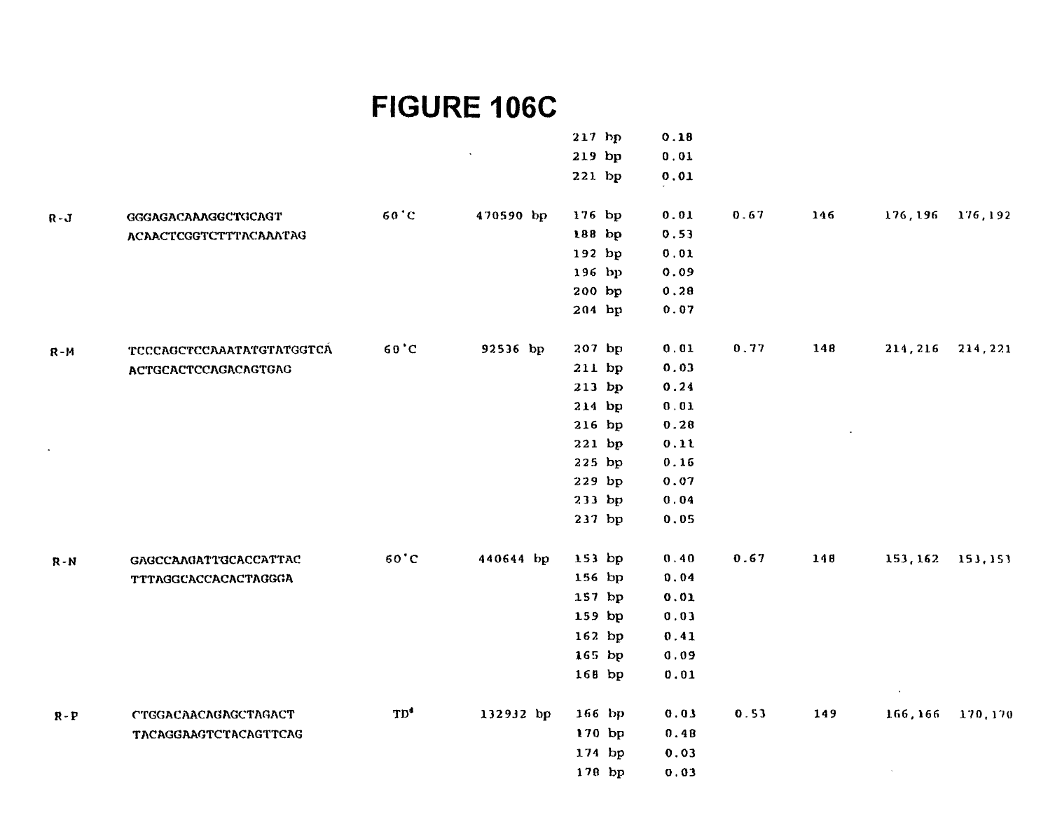

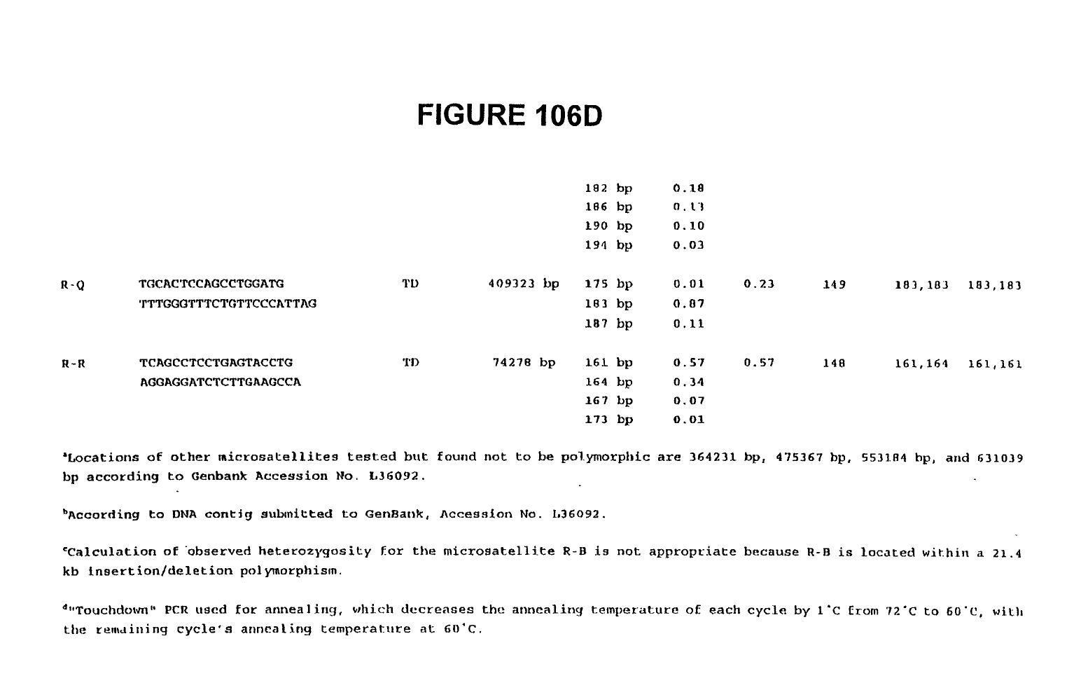

Fourteen novel and polymorphic TCRβ microsatellites (and four that were not polymorphic) are listed in FIGS. 106A-D (and footnote). To more thoroughly survey the extent of allelic polymorphism for each microsatellite, a total of 150 unrelated Caucasian CEPH chromosomes are genotyped for each polymorphism by electrophoresis, each microsatellite on a single gel. These microsatellite polymorphisms had an average of seven differently sized alleles, with a range from three to fifteen alleles. The observed frequency of heterozygosity ranged from 0.23 to 0.82 and appeared to be more related to the distribution of allele frequencies rather than to the number of alleles at a locus (e.g, repeats R-A versus R-D). Examination of the genotype frequencies showed all microsatellites to be in Hardy-Weinberg equilibrium.

B. Statistical Analysis

Genotypes are collected using the polymerase chain reaction from 72-75 Centre d'Etude du Polymorphisme (CEPH) family Caucasian parental DNAs. From these genotypes, allele and observed heterozygosity frequencies are calculated. In particular, the computer program ASSOC (Ott, Genet Epidemiol 2:79-84, 1985) is used to calculate the deviation of the multiallelic microsatellite genotype frequencies from Hardy-Weinberg expectations. Two-locus linkage disequilibrium is assessed by using haplotypes for which phase could be determined using the genotypes collected (i.e., from individuals who were homozygous at both loci, or heterozygous at not more than one of the loci). Estimations of the “overall” linkage disequilibrium between the microsatellites and certain bi-allelic polymorphisms are calculated using a chi-square statistic to compare the observed haplotype frequencies with the haplotype frequencies expected based on random association at the two loci, with (r-1)(c-1) degrees of freedom (Weir, Genetic Data Analysis, Sinauer Associates, Sunderland, Massachusetts, pg. 93-94, 1990). Classes with expected values of <5 were combined to avoid inflated statistical differences. To separately test the level of linkage disequilibrium for individual microsatellite alleles (“allelic LD”), each allele was separately compared to all other microsatellite alleles combined using a 2×2 chi-square analysis. To compensate for the large number of pairwise analyses performed, the alpha level of statistical significance was lowered to p<0.0001.

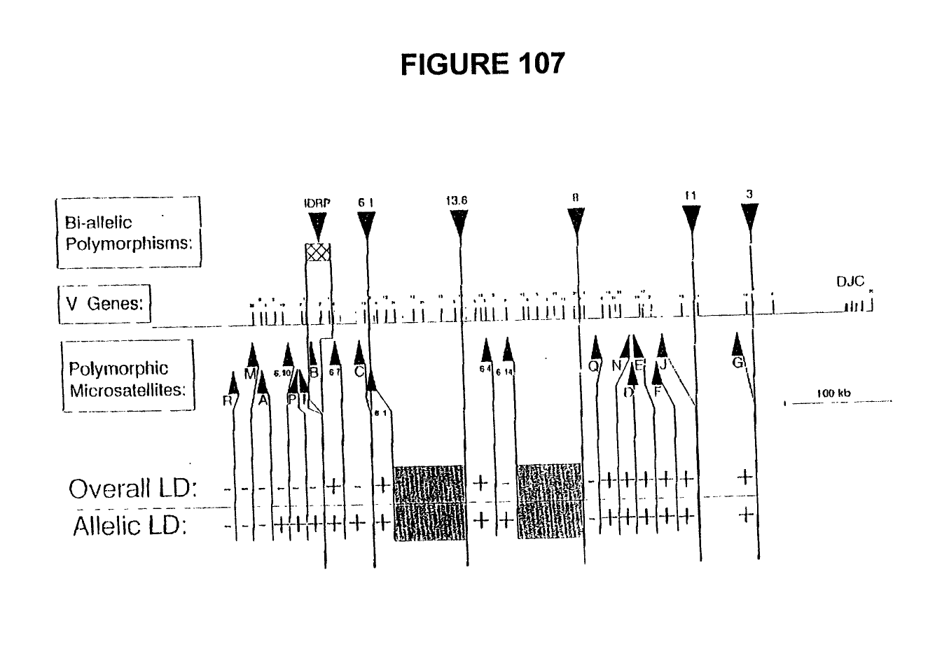

Each TCRβ microsatellite is assayed for linkage disequilibrium with nearly bi-allelic polymorphisms using the genotypes collected as an approach to assessing the usefulness of the microsatellites for showing an association with a disease susceptibility allele (FIG. 107). Six bi-allelic polymorphisms are used to span and divide up the TCRβ gene complex. Each microsatellite is statistically tested for an overall distribution difference as they existed in combination with the two alleles at an adjacent bi-allelic polymorphism. Nine of the eighteen microsatellites demonstrated significant overall linkage disequilibrium with a bi-allelic polymorphism. All but one microsatellite (likely due to its low heterozygosity, and thus power) between the BV8 and BV11 RFLPs showed very strong (p<10⁻⁶) disequilibrium with one or both of these RFLPs. At the other extreme, none of the microsatellites 5-prime (left) of the IDRP showed any detectable overall disequilibrium with the IDRP (FIG. 107).

For a more specific analysis, each microsatellite allele is individually tested for a statistically significant difference in distribution in conjunction with the two alleles at an adjacent bi-allelic polymorphism. This analysis not only reveals which haplotypes make the greatest contribution to an overall different distribution, but can also detect evidence of disequilibrium that may be diluted by analyzing all haplotypes simultaneously. In this way, five additional TCRβ microsatellites showed evidence of significant disequilibrium with these bi-allelic polymorphisms.

By separately testing individual microsatellite alleles, all but four (79%) showed non-random association with at least one other marker. In addition, the R-M, R-R, and R-A markers are not in detectable linkage disequilibrium with each other, or other microsatellite markers nearby. (These three SSRs, at the 5-most end of the TCRβ map, may be isolated from each other and the other markers tested by recombination hot spots.) Alternatively, the SSR R-M may have a mutation rate high enough that the haplotype relationships are no longer detectable, possibly also suggested by R-M's high heterozygosity (Weber, Genomics 7:524-530, 1990; Bowcock et al., Genomics 15:376-386, 1993). The R-A and R-Q markers have the lowest heterozygosity of all the polymorphic microsatellites tested and thus may not have the statistical power to detect linkage disequilibrium.

Based on the remaining gaps not covered by detectable linkage disequilibrium using the above-noted set of polymorphic SSRs, complete saturation requires additional markers. Thus, additional SSRs sequenced but of smaller size than those yet examined (e.g., n=8) may likewise be utilized in order to ensure complete saturation of the TCRβ complex.

In summary, 685 kb of contiguous DNA sequence may be utilized to investigate the occurrence of potentially polymorphic microsatellites as derived from such long stretches of sequenced genomic DNA. Of the 29 SSRs (n≧9) discussed above, a majority of these were polymorphic, spanning a majority of the TCRβ sequence. The use of even a few of these polymorphisms is sufficient for family segregation studies. As an additional application, the majority of these markers appear capable of detecting linkage disequilibrium with other nearby markers, including possible disease susceptibility markers. Given the high marker density utilized in this study, as well as the primer pairs provided elsewhere in this application (e.g., FIG. 104), or which may be made given the disclosure provided herein, it is possible to span the entire TCRβ complex with a panel of markers that are in linkage disequilibrium with the nearest flanking markers.

From the foregoing, it will be appreciated that, although specific embodiments of the invention have been described herein for purposes of illustration, various modifications may be made without deviating from the spirit and scope of the invention.

1. A kit comprising a panel of nucleic acid primers capable of specifically priming and allowing amplification of each and every Vβ gene.

2. A pair of nucleic acid primers capable of specifically priming and allowing amplification of Vβ genomic DNA.

3. A kit comprising a panel of nucleic acid primers capable of specifically priming and allowing amplification of each and every VβRNA or cDNA.

4. A pair of nucleic acid primers capable of specifically priming and allowing amplification of any one of the polymorphic sequences set forth in FIGS. 89 to 100.

5. A kit comprising a panel of antibodies which are capable of specifically binding to each and every unique β chain of a T cell receptor.

6. A method for determining a correlation between a disease or disease susceptibility and a selected polymorphism, comprising:
(a) obtaining biological samples containing nucleated cells from a population, said population having individuals with a selected disease or disease susceptibility and individuals without said disease or disease susceptibility or individuals who are in remission from said selected disease;
(b) extracting nucleic acids from said cells;
(c) contacting said extracted nucleic acids with primers capable of specifically priming and allowing amplification of a selected polymorphism;
(d) amplifying said selected polymorphism; and
(e) detecting the presence of said polymorphism, and thereby determining a correlation between said disease or disease susceptibility and said selected polymorphism.

7. A method for determining a correlation between a disease and a selected polymorphism. comprising:
(a) obtaining biological samples containing nucleated cells from a population, said population having individuals with a selected disease and individuals without said disease;
(b) extracting ribonucleic acids from said cells;
(c) reverse transcribing cDNA from said ribonucleic acids;
(d) contacting said cDNA with primers capable of specifically priming and allowing amplification of a selected polymorphism;
(e) amplifying said selected polymorphism; and
(f) detecting the presence of said polymorphism, and thereby determining a correlation between said disease and said selected polymorphism.

8. A method for determining a correlation between a disease and a selected polymorphism, comprising:
(a) obtaining biological samples containing nucleated cells from a population, said population having individuals with a selected disease and individuals without said disease;
(b) extracting nucleic acids from said cells; and
(c) detecting the presence of said polymorphism, and thereby determining a correlation between said disease or disease susceptibility and said selected polymorphism.

9. The method according to any one of claims 6 to 8 wherein said polymorphism is a restriction fragment length polymorphism.

10. The method according to any one of claims 6 to 8 wherein said polymorphism is a length difference of a simple repeat sequence.

11. The method according to any one of claims 6 to 8 wherein said polymorphism is a specific nucleotide substitution, deletion or insertion.

12. A method according to any one of claims 6 to 8 wherein said disease or disease susceptibility is selected from the group consisting of Addison's disease, atrophic gastritis, autoimmune hemolytic anemia, autoimmune neutropenia, bullous pemphigoid, Crohn's disease, coeliac disease, demyelinating neuropathies, dermatomyositis, Goodpasture's syndrome, Graves' disease, hemolytic anemia, idiopathic thrombocytopenia purpura, inflammatory bowel disease, insulin-dependent diabetes mellitus, juvenile diabetes, multiple sclerosis. myasthenia gravis, myocarditis, myositis, myxedema, pemphigus vulgaris, pernicious anaemia, primary glomerulonephritis, rheumatoid arthritis, scleritis, scleroderma, Sjogren's syndrome, systemic lupus erythematosus, and type I diabetes.

13. A method for determining a correlation between a disease resistance or disease susceptibility and a genetic marker, comprising:
(a) obtaining biological samples containing nucleated cells from a population, said population having individuals with a selected disease resistance or disease susceptibility and individuals without said disease resistance or disease susceptibility;
(b) extracting nucleic acids from said cells;
(c) contacting said extracted nucleic acids with primers which are capable of specifically priming and allowing amplification of a series of selected genetic markers in the T cell receptor β gene region, said markers being selected such that they are in linkage disequilibrium with each other;
(d) amplifying said genetic markers; and
(e) determining the length of said amplified material, and thereby determining the correlation between a disease resistance or disease susceptibility and a genetic marker.

14. The method of claim 13 wherein said series of genetic markers are at least 5 to 35 kb apart.

15. The method of claim 13 wherein said series of genetic markers are at least 10 to 20 kb apart.

16. A kit comprising a battery of primer pairs capable of specifically priming and allowing amplification of a series of selected markers in the T cell receptor β gene region, said markers being selected such that they are in linkage disequilibrium with each other.

17. The kit of claim 16 wherein said series of genetic markers are at least 5 to 35 kb apart.

18. The kit of claim 16 wherein said series of genetic markers are at least 10 to 20 kb apart.
